(19) United States
(12) Patent Application Publication
(10) Pub. No.: US 2019/0062305 A1
(43) Pub. Date: Feb. 28, 2019

(54) PYRIDYL OR PYRAZINYL COMPOUNDS CARRYING A METHYL-BOUND ALPHA-AMINO ACID AMIDE GROUP

(71) Applicant: ABBVIE DEUTSCHLAND GMBH & CO. KG, Wiesbaden (DE)

(72) Inventors: Gisela BACKFISCH, Ludwigshafen (DE); Margaretha BAKKER, Ludwigshafen (DE); Günter BLAICH, Ludwigshafen (DE); Wilfried BRAJE, Ludwigshafen (DE); Karla DRESCHER, Ludwigshafen (DE); Thomas ERHARD, Ludwigshafen (DE); Andreas HAUPT, Ludwigshafen (DE); Carolin HOFT, Ludwigshafen (DE); Andreas KLING, Ludwigshafen (DE); Viktor LAKICS, Ludwigshafen (DE); Helmut MACK, Ludwigshafen (DE); Frank OELLIEN, Ludwigshafen (DE); Raimund PETER, Ludwigshafen (DE); Frauke POHLKI, Ludwigshafen (DE); Ana Lucia RELO, Ludwigshafen (DE)

(21) Appl. No.: 15/761,735

(22) PCT Filed: Sep. 21, 2016
(86) PCT No.: PCT/EP2016/072387

§ 371 Date: Mar. 20, 2018

Related U.S. Application Data
(60) Provisional application No. 62/221,350, filed on Sep. 21, 2015.

(51) Int. Cl. C07D 401/12; C07D 401/14

(52) U.S. Cl. CPC C07D 401/12; C07D 401/14

(57) ABSTRACT

The present invention relates to pyridyl or pyrazinyl compounds carrying a methyl-bound N-amide moiety derived from an α-amino acid of formula I where the variables are as defined in the claims and the description. The invention further relates to a pharmaceutical composition containing such compounds, to their use as modulators, especially agonists or partial agonists, of the 5-HT_{2C} receptor, and to their use for preparing a medicament for the prevention or treatment of conditions and disorders which respond to the modulation of the 5-HT_{2C} receptor.

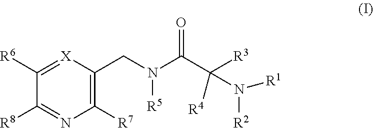

### FIELD OF THE INVENTION

The present invention relates to pyridyl or pyrazinyl compounds carrying a methyl-bound N-amide moiety derived from an α-amino acid, to a pharmaceutical composition containing such compounds, to their use as modulators, especially agonists or partial agonists, of the 5-HT_{2C} receptor, their use for preparing a medicament for the prevention or treatment of conditions and disorders which respond to the modulation of 5-HT_{2C} receptor, to a method for preventing or treating conditions and disorders which respond to the modulation of the 5-HT_{2C} receptor, and processes for preparing such compounds and compositions.

### BACKGROUND OF THE INVENTION

Diseases, disorders and conditions where 5-HT_{2C} modulation is desired are for example depression, anxiety, schizophrenia, bipolar disorder, obsessive compulsive disorder, migraine, pain, epilepsy, substance abuse, eating disorders, obesity, diabetes, erectile dysfunction and others.

Serotonin (5-hydroxytryptamine, 5-HT), a monoamine neurotransmitter and local hormone, is formed by the hydroxylation and decarboxylation of tryptophan. The greatest concentration is found in the enterochromaffin cells of the gastrointestinal tract, the remainder being predominantly present in platelets and in the Central Nervous System (CNS). 5-HT is implicated in a vast array of physiological and pathophysiological pathways. In the periphery, it contracts a number of smooth muscles and induces endothelium-dependent vasodilation. In the CNS, it is believed to be involved in a wide range of functions, including the control of appetite, mood, anxiety, hallucinations, sleep, vomiting and pain perception.

Neurons that secrete 5-HT are termed serotonergic. The function of 5-HT is exerted upon its interaction with specific (serotonergic) neurons. Seven types of 5-HT receptors have been identified: 5-HT₁ (with subtypes 5-HT_{1A}, 5-HT_{1B}, 5-HT_{1D}, 5-HT_{1E} and 5-HT_{1F}), 5-HT₂ (with subtypes 5-HT_{2A}, 5-HT_{2B} and 5-HT_{2C}), 5-HT₃, 5-HT₄, 5-HT₅ (with subtypes 5-HT_{5A} and 5-HT_{5B}), 5-HT₆ and 5-HT₇. Most of these receptors are coupled to G-proteins that affect the activities of adenylate cyclase or phospholipase Cγ.

Alterations in the activity of multiple neurotransmitter receptor systems (dopamine, serotonin, glutamate, GABA, acetylcholine) have been implicated in the manifestation of the symptoms of schizophrenia. The most widely accepted “Dopamine Hypothesis of Schizophrenia” in its simplest form states that the positive symptoms of this pathology relate to a functional hyperactivity of the mesolimbic dopaminergic system, while the negative and cognitive aspects can be traced to a functional hypoactivity of the mesocortical dopaminergic projections. Atypical antipsychotics block the mesolimbic dopaminergic neurotransmission, thereby controlling positive symptoms, with little or no effect on the nigrostriatal system, leading to less induction of extrapyramidal side effects (EPS).

Primary negative and cognitive symptoms of schizophrenia reflect a dysfunction of the frontal cortex (“hypofrontality”), which is thought to be induced by a decreased tone in the mesocortical dopaminergic projection field [Davis K L, Kahn R S, Ko G and Davidson M (1991). Dopamine in schizophrenia: a review and re-conceptualization. Am J Psychiatry 148: 1474-86. Weinberger D R and Berman K F (1996). Prefrontal function in schizophrenia: confounds and controversies. Philos Trans R Soc Lond B Biol Sci 351: 1495-503]. Agents that selectively enhance dopamine levels in the cortex have the potential to address the negative symptoms of this disorder. Atypical antipsychotics lack robust efficacy against negative and cognitive components of the schizophrenic syndrome.

The schizophrenic symptomatology is further complicated by the occurrence of drug-induced so-called secondary negative symptoms and cognitive impairment, which are difficult to distinguish from primary negative and cognitive symptoms [Remington G and Kapur S (2000). Atypical antipsychotics: are some more atypical than others?Psychopharmacol 148: 3-15]. The occurrence of secondary negative symptoms not only limits therapeutic efficacy but also, together with these side effects, negatively affects patient compliance.

It may thus be hypothesized that a novel mechanistic approach that blocks dopaminergic neurotransmission in the limbic system but does not affect the striatal and pituitary projection fields, and stimulates frontocortical projection fields, would provide an efficacious treatment for all parts of the schizophrenic pathology, including its positive, negative and cognitive symptoms. Moreover, a selective compound that is substantially free of the ancillary pharmacology that characterizes current agents would be expected to avoid a variety of off-target side effects that plague current treatments such as extrapyramidal side effects (EPS) and weight gain.

The 5-HT_{2C} receptor, previously named 5-HT1C, is a G-protein-coupled receptor, which couples to multiple cellular effector systems including the phospholipase C, A and D pathways. It is found primarily in the brain and its distribution is particularly high in the plexus choroideus, where it is assumed to control cerebrospinal fluid production [Kaufman M J, Hirata F (1996) Cyclic GMP inhibits phosphoinositide turnover in choroid plexus: evidence for interactions between second messengers concurrently triggered by 5-HT_{2C} receptors. Neurosci Lett 206:153-156]. Very high levels were also found in the retrosplenial, piriform and entorhinal cortex, anterior olfactory nucleus, lateral septal nucleus, subthalamic nucleus, amygdala, subiculum and ventral part of CA3, lateral habenula, substantia nigra pars compacta, several brainstem nuclei and the whole grey matter of the spinal cord [Pompeiano M, Palacios J M, Mengod G (1994). Distribution of the serotonin 5-HT2 receptor family mRNAs: comparison between 5-HT_{2A} and 5-HT_{2C} receptors. Brain Res Mol Brain Res 23:163-178]. A comparison of the distribution of 5-HT_{2C} mRNA with that of 5-HT_{2C} protein in monkey and human brains has revealed both pre- and postsynaptic localization [Lopez-Gimenez J F, Mengod G, Palacios J M, Vilaro M T (2001) Regional distribution and cellular localization of 5-HT_{2C} receptor mRNA in monkey brain: comparison with [³H]mesulergine binding sites and choline acetyltransferase mRNA. Synapse 42:12-26].

It is anticipated that modulation of the 5-HT_{2C} receptor will improve disorders such as depression, anxiety, schizophrenia, cognitive deficits of schizophrenia, obsessive compulsive disorder, bipolar disorder, neuropsychiatric symptoms in Parkinson' disease, in Alzheimer's disease or Lewy Body dementia, migraine, epilepsy, substance abuse, eating disorders, obesity, diabetes, sexual dysfunction/erectile dysfunction, sleep disorders, psoriasis, Parkinson's disease, pain conditions and disorders, and spinal cord injury, smoking cessation, ocular hypertension and Alzheimer's disease. Modulators of the 5-HT_{2C} receptor are also shown to be useful in the modulation of bladder function, including the prevention or treatment of urinary incontinence.

Compounds with a structure similar to the compounds of the present invention have been described in WO 2012/053186, WO 2014/177982, WO 2014/151142, WO 2014/062838, WO 2014/049488, WO 2013/120104, WO 2012/142513, WO 2012/142504, WO 2010/137351, WO 2006/055184 and WO 2004/074259.

K. K.-C. Liu et al. describe in Bioorganic & Medicinal Chemistry Letters 2010, 20, 2365-2369 substituted N-benzyl proline amides to be highly selective 5-HT_{2c} agonists and useful for the treatment of obesity.

There is an ongoing need for providing compounds having high affinity and preferably also selectivity for the 5-HT_{2C} receptor. In particular the compounds should have low affinity to adrenergic receptors, such as the α₁-adrenergic receptor, histamine receptors, such as the H₁-receptor, and dopaminergic receptors, such as the D₂-receptor, in order to avoid or reduce side effects associated with modulation of these receptors, such as postural hypotension, reflex tachycardia, potentiation of the antihypertensive effect of prazosin, terazosin, doxazosin and labetalol or dizziness associated with the blockade of the α₁-adrenergic receptor, weight gain, sedation, drowsiness or potentiation of central depressant drugs associated with the blockade of the H₁-receptor, or extrapyramidal movement disorder, such as dystonia, parkinsonism, akathisia, tardive dyskinesia or rabbit syndrome, or endocrine effects, such as prolactin elevation (galactorrhea, gynecomastia, mentstrual changes, sexual dysfunction in males), associated with the blockade of the D₂-receptor, and even more important no induction of weight gain in combination with severe metabolic dysfunction found for marketed antipsychotic drugs.

It is moreover desirable that the compounds have low affinity or alternatively an antagonistic effect to/on other serotonergic receptors, especially the 5-HT_{2A} and/or 5-HT_{2B} receptors, in order to avoid or reduce side effects associated with modulation of these receptors, such as changes (thickening) of the heart tissue associated with agonism at the 5-HT_{2B} receptor, and psychotomimetic effect induced by agonism at the 5-HT_{2A} receptor. Ideally they should show an agonistic action on the 5-HT_{2C} receptor, an antagonistic action on the 5-HT_{2A} receptor or alternatively no affinity to the 5-HT_{2A} receptor and no affinity to the 5-HT_{2B} receptor or alternatively an antagonistic action on the 5-HT_{2B} receptor. Even more ideally the compounds should display an agonistic action on the 5-HT_{2C} receptor in combination with an antagonistic action on the 5-HT_{2A} receptor and no affinity to the 5-HT_{2B} receptor.

Besides the affinity and selectivity for the 5-HT_{2C} receptor, further properties may be advantageous for the treatment and/or prophylaxis of 5-HT_{2C}-related disorders, such as, for example:

1.) the metabolic stability, for example determined from the half-lives, measured in vitro, in liver microsomes from various species (e.g. rat or human);

2.) no or only low inhibition of cytochrome P450 (CYP) enzymes: cytochrome P450 (CYP) is the name for a superfamily of heme proteins having enzymatic activity (oxidase). They are also particularly important for the degradation (metabolism) of foreign substances such as drugs or xenobiotics in mammalian organisms. The principal representatives of the types and subtypes of CYP in the human body are: CYP 1A2, CYP 2C9, CYP 2D6 and CYP 3A4. If CYP 3A4 inhibitors (e.g. grapefruit juice, cimetidine, erythromycin) are used at the same time as medicinal substances which are degraded by this enzyme system and thus compete for the same binding site on the enzyme, the degradation thereof may be slowed down and thus effects and side effects of the administered medicinal substance may be undesirably enhanced;

3.) a suitable solubility in water (in mg/mL);

4.) suitable pharmacokinetics (time course of the concentration of the compound of the invention in plasma or in tissue, for example brain). The pharmacokinetics can be described by the following parameters: half-life (in h), volume of distribution (in l·kg-1), plasma clearance (in l·h-1·kg-1), AUC (area under the curve, area under the concentration-time curve, in ng·h·l-1), oral bioavailability (the dose-normalized ratio of AUC after oral administration and AUC after intravenous administration), the so-called brain-plasma ratio (the ratio of AUC in brain tissue and AUC in plasma);

5.) no or only low blockade of the hERG channel: compounds which block the hERG channel may cause a prolongation of the QT interval and thus lead to serious disturbances of cardiac rhythm (for example so-called “torsade de pointes”). The potential of compounds to block the hERG channel can be determined by means of the displacement assay with radiolabelled dofetilide which is described in the literature (G. J. Diaz et al., Journal of Pharmacological and Toxicological Methods, 50 (2004), 187 199). A smaller IC50 in this dofetilide assay means a greater probability of potent hERG blockade. In addition, the blockade of the hERG channel can be measured by electrophysiological experiments on cells which have been transfected with the hERG channel, by so-called whole-cell patch clamping (G. J. Diaz et al., Journal of Pharmacological and Toxicological Methods, 50 (2004), 187-199).

It was an object of the present invention to provide compounds for the treatment or prophylaxis of various 5-HT_{2C}-related diseases. The compounds were intended to have a high affinity to the 5-HT_{2C} receptor and be potent and efficacious 5-HT_{2C} agonists. In addition, the compounds of the invention were intended to have one or more of the aforementioned advantages, namely low affinity on other serotonergic receptors, and especially the lack of potent agonistic effect (antagonism preferred) on the 5-HT_{2A} and/or 5-HT_{2B} receptors, and additionally one or more of those advantages mentioned under 1.) to 5.), and especially under 1.) (metabolic stability) and 4.) (oral bioavailability in vivo).

The present invention provides compounds which have an affinity for the 5-HT_{2C} receptor, thus allowing the treatment of disorders related to or affected by the 5-HT_{2C} receptor.

### SUMMARY OF THE INVENTION

The present invention relates to pyridyl or pyrazinyl compounds carrying a methyl-bound N-amide moiety derived from an α-amino acid, to a pharmaceutical composition containing such compounds, to their use as modulators, especially agonists or partial agonists, of the 5-HT_{2C} receptor, their use for preparing a medicament for the prevention or treatment of conditions and disorders which respond to the modulation of 5-HT_{2C} receptor, to a method for preventing or treating conditions and disorders which respond to the modulation of 5-HT_{2C} receptor, and processes for preparing such compounds and compositions.

In one aspect, the present invention relates to compounds of the formula (I):

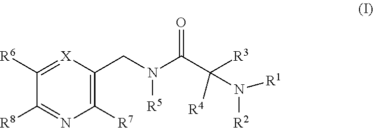

wherein
X is CH or N;
R¹ is selected from the group consisting of hydrogen and C₁-C₄-alkyl;
R² is selected from the group consisting of hydrogen and C₁-C₄-alkyl; or
R¹ and R², together with the nitrogen atom they are bound to, form an N-bound 4-, 5- or 6-membered saturated heterocyclic ring which may carry 1 or 2 substituents selected from the group consisting of halogen, C₁-C₄-alkyl and fluorinated C₁-C₄-alkyl;
R³ is selected from the group consisting of hydrogen and C₁-C₄-alkyl; or
R² and R³, together with the atoms they are bound to, form a 4-, 5- or 6-membered saturated or partially unsaturated heteromonocyclic ring which may carry 1 or 2 substituents selected from the group consisting of halogen, C₁-C₄-alkyl and fluorinated C₁-C₄-alkyl, or form a 4-, 5-, 6-, 7-, 8-, 9 or 10-membered saturated or partially unsaturated heterobicyclic ring which may carry 1 or 2 substituents selected from the group consisting of halogen, C₁-C₄-alkyl and fluorinated C₁-C₄-alkyl;
R⁴ is selected from the group consisting of hydrogen, C₁-C₄-alkyl and C₃-C₆-cycloalkyl;
R⁵ is selected from the group consisting of hydrogen and C₁-C₄-alkyl;
R⁶ is hydrogen or halogen;
R⁷ is selected from the group consisting of C₁-C₄-alkoxy, fluorinated C₁-C₄-alkoxy, hydroxyl, C₁-C₄-alkyl and fluorinated C₁-C₄-alkyl;
R⁸ is selected from the group consisting of —OR^{8a}, —NR^{8b}R^{8c} and —R^{8d};
R^{8a} is selected from the group consisting of C₁-C₈-alkyl, C₁-C₄-alkyl which carries a radical R^{9a}; fluorinated C₁-C₈-alkyl, C₃-C₆-cycloalkyl which may carry one or more substituents R¹⁰; C₅-C₁₀-bicycloalkyl which may carry one or more substituents R¹⁰; and phenyl which may carry one or more substituents R¹²;
R^{8b} is selected from the group consisting of hydrogen, C₁-C₄-alkyl and C₂-C₄-alkenyl;
R^{8c} is selected from the group consisting of C₁-C₄-alkyl which carries a radical R^{9b}; C₃-C₆-cycloalkyl which may carry one or more substituents R¹¹; and phenyl which may carry one or more substituents R¹²; or
R^{8b} and R^{8c}, together with the nitrogen atom they are bound to, form a 4-, 5-, 6- or 7-membered saturated (of course N-bound) heteromonocyclic ring which may additionally contain a further heteroatom selected from the group consisting of N, O and S as ring member, where the heteromonocyclic ring may carry one or more substituents R¹⁴; or form a 6-, 7-, 8-, 9- or 10-membered saturated or unsaturated heterobicyclic ring which may carry one or more substituents R¹⁴;
R^{8d} is a C-bound radical selected from the group consisting of C₁-C₄-alkyl which carries a radical R^{9c}; C₁-C₄-haloalkyl which carries a radical R^{9c}; C₃-C₆-cycloalkyl which may carry one or more substituents R¹¹; C₃-C₆-cycloalkenyl which may carry one or more substituents R¹¹; phenyl which may carry one or more substituents R¹²; and a C-bound 5- or 6-membered saturated heteromonocyclic ring containing a heteroatom selected from the group consisting of N, O and S as ring member, where the heteromonocyclic ring may carry one or more substituents R¹²;
R^{9a} is selected from the group consisting of C₃-C₆-cycloalkyl which may carry one or more substituents R¹¹; C₅-C₁₀-bicycloalkyl which may carry one or more substituents R¹¹; adamantyl which may carry one or more substituents R¹¹; and phenyl which may carry one or more substituents R¹²;
R^{9b} is selected from the group consisting of C₃-C₆-cycloalkyl which may carry one or more substituents R¹¹; and phenyl which may carry one or more substituents R¹²;
R^{9c} is selected from the group consisting of C₁-C₄-alkoxy, C₁-C₄-haloalkoxy, C₃-C₆-cycloalkyl which may carry one or more substituents R¹¹; phenyl which may carry one or more substituents R¹²; and a 5- or 6-membered saturated heteromonocyclic ring containing a heteroatom selected from the group consisting of N, O and S as ring member, where the heteromonocyclic ring may carry one or more substituents R¹²;
each R¹⁰ is independently selected from the group consisting of halogen, hydroxyl, C₁-C₄-alkyl, C₁-C₄-haloalkyl, C₁-C₄-alkoxy, C₁-C₄-haloalkoxy and phenyl which may carry 1, 2, 3, 4 or 5 substituents R¹³; or
   two substituents R¹⁰ bound on the same carbon atom of the cycloalkyl or bicycloalkyl ring, together with the carbon atom they are bound to, form a 3-, 4-, 5- or 6-membered saturated (spiro-bound) carbocyclic ring, where the carbocyclic ring may carry one or more substituents selected from the group consisting of halogen, C₁-C₄-alkyl and C₁-C₄-haloalkyl;
   where two substituents R¹⁰ bound on the same carbon atom of the cycloalkyl or bicycloalkyl ring R^{8a} are not both C₁-C₄-alkyl;
each R¹¹ is independently selected from the group consisting of halogen, C₁-C₄-alkyl, C₁-C₄-haloalkyl, C₁-C₄-alkoxy and C₁-C₄-haloalkoxy;
R¹² and R¹³, independently of each other and independently of each occurrence, are selected from the group consisting of halogen, CN, C₁-C₄-alkyl, C₁-C₄-haloalkyl, C₁-C₄-alkoxy and C₁-C₄-haloalkoxy;
each R¹⁴ is independently selected from the group consisting of halogen, C₁-C₄-alkyl, C₁-C₄-haloalkyl, C₃-C₆-cycloalkyl which may carry one or more substituents selected from the group consisting of halogen and C₁-C₄-haloalkyl; phenyl which may carry one or more substituents R¹²; phenoxy which may carry one or more substituents R¹²; and benzyl which may carry one or more substituents R¹²;
   and the N-oxides, tautomeric forms, stereoisomers and pharmaceutically acceptable salts thereof, and the compound of the general formula I, wherein at least one of the atoms has been replaced by its stable, non-radioactive isotope.

The N-bound heterocyclic ring formed by R¹, R² and the nitrogen atom they are bound to does not contain any further heteroatom as ring member; i.e. the remaining ring members (apart from the mandatory nitrogen ring atom) are carbon atoms.

The same applies to the heteromono- or heterobicyclic ring formed by R², R³ and the atoms they are bound to; i.e. apart from the mandatory nitrogen ring atom all other ring members are carbon atoms.

In case that R² and R³, together with the atoms they are bound to, form a partially unsaturated heteromono- or heterobicyclic ring and the nitrogen ring atom is part of a double bond, R¹ is of course absent. In case that the carbon ring atom carrying R³ is part of a double bond, R⁴ is of course absent.

In case that R² and R³, together with the atoms they are bound to, form a heterobicyclic ring and the ring nitrogen atom is a fusion point of the bicyclic ring, R¹ is of course absent. Analogously, if the carbon atom carrying R³ (i.e. that carbon atom which forms the attachment point of the ring to the remainder of the molecule, i.e. to the C(O) group), is a fusion point of the bicyclic ring, R⁴ is of course absent.

In the heterocyclic ring formed by R¹, R² and the nitrogen atom they are bound to as well as in the heteromono- or heterobicyclic ring formed by R², R³ and the atoms they are bound to, the substituents, if present, may be bound to a carbon or to the nitrogen ring atom. In case that the nitrogen ring atom carries a substituent, this is not halogen.

In another aspect, the invention relates to a pharmaceutical composition comprising a therapeutically effective amount of at least one compound of formula I or an N-oxide, a tautomeric form, a stereoisomer or a pharmaceutically acceptable salt thereof, in combination with at least one pharmaceutically acceptable carrier and/or auxiliary substance.

In yet another aspect, the invention relates to a compound of formula I or an N-oxide, a tautomeric form, a stereoisomer or a pharmaceutically acceptable salt thereof for use as a medicament.

In yet another aspect, the invention relates to a compound of formula I or an N-oxide, a tautomeric form, a stereoisomer or a pharmaceutically acceptable salt thereof for use in the treatment of disorders which responds to the modulation of the 5-HT_{2C} receptor.

In yet another aspect, the invention relates to a compound of formula I or an N-oxide, a tautomeric form, a stereoisomer or a pharmaceutically acceptable salt thereof for use in the treatment of disorders selected from the group consisting of damage of the central nervous system, disorders of the central nervous system, eating disorders, ocular hypertension, cardiovascular disorders, gastrointestinal disorders and diabetes, and especially from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

In yet another aspect, the invention relates to the use of a compound of formula I or of an N-oxide, a tautomeric form, a stereoisomer or a pharmaceutically acceptable salt thereof for the manufacture of a medicament for the treatment of disorders which respond to the modulation of the 5-HT_{2C} receptor.

In yet another aspect, the invention relates to the use of a compound of formula I or of an N-oxide, a tautomeric form, a stereoisomer or a pharmaceutically acceptable salt thereof for the manufacture of a medicament for the treatment of disorders selected from the group consisting of damage of the central nervous system, disorders of the central nervous system, eating disorders, ocular hypertension, cardiovascular disorders, gastrointestinal disorders and diabetes, and especially from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

In yet another aspect, the invention relates to a method for treating disorders which respond to the modulation of the 5-HT_{2C} receptor, which method comprises administering to a subject in need thereof at least one compound of formula I or an N-oxide, a tautomeric form, a stereoisomer or a pharmaceutically acceptable salt thereof.

In yet another aspect, the invention relates to a method for treating disorders selected from the group consisting of damage of the central nervous system, disorders of the central nervous system, eating disorders, ocular hypertension, cardiovascular disorders, gastrointestinal disorders and diabetes, and especially from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance, which method comprises administering to a subject in need thereof at least one compound of formula I or an N-oxide, a tautomeric form, a stereoisomer or a pharmaceutically acceptable salt thereof.

In yet another aspect, the invention relates to a method for modulating 5HT_{2C} receptor activity in a subject, in particular in a subject suffering of one of the above-listed disorders.

### DETAILED DESCRIPTION

The compounds of the formula I may exist in different spatial arrangements. For example, if the compounds possess one or more centers of asymmetry, polysubstituted rings or double bonds, or may exist as different tautomers, the present invention contemplates the possible use of enantiomeric mixtures, in particular racemates, diastereomeric mixtures and tautomeric mixtures, as well as the respective essentially pure enantiomers, diastereomers and/or tautomers of the compounds of formula I and/or their salts.

One center of chirality is for example the carbon atom carrying radicals R³ and R⁴ (if these are different, of course). Other centers of chirality are for example asymmetry centers in the radical R⁸.

It is likewise possible to use physiologically tolerated salts of the compounds of the formula I, especially acid addition salts with physiologically tolerated acids. Examples of suitable physiologically tolerated organic and inorganic acids are hydrochloric acid, hydrobromic acid, phosphoric acid, sulfuric acid, acetic acid, trifluoroacetic acid, C₁-C₄-alkylsulfonic acids, such as methanesulfonic acid, aromatic sulfonic acids, such as benzenesulfonic acid and toluenesulfonic acid, oxalic acid, maleic acid, fumaric acid, lactic acid, tartaric acid, adipic acid and benzoic acid. Other utilizable acids are described in Fortschritte der Arzneimittelforschung [Advances in drug research], Volume 10, pages 224 et seq., Birkhäuser Verlag, Basel and Stuttgart, 1966.

The compounds of formula I may also be present in the form of tautomers. In one aspect, tautomerism may be present in compounds I wherein R⁷ is OH. Such compounds may have the following tautomeric formulae:

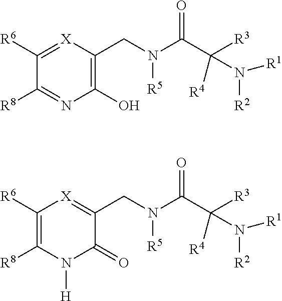

Tautomers may also be present in compounds I wherein R⁵ is H (amide/imidic acid tautomerism).

The organic moieties mentioned in the above definitions of the variables are, like the term halogen, collective terms for individual listings of the individual group members. The prefix Cₙ-Cₘ indicates in each case the possible number of carbon atoms in the group.

The term “halogen” denotes in each case fluorine, bromine, chlorine or iodine. In one aspect, the halogen may be fluorine, chlorine or bromine.

The term “alkyl” as used herein and in the alkyl moieties of alkoxy and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”), 1 to 6 (“C₁-C₆-alkyl”) or 1 to 8 (“C₁-C₈-alkyl”) carbon atoms. C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl and the position isomers thereof.

The term “fluorinated alkyl” as used herein refers to straight-chain or branched alkyl groups having 1 to 2 (“fluorinated C₁-C₂-alkyl”), 1 to 3 (“fluorinated C₁-C₃-alkyl”), 1 to 4 (“fluorinated C₁-C₄-alkyl”), 1 to 6 (“fluorinated C₁-C₆-alkyl”) or 1 to 8 (“fluorinated C₁-C₈-alkyl”) carbon atoms (as mentioned above), where some or all of the hydrogen atoms in these groups are replaced by fluorine atoms. Fluorinated C₁-C₂-alkyl is an alkyl group having 1 or 2 carbon atoms (as mentioned above), where at least one of the hydrogen atoms, e.g. 1, 2, 3, 4 or 5 hydrogen atoms in these groups are replaced by fluorine atoms, such as fluoromethyl, difluoromethyl, trifluoromethyl, 1-fluoroethyl, (R)-1-fluoroethyl, (S)-1-fluoroethyl, 2-fluoroethyl, 2,2-difluoroethyl, 2,2,2-trifluoroethyl, or pentafluoroethyl. Fluorinated C₁-C₄-alkyl is a straight-chain or branched alkyl group having 1 to 4 carbon atoms (as mentioned above), where at least one of the hydrogen atoms, e.g. 1, 2, 3, 4 or 5 hydrogen atoms in these groups are replaced by fluorine atoms. Examples are, apart those listed above for fluorinated C₁-C₂-alkyl, 1-fluoropropyl, (R)-1-fluoropropyl, (S)-1-fluoropropyl, 2-fluoropropyl, (R)-2-fluoropropyl, (S)-2-fluoropropyl, 3-fluoropropyl, 1,1-difluoropropyl, 2,2-difluoropropyl, 1,2-difluoropropyl, 2,3-difluoropropyl, 1,3-difluoropropyl, 3,3-difluoropropyl, 1,1,2-trifluoropropyl, 1,2,2-trifluoropropyl, 1,2,3-trifluoropropyl, 2,2,3-trifluoropropyl, 3,3,3-trifluoropropyl, 1,1,1-trifluoroprop-2-yl, 2-fluoro-1-methylethyl, (R)-2-fluoro-1-methylethyl, (S)-2-fluoro-1-methylethyl, 2,2-difluoro-1-methylethyl, (R)-2,2-difluoro-1-methylethyl, (S)-2,2-difluoro-1-methylethyl, 1,2-difluoro-1-methylethyl, (R)-1,2-difluoro-1-methylethyl, (S)-1,2-difluoro-1-methylethyl, 2,2,2-trifluoro-1-methylethyl, (R)-2,2,2-trifluoro-1-methylethyl, (S)-2,2,2-trifluoro-1-methylethyl, 2-fluoro-1-(fluoromethyl)ethyl, 1-(difluoromethyl)-2,2-difluoroethyl, 1-(trifluoromethyl)-2,2,2-trifluoroethyl, 1-(trifluoromethyl)-1,2,2,2-tetrafluoroethyl, 1-fluorobutyl, (R)-1-fluorobutyl, (S)-1-fluorobutyl, 2-fluorobutyl, (R)-2-fluorobutyl, (S)-2-fluorobutyl, 3-fluorobutyl, (R)-3-fluorobutyl, (S)-3-fluorobutyl, 4-fluorobutyl, 1,1-difluorobutyl, 2,2-difluorobutyl, 3,3-difluorobutyl, 4,4-difluorobutyl, 4,4,4-trifluorobutyl and the like. Fluorinated C₁-C₆-alkyl is a straight-chain or branched alkyl group having 1 to 6 carbon atoms (as mentioned above), where at least one of the hydrogen atoms, e.g. 1, 2, 3, 4 or 5 hydrogen atoms in these groups are replaced by fluorine atoms. Examples are, apart those listed above for fluorinated C₁-C₄-alkyl, 1-fluoropentyl, (R)-1-fluoropentyl, (S)-1-fluoropentyl, 2-fluoropentyl, (R)-2-fluoropentyl, (S)-2-fluoropentyl, 3-fluoropentyl, (R)-3-fluoropentyl, (S)-3-fluoropentyl, 4-fluoropentyl, (R)-4-fluoropentyl, (S)-4-fluoropentyl, 5-fluoropentyl, (R)-5-fluoropentyl, (S)-5-fluoropentyl, 1-fluorohexyl, (R)-1-fluorohexyl, (S)-1-fluorohexyl, 2-fluorohexyl, (R)-2-fluorohexyl, (S)-2-fluorohexyl, 3-fluorohexyl, (R)-3-fluorohexyl, (S)-3-fluorohexyl, 4-fluorohexyl, (R)-4-fluorohexyl, (S)-4-fluorohexyl, 5-fluorohexyl, (R)-5-fluorohexyl, (S)-5-fluorohexyl, 6-fluorohexyl, (R)-6-fluorohexyl, (S)-6-fluorohexyl, and the like. Fluorinated C₁-C₈-alkyl is a straight-chain or branched alkyl group having 1 to 8 carbon atoms (as mentioned above), where at least one of the hydrogen atoms, e.g. 1, 2, 3, 4 or 5 hydrogen atoms in these groups are replaced by fluorine atoms.

The term “haloalkyl” as used herein, which may also be expressed as “alkyl which is partially or fully halogenated”, refers to straight-chain or branched alkyl groups having 1 to 2 (“C₁-C₂-haloalkyl”), 1 to 3 (“C₁-C₃-haloalkyl”) or 1 to 4 (“C₁-C₄-haloalkyl”) carbon atoms (as mentioned above), where some or all of the hydrogen atoms in these groups are replaced by halogen atoms as mentioned above. Examples for C₁-C₂-haloalkyl are, apart those listed above for fluorinated C₁-C₂-alkyl, chloromethyl, bromomethyl, dichloromethyl, trichloromethyl, chlorofluoromethyl, dichlorofluoromethyl, chlorodifluoromethyl, 1-chloroethyl, 1-bromoethyl, 2-chloro-2-fluoroethyl, 2-chloro-2,2-difluoroethyl, 2,2-dichloro-2-fluoroethyl or 2,2,2-trichloroethyl. Examples for C₁₋C₃-haloalkyl are, apart those listed above for C₁-C₂-haloalkyl and for fluorinated C₁-C₃-alkyl, 3-chloropropyl and the like. Examples for C₁-C₄-haloalkyl are, apart those mentioned above for C₁-C₃-haloalkyl and for fluorinated C₁-C₄-alkyl, 4-chlorobutyl and the like.

The term “alkenyl” as used herein refers to monounsaturated straight-chain or branched hydrocarbon radicals having 2 to 3 (“C₂-C₃-alkenyl”) or 2 to 4 (“C₂-C₄-alkenyl”) carbon atoms and a double bond in any position. Examples for C₂-C₃-alkenyl are ethenyl, 1-propenyl, 2-propenyl or 1-methylethenyl. Examples for C₂-C₄-alkenyl are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl.

The term “cycloalkyl” as used herein refers to monocyclic saturated hydrocarbon radicals having 3 to 6 carbon atoms (“C₃-C₆-cycloalkyl”). Examples of C₃-C₆-cycloalkyl are cyclopropyl, cyclobutyl, cyclopentyl and cyclohexyl.

The term “bicycloalkyl” as used herein refers to bicyclic saturated hydrocarbon radicals having 5 to 10 carbon atoms (“C₅-C₁₀-bicycloalkyl”). Examples are bicyclo[1.1.1]pentyl, bicyclo[3.1.0]hexyl, bicyclo[2.1.1]hexyl, bicyclo[4.1.0]heptyl, bicyclo[2.2.1]heptyl, bicyclo[3.1.1]heptyl, bicyclo[2.2.2]octyl, bicyclo[3.2.1]octyl, 1,2,3,3a,4,5,6,6a-octahydropentalenyl, 2,3,3a,4,5,6,7,7a-octahydro-1H-indenyl, decalinyl (decahydronaphthalenyl, bicyclo[4.4.0]decanyl) and the like.

The term “cycloalkenyl” as used herein refers to monocyclic partially unsaturated, non-aromatic hydrocarbon radicals having 3 to 6 (“C₃-C₆-cycloalkenyl”) carbon atoms and one or more non-cumulative, preferably one, C—C double bonds in the ring. Examples for C₃-C₆-cycloalkenyl are cycloprop-1-en-1-yl, cycloprop-1-en-3-yl, cyclobut-1-en-1-yl, cyclobut-1-en-3-yl, cyclopent-1-en-1-yl, cyclopent-1-en-3-yl, cyclopent-1-en-4-yl, cyclopenta-1,3-dien-1-yl, cyclopenta-1,3-dien-2-yl, cyclopenta-1,3-dien-5-yl, cyclohex-1-en-1-yl, cyclohex-1-en-3-yl, cyclohex-1-en-4-yl, cyclohexa-1,3-dien-1-yl, cyclohexa-1,3-dien-2-yl, cyclohexa-1,3-dien-5-yl, cyclohexa-1,4-dien-1-yl and cyclohexa-1,4-dien-3-yl. C₆-cycloalkenyl is for example cyclohex-1-en-1-yl, cyclohex-1-en-3-yl, cyclohex-1-en-4-yl, cyclohexa-1,3-dien-1-yl, cyclohexa-1,3-dien-2-yl, cyclohexa-1,3-dien-5-yl, cyclohexa-1,4-dien-1-yl and cyclohexa-1,4-dien-3-yl.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy).

The term “fluorinated C₁-C₂-alkoxy” is a fluorinated C₁-C₂-alkyl group, as defined above, attached via an oxygen atom. The term “fluorinated C₁-C₃-alkoxy” is a fluorinated C₁-C₃-alkyl group, as defined above, attached via an oxygen atom. The term “fluorinated C₁-C₄-haloalkoxy” is a fluorinated C₁-C₄-alkyl group, as defined above, attached via an oxygen atom. Fluorinated C₁-C₂-alkoxy is, for example, OCH₂F, OCHF₂, OCF₃, 1-fluoroethoxy, (R)-1-fluoroethoxy, (S)-1-fluoroethoxy, 2-fluoroethoxy, 1,1-difluoroethoxy, 1,2-difluoroethoxy, 2,2-difluoroethoxy, 1,1,2-trifluoroethoxy, 1,2,2-trifluoroethoxy, 2,2,2-trifluoroethoxy, 1,1,2,2-tetrafluoroethoxy or OC₂F₅. Fluorinated C₁-C₃-alkoxy is additionally, for example, 1-fluoropropoxy, (R)-1-fluoropropoxy, (S)-1-fluoropropoxy, 2-fluoropropoxy, (R)-2-fluoropropoxy, (S)-2-fluoropropoxy, 3-fluoropropoxy, 1,1-difluoropropoxy, 2,2-difluoropropoxy, 2,3-difluoropropoxy, 3,3-difluoropropoxy, 3,3,3-trifluoropropoxy, (R)-2-fluoro-1-methylethoxy, (S)-2-fluoro-1-methylethoxy, (R)-2,2-difluoro-1-methylethoxy, (S)-2,2-difluoro-1-methylethoxy, (R)-1,2-difluoro-1-methylethoxy, (S)-1,2-difluoro-1-methylethoxy, (R)-2,2,2-trifluoro-1-methylethoxy, (S)-2,2,2-trifluoro-1-methylethoxy, 2-fluoro-1-(fluoromethyl)ethoxy, 1-(difluoromethyl)-2,2-difluoroethoxy, OCH₂—C₂F₅, OCF₂—C₂F₅ or 1-(CH₂F)-2-fluoroethoxy. Fluorinated C₁-C₄-alkoxy is additionally, for example, 1-fluorobutoxy, (R)-1-fluorobutoxy, (S)-1-fluorobutoxy, 2-fluorobutoxy, 3-fluorobutoxy, 4-fluorobutoxy, 1,1-difluorobutoxy, 2,2-difluorobutoxy, 3,3-difluorobutoxy, 4,4-difluorobutoxy, 4,4,4-trifluorobutoxy or nonafluorobutoxy.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. Examples for C₁-C₂-haloalkoxy are, apart those mentioned above for fluorinated C₁-C₂-alkoxy, OCH₂C1, OCHCl₂, OCCl₃, chlorofluoromethoxy, dichlorofluoromethoxy, chlorodifluoromethoxy, 2-chloroethoxy, 2-bromoethoxy, 2-iodoethoxy, 2-chloro-2-fluoroethoxy, 2-chloro-2,2-difluoroethoxy, 2,2-dichloro-2-fluoroethoxy or 2,2,2-trichloroethoxy. Examples for C₁-C₃-haloalkoxy are, apart those mentioned above for C₁-C₂-haloalkoxy and for fluorinated C₁-C₃-alkoxy, 2-chloropropoxy, 3-chloropropoxy, 2,3-dichloropropoxy, 2-bromopropoxy, 3-bromopropoxy, 3,3,3-trichloropropoxy, 1-(CH₂Cl)-2-chloroethoxy or 1-(CH₂Br)-2-bromoethoxy. Examples for C₁-C₄-haloalkoxy are, apart those mentioned above for C₁-C₃-haloalkoxy and for fluorinated C₁-C₄-alkoxy, for example, 4-chlorobutoxy or 4-bromobutoxy.

Phenoxy is a phenyl ring bound via an oxygen atom to the remainder of the molecule.

Benzyl is a phenyl ring bound via a CH₂ group to the remainder of the molecule.

Unsaturated heterocyclic rings contain at least one C—C and/or C—N double bond(s). Partially unsaturated rings contain less conjugated C—C and/or C—N double bonds than maximally allowed by the ring size.

Examples for N-bound 4-, 5- or 6-membered saturated heterocyclic rings formed by R¹ and R² together with the nitrogen atom they are bound to are azetidin-1-yl, pyrrolidin-1-yl and piperidin-1-yl.

Examples for 4-, 5- or 6-membered saturated or partially unsaturated heteromonocyclic rings formed by R² and R³ together with the atoms they are bound to are azetidin-2-yl, pyrrolidin-2-yl, piperidin-2-yl, 1,2-dihydroazet-2-yl, 1,2-dihydroazet-4-yl, 2,3-dihydroazet-4-yl, 2,3-dihydroazet-2-yl, 2,3-dihydro-1H-pyrrol-5-yl, 2,5-dihydro-1H-pyrrol-2-yl, 2,3-dihydro-1H-pyrrol-2-yl, 3,4-dihydro-2H-pyrrol-2-yl, 3,4-dihydro-2H-pyrrol-5-yl, 1,2,3,4-tetrahydropyridin-6-yl, 1,2,3,6-tetrahydropyridin-6-yl, 1,2,3,6-tetrahydropyridin-2-yl, 1,2,3,4-tetrahydropyridin-2-yl, 2,3,4,5-tetrahydropyridin-2-yl, 2,3,4,5-tetrahydropyridin-6-yl, 1,2-dihydropyridin-6-yl, 1,4-dihydropyridin-2-yl, 3,4-dihydropyridin-6-yl, 1,2-dihydropyridin-2-yl, 2,5-dihydropyridin-2-yl, 2,3-dihydropyridin-6-yl, 2,3-dihydropyridin-2-yl, 2,5-dihydropyridin-6-yl, 3,4-dihydropyridin-2-yl and the like.

Examples for 4-, 5-, 6-, 7-, 8-, 9 or 10-membered saturated or partially unsaturated heterobicyclic rings formed by R² and R³ together with the atoms they are bound to are,

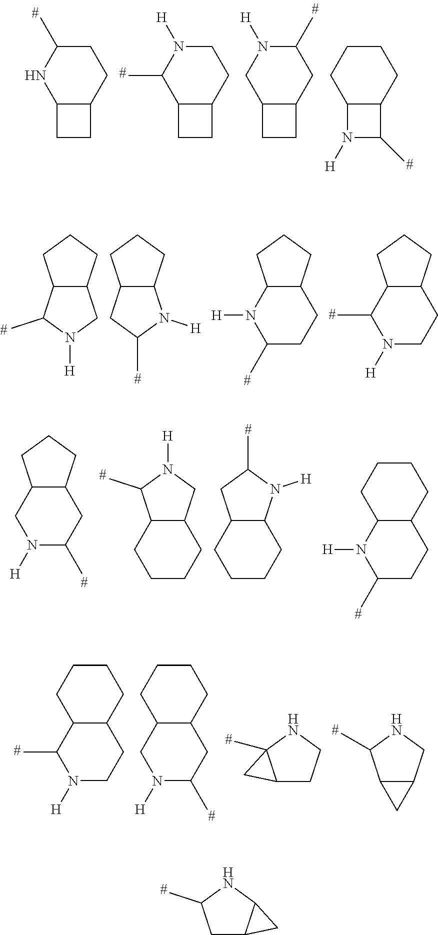

and partially unsaturated analogues thereof.

4-, 5-, 6- or 7-membered saturated heteromonocyclic rings formed by R^{8b} and R^{8c}, together with the nitrogen atom they are bound to, which may additionally contain a further heteroatom selected from the group consisting of N, O and S as ring member, are N-bound rings. Examples are azetidin-1-yl, pyrrolidin-1-yl, piperidin-1-yl, azepan-1-yl, pyrazolidin-1-yl, imidazolidin-1-yl, oxazolidin-3-yl, isoxazolidin-2-yl, thiazolidin-3-yl, isothiazolidin-2-yl, piperazin-1-yl, morpholin-1-yl, thiomorpholin-1-yl and the like.

6-, 7-, 8-, 9- or 10-membered saturated or unsaturated heterobicyclic rings formed by R^{8b} and R^{8c}, together with the nitrogen atom they are bound to are N-bound rings. The bicyclic rings can be fused systems, spiro systems or bridged systems, saturated, partially unsaturated, or completely unsaturated, including aromatic. In fused, or condensed, bicyclic systems, the two rings contain two common adjacent ring atoms. In bicyclic spiro systems the two rings contain one ring atom in common. In bridged systems the two rings contain two common non-adjacent ring atoms.

Examples for N-bound fused bicyclic rings are

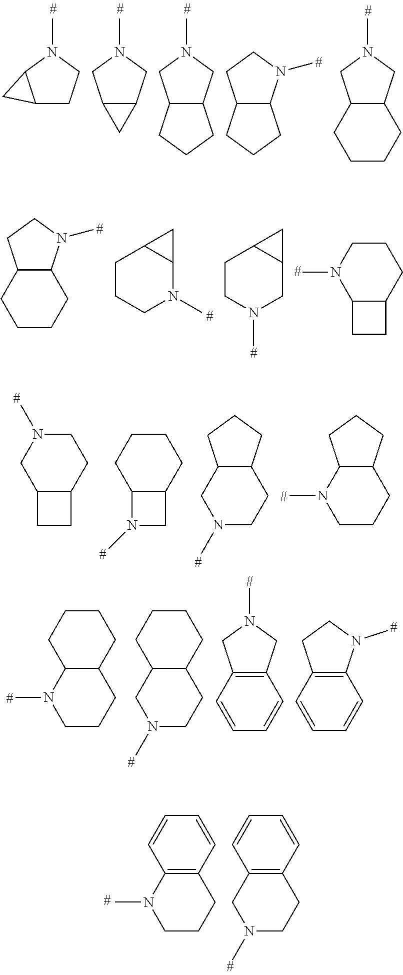

Examples for N-bound bicyclic spiro systems are:

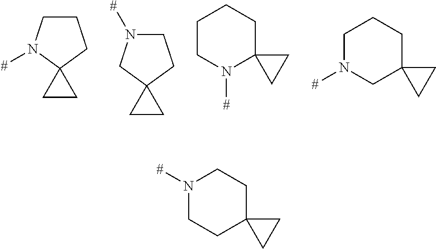

Examples for N-bound bridged systems are:

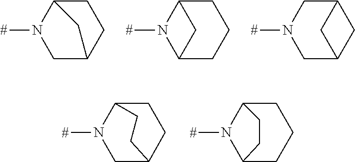

In the above structures # denotes the attachment point to the remainder of the molecule.

5- or 6-membered saturated heteromonocyclic ring containing a heteroatom selected from the group consisting of N, O and S as ring member are for example tetrahydrofuran-2-yl, tetrahydrofuran-3-yl, tetrahydrothiophen-2-yl, tetrahydrothiophen-3-yl, pyrrolidin-1-yl, pyrrolidin-2-yl, pyrrolidin-3-yl, tetrahydropyran-2-yl, tetrahydropyran-3-yl, tetrahydropyran-4-yl, tetrahydrothiopyran-2-yl, tetrahydrothiopyran-3-yl, tetrahydrothiopyran-4-yl, piperidine-1-yl, piperidine-2-yl, piperidine-3-yl or piperidine-4-yl.

C-bound 5- or 6-membered saturated heteromonocyclic ring containing a heteroatom selected from the group consisting of N, O and S as ring member are for example tetrahydrofuran-2-yl, tetrahydrofuran-3-yl, tetrahydrothiophen-2-yl, tetrahydrothiophen-3-yl, pyrrolidin-2-yl, pyrrolidin-3-yl, tetrahydropyran-2-yl, tetrahydropyran-3-yl, tetrahydropyran-4-yl, tetrahydrothiopyran-2-yl, tetrahydrothiopyran-3-yl, tetrahydrothiopyran-4-yl, piperidine-2-yl, piperidine-3-yl or piperidine-4-yl.

A C-bound 6-membered saturated heteromonocyclic ring containing an oxygen atom as ring member is for example tetrahydropyran-2-yl, tetrahydropyran-3-yl or tetrahydropyran-4-yl.

The remarks made above and in the following with respect to preferred aspects of the invention, e.g. to preferred meanings of the variables R¹, R², R³, R⁴, R⁵, R⁶, R⁷, R⁸, R^{8a}, R^{8b}, R^{8c}, R^{8d}, R^{9a}, R^{9b}, R^{9c}, R¹⁰, R^{11a}, R¹², R¹³ and R¹⁴ of compounds I, to preferred compounds I and to preferred embodiments of the method or the use according to the invention, apply in each case on their own or in particular to combinations thereof.

In a preferred embodiment, the moiety

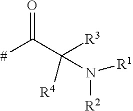

is derived from an L-α-amino acid.

In a preferred embodiment (embodiment 1), R³ and R⁴ are independently of each other hydrogen or methyl. In a specific embodiment (embodiment 1.1), both are hydrogen or one is hydrogen and the other is methyl.

In a preferred embodiment (embodiment 2), R¹ and R², together with the nitrogen atom they are bound to, form an N-bound 4-, 5- or 6-membered saturated heterocyclic ring (i.e. form together azetidin-1-yl, pyrrolidin-1-yl or piperidin-1-yl) which may carry 1 substituent selected from halogen, C₁-C₂-alkyl and fluorinated C₁-C₂-alkyl; and which specifically is unsubstituted. In particular (embodiment 2.1), R¹ and R², together with the nitrogen atom they are bound to, form an N-bound 4- or 5-membered saturated heterocyclic ring (i.e. form together azetidin-1-yl or pyrrolidin-1-yl) which may carry 1 substituent selected from halogen, C₁-C₂-alkyl and fluorinated C₁-C₂-alkyl; and which specifically is unsubstituted. Specifically (embodiment 2.1.1), R¹ and R², together with the nitrogen atom they are bound to, form an N-bound 4-membered saturated heterocyclic ring (i.e. form azetidin-1-yl) which may carry 1 substituent selected from halogen, C₁-C₂-alkyl and fluorinated C₁-C₂-alkyl; and which specifically is unsubstituted. In these embodiments 2, 2.1 and 2.1.1 R³ and R⁴ are in particular simultaneously as defined in embodiments 1 and 1.1.

Thus, in a particular embodiment (embodiment 2.2), R³ and R⁴ are independently of each other hydrogen or methyl and R¹ and R², together with the nitrogen atom they are bound to, form an N-bound 4- or 5-membered saturated heterocyclic ring which may carry 1 substituent selected from halogen, C₁-C₂-alkyl and fluorinated C₁-C₂-alkyl; and which specifically is unsubstituted. In a more particular embodiment (embodiment 2.2.1) R³ and R⁴ are both hydrogen or one is hydrogen and the other is methyl, and R¹ and R², together with the nitrogen atom they are bound to, form an N-bound 4-membered saturated heterocyclic ring (i.e. form azetidin-1-yl) which may carry 1 substituent selected from halogen, C₁-C₂-alkyl and fluorinated C₁-C₂-alkyl; and which specifically is unsubstituted.

In an alternatively preferred embodiment (embodiment 3), R² and R³, together with the atoms they are bound to, form a 4-, 5- or 6-membered saturated or partially unsaturated heteromonocyclic ring (forming thus, for example, azetidin-2-yl, pyrrolidin-2-yl, pyrrolin-2-yl, piperidin-2-yl, dihydropyridin-2-yl or tetrahydropyridin-2-yl) which may carry 1 substituent selected from halogen, C₁-C₄-alkyl and fluorinated C₁-C₄-alkyl, where R¹ and R⁴, if present, have one of the above general or one of the below definitions.

In a particular embodiment (embodiment 3.1),
R² and R³, together with the atoms they are bound to, form a 4- or 5-membered saturated or partially unsaturated heteromonocyclic ring (forming thus, for example, azetidin-2-yl, pyrrolidin-2-yl or pyrrolin-2-yl) which may carry 1 substituent selected from halogen, C₁-C₄-alkyl and fluorinated C₁-C₄-alkyl;
R¹ is hydrogen or methyl (if the nitrogen atom is part of a double bond, R¹ is of course absent); and
R⁴ is hydrogen or methyl (if the carbon atom carrying R³ is part of a double bond, R⁴ is of course absent); and is in particular hydrogen.

In a more particular embodiment (embodiment 3.1.1),
R² and R³, together with the atoms they are bound to, form a 5-membered saturated heteromonocyclic ring (forming thus pyrrolidin-2-yl) which may carry 1 substituent selected from halogen, C₁-C₄-alkyl and fluorinated C₁-C₄-alkyl;
R¹ is hydrogen or methyl; and
R⁴ is hydrogen or methyl, and is in particular hydrogen.

In a specific embodiment (embodiment 3.1.1.1) the compound I is a compound of formula I.1

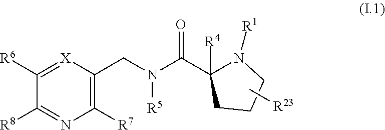

wherein
X, R⁵, R⁶, R⁷ and R⁸ have one of the above general or, in particular, one of the below preferred meanings;
R¹ and R⁴ have one of the above general meanings or, in particular, one of the meanings given in embodiment 3.1.1; and
R²³ is selected from hydrogen, halogen, C₁-C₄-alkyl and fluorinated C₁-C₄-alkyl, preferably from hydrogen, F and methyl; and is in particular hydrogen.

In an alternatively preferred embodiment (embodiment 4)
R² and R³, together with the atoms they are bound to, form a 6-, 7-, 8-, 9- or 10-membered saturated heterobicyclic ring which may carry 1 substituent selected from halogen, C₁-C₄-alkyl and fluorinated C₁-C₄-alkyl, and form in particular a 6-membered saturated heterobicyclic ring;
R¹ is hydrogen or methyl (if the nitrogen atom is a fusion point of the bicyclic ring, R¹ is of course absent); and
R⁴ is hydrogen or methyl (if the carbon atom which is the attachment point of the bicyclic ring to the C(O)NR⁵ group is a fusion point, R⁴ is of course absent).

In a particular embodiment (embodiment 4.1)
R² and R³, together with the atoms they are bound to, form a 6-membered saturated heterobicyclic ring;
R¹ is hydrogen or methyl, in particular hydrogen (if the nitrogen atom is a fusion point of the bicyclic ring, R¹ is of course absent); and
R⁴ is hydrogen or methyl, in particular hydrogen (if the carbon atom which is the attachment point of the bicyclic ring to the C(O)NR⁵ group is a fusion point, R⁴ is of course absent).

Specifically (embodiment 4.1.1), the 6-membered saturated heterobicyclic ring is a pyrrolidine ring condensed with a cyclopropyl ring via two carbon atoms; i.e. a ring of one of the following formulae:

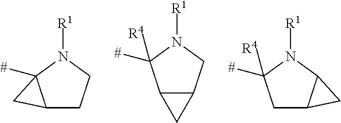

where R¹ and R⁴ are independently hydrogen or methyl and specifically hydrogen.

In a particular embodiment (embodiment 5), R⁵ is H or methyl. In particular, embodiment 5 relates to compounds I wherein R⁵ is H or methyl, and R¹, R², R³ and R⁴ are as defined in embodiments 1, 1.1, 2, 2.1, 2.1.1, 2.2, 2.2.1, 3, 3.1, 3.1.1, 3.1.1.1, 4, 4.1 or 4.1.1. In a more particular embodiment (embodiment 5.1), R⁵ is H. In particular, embodiment 5.1 relates to compounds I wherein R⁵ is H, and R¹, R², R³ and R⁴ are as defined in embodiments 1, 1.1, 2, 2.1, 2.1.1, 2.2, 2.2.1, 3, 3.1, 3.1.1, 3.1.1.1, 4, 4.1 or 4.1.1.

In a preferred embodiment (embodiment 6), R⁶ is F if X is CH and simultaneously R⁷ is C₁-C₄-alkoxy, fluorinated C₁-C₄-alkoxy or hydroxyl; and is hydrogen if X is N or if X is CH and simultaneously R⁷ is C₁-C₄-alkyl or fluorinated C₁-C₄-alkyl. In particular, embodiment 6 relates to compounds I wherein R⁶ is F if X is CH and simultaneously R⁷ is C₁-C₄-alkoxy, fluorinated C₁-C₄-alkoxy or hydroxyl; and is hydrogen if X is N or if X is CH and simultaneously R⁷ is C₁-C₄-alkyl or fluorinated C₁-C₄-alkyl, and R¹, R², R³, R⁴ and R⁵ are as defined in embodiments 1, 1.1, 2, 2.1, 2.1.1, 2.2, 2.2.1, 3, 3.1, 3.1.1, 3.1.1.1, 4, 4.1, 4.1.1, 5 or 5.1.

In a preferred embodiment (embodiment 7), R⁷ is C₁-C₄-alkoxy. In particular, embodiment 7 relates to compounds I wherein R⁷ is C₁-C₄-alkoxy and R¹, R², R³, R⁴, R⁵ and R⁶ are as defined in embodiments 1, 1.1, 2, 2.1, 2.1.1, 2.2, 2.2.1, 3, 3.1, 3.1.1, 3.1.1.1, 4, 4.1, 4.1.1, 5, 5.1 or 6. In a particular embodiment (embodiment 7.1), R⁷ is methoxy. In particular, embodiment 7.1 relates to compounds I wherein R⁷ is methoxy and R¹, R², R³, R⁴, R⁵ and R⁶ are as defined in embodiments 1, 1.1, 2, 2.1, 2.1.1, 2.2, 2.2.1, 3, 3.1, 3.1.1, 3.1.1.1, 4, 4.1, 4.1.1, 5, 5.1 or 6.

In an alternatively preferred embodiment (embodiment 8), R⁷ is C₁-C₄-alkyl. In particular, embodiment 8 relates to compounds I wherein R⁷ is C₁-C₄-alkyl and R¹, R², R³, R⁴, R⁵ and R⁶ are as defined in embodiments 1, 1.1, 2, 2.1, 2.1.1, 2.2, 2.2.1, 3, 3.1, 3.1.1, 3.1.1.1, 4, 4.1, 4.1.1, 5, 5.1 or 6. In a particular embodiment (embodiment 8.1), R⁷ is methyl. In particular, embodiment 8.1 relates to compounds I wherein R⁷ is methyl and R¹, R², R³, R⁴, R⁵ and R⁶ are as defined in embodiments 1, 1.1, 2, 2.1, 2.1.1, 2.2, 2.2.1, 3, 3.1, 3.1.1, 3.1.1.1, 4, 4.1, 4.1.1, 5, 5.1 or 6.

In a particular embodiment (embodiment 9), X is CH. In particular, embodiment 9 relates to compounds I wherein X is CH and R¹, R², R³, R⁴, R⁵, R⁶ and R⁷ are as defined in embodiments 1, 1.1, 2, 2.1, 2.1.1, 2.2, 2.2.1, 3, 3.1, 3.1.1, 3.1.1.1, 4, 4.1, 4.1.1, 5, 5.1, 6, 7, 7.1 8 or 8.1.

In a preferred embodiment (embodiment 10), R⁸ is a radical —OR^{8a}.

In this embodiment,
R^{8a} is selected from C₁-C₈-alkyl, C₁-C₄-alkyl which carries a radical R^{9a}; fluorinated C₁-C₈-alkyl, C₃-C₆-cycloalkyl which may carry one or more substituents R¹⁰; C₅-C₁₀-bicycloalkyl which may carry one or more substituents R¹⁰; and phenyl which may carry one or more substituents R¹²;
R^{9a} is selected from C₃-C₆-cycloalkyl which may carry one or more substituents R¹¹; C₅-C₁₀-bicycloalkyl which may carry one or more substituents R¹¹; adamantyl which may carry one or more substituents R¹¹; and phenyl which may carry one or more substituents R¹²;
each R¹⁰ is independently selected from halogen, hydroxyl, C₁-C₄-alkyl, C₁-C₄-haloalkyl, C₁-C₄-alkoxy, C₁-C₄-haloalkoxy and phenyl which may carry 1, 2, 3, 4 or 5 substituents R¹³; or
two substituents R¹⁰ bound on the same carbon atom of the cycloalkyl or bicycloalkyl ring, together with the carbon atom they are bound to, form a 3-, 4-, 5- or 6-membered saturated carbocyclic ring, where the carbocyclic ring may carry one or more substituents selected from halogen, C₁-C₄-alkyl and C₁-C₄-haloalkyl;
where two substituents R¹⁰ bound on the same carbon atom of the cycloalkyl or bicycloalkyl ring R⁸ are not both C₁-C₄-alkyl;
each R¹¹ is independently selected from halogen, C₁-C₄-alkyl, C₁-C₄-haloalkyl, C₁-C₄-alkoxy and C₁-C₄-haloalkoxy; and
R¹² and R¹³, independently of each other and independently of each occurrence, are selected from halogen, CN, C₁-C₄-alkyl, C₁-C₄-haloalkyl, C₁-C₄-alkoxy and C₁-C₄-haloalkoxy.

Preferably (embodiment 10.1), R^{8a} is selected from C₁-C₂-alkyl which carries a radical R^{9a}; fluorinated C₁-C₈-alkyl, C₃-C₆-cycloalkyl which carries one or more substituents R¹⁰; and C₆-C₁₀-bicycloalkyl which carries one or more substituents R¹⁰; where R^{9a} and R¹⁰ have one of the above general meanings. In particular, embodiment 10.1 relates to compounds I wherein R⁸ is a radical —OR^{8a}, wherein R^{8a} is as defined herein and R¹, R², R³, R⁴, R⁵, R⁶, R⁷ and X are as defined in embodiments 1, 1.1, 2, 2.1, 2.1.1, 2.2, 2.2.1, 3, 3.1, 3.1.1, 3.1.1.1, 4, 4.1, 4.1.1, 5, 6, 7, 7.1, 8, 8.1 or 9.

In a more preferred embodiment (embodiment 10.1.1),
R^{8a} is selected from C₁-C₂-alkyl which carries a radical R^{9a}; fluorinated C₁-C₈-alkyl, C₃-C₆-cycloalkyl which carries one or more substituents R¹⁰; and C₆-C₁₀-bicycloalkyl which carries one or more substituents R¹⁰; where
   R^{9a} is selected from C₃-C₆-cycloalkyl which may carry 1, 2, 3, 4, 5 or 6 substituents R¹¹; C₅-C₁₀-bicycloalkyl which may carry 1, 2, 3, 4, 5 or 6 substituents R¹¹; adamantly; and phenyl which may carry 1, 2 or 3 substituents R¹²;
   each R¹⁰ is independently selected from fluorine, hydroxyl, fluorinated C₁-C₄-alkyl, C₁-C₄-alkoxy and fluorinated C₁-C₄-alkoxy; or
      two substituents R¹⁰ bound on the same carbon atom of the cycloalkyl or bicycloalkyl ring, together with the carbon atom they are bound to, form a 3- or 4-membered (spiro-bound) saturated carbocyclic ring, where the carbocyclic ring may carry one or more substituents selected from halogen, C₁-C₄-alkyl and C₁-C₄-haloalkyl;
   each R¹¹ is independently selected from fluorine and fluorinated C₁-C₄-alkyl; and
   each R¹² is independently selected from halogen, CN, C₁-C₄-alkyl, C₁-C₄-haloalkyl, C₁-C₄-alkoxy and C₁-C₄-haloalkoxy.

In particular, embodiment 10.1.1 relates to compounds I wherein R⁸ is a radical —OR^{8a}, wherein R^{8a}, R^{9a}, R¹⁰, R¹¹ and R¹² are as defined herein and R¹, R², R³, R⁴, R⁵, R⁶, R⁷ and X are as defined in embodiments 1, 1.1, 2, 2.1, 2.1.1, 2.2, 2.2.1, 3, 3.1, 3.1.1, 3.1.1.1, 4, 4.1, 4.1.1, 5, 6, 7, 7.1, 8, 8.1 or 9.

In a particular embodiment (embodiment 10.1.1.1),
R^{8a} is selected from —CH₂—R^{9a}; fluorinated C₁-C₆-alkyl, C₃-C₆-cycloalkyl which carries 1, 2, 3 or 4 substituents R¹⁰; and C₆-C₁₀-bicycloalkyl which carries 1, 2, 3 or 4 substituents R¹⁰; where
   R^{9a} is selected from C₃-C₆-cycloalkyl which may carry 1, 2, 3, 4, 5 or 6 substituents R¹¹; C₅-C₁₀-bicycloalkyl which may carry 1, 2, 3 or 4 substituents R¹¹; adamantly; and phenyl which may carry 1, 2 or 3 substituents R¹²
   each R¹⁰ is independently selected from fluorine, fluorinated C₁-C₄-alkyl, C₁-C₄-alkoxy and fluorinated C₁-C₄-alkoxy; or
      two substituents R¹⁰ bound on the same carbon atom of the cycloalkyl or bicycloalkyl ring, together with the carbon atom they are bound to, form a 3-membered (spiro-bound) saturated carbocyclic ring, where the carbocyclic ring may carry one or more substituents selected from halogen;
   each R¹¹ is independently selected from fluorine and fluorinated C₁-C₄-alkyl; and
   each R¹² is independently selected from halogen.

In particular, embodiment 10.1.1.1 relates to compounds I wherein R⁸ is a radical —OR^{8a}, wherein R^{8a}, R^{9a}, R¹⁰, R¹¹ and R¹² are as defined herein and R¹, R², R³, R⁴, R⁵, R⁶, R⁷ and X are as defined in embodiments 1, 1.1, 2, 2.1, 2.1.1, 2.2, 2.2.1, 3, 3.1, 3.1.1, 3.1.1.1, 4, 4.1, 4.1.1, 5, 6, 7, 7.1, 8, 8.1 or 9.

In embodiments 10, 10.1, 10.1.1 and 10.1.1.1 in the definition of R^{8a} bicycloalkyl is especially bicyclo[3.1.0]hexyl or bicyclo[4.1.0]heptyl, and in the definition of R^{9a} bicycloalkyl is especially bicyclo[1.1.1]pentyl, bicyclo[3.1.0]hexyl or bicyclo[4.1.0]heptyl.

In another preferred embodiment (embodiment 11), R⁸ is a radical —NR^{8b}R^{8c}.

Preferably (embodiment 11.1),
R^{8b} is selected from the group consisting of hydrogen, C₁-C₄-alkyl and C₂-C₃-alkenyl;
R^{8c} is selected from the group consisting of C₁-C₃-alkyl which carries a radical R^{9b}; C₃-C₆-cycloalkyl which may carry 1 to 6 substituents R¹¹; and phenyl which may carry one or more halogen atoms; or
R^{8b} and R^{8c}, together with the nitrogen atom they are bound to, form a 4-, 5-, 6- or 7-membered saturated heteromonocyclic ring which may additionally contain an oxygen atom as ring member, where the heteromonocyclic ring may carry one or more substituents R¹⁴; or form a 6-, 7-, 8-, 9- or 10-membered saturated or unsaturated heterobicyclic ring which may carry one or more substituents R¹⁴; where
   R^{9b} is selected from the group consisting of C₃-C₆-cycloalkyl which may carry one or more substituents selected from the group consisting of halogen and C₁-C₂-haloalkyl; and phenyl which may carry one or more halogen atoms;
   each R¹¹ is independently selected from the group consisting of halogen and C₁-C₂-haloalkyl; and
   each R¹⁴ is independently selected from the group consisting of halogen, C₁-C₂-alkyl, C₁-C₂-haloalkyl, C₃-C₆-cycloalkyl which may carry one or more substituents selected from the group consisting of halogen and C₁-C₂-haloalkyl; phenyl, phenoxy and benzyl, where the phenyl ring in the three last-mentioned radicals may carry one or more halogen atoms.

In particular, embodiment 11.1 relates to compounds I wherein R⁸ is a radical —NR^{8b}R^{8c}, wherein R^{8b} and R^{8c} are as defined herein and R¹, R², R³, R⁴, R⁵, R⁶, R⁷ and X are as defined in embodiments 1, 1.1, 2, 2.1, 2.1.1, 2.2, 2.2.1, 3, 3.1, 3.1.1, 3.1.1.1, 4, 4.1, 4.1.1, 5, 6, 7, 7.1, 8, 8.1 or 9.

In another preferred embodiment (embodiment 12), R⁸ is a radical —R^{8d}.

Preferably (embodiment 12.1),
R^{8d} is a C-bound radical selected from the group consisting of C₁-C₄-alkyl which carries a radical R^{9c}; C₁-C₂-haloalkyl which carries a radical R^{9c}; C₃-C₆-cycloalkyl which may carry one or more substituents R¹¹; C₆-cycloalkenyl which may carry one or more substituents R¹¹; phenyl; and a C-bound 6-membered saturated heteromonocyclic ring containing an oxygen atom as ring member; where
   R^{9c} is selected from the group consisting of C₁-C₄-alkoxy, C₃-C₆-cycloalkyl which may carry one or more substituents selected from the group consisting of halogen and C₁-C₂-haloalkyl; phenyl; and a 6-membered saturated heteromonocyclic ring containing an oxygen atom as ring member; and
   each R¹¹ is independently selected from the group consisting of C₁-C₂-haloalkyl and C₁-C₂-alkoxy.

In particular, embodiment 12.1 relates to compounds I wherein R⁸ is a radical —R^{8d}, wherein R^{8d} is as defined herein and R¹, R², R³, R⁴, R⁵, R⁶, R⁷ and X are as defined in embodiments 1, 1.1, 2, 2.1, 2.1.1, 2.2, 2.2.1, 3, 3.1, 3.1.1, 3.1.1.1, 4, 4.1, 4.1.1, 5, 6, 7, 7.1, 8, 8.1 or 9.

Examples of preferred compounds are compounds of the following formulae Ia.1 to Ia.24 and the stereoisomers thereof, where the variables have one of the general or preferred meanings given above. Examples of preferred compounds are the individual compounds compiled in the tables 1 to 11256 below. Moreover, the meanings mentioned below for the individual variables in the tables are per se, independently of the combination in which they are mentioned, a particularly preferred embodiment of the substituents in question.

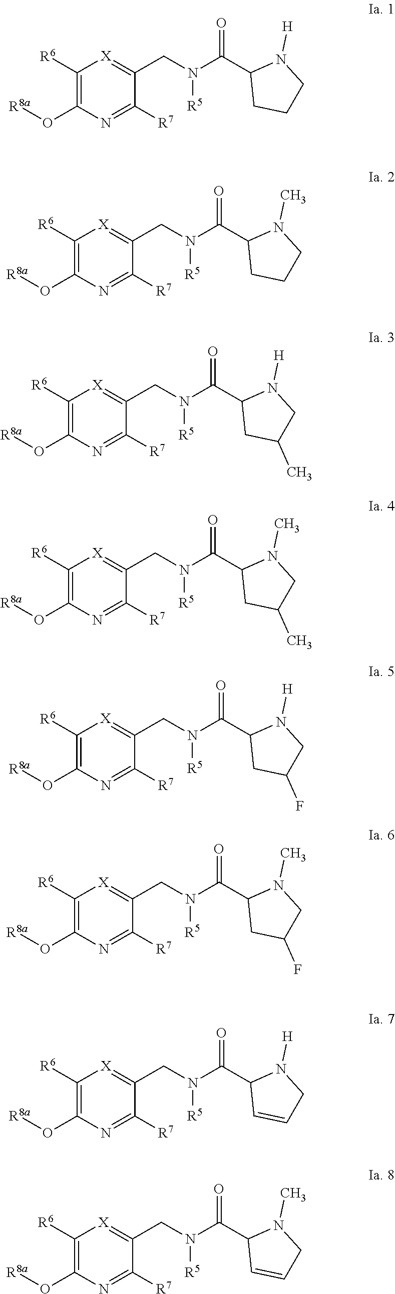

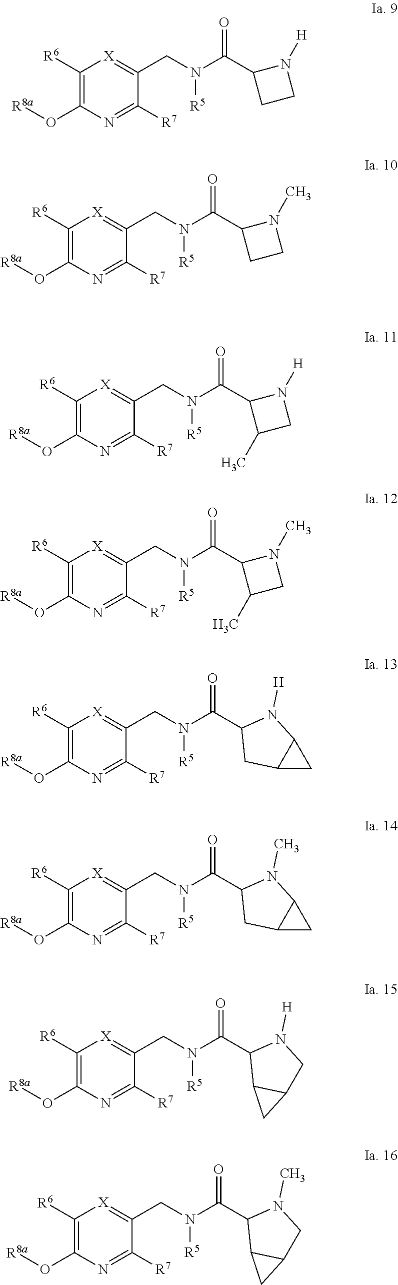

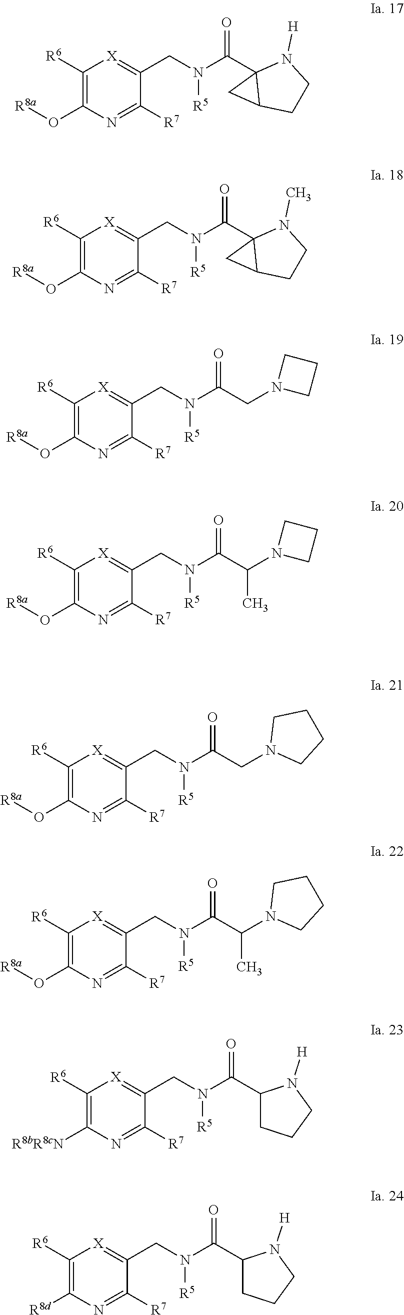

### Table 1

Compounds of the formula Ia.1 in which X is CH, R^{8a} is CH₂F and R⁵, R⁶ and R⁷ for a compound corresponds in each case to one row of Table A

### Table 2

Compounds of the formula Ia.1 in which X is CH, R^{8a} is CHF₂ and R⁵, R⁶ and R⁷ for a compound corresponds in each case to one row of Table A

### Table 3

Compounds of the formula Ia.1 in which X is CH, R^{8a} is CF₃ and R⁵, R⁶ and R⁷ for a compound corresponds in each case to one row of Table A

### Table 4

Compounds of the formula Ia.1 in which X is CH, R^{8a} is CH₂CH₂F and R⁵, R⁶ and R⁷ for a compound corresponds in each case to one row of Table A

### Table 5

Compounds of the formula Ia.1 in which X is CH, R^{8a} is CH₂CHF₂ and R⁵, R⁶ and R⁷ for a compound corresponds in each case to one row of Table A

### Table 6

Compounds of the formula Ia.1 in which X is CH, R^{8a} is CH₂CF₃ and R⁵, R⁶ and R⁷ for a compound corresponds in each case to one row of Table A

### Table 7

Compounds of the formula Ia.1 in which X is CH, R^{8a} is CF₂CH₃ and R⁵, R⁶ and R⁷ for a compound corresponds in each case to one row of Table A

### Table 8

Compounds of the formula Ia.1 in which X is CH, R^{8a} is CF₂CF₃ and R⁵, R⁶ and R⁷ for a compound corresponds in each case to one row of Table A

### Table 9

Compounds of the formula Ia.1 in which X is CH, R^{8a} is CH₂CH₂CHF₂ and R⁵, R⁶ and R⁷ for a compound corresponds in each case to one row of Table A

### Table 10

Compounds of the formula Ia.1 in which X is CH, R^{8a} is CH₂CH₂CF₃ and R⁵, R⁶ and R⁷ for a compound corresponds in each case to one row of Table A

### Table 11

Compounds of the formula Ia.1 in which X is CH, R^{8a} is CH₂CF₂CH₃ and R⁵, R⁶ and R⁷ for a compound corresponds in each case to one row of Table A

### Table 12

Compounds of the formula Ia.1 in which X is CH, R^{8a} is CH₂CF₂CHF₂ and R⁵, R⁶ and R⁷ for a compound corresponds in each case to one row of Table A

### Table 13

Compounds of the formula Ia.1 in which X is CH, R^{8a} is CH₂CF₂CF₃ and R⁵, R⁶ and R⁷ for a compound corresponds in each case to one row of Table A

### Table 14

Compounds of the formula Ia.1 in which X is CH, R^{8a} is CH(CH₃)CF₃ and R⁵, R⁶ and R⁷ for a compound corresponds in each case to one row of Table A

### Table 15

Compounds of the formula Ia.1 in which X is CH, R^{8a} is CF(CH₃)CF₃ and R⁵, R⁶ and R⁷ for a compound corresponds in each case to one row of Table A

### Table 16

Compounds of the formula Ia.1 in which X is CH, R^{8a} is CH(CF₃)₂ and R⁵, R⁶ and R⁷ for a compound corresponds in each case to one row of Table A

### Table 17

Compounds of the formula Ia.1 in which X is CH, R^{8a} is CF(CF₃)₂ and R⁵, R⁶ and R⁷ for a compound corresponds in each case to one row of Table A

### Table 18

Compounds of the formula Ia.1 in which X is CH, R^{8a} is CH₂CH₂CH₂CHF₂ and R⁵, R⁶ and R⁷ for a compound corresponds in each case to one row of Table A

### Table 19

Compounds of the formula Ia.1 in which X is CH, R^{8a} is CH₂CH₂CH₂CF₃ and R⁵, R⁶ and R⁷ for a compound corresponds in each case to one row of Table A

### Table 20

Compounds of the formula Ia.1 in which X is CH, R^{8a} is CH₂CH₂CF₂CH₃ and R⁵, R⁶ and R⁷ for a compound corresponds in each case to one row of Table A

### Table 21

Compounds of the formula Ia.1 in which X is CH, R^{8a} is CH₂CH₂CF₂CF₃ and R⁵, R⁶ and R⁷ for a compound corresponds in each case to one row of Table A

### Table 22

Compounds of the formula Ia.1 in which X is CH, R^{8a} is CH₂CF₂CF₂CF₃ and R⁵, R⁶ and R⁷ for a compound corresponds in each case to one row of Table A

### Table 23

Compounds of the formula Ia.1 in which X is CH, R^{8a} is CH(CH₃)CF₂CHF₂ and R⁵, R⁶ and R⁷ for a compound corresponds in each case to one row of Table A

### Table 24

Compounds of the formula Ia.1 in which X is CH, R^{8a} is CH(CH₃)CF₂CF₃ and R⁵, R⁶ and R⁷ for a compound corresponds in each case to one row of Table A

### Table 25

Compounds of the formula Ia.1 in which X is CH, R^{8a} is CH₂CH(CH₃)CH₂CHF₂ and R⁵, R⁶ and R⁷ for a compound corresponds in each case to one row of Table A

### Table 26

Compounds of the formula Ia.1 in which X is CH, R^{8a} is CH₂CH(CH₃)CH₂CF₃ and R⁵, R⁶ and R⁷ for a compound corresponds in each case to one row of Table A

### Table 27

Compounds of the formula Ia.1 in which X is CH, R^{8a} is CH₂CH(CH₃)CF₂CF₃ and R⁵, R⁶ and R⁷ for a compound corresponds in each case to one row of Table A

### Table 28

Compounds of the formula Ia.1 in which X is CH, R^{8a} is CH₂CF(CF₃)₂ and R⁵, R⁶ and R⁷ for a compound corresponds in each case to one row of Table A

### Table 29

Compounds of the formula Ia.1 in which X is CH, R^{8a} is C(CH₃)(CF₃)₂ and R⁵, R⁶ and R⁷ for a compound corresponds in each case to one row of Table A

### Table 30

Compounds of the formula Ia.1 in which X is CH, R^{8a} is C(CF₃)(CH₃)₂ and R⁵, R⁶ and R⁷ for a compound corresponds in each case to one row of Table A

### Table 31

Compounds of the formula Ia.1 in which X is CH, R^{8a} is C(CF₃)(CF₃)₂ and R⁵, R⁶ and R⁷ for a compound corresponds in each case to one row of Table A

### Table 32

Compounds of the formula Ia.1 in which X is CH, R^{8a} is A.1 and R⁵, R⁶ and R⁷ for a compound corresponds in each case to one row of Table A

### Table 33

Compounds of the formula Ia.1 in which X is CH, R^{8a} is A.2 and R⁵, R⁶ and R⁷ for a compound corresponds in each case to one row of Table A

### Table 34

Compounds of the formula Ia.1 in which X is CH, R^{8a} is A.3 and R⁵, R⁶ and R⁷ for a compound corresponds in each case to one row of Table A

### Table 35

Compounds of the formula Ia.1 in which X is CH, R^{8a} is A.4 and R⁵, R⁶ and R⁷ for a compound corresponds in each case to one row of Table A

### Table 36

Compounds of the formula Ia.1 in which X is CH, R^{8a} is A.5 and R⁵, R⁶ and R⁷ for a compound corresponds in each case to one row of Table A

### Table 37

Compounds of the formula Ia.1 in which X is CH, R^{8a} is A.6 and R⁵, R⁶ and R⁷ for a compound corresponds in each case to one row of Table A

### Table 38

Compounds of the formula Ia.1 in which X is CH, R^{8a} is A.7 and R⁵, R⁶ and R⁷ for a compound corresponds in each case to one row of Table A

### Table 39

Compounds of the formula Ia.1 in which X is CH, R^{8a} is A.8 and R⁵, R⁶ and R⁷ for a compound corresponds in each case to one row of Table A

### Table 40

Compounds of the formula Ia.1 in which X is CH, R^{8a} is A.9 and R⁵, R⁶ and R⁷ for a compound corresponds in each case to one row of Table A

### Table 41

Compounds of the formula Ia.1 in which X is CH, R^{8a} is A.10 and R⁵, R⁶ and R⁷ for a compound corresponds in each case to one row of Table A

### Table 42

Compounds of the formula Ia.1 in which X is CH, R^{8a} is A.11 and R⁵, R⁶ and R⁷ for a compound corresponds in each case to one row of Table A

### Table 43

Compounds of the formula Ia.1 in which X is CH, R^{8a} is A.12 and R⁵, R⁶ and R⁷ for a compound corresponds in each case to one row of Table A

### Table 44

Compounds of the formula Ia.1 in which X is CH, R^{8a} is A.13 and R⁵, R⁶ and R⁷ for a compound corresponds in each case to one row of Table A

### Table 45

Compounds of the formula Ia.1 in which X is CH, R^{8a} is A.14 and R⁵, R⁶ and R⁷ for a compound corresponds in each case to one row of Table A

### Table 46

Compounds of the formula Ia.1 in which X is CH, R^{8a} is A.15 and R⁵, R⁶ and R⁷ for a compound corresponds in each case to one row of Table A

### Table 47

Compounds of the formula Ia.1 in which X is CH, R^{8a} is A.16 and R⁵, R⁶ and R⁷ for a compound corresponds in each case to one row of Table A

### Table 48

Compounds of the formula Ia.1 in which X is CH, R^{8a} is A.17 and R⁵, R⁶ and R⁷ for a compound corresponds in each case to one row of Table A

### Table 49

Compounds of the formula Ia.1 in which X is CH, R^{8a} is A.18 and R⁵, R⁶ and R⁷ for a compound corresponds in each case to one row of Table A

### Table 50

Compounds of the formula Ia.1 in which X is CH, R^{8a} is A.19 and R⁵, R⁶ and R⁷ for a compound corresponds in each case to one row of Table A

### Table 51

Compounds of the formula Ia.1 in which X is CH, R^{8a} is A.20 and R⁵, R⁶ and R⁷ for a compound corresponds in each case to one row of Table A

### Table 52

Compounds of the formula Ia.1 in which X is CH, R^{8a} is A.21 and R⁵, R⁶ and R⁷ for a compound corresponds in each case to one row of Table A

### Table 53

Compounds of the formula Ia.1 in which X is CH, R^{8a} is A.22 and R⁵, R⁶ and R⁷ for a compound corresponds in each case to one row of Table A

### Table 54

Compounds of the formula Ia.1 in which X is CH, R^{8a} is A.23 and R⁵, R⁶ and R⁷ for a compound corresponds in each case to one row of Table A

### Table 55

Compounds of the formula Ia.1 in which X is CH, R^{8a} is A.24 and R⁵, R⁶ and R⁷ for a compound corresponds in each case to one row of Table A

### Table 56

Compounds of the formula Ia.1 in which X is CH, R^{8a} is A.25 and R⁵, R⁶ and R⁷ for a compound corresponds in each case to one row of Table A

### Table 57

Compounds of the formula Ia.1 in which X is CH, R^{8a} is A.26 and R⁵, R⁶ and R⁷ for a compound corresponds in each case to one row of Table A

### Table 58

Compounds of the formula Ia.1 in which X is CH, R^{8a} is A.27 and R⁵, R⁶ and R⁷ for a compound corresponds in each case to one row of Table A

### Table 59

Compounds of the formula Ia.1 in which X is CH, R^{8a} is A.28 and R⁵, R⁶ and R⁷ for a compound corresponds in each case to one row of Table A

### Table 60

Compounds of the formula Ia.1 in which X is CH, R^{8a} is A.29 and R⁵, R⁶ and R⁷ for a compound corresponds in each case to one row of Table A

### Table 61

Compounds of the formula Ia.1 in which X is CH, R^{8a} is A.30 and R⁵, R⁶ and R⁷ for a compound corresponds in each case to one row of Table A

### Table 62

Compounds of the formula Ia.1 in which X is CH, R^{8a} is A.31 and R⁵, R⁶ and R⁷ for a compound corresponds in each case to one row of Table A

### Table 63

Compounds of the formula Ia.1 in which X is CH, R^{8a} is A.32 and R⁵, R⁶ and R⁷ for a compound corresponds in each case to one row of Table A

### Table 64

Compounds of the formula Ia.1 in which X is CH, R^{8a} is A.33 and R⁵, R⁶ and R⁷ for a compound corresponds in each case to one row of Table A

### Table 65

Compounds of the formula Ia.1 in which X is CH, R^{8a} is A.34 and R⁵, R⁶ and R⁷ for a compound corresponds in each case to one row of Table A

### Table 66

Compounds of the formula Ia.1 in which X is CH, R^{8a} is A.35 and R⁵, R⁶ and R⁷ for a compound corresponds in each case to one row of Table A

### Table 67

Compounds of the formula Ia.1 in which X is CH, R^{8a} is A.36 and R⁵, R⁶ and R⁷ for a compound corresponds in each case to one row of Table A

### Table 68

Compounds of the formula Ia.1 in which X is CH, R^{8a} is A.37 and R⁵, R⁶ and R⁷ for a compound corresponds in each case to one row of Table A

### Table 69

Compounds of the formula Ia.1 in which X is CH, R^{8a} is A.38 and R⁵, R⁶ and R⁷ for a compound corresponds in each case to one row of Table A

### Table 70

Compounds of the formula Ia.1 in which X is CH, R^{8a} is A.39 and R⁵, R⁶ and R⁷ for a compound corresponds in each case to one row of Table A

### Table 71

Compounds of the formula Ia.1 in which X is CH, R^{8a} is A.40 and R⁵, R⁶ and R⁷ for a compound corresponds in each case to one row of Table A

### Table 72

Compounds of the formula Ia.1 in which X is CH, R^{8a} is A.41 and R⁵, R⁶ and R⁷ for a compound corresponds in each case to one row of Table A

### Table 73

Compounds of the formula Ia.1 in which X is CH, R^{8a} is A.42 and R⁵, R⁶ and R⁷ for a compound corresponds in each case to one row of Table A

### Table 74

Compounds of the formula Ia.1 in which X is CH, R^{8a} is A.43 and R⁵, R⁶ and R⁷ for a compound corresponds in each case to one row of Table A

### Table 75

Compounds of the formula Ia.1 in which X is CH, R^{8a} is A.44 and R⁵, R⁶ and R⁷ for a compound corresponds in each case to one row of Table A

### Table 76

Compounds of the formula Ia.1 in which X is CH, R^{8a} is A.45 and R⁵, R⁶ and R⁷ for a compound corresponds in each case to one row of Table A

### Table 77

Compounds of the formula Ia.1 in which X is CH, R^{8a} is A.46 and R⁵, R⁶ and R⁷ for a compound corresponds in each case to one row of Table A

### Table 78

Compounds of the formula Ia.1 in which X is CH, R^{8a} is A.47 and R⁵, R⁶ and R⁷ for a compound corresponds in each case to one row of Table A

### Table 79

Compounds of the formula Ia.1 in which X is CH, R^{8a} is A.48 and R⁵, R⁶ and R⁷ for a compound corresponds in each case to one row of Table A

### Table 80

Compounds of the formula Ia.1 in which X is CH, R^{8a} is A.49 and R⁵, R⁶ and R⁷ for a compound corresponds in each case to one row of Table A

### Table 81

Compounds of the formula Ia.1 in which X is CH, R^{8a} is A.50 and R⁵, R⁶ and R⁷ for a compound corresponds in each case to one row of Table A

### Table 82

Compounds of the formula Ia.1 in which X is CH, R^{8a} is A.51 and R⁵, R⁶ and R⁷ for a compound corresponds in each case to one row of Table A

### Table 83

Compounds of the formula Ia.1 in which X is CH, R^{8a} is A.52 and R⁵, R⁶ and R⁷ for a compound corresponds in each case to one row of Table A

### Table 84

Compounds of the formula Ia.1 in which X is CH, R^{8a} is A.53 and R⁵, R⁶ and R⁷ for a compound corresponds in each case to one row of Table A

### Table 85

Compounds of the formula Ia.1 in which X is CH, R^{8a} is A.54 and R⁵, R⁶ and R⁷ for a compound corresponds in each case to one row of Table A

### Table 86

Compounds of the formula Ia.1 in which X is CH, R^{8a} is A.55 and R⁵, R⁶ and R⁷ for a compound corresponds in each case to one row of Table A

### Table 87

Compounds of the formula Ia.1 in which X is CH, R^{8a} is A.56 and R⁵, R⁶ and R⁷ for a compound corresponds in each case to one row of Table A

### Table 88

Compounds of the formula Ia.1 in which X is CH, R^{8a} is A.57 and R⁵, R⁶ and R⁷ for a compound corresponds in each case to one row of Table A

### Table 89

Compounds of the formula Ia.1 in which X is CH, R^{8a} is A.58 and R⁵, R⁶ and R⁷ for a compound corresponds in each case to one row of Table A

### Table 90

Compounds of the formula Ia.1 in which X is CH, R^{8a} is A.59 and R⁵, R⁶ and R⁷ for a compound corresponds in each case to one row of Table A

### Table 91

Compounds of the formula Ia.1 in which X is CH, R^{8a} is A.60 and R⁵, R⁶ and R⁷ for a compound corresponds in each case to one row of Table A

### Table 92

Compounds of the formula Ia.1 in which X is CH, R^{8a} is A.61 and R⁵, R⁶ and R⁷ for a compound corresponds in each case to one row of Table A

### Table 93

Compounds of the formula Ia.1 in which X is CH, R^{8a} is A.62 and R⁵, R⁶ and R⁷ for a compound corresponds in each case to one row of Table A

### Table 94

Compounds of the formula Ia.1 in which X is CH, R^{8a} is A.63 and R⁵, R⁶ and R⁷ for a compound corresponds in each case to one row of Table A

### Table 95

Compounds of the formula Ia.1 in which X is CH, R^{8a} is A.64 and R⁵, R⁶ and R⁷ for a compound corresponds in each case to one row of Table A

### Table 96

Compounds of the formula Ia.1 in which X is CH, R^{8a} is A.65 and R⁵, R⁶ and R⁷ for a compound corresponds in each case to one row of Table A

### Table 97

Compounds of the formula Ia.1 in which X is CH, R^{8a} is A.66 and R⁵, R⁶ and R⁷ for a compound corresponds in each case to one row of Table A

### Table 98

Compounds of the formula Ia.1 in which X is CH, R^{8a} is A.67 and R⁵, R⁶ and R⁷ for a compound corresponds in each case to one row of Table A

### Table 99

Compounds of the formula Ia.1 in which X is CH, R^{8a} is A.68 and R⁵, R⁶ and R⁷ for a compound corresponds in each case to one row of Table A

### Table 100

Compounds of the formula Ia.1 in which X is CH, R^{8a} is A.69 and R⁵, R⁶ and R⁷ for a compound corresponds in each case to one row of Table A

### Table 101

Compounds of the formula Ia.1 in which X is CH, R^{8a} is A.70 and R⁵, R⁶ and R⁷ for a compound corresponds in each case to one row of Table A

### Table 102

Compounds of the formula Ia.1 in which X is CH, R^{8a} is A.71 and R⁵, R⁶ and R⁷ for a compound corresponds in each case to one row of Table A

### Table 103

Compounds of the formula Ia.1 in which X is CH, R^{8a} is A.72 and R⁵, R⁶ and R⁷ for a compound corresponds in each case to one row of Table A

### Table 104

Compounds of the formula Ia.1 in which X is CH, R^{8a} is A.73 and R⁵, R⁶ and R⁷ for a compound corresponds in each case to one row of Table A

### Table 105

Compounds of the formula Ia.1 in which X is CH, R^{8a} is A.74 and R⁵, R⁶ and R⁷ for a compound corresponds in each case to one row of Table A

### Table 106

Compounds of the formula Ia.1 in which X is CH, R^{8a} is A.75 and R⁵, R⁶ and R⁷ for a compound corresponds in each case to one row of Table A

### Table 107

Compounds of the formula Ia.1 in which X is CH, R^{8a} is A.76 and R⁵, R⁶ and R⁷ for a compound corresponds in each case to one row of Table A

### Table 108

Compounds of the formula Ia.1 in which X is CH, R^{8a} is A.77 and R⁵, R⁶ and R⁷ for a compound corresponds in each case to one row of Table A

### Table 109

Compounds of the formula Ia.1 in which X is CH, R^{8a} is A.78 and R⁵, R⁶ and R⁷ for a compound corresponds in each case to one row of Table A

### Table 110

Compounds of the formula Ia.1 in which X is CH, R^{8a} is A.79 and R⁵, R⁶ and R⁷ for a compound corresponds in each case to one row of Table A

### Table 111

Compounds of the formula Ia.1 in which X is CH, R^{8a} is A.80 and R⁵, R⁶ and R⁷ for a compound corresponds in each case to one row of Table A

### Table 112

Compounds of the formula Ia.1 in which X is CH, R^{8a} is A.81 and R⁵, R⁶ and R⁷ for a compound corresponds in each case to one row of Table A

### Table 113

Compounds of the formula Ia.1 in which X is CH, R^{8a} is A.82 and R⁵, R⁶ and R⁷ for a compound corresponds in each case to one row of Table A

### Table 114

Compounds of the formula Ia.1 in which X is CH, R^{8a} is A.83 and R⁵, R⁶ and R⁷ for a compound corresponds in each case to one row of Table A

### Table 115

Compounds of the formula Ia.1 in which X is CH, R^{8a} is A.84 and R⁵, R⁶ and R⁷ for a compound corresponds in each case to one row of Table A

### Table 116

Compounds of the formula Ia.1 in which X is CH, R^{8a} is A.85 and R⁵, R⁶ and R⁷ for a compound corresponds in each case to one row of Table A

### Table 117

Compounds of the formula Ia.1 in which X is CH, R^{8a} is A.86 and R⁵, R⁶ and R⁷ for a compound corresponds in each case to one row of Table A

### Table 118

Compounds of the formula Ia.1 in which X is CH, R^{8a} is A.87 and R⁵, R⁶ and R⁷ for a compound corresponds in each case to one row of Table A

### Table 119

Compounds of the formula Ia.1 in which X is CH, R^{8a} is A.88 and R⁵, R⁶ and R⁷ for a compound corresponds in each case to one row of Table A

### Table 120

Compounds of the formula Ia.1 in which X is CH, R^{8a} is A.89 and R⁵, R⁶ and R⁷ for a compound corresponds in each case to one row of Table A

### Table 121

Compounds of the formula Ia.1 in which X is CH, R^{8a} is A.90 and R⁵, R⁶ and R⁷ for a compound corresponds in each case to one row of Table A

### Table 122

Compounds of the formula Ia.1 in which X is CH, R^{8a} is A.91 and R⁵, R⁶ and R⁷ for a compound corresponds in each case to one row of Table A

### Table 123

Compounds of the formula Ia.1 in which X is CH, R^{8a} is adamantan-1-yl and R⁵, R⁶ and R⁷ for a compound corresponds in each case to one row of Table A

### Table 124

Compounds of the formula Ia.1 in which X is CH, R^{8a} is —CH₂-A.1 and R⁵, R⁶ and R⁷ for a compound corresponds in each case to one row of Table A

### Table 125

Compounds of the formula Ia.1 in which X is CH, R^{8a} is —CH₂-A.2 and R⁵, R⁶ and R⁷ for a compound corresponds in each case to one row of Table A

### Table 126

Compounds of the formula Ia.1 in which X is CH, R^{8a} is —CH₂-A.3 and R⁵, R⁶ and R⁷ for a compound corresponds in each case to one row of Table A

### Table 127

Compounds of the formula Ia.1 in which X is CH, R^{8a} is —CH₂-A.4 and R⁵, R⁶ and R⁷ for a compound corresponds in each case to one row of Table A

### Table 128

Compounds of the formula Ia.1 in which X is CH, R^{8a} is —CH₂-A.5 and R⁵, R⁶ and R⁷ for a compound corresponds in each case to one row of Table A

### Table 129

Compounds of the formula Ia.1 in which X is CH, R^{8a} is —CH₂-A.6 and R⁵, R⁶ and R⁷ for a compound corresponds in each case to one row of Table A

### Table 130

Compounds of the formula Ia.1 in which X is CH, R^{8a} is —CH₂-A.7 and R⁵, R⁶ and R⁷ for a compound corresponds in each case to one row of Table A

### Table 131

Compounds of the formula Ia.1 in which X is CH, R^{8a} is —CH₂-A.8 and R⁵, R⁶ and R⁷ for a compound corresponds in each case to one row of Table A

### Table 132

Compounds of the formula Ia.1 in which X is CH, R^{8a} is —CH₂-A.9 and R⁵, R⁶ and R⁷ for a compound corresponds in each case to one row of Table A

### Table 133

Compounds of the formula Ia.1 in which X is CH, R^{8a} is —CH₂-A.10 and R⁵, R⁶ and R⁷ for a compound corresponds in each case to one row of Table A

### Table 134

Compounds of the formula Ia.1 in which X is CH, R^{8a} is —CH₂-A.11 and R⁵, R⁶ and R⁷ for a compound corresponds in each case to one row of Table A

### Table 135

Compounds of the formula Ia.1 in which X is CH, R^{8a} is —CH₂-A.12 and R⁵, R⁶ and R⁷ for a compound corresponds in each case to one row of Table A

### Table 136

Compounds of the formula Ia.1 in which X is CH, R^{8a} is —CH₂-A.13 and R⁵, R⁶ and R⁷ for a compound corresponds in each case to one row of Table A

### Table 137

Compounds of the formula Ia.1 in which X is CH, R^{8a} is —CH₂-A.14 and R⁵, R⁶ and R⁷ for a compound corresponds in each case to one row of Table A

### Table 138

Compounds of the formula Ia.1 in which X is CH, R^{8a} is —CH₂-A.15 and R⁵, R⁶ and R⁷ for a compound corresponds in each case to one row of Table A

### Table 139

Compounds of the formula Ia.1 in which X is CH, R^{8a} is —CH₂-A.16 and R⁵, R⁶ and R⁷ for a compound corresponds in each case to one row of Table A

### Table 140

Compounds of the formula Ia.1 in which X is CH, R^{8a} is —CH₂-A.17 and R⁵, R⁶ and R⁷ for a compound corresponds in each case to one row of Table A

### Table 141

Compounds of the formula Ia.1 in which X is CH, R^{8a} is —CH₂-A.18 and R⁵, R⁶ and R⁷ for a compound corresponds in each case to one row of Table A

### Table 142

Compounds of the formula Ia.1 in which X is CH, R^{8a} is —CH₂-A.19 and R⁵, R⁶ and R⁷ for a compound corresponds in each case to one row of Table A

### Table 143

Compounds of the formula Ia.1 in which X is CH, R^{8a} is —CH₂-A.20 and R⁵, R⁶ and R⁷ for a compound corresponds in each case to one row of Table A

### Table 144

Compounds of the formula Ia.1 in which X is CH, R^{8a} is —CH₂-A.21 and R⁵, R⁶ and R⁷ for a compound corresponds in each case to one row of Table A

### Table 145

Compounds of the formula Ia.1 in which X is CH, R^{8a} is —CH₂-A.22 and R⁵, R⁶ and R⁷ for a compound corresponds in each case to one row of Table A

### Table 146

Compounds of the formula Ia.1 in which X is CH, R^{8a} is —CH₂-A.23 and R⁵, R⁶ and R⁷ for a compound corresponds in each case to one row of Table A

### Table 147

Compounds of the formula Ia.1 in which X is CH, R^{8a} is —CH₂-A.24 and R⁵, R⁶ and R⁷ for a compound corresponds in each case to one row of Table A

### Table 148

Compounds of the formula Ia.1 in which X is CH, R^{8a} is —CH₂-A.25 and R⁵, R⁶ and R⁷ for a compound corresponds in each case to one row of Table A

### Table 149

Compounds of the formula Ia.1 in which X is CH, R^{8a} is —CH₂-A.26 and R⁵, R⁶ and R⁷ for a compound corresponds in each case to one row of Table A

### Table 150

Compounds of the formula Ia.1 in which X is CH, R^{8a} is —CH₂-A.27 and R⁵, R⁶ and R⁷ for a compound corresponds in each case to one row of Table A

### Table 151

Compounds of the formula Ia.1 in which X is CH, R^{8a} is —CH₂-A.28 and R⁵, R⁶ and R⁷ for a compound corresponds in each case to one row of Table A

### Table 152

Compounds of the formula Ia.1 in which X is CH, R^{8a} is —CH₂-A.29 and R⁵, R⁶ and R⁷ for a compound corresponds in each case to one row of Table A

### Table 153

Compounds of the formula Ia.1 in which X is CH, R^{8a} is —CH₂-A.30 and R⁵, R⁶ and R⁷ for a compound corresponds in each case to one row of Table A

### Table 154

Compounds of the formula Ia.1 in which X is CH, R^{8a} is —CH₂-A.31 and R⁵, R⁶ and R⁷ for a compound corresponds in each case to one row of Table A

### Table 155

Compounds of the formula Ia.1 in which X is CH, R^{8a} is —CH₂-A.32 and R⁵, R⁶ and R⁷ for a compound corresponds in each case to one row of Table A

### Table 156

Compounds of the formula Ia.1 in which X is CH, R^{8a} is —CH₂-A.33 and R⁵, R⁶ and R⁷ for a compound corresponds in each case to one row of Table A

### Table 157

Compounds of the formula Ia.1 in which X is CH, R^{8a} is —CH₂-A.34 and R⁵, R⁶ and R⁷ for a compound corresponds in each case to one row of Table A

### Table 158

Compounds of the formula Ia.1 in which X is CH, R^{8a} is —CH₂-A.35 and R⁵, R⁶ and R⁷ for a compound corresponds in each case to one row of Table A

### Table 159

Compounds of the formula Ia.1 in which X is CH, R^{8a} is —CH₂-A.36 and R⁵, R⁶ and R⁷ for a compound corresponds in each case to one row of Table A

### Table 160

Compounds of the formula Ia.1 in which X is CH, R^{8a} is —CH₂-A.37 and R⁵, R⁶ and R⁷ for a compound corresponds in each case to one row of Table A

### Table 161

Compounds of the formula Ia.1 in which X is CH, R^{8a} is —CH₂-A.38 and R⁵, R⁶ and R⁷ for a compound corresponds in each case to one row of Table A

### Table 162

Compounds of the formula Ia.1 in which X is CH, R^{8a} is —CH₂-A.39 and R⁵, R⁶ and R⁷ for a compound corresponds in each case to one row of Table A

### Table 163

Compounds of the formula Ia.1 in which X is CH, R^{8a} is —CH₂-A.40 and R⁵, R⁶ and R⁷ for a compound corresponds in each case to one row of Table A

### Table 164

Compounds of the formula Ia.1 in which X is CH, R^{8a} is —CH₂-A.41 and R⁵, R⁶ and R⁷ for a compound corresponds in each case to one row of Table A

### Table 165

Compounds of the formula Ia.1 in which X is CH, R^{8a} is —CH₂-A.42 and R⁵, R⁶ and R⁷ for a compound corresponds in each case to one row of Table A

### Table 166

Compounds of the formula Ia.1 in which X is CH, R^{8a} is —CH₂-A.43 and R⁵, R⁶ and R⁷ for a compound corresponds in each case to one row of Table A

### Table 167

Compounds of the formula Ia.1 in which X is CH, R^{8a} is —CH₂-A.44 and R⁵, R⁶ and R⁷ for a compound corresponds in each case to one row of Table A

### Table 168

Compounds of the formula Ia.1 in which X is CH, R^{8a} is —CH₂-A.45 and R⁵, R⁶ and R⁷ for a compound corresponds in each case to one row of Table A

### Table 169

Compounds of the formula Ia.1 in which X is CH, R^{8a} is —CH₂-A.46 and R⁵, R⁶ and R⁷ for a compound corresponds in each case to one row of Table A

### Table 170

Compounds of the formula Ia.1 in which X is CH, R^{8a} is —CH₂-A.47 and R⁵, R⁶ and R⁷ for a compound corresponds in each case to one row of Table A

### Table 171

Compounds of the formula Ia.1 in which X is CH, R^{8a} is —CH₂-A.48 and R⁵, R⁶ and R⁷ for a compound corresponds in each case to one row of Table A

### Table 172

Compounds of the formula Ia.1 in which X is CH, R^{8a} is —CH₂-A.49 and R⁵, R⁶ and R⁷ for a compound corresponds in each case to one row of Table A

### Table 173

Compounds of the formula Ia.1 in which X is CH, R^{8a} is —CH₂-A.50 and R⁵, R⁶ and R⁷ for a compound corresponds in each case to one row of Table A

### Table 174

Compounds of the formula Ia.1 in which X is CH, R^{8a} is —CH₂-A.51 and R⁵, R⁶ and R⁷ for a compound corresponds in each case to one row of Table A

### Table 175

Compounds of the formula Ia.1 in which X is CH, R^{8a} is —CH₂-A.52 and R⁵, R⁶ and R⁷ for a compound corresponds in each case to one row of Table A

### Table 176

Compounds of the formula Ia.1 in which X is CH, R^{8a} is —CH₂-A.53 and R⁵, R⁶ and R⁷ for a compound corresponds in each case to one row of Table A

### Table 177

Compounds of the formula Ia.1 in which X is CH, R^{8a} is —CH₂-A.54 and R⁵, R⁶ and R⁷ for a compound corresponds in each case to one row of Table A

### Table 178

Compounds of the formula Ia.1 in which X is CH, R^{8a} is —CH₂-A.55 and R⁵, R⁶ and R⁷ for a compound corresponds in each case to one row of Table A

### Table 179

Compounds of the formula Ia.1 in which X is CH, R^{8a} is —CH₂-A.56 and R⁵, R⁶ and R⁷ for a compound corresponds in each case to one row of Table A

### Table 180

Compounds of the formula Ia.1 in which X is CH, R^{8a} is —CH₂-A.57 and R⁵, R⁶ and R⁷ for a compound corresponds in each case to one row of Table A

### Table 181

Compounds of the formula Ia.1 in which X is CH, R^{8a} is —CH₂-A.58 and R⁵, R⁶ and R⁷ for a compound corresponds in each case to one row of Table A

### Table 182

Compounds of the formula Ia.1 in which X is CH, R^{8a} is —CH₂-A.59 and R⁵, R⁶ and R⁷ for a compound corresponds in each case to one row of Table A

### Table 183

Compounds of the formula Ia.1 in which X is CH, R^{8a} is —CH₂-A.60 and R⁵, R⁶ and R⁷ for a compound corresponds in each case to one row of Table A

### Table 184

Compounds of the formula Ia.1 in which X is CH, R^{8a} is —CH₂-A.61 and R⁵, R⁶ and R⁷ for a compound corresponds in each case to one row of Table A

### Table 185

Compounds of the formula Ia.1 in which X is CH, R^{8a} is —CH₂-A.62 and R⁵, R⁶ and R⁷ for a compound corresponds in each case to one row of Table A

### Table 186

Compounds of the formula Ia.1 in which X is CH, R^{8a} is —CH₂-A.63 and R⁵, R⁶ and R⁷ for a compound corresponds in each case to one row of Table A

### Table 187

Compounds of the formula Ia.1 in which X is CH, R^{8a} is —CH₂-A.64 and R⁵, R⁶ and R⁷ for a compound corresponds in each case to one row of Table A

### Table 188

Compounds of the formula Ia.1 in which X is CH, R^{8a} is —CH₂-A.65 and R⁵, R⁶ and R⁷ for a compound corresponds in each case to one row of Table A

### Table 189

Compounds of the formula Ia.1 in which X is CH, R^{8a} is —CH₂-A.66 and R⁵, R⁶ and R⁷ for a compound corresponds in each case to one row of Table A

### Table 190

Compounds of the formula Ia.1 in which X is CH, R^{8a} is —CH₂-A.67 and R⁵, R⁶ and R⁷ for a compound corresponds in each case to one row of Table A

### Table 191

Compounds of the formula Ia.1 in which X is CH, R^{8a} is —CH₂-A.68 and R⁵, R⁶ and R⁷ for a compound corresponds in each case to one row of Table A

### Table 192

Compounds of the formula Ia.1 in which X is CH, R^{8a} is —CH₂-A.69 and R⁵, R⁶ and R⁷ for a compound corresponds in each case to one row of Table A

### Table 193

Compounds of the formula Ia.1 in which X is CH, R^{8a} is —CH₂-A.70 and R⁵, R⁶ and R⁷ for a compound corresponds in each case to one row of Table A

### Table 194

Compounds of the formula Ia.1 in which X is CH, R^{8a} is —CH₂-A.71 and R⁵, R⁶ and R⁷ for a compound corresponds in each case to one row of Table A

### Table 195

Compounds of the formula Ia.1 in which X is CH, R^{8a} is —CH₂-A.72 and R⁵, R⁶ and R⁷ for a compound corresponds in each case to one row of Table A

### Table 196

Compounds of the formula Ia.1 in which X is CH, R^{8a} is —CH₂-A.73 and R⁵, R⁶ and R⁷ for a compound corresponds in each case to one row of Table A

### Table 197

Compounds of the formula Ia.1 in which X is CH, R^{8a} is —CH₂-A.74 and R⁵, R⁶ and R⁷ for a compound corresponds in each case to one row of Table A

### Table 198

Compounds of the formula Ia.1 in which X is CH, R^{8a} is —CH₂-A.75 and R⁵, R⁶ and R⁷ for a compound corresponds in each case to one row of Table A

### Table 199

Compounds of the formula Ia.1 in which X is CH, R^{8a} is —CH₂-A.76 and R⁵, R⁶ and R⁷ for a compound corresponds in each case to one row of Table A

### Table 200

Compounds of the formula Ia.1 in which X is CH, R^{8a} is —CH₂-A.77 and R⁵, R⁶ and R⁷ for a compound corresponds in each case to one row of Table A

### Table 201

Compounds of the formula Ia.1 in which X is CH, R^{8a} is —CH₂-A.78 and R⁵, R⁶ and R⁷ for a compound corresponds in each case to one row of Table A

### Table 202

Compounds of the formula Ia.1 in which X is CH, R^{8a} is —CH₂-A.79 and R⁵, R⁶ and R⁷ for a compound corresponds in each case to one row of Table A

### Table 203

Compounds of the formula Ia.1 in which X is CH, R^{8a} is —CH₂-A.80 and R⁵, R⁶ and R⁷ for a compound corresponds in each case to one row of Table A

### Table 204

Compounds of the formula Ia.1 in which X is CH, R^{8a} is —CH₂-A.81 and R⁵, R⁶ and R⁷ for a compound corresponds in each case to one row of Table A

### Table 205

Compounds of the formula Ia.1 in which X is CH, R^{8a} is —CH₂-A.82 and R⁵, R⁶ and R⁷ for a compound corresponds in each case to one row of Table A

### Table 206

Compounds of the formula Ia.1 in which X is CH, R^{8a} is —CH₂-A.83 and R⁵, R⁶ and R⁷ for a compound corresponds in each case to one row of Table A

### Table 207

Compounds of the formula Ia.1 in which X is CH, R^{8a} is —CH₂-A.84 and R⁵, R⁶ and R⁷ for a compound corresponds in each case to one row of Table A

### Table 208

Compounds of the formula Ia.1 in which X is CH, R^{8a} is —CH₂-A.85 and R⁵, R⁶ and R⁷ for a compound corresponds in each case to one row of Table A

### Table 209

Compounds of the formula Ia.1 in which X is CH, R^{8a} is —CH₂-A.86 and R⁵, R⁶ and R⁷ for a compound corresponds in each case to one row of Table A

### Table 210

Compounds of the formula Ia.1 in which X is CH, R^{8a} is —CH₂-A.87 and R⁵, R⁶ and R⁷ for a compound corresponds in each case to one row of Table A

### Table 211

Compounds of the formula Ia.1 in which X is CH, R^{8a} is —CH₂-A.88 and R⁵, R⁶ and R⁷ for a compound corresponds in each case to one row of Table A

### Table 212

Compounds of the formula Ia.1 in which X is CH, R^{8a} is —CH₂-A.89 and R⁵, R⁶ and R⁷ for a compound corresponds in each case to one row of Table A

### Table 213

Compounds of the formula Ia.1 in which X is CH, R^{8a} is —CH₂-A.90 and R⁵, R⁶ and R⁷ for a compound corresponds in each case to one row of Table A

### Table 214

Compounds of the formula Ia.1 in which X is CH, R^{8a} is —CH₂-A.91 and R⁵, R⁶ and R⁷ for a compound corresponds in each case to one row of Table A

### Table 215

Compounds of the formula Ia.1 in which X is CH, R^{8a} is —CH₂-adamantan-1-yl and R⁵, R⁶ and R⁷ for a compound corresponds in each case to one row of Table A

### Tables 216 to 430

Compounds of the formula Ia.1 in which X is N, R^{8a} is as defined in any of tables 1 to 215 and R⁵, R⁶ and R⁷ for a compound corresponds in each case to one row of Table A

### Tables 431 to 860

Compounds of the formula Ia.2 in which X and R^{8a} are as defined in any of tables 1 to 430 and R⁵, R⁶ and R⁷ for a compound corresponds in each case to one row of Table A

### Tables 861 to 1290

Compounds of the formula Ia.3 in which X and R^{8a} are as defined in any of tables 1 to 430 and R⁵, R⁶ and R⁷ for a compound corresponds in each case to one row of Table A

### Tables 1291 to 1720

Compounds of the formula Ia.4 in which X and R^{8a} are as defined in any of tables 1 to 430 and R⁵, R⁶ and R⁷ for a compound corresponds in each case to one row of Table A

### Tables 1721 to 2150

Compounds of the formula Ia.5 in which X and R^{8a} are as defined in any of tables 1 to 430 and R⁵, R⁶ and R⁷ for a compound corresponds in each case to one row of Table A

### Tables 2151 to 2580

Compounds of the formula Ia.6 in which X and R^{8a} are as defined in any of tables 1 to 430 and R⁵, R⁶ and R⁷ for a compound corresponds in each case to one row of Table A

### Tables 2581 to 3010

Compounds of the formula Ia.7 in which X and R^{8a} are as defined in any of tables 1 to 430 and R⁵, R⁶ and R⁷ for a compound corresponds in each case to one row of Table A

### Tables 3011 to 3440

Compounds of the formula Ia.8 in which X and R^{8a} are as defined in any of tables 1 to 430 and R⁵, R⁶ and R⁷ for a compound corresponds in each case to one row of Table A

### Tables 3441 to 3870

Compounds of the formula Ia.9 in which X and R^{8a} are as defined in any of tables 1 to 430 and R⁵, R⁶ and R⁷ for a compound corresponds in each case to one row of Table A

### Tables 3871 to 4300

Compounds of the formula Ia.10 in which X and R^{8a} are as defined in any of tables 1 to 430 and R⁵, R⁶ and R⁷ for a compound corresponds in each case to one row of Table A

### Tables 4301 to 4730

Compounds of the formula Ia.11 in which X and R^{8a} are as defined in any of tables 1 to 430 and R⁵, R⁶ and R⁷ for a compound corresponds in each case to one row of Table A

### Tables 4731 to 5160

Compounds of the formula Ia.12 in which X and R^{8a} are as defined in any of tables 1 to 430 and R⁵, R⁶ and R⁷ for a compound corresponds in each case to one row of Table A

### Tables 5161 to 5590

Compounds of the formula Ia.13 in which X and R^{8a} are as defined in any of tables 1 to 430 and R⁵, R⁶ and R⁷ for a compound corresponds in each case to one row of Table A

### Tables 5591 to 6020

Compounds of the formula Ia.14 in which X and R^{8a} are as defined in any of tables 1 to 430 and R⁵, R⁶ and R⁷ for a compound corresponds in each case to one row of Table A

### Tables 6021 to 6450

Compounds of the formula Ia.15 in which X and R^{8a} are as defined in any of tables 1 to 430 and R⁵, R⁶ and R⁷ for a compound corresponds in each case to one row of Table A

### Tables 6451 to 6880

Compounds of the formula Ia.16 in which X and R^{8a} are as defined in any of tables 1 to 430 and R⁵, R⁶ and R⁷ for a compound corresponds in each case to one row of Table A

### Tables 6881 to 7310

Compounds of the formula Ia.17 in which X and R^{8a} are as defined in any of tables 1 to 430 and R⁵, R⁶ and R⁷ for a compound corresponds in each case to one row of Table A

### Tables 7311 to 7740

Compounds of the formula Ia.18 in which X and R^{8a} are as defined in any of tables 1 to 430 and R⁵, R⁶ and R⁷ for a compound corresponds in each case to one row of Table A

### Tables 7741 to 8170

Compounds of the formula Ia.19 in which X and R^{8a} are as defined in any of tables 1 to 430 and R⁵, R⁶ and R⁷ for a compound corresponds in each case to one row of Table A

### Tables 8171 to 8600

Compounds of the formula Ia.20 in which X and R^{8a} are as defined in any of tables 1 to 430 and R⁵, R⁶ and R⁷ for a compound corresponds in each case to one row of Table A

### Tables 8601 to 9030

Compounds of the formula Ia.21 in which X and R^{8a} are as defined in any of tables 1 to 430 and R⁵, R⁶ and R⁷ for a compound corresponds in each case to one row of Table A

### Tables 9031 to 9460

Compounds of the formula Ia.22 in which X and R^{8a} are as defined in any of tables 1 to 430 and R⁵, R⁶ and R⁷ for a compound corresponds in each case to one row of Table A

### Table 9461

Compounds of the formula Ia.23 in which X is CH, R^{8b} is H, R^{8c} is A.1 and R⁵, R⁶ and R⁷ for a compound corresponds in each case to one row of Table A

### Table 9462

Compounds of the formula Ia.23 in which X is CH, R^{8b} is H, R^{8c} is A.2 and R⁵, R⁶ and R⁷ for a compound corresponds in each case to one row of Table A

### Table 9463

Compounds of the formula Ia.23 in which X is CH, R^{8b} is H, R^{8c} is A.3 and R⁵, R⁶ and R⁷ for a compound corresponds in each case to one row of Table A

### Table 9464

Compounds of the formula Ia.23 in which X is CH, R^{8b} is H, R^{8c} is A.4 and R⁵, R⁶ and R⁷ for a compound corresponds in each case to one row of Table A

### Table 9465

Compounds of the formula Ia.23 in which X is CH, R^{8b} is H, R^{8c} is A.5 and R⁵, R⁶ and R⁷ for a compound corresponds in each case to one row of Table A

### Table 9466

Compounds of the formula Ia.23 in which X is CH, R^{8b} is H, R^{8c} is A.6 and R⁵, R⁶ and R⁷ for a compound corresponds in each case to one row of Table A

### Table 9467

Compounds of the formula Ia.23 in which X is CH, R^{8b} is H, R^{8c} is A.7 and R⁵, R⁶ and R⁷ for a compound corresponds in each case to one row of Table A

### Table 9468

Compounds of the formula Ia.23 in which X is CH, R^{8b} is H, R^{8c} is A.8 and R⁵, R⁶ and R⁷ for a compound corresponds in each case to one row of Table A

### Table 9469

Compounds of the formula Ia.23 in which X is CH, R^{8b} is H, R^{8c} is A.9 and R⁵, R⁶ and R⁷ for a compound corresponds in each case to one row of Table A

### Table 9470

Compounds of the formula Ia.23 in which X is CH, R^{8b} is H, R^{8′} is A.10 and R⁵, R⁶ and R⁷ for a compound corresponds in each case to one row of Table A

### Table 9471

Compounds of the formula Ia.23 in which X is CH, R^{8b} is H, R^{8c} is A.11 and R⁵, R⁶ and R⁷ for a compound corresponds in each case to one row of Table A

### Table 9472

Compounds of the formula Ia.23 in which X is CH, R^{8b} is H, R^{8c} is A.12 and R⁵, R⁶ and R⁷ for a compound corresponds in each case to one row of Table A

### Table 9473

Compounds of the formula Ia.23 in which X is CH, R^{8b} is H, R^{8c} is A.13 and R⁵, R⁶ and R⁷ for a compound corresponds in each case to one row of Table A

### Table 9474

Compounds of the formula Ia.23 in which X is CH, R^{8b} is H, R^{8c} is A.14 and R⁵, R⁶ and R⁷ for a compound corresponds in each case to one row of Table A

### Table 9475

Compounds of the formula Ia.23 in which X is CH, R^{8b} is H, R^{8c} is A.15 and R⁵, R⁶ and R⁷ for a compound corresponds in each case to one row of Table A

### Table 9476

Compounds of the formula Ia.23 in which X is CH, R^{8b} is H, R^{8c} is A.16 and R⁵, R⁶ and R⁷ for a compound corresponds in each case to one row of Table A

### Table 9477

Compounds of the formula Ia.23 in which X is CH, R^{8b} is H, R^{8c} is A.17 and R⁵, R⁶ and R⁷ for a compound corresponds in each case to one row of Table A

### Table 9478

Compounds of the formula Ia.23 in which X is CH, R^{8b} is H, R^{8′} is A.18 and R⁵, R⁶ and R⁷ for a compound corresponds in each case to one row of Table A

### Table 9479

Compounds of the formula Ia.23 in which X is CH, R^{8b} is H, R^{8c} is A.19 and R⁵, R⁶ and R⁷ for a compound corresponds in each case to one row of Table A

### Table 9480

Compounds of the formula Ia.23 in which X is CH, R^{8b} is H, R^{8c} is A.20 and R⁵, R⁶ and R⁷ for a compound corresponds in each case to one row of Table A

### Table 9481

Compounds of the formula Ia.23 in which X is CH, R^{8b} is H, R^{8c} is A.21 and R⁵, R⁶ and R⁷ for a compound corresponds in each case to one row of Table A

### Table 9482

Compounds of the formula Ia.23 in which X is CH, R^{8b} is H, R^{8c} is A.22 and R⁵, R⁶ and R⁷ for a compound corresponds in each case to one row of Table A

### Table 9483

Compounds of the formula Ia.23 in which X is CH, R^{8b} is H, R^{8c} is A.23 and R⁵, R⁶ and R⁷ for a compound corresponds in each case to one row of Table A

### Table 9484

Compounds of the formula Ia.23 in which X is CH, R^{8b} is H, R^{8c} is A.24 and R⁵, R⁶ and R⁷ for a compound corresponds in each case to one row of Table A

### Table 9485

Compounds of the formula Ia.23 in which X is CH, R^{8b} is H, R^{8c} is A.25 and R⁵, R⁶ and R⁷ for a compound corresponds in each case to one row of Table A

### Table 9486

Compounds of the formula Ia.23 in which X is CH, R^{8b} is H, R^{8c} is A.26 and R⁵, R⁶ and R⁷ for a compound corresponds in each case to one row of Table A

### Table 9487

Compounds of the formula Ia.23 in which X is CH, R^{8b} is H, R^{8c} is A.27 and R⁵, R⁶ and R⁷ for a compound corresponds in each case to one row of Table A

### Table 9488

Compounds of the formula Ia.23 in which X is CH, R^{8b} is H, R^{8c} is A.28 and R⁵, R⁶ and R⁷ for a compound corresponds in each case to one row of Table A

### Table 9489

Compounds of the formula Ia.23 in which X is CH, R^{8b} is H, R^{8c} is A.29 and R⁵, R⁶ and R⁷ for a compound corresponds in each case to one row of Table A

### Table 9490

Compounds of the formula Ia.23 in which X is CH, R^{8b} is H, R^{8c} is A.30 and R⁵, R⁶ and R⁷ for a compound corresponds in each case to one row of Table A

### Table 9491

Compounds of the formula Ia.23 in which X is CH, R^{8b} is H, R^{8c} is A.31 and R⁵, R⁶ and R⁷ for a compound corresponds in each case to one row of Table A

### Table 9492

Compounds of the formula Ia.23 in which X is CH, R^{8b} is H, R^{8c} is A.32 and R⁵, R⁶ and R⁷ for a compound corresponds in each case to one row of Table A

### Table 9493

Compounds of the formula Ia.23 in which X is CH, R^{8b} is H, R^{8c} is A.33 and R⁵, R⁶ and R⁷ for a compound corresponds in each case to one row of Table A

### Table 9494

Compounds of the formula Ia.23 in which X is CH, R^{8b} is H, R^{8c} is A.34 and R⁵, R⁶ and R⁷ for a compound corresponds in each case to one row of Table A

### Table 9495

Compounds of the formula Ia.23 in which X is CH, R^{8b} is H, R^{8c} is A.35 and R⁵, R⁶ and R⁷ for a compound corresponds in each case to one row of Table A

### Table 9496

Compounds of the formula Ia.23 in which X is CH, R^{8b} is H, R^{8c} is A.36 and R⁵, R⁶ and R⁷ for a compound corresponds in each case to one row of Table A

### Table 9497

Compounds of the formula Ia.23 in which X is CH, R^{8b} is H, R^{8c} is A.37 and R⁵, R⁶ and R⁷ for a compound corresponds in each case to one row of Table A

### Table 9498

Compounds of the formula Ia.23 in which X is CH, R^{8b} is H, R^{8c} is A.38 and R⁵, R⁶ and R⁷ for a compound corresponds in each case to one row of Table A

### Table 9499

Compounds of the formula Ia.23 in which X is CH, R^{8b} is H, R^{8c} is A.39 and R⁵, R⁶ and R⁷ for a compound corresponds in each case to one row of Table A

### Table 9500

Compounds of the formula Ia.23 in which X is CH, R^{8b} is H, R^{8c} is A.40 and R⁵, R⁶ and R⁷ for a compound corresponds in each case to one row of Table A

### Table 9501

Compounds of the formula Ia.23 in which X is CH, R^{8b} is H, R^{8c} is A.41 and R⁵, R⁶ and R⁷ for a compound corresponds in each case to one row of Table A

### Table 9502

Compounds of the formula Ia.23 in which X is CH, R^{8b} is H, R^{8c} is A.42 and R⁵, R⁶ and R⁷ for a compound corresponds in each case to one row of Table A

### Table 9503

Compounds of the formula Ia.23 in which X is CH, R^{8b} is H, R^{8c} is A.43 and R⁵, R⁶ and R⁷ for a compound corresponds in each case to one row of Table A

### Table 9504

Compounds of the formula Ia.23 in which X is CH, R^{8b} is H, R^{8c} is A.44 and R⁵, R⁶ and R⁷ for a compound corresponds in each case to one row of Table A

### Table 9505

Compounds of the formula Ia.23 in which X is CH, R^{8b} is H, R^{8c} is A.45 and R⁵, R⁶ and R⁷ for a compound corresponds in each case to one row of Table A

### Table 9506

Compounds of the formula Ia.23 in which X is CH, R^{8b} is H, R^{8c} is A.46 and R⁵, R⁶ and R⁷ for a compound corresponds in each case to one row of Table A

### Table 9507

Compounds of the formula Ia.23 in which X is CH, R^{8b} is H, R^{8c} is A.47 and R⁵, R⁶ and R⁷ for a compound corresponds in each case to one row of Table A

### Table 9508

Compounds of the formula Ia.23 in which X is CH, R^{8b} is H, R^{8c} is A.48 and R⁵, R⁶ and R⁷ for a compound corresponds in each case to one row of Table A

### Table 9509

Compounds of the formula Ia.23 in which X is CH, R^{8b} is H, R^{8c} is A.49 and R⁵, R⁶ and R⁷ for a compound corresponds in each case to one row of Table A

### Table 9510

Compounds of the formula Ia.23 in which X is CH, R^{8b} is H, R^{8c} is A.50 and R⁵, R⁶ and R⁷ for a compound corresponds in each case to one row of Table A

### Table 9511

Compounds of the formula Ia.23 in which X is CH, R^{8b} is H, R^{8c} is A.51 and R⁵, R⁶ and R⁷ for a compound corresponds in each case to one row of Table A

### Table 9512

Compounds of the formula Ia.23 in which X is CH, R^{8b} is H, R^{8c} is A.52 and R⁵, R⁶ and R⁷ for a compound corresponds in each case to one row of Table A

### Table 9513

Compounds of the formula Ia.23 in which X is CH, R^{8b} is H, R^{8c} is A.53 and R⁵, R⁶ and R⁷ for a compound corresponds in each case to one row of Table A

### Table 9514

Compounds of the formula Ia.23 in which X is CH, R^{8b} is H, R^{8c} is A.54 and R⁵, R⁶ and R⁷ for a compound corresponds in each case to one row of Table A

### Table 9515

Compounds of the formula Ia.23 in which X is CH, R^{8b} is H, R^{8c} is A.55 and R⁵, R⁶ and R⁷ for a compound corresponds in each case to one row of Table A

### Table 9516

Compounds of the formula Ia.23 in which X is CH, R^{8b} is H, R^{8c} is A.56 and R⁵, R⁶ and R⁷ for a compound corresponds in each case to one row of Table A

### Table 9517

Compounds of the formula Ia.23 in which X is CH, R^{8b} is H, R^{8c} is A.57 and R⁵, R⁶ and R⁷ for a compound corresponds in each case to one row of Table A

### Table 9518

Compounds of the formula Ia.23 in which X is CH, R^{8b} is H, R^{8c} is A.58 and R⁵, R⁶ and R⁷ for a compound corresponds in each case to one row of Table A

### Table 9519

Compounds of the formula Ia.23 in which X is CH, R^{8b} is H, R^{8c} is A.59 and R⁵, R⁶ and R⁷ for a compound corresponds in each case to one row of Table A

### Table 9520

Compounds of the formula Ia.23 in which X is CH, R^{8b} is H, R^{8c} is A.60 and R⁵, R⁶ and R⁷ for a compound corresponds in each case to one row of Table A

### Table 9521

Compounds of the formula Ia.23 in which X is CH, R^{8b} is H, R^{8c} is A.61 and R⁵, R⁶ and R⁷ for a compound corresponds in each case to one row of Table A

### Table 9522

Compounds of the formula Ia.23 in which X is CH, R^{8b} is H, R^{8c} is A.62 and R⁵, R⁶ and R⁷ for a compound corresponds in each case to one row of Table A

### Table 9523

Compounds of the formula Ia.23 in which X is CH, R^{8b} is H, R^{8c} is A.63 and R⁵, R⁶ and R⁷ for a compound corresponds in each case to one row of Table A

### Table 9524

Compounds of the formula Ia.23 in which X is CH, R^{8b} is H, R^{8c} is A.64 and R⁵, R⁶ and R⁷ for a compound corresponds in each case to one row of Table A

### Table 9525

Compounds of the formula Ia.23 in which X is CH, R^{8b} is H, R^{8c} is A.65 and R⁵, R⁶ and R⁷ for a compound corresponds in each case to one row of Table A

### Table 9526

Compounds of the formula Ia.23 in which X is CH, R^{8b} is H, R^{8c} is A.66 and R⁵, R⁶ and R⁷ for a compound corresponds in each case to one row of Table A

### Table 9527

Compounds of the formula Ia.23 in which X is CH, R^{8b} is H, R^{8c} is A.67 and R⁵, R⁶ and R⁷ for a compound corresponds in each case to one row of Table A

### Table 9528

Compounds of the formula Ia.23 in which X is CH, R^{8b} is H, R^{8c} is A.68 and R⁵, R⁶ and R⁷ for a compound corresponds in each case to one row of Table A

### Table 9529

Compounds of the formula Ia.23 in which X is CH, R^{8b} is H, R^{8c} is A.69 and R⁵, R⁶ and R⁷ for a compound corresponds in each case to one row of Table A

### Table 9530

Compounds of the formula Ia.23 in which X is CH, R^{8b} is H, R^{8c} is A.70 and R⁵, R⁶ and R⁷ for a compound corresponds in each case to one row of Table A

### Table 9531

Compounds of the formula Ia.23 in which X is CH, R^{8b} is H, R^{8c} is —CH₂-A.1 and R⁵, R⁶ and R⁷ for a compound corresponds in each case to one row of Table A

### Table 9532

Compounds of the formula Ia.23 in which X is CH, R^{8b} is H, R^{8c} is —CH₂-A.2 and R⁵, R⁶ and R⁷ for a compound corresponds in each case to one row of Table A

### Table 9533

Compounds of the formula Ia.23 in which X is CH, R^{8b} is H, R^{8c} is —CH₂-A.3 and R⁵, R⁶ and R⁷ for a compound corresponds in each case to one row of Table A

### Table 9534

Compounds of the formula Ia.23 in which X is CH, R^{8b} is H, R^{8c} is —CH₂-A.4 and R⁵, R⁶ and R⁷ for a compound corresponds in each case to one row of Table A

### Table 9535

Compounds of the formula Ia.23 in which X is CH, R^{8b} is H, R^{8c} is —CH₂-A.5 and R⁵, R⁶ and R⁷ for a compound corresponds in each case to one row of Table A

### Table 9536

Compounds of the formula Ia.23 in which X is CH, R^{8b} is H, R^{8c} is —CH₂-A.6 and R⁵, R⁶ and R⁷ for a compound corresponds in each case to one row of Table A

### Table 9537

Compounds of the formula Ia.23 in which X is CH, R^{8b} is H, R^{8c} is —CH₂-A.7 and R⁵, R⁶ and R⁷ for a compound corresponds in each case to one row of Table A

### Table 9538

Compounds of the formula Ia.23 in which X is CH, R^{8b} is H, R^{8c} is —CH₂-A.8 and R⁵, R⁶ and R⁷ for a compound corresponds in each case to one row of Table A

### Table 9539

Compounds of the formula Ia.23 in which X is CH, R^{8b} is H, R^{8c} is —CH₂-A.9 and R⁵, R⁶ and R⁷ for a compound corresponds in each case to one row of Table A

### Table 9540

Compounds of the formula Ia.23 in which X is CH, R^{8b} is H, R^{8c} is —CH₂-A.10 and R⁵, R⁶ and R⁷ for a compound corresponds in each case to one row of Table A

### Table 9541

Compounds of the formula Ia.23 in which X is CH, R^{8b} is H, R^{8c} is —CH₂-A.11 and R⁵, R⁶ and R⁷ for a compound corresponds in each case to one row of Table A

### Table 9542

Compounds of the formula Ia.23 in which X is CH, R^{8b} is H, R^{8c} is —CH₂-A.12 and R⁵, R⁶ and R⁷ for a compound corresponds in each case to one row of Table A

### Table 9543

Compounds of the formula Ia.23 in which X is CH, R^{8b} is H, R^{8c} is —CH₂-A.13 and R⁵, R⁶ and R⁷ for a compound corresponds in each case to one row of Table A

### Table 9544

Compounds of the formula Ia.23 in which X is CH, R^{8b} is H, R^{8c} is —CH₂-A.14 and R⁵, R⁶ and R⁷ for a compound corresponds in each case to one row of Table A

### Table 9545

Compounds of the formula Ia.23 in which X is CH, R^{8b} is H, R^{8c} is —CH₂-A.15 and R⁵, R⁶ and R⁷ for a compound corresponds in each case to one row of Table A

### Table 9546

Compounds of the formula Ia.23 in which X is CH, R^{8b} is H, R^{8c} is —CH₂-A.16 and R⁵, R⁶ and R⁷ for a compound corresponds in each case to one row of Table A

### Table 9547

Compounds of the formula Ia.23 in which X is CH, R^{8b} is H, R^{8c} is —CH₂-A.17 and R⁵, R⁶ and R⁷ for a compound corresponds in each case to one row of Table A

### Table 9548

Compounds of the formula Ia.23 in which X is CH, R^{8b} is H, R^{8c} is —CH₂-A.18 and R⁵, R⁶ and R⁷ for a compound corresponds in each case to one row of Table A

### Table 9549

Compounds of the formula Ia.23 in which X is CH, R^{8b} is H, R^{8c} is —CH₂-A.19 and R⁵, R⁶ and R⁷ for a compound corresponds in each case to one row of Table A

### Table 9550

Compounds of the formula Ia.23 in which X is CH, R^{8b} is H, R^{8c} is —CH₂-A.20 and R⁵, R⁶ and R⁷ for a compound corresponds in each case to one row of Table A

### Table 9551

Compounds of the formula Ia.23 in which X is CH, R^{8b} is H, R^{8c} is —CH₂-A.21 and R⁵, R⁶ and R⁷ for a compound corresponds in each case to one row of Table A

### Table 9552

Compounds of the formula Ia.23 in which X is CH, R^{8b} is H, R^{8c} is —CH₂-A.22 and R⁵, R⁶ and R⁷ for a compound corresponds in each case to one row of Table A

### Table 9553

Compounds of the formula Ia.23 in which X is CH, R^{8b} is H, R^{8c} is —CH₂-A.23 and R⁵, R⁶ and R⁷ for a compound corresponds in each case to one row of Table A

### Table 9554

Compounds of the formula Ia.23 in which X is CH, R^{8b} is H, R^{8c} is —CH₂-A.24 and R⁵, R⁶ and R⁷ for a compound corresponds in each case to one row of Table A

### Table 9555

Compounds of the formula Ia.23 in which X is CH, R^{8b} is H, R^{8c} is —CH₂-A.25 and R⁵, R⁶ and R⁷ for a compound corresponds in each case to one row of Table A

### Table 9556

Compounds of the formula Ia.23 in which X is CH, R^{8b} is H, R^{8c} is —CH₂-A.26 and R⁵, R⁶ and R⁷ for a compound corresponds in each case to one row of Table A

### Table 9557

Compounds of the formula Ia.23 in which X is CH, R^{8b} is H, R^{8c} is —CH₂-A.27 and R⁵, R⁶ and R⁷ for a compound corresponds in each case to one row of Table A

### Table 9558

Compounds of the formula Ia.23 in which X is CH, R^{8b} is H, R^{8c} is —CH₂-A.28 and R⁵, R⁶ and R⁷ for a compound corresponds in each case to one row of Table A

### Table 9559

Compounds of the formula Ia.23 in which X is CH, R^{8b} is H, R^{8c} is —CH₂-A.29 and R⁵, R⁶ and R⁷ for a compound corresponds in each case to one row of Table A

### Table 9560

Compounds of the formula Ia.23 in which X is CH, R^{8b} is H, R^{8c} is —CH₂-A.30 and R⁵, R⁶ and R⁷ for a compound corresponds in each case to one row of Table A

### Table 9561

Compounds of the formula Ia.23 in which X is CH, R^{8b} is H, R^{8c} is —CH₂-A.31 and R⁵, R⁶ and R⁷ for a compound corresponds in each case to one row of Table A

### Table 9562

Compounds of the formula Ia.23 in which X is CH, R^{8b} is H, R^{8c} is —CH₂-A.32 and R⁵, R⁶ and R⁷ for a compound corresponds in each case to one row of Table A

### Table 9563

Compounds of the formula Ia.23 in which X is CH, R^{8b} is H, R^{8c} is —CH₂-A.33 and R⁵, R⁶ and R⁷ for a compound corresponds in each case to one row of Table A

### Table 9564

Compounds of the formula Ia.23 in which X is CH, R^{8b} is H, R^{8c} is —CH₂-A.34 and R⁵, R⁶ and R⁷ for a compound corresponds in each case to one row of Table A

### Table 9565

Compounds of the formula Ia.23 in which X is CH, R^{8b} is H, R^{8c} is —CH₂-A.35 and R⁵, R⁶ and R⁷ for a compound corresponds in each case to one row of Table A

### Table 9566

Compounds of the formula Ia.23 in which X is CH, R^{8b} is H, R^{8c} is —CH₂-A.36 and R⁵, R⁶ and R⁷ for a compound corresponds in each case to one row of Table A

### Table 9567

Compounds of the formula Ia.23 in which X is CH, R^{8b} is H, R^{8c} is —CH₂-A.37 and R⁵, R⁶ and R⁷ for a compound corresponds in each case to one row of Table A

### Table 9568

Compounds of the formula Ia.23 in which X is CH, R^{8b} is H, R^{8c} is —CH₂-A.38 and R⁵, R⁶ and R⁷ for a compound corresponds in each case to one row of Table A

### Table 9569

Compounds of the formula Ia.23 in which X is CH, R^{8b} is H, R^{8c} is —CH₂-A.39 and R⁵, R⁶ and R⁷ for a compound corresponds in each case to one row of Table A

### Table 9570

Compounds of the formula Ia.23 in which X is CH, R^{8b} is H, R^{8c} is —CH₂-A.40 and R⁵, R⁶ and R⁷ for a compound corresponds in each case to one row of Table A

### Table 9571

Compounds of the formula Ia.23 in which X is CH, R^{8b} is H, R^{8c} is —CH₂-A.41 and R⁵, R⁶ and R⁷ for a compound corresponds in each case to one row of Table A

### Table 9572

Compounds of the formula Ia.23 in which X is CH, R^{8b} is H, R^{8c} is —CH₂-A.42 and R⁵, R⁶ and R⁷ for a compound corresponds in each case to one row of Table A

### Table 9573

Compounds of the formula Ia.23 in which X is CH, R^{8b} is H, R^{8c} is —CH₂-A.43 and R⁵, R⁶ and R⁷ for a compound corresponds in each case to one row of Table A

### Table 9574

Compounds of the formula Ia.23 in which X is CH, R^{8b} is H, R^{8c} is —CH₂-A.44 and R⁵, R⁶ and R⁷ for a compound corresponds in each case to one row of Table A

### Table 9575

Compounds of the formula Ia.23 in which X is CH, R^{8b} is H, R^{8c} is —CH₂-A.45 and R⁵, R⁶ and R⁷ for a compound corresponds in each case to one row of Table A

### Table 9576

Compounds of the formula Ia.23 in which X is CH, R^{8b} is H, R^{8c} is —CH₂-A.46 and R⁵, R⁶ and R⁷ for a compound corresponds in each case to one row of Table A

### Table 9577

Compounds of the formula Ia.23 in which X is CH, R^{8b} is H, R^{8c} is —CH₂-A.47 and R⁵, R⁶ and R⁷ for a compound corresponds in each case to one row of Table A

### Table 9578

Compounds of the formula Ia.23 in which X is CH, R^{8b} is H, R^{8c} is —CH₂-A.48 and R⁵, R⁶ and R⁷ for a compound corresponds in each case to one row of Table A

### Table 9579

Compounds of the formula Ia.23 in which X is CH, R^{8b} is H, R^{8c} is —CH₂-A.49 and R⁵, R⁶ and R⁷ for a compound corresponds in each case to one row of Table A

### Table 9580

Compounds of the formula Ia.23 in which X is CH, R^{8b} is H, R^{8c} is —CH₂-A.50 and R⁵, R⁶ and R⁷ for a compound corresponds in each case to one row of Table A

### Table 9581

Compounds of the formula Ia.23 in which X is CH, R^{8b} is H, R^{8c} is —CH₂-A.51 and R⁵, R⁶ and R⁷ for a compound corresponds in each case to one row of Table A

### Table 9582

Compounds of the formula Ia.23 in which X is CH, R^{8b} is H, R^{8c} is —CH₂-A.52 and R⁵, R⁶ and R⁷ for a compound corresponds in each case to one row of Table A

### Table 9583

Compounds of the formula Ia.23 in which X is CH, R^{8b} is H, R^{8c} is —CH₂-A.53 and R⁵, R⁶ and R⁷ for a compound corresponds in each case to one row of Table A

### Table 9584

Compounds of the formula Ia.23 in which X is CH, R^{8b} is H, R^{8c} is —CH₂-A.54 and R⁵, R⁶ and R⁷ for a compound corresponds in each case to one row of Table A

### Table 9585

Compounds of the formula Ia.23 in which X is CH, R^{8b} is H, R^{8c} is —CH₂-A.55 and R⁵, R⁶ and R⁷ for a compound corresponds in each case to one row of Table A

### Table 9586

Compounds of the formula Ia.23 in which X is CH, R^{8b} is H, R^{8c} is —CH₂-A.56 and R⁵, R⁶ and R⁷ for a compound corresponds in each case to one row of Table A

### Table 9587

Compounds of the formula Ia.23 in which X is CH, R^{8b} is H, R^{8c} is —CH₂-A.57 and R⁵, R⁶ and R⁷ for a compound corresponds in each case to one row of Table A

### Table 9588

Compounds of the formula Ia.23 in which X is CH, R^{8b} is H, R^{8c} is —CH₂-A.58 and R⁵, R⁶ and R⁷ for a compound corresponds in each case to one row of Table A

### Table 9589

Compounds of the formula Ia.23 in which X is CH, R^{8b} is H, R^{8c} is —CH₂-A.59 and R⁵, R⁶ and R⁷ for a compound corresponds in each case to one row of Table A

### Table 9590

Compounds of the formula Ia.23 in which X is CH, R^{8b} is H, R^{8c} is —CH₂-A.60 and R⁵, R⁶ and R⁷ for a compound corresponds in each case to one row of Table A

### Table 9591

Compounds of the formula Ia.23 in which X is CH, R^{8b} is H, R^{8c} is —CH₂-A.61 and R⁵, R⁶ and R⁷ for a compound corresponds in each case to one row of Table A

### Table 9592

Compounds of the formula Ia.23 in which X is CH, R^{8b} is H, R^{8c} is —CH₂-A.62 and R⁵, R⁶ and R⁷ for a compound corresponds in each case to one row of Table A

### Table 9593

Compounds of the formula Ia.23 in which X is CH, R^{8b} is H, R^{8c} is —CH₂-A.63 and R⁵, R⁶ and R⁷ for a compound corresponds in each case to one row of Table A

### Table 9594

Compounds of the formula Ia.23 in which X is CH, R^{8b} is H, R^{8c} is —CH₂-A.64 and R⁵, R⁶ and R⁷ for a compound corresponds in each case to one row of Table A

### Table 9595

Compounds of the formula Ia.23 in which X is CH, R^{8b} is H, R^{8c} is —CH₂-A.65 and R⁵, R⁶ and R⁷ for a compound corresponds in each case to one row of Table A

### Table 9596

Compounds of the formula Ia.23 in which X is CH, R^{8b} is H, R^{8c} is —CH₂-A.66 and R⁵, R⁶ and R⁷ for a compound corresponds in each case to one row of Table A

### Table 9597

Compounds of the formula Ia.23 in which X is CH, R^{8b} is H, R^{8c} is —CH₂-A.67 and R⁵, R⁶ and R⁷ for a compound corresponds in each case to one row of Table A

### Table 9598

Compounds of the formula Ia.23 in which X is CH, R^{8b} is H, R^{8c} is —CH₂-A.68 and R⁵, R⁶ and R⁷ for a compound corresponds in each case to one row of Table A

### Table 9599

Compounds of the formula Ia.23 in which X is CH, R^{8b} is H, R^{8c} is —CH₂-A.69 and R⁵, R⁶ and R⁷ for a compound corresponds in each case to one row of Table A

### Table 9600

Compounds of the formula Ia.23 in which X is CH, R^{8b} is H, R^{8c} is —CH₂-A.70 and R⁵, R⁶ and R⁷ for a compound corresponds in each case to one row of Table A

### Tables 9601 to 9740

Compounds of the formula Ia.23 in which X is CH, R^{8b} is methyl, R^{8a} is as defined in any of tables 9461 to 9600 and R⁵, R⁶ and R⁷ for a compound corresponds in each case to one row of Table A

### Tables 9741 to 9880

Compounds of the formula Ia.23 in which X is CH, R^{8b} is ethyl, R^{8c} is as defined in any of tables 9461 to 9600 and R⁵, R⁶ and R⁷ for a compound corresponds in each case to one row of Table A

### Tables 9881 to 10020

Compounds of the formula Ia.23 in which X is CH, R^{8b} is propyl, R^{8c} is as defined in any of tables 9461 to 9600 and R⁵, R⁶ and R⁷ for a compound corresponds in each case to one row of Table A

### Tables 10021 to 10160

Compounds of the formula Ia.23 in which X is CH, R^{8b} is allyl, R^{8c} is as defined in any of tables 9461 to 9600 and R⁵, R⁶ and R⁷ for a compound corresponds in each case to one row of Table A

### Tables 10161 to 10860

Compounds of the formula Ia.23 in which X is N, R^{8b} and R^{8c} are as defined in any of tables 9461 to 10160 and R⁵, R⁶ and R⁷ for a compound corresponds in each case to one row of Table A

### Table 10861

Compounds of the formula Ia.23 in which X is CH, —NR^{8b}R^{8c} is a ring A.97 and R⁵, R⁶ and R⁷ for a compound corresponds in each case to one row of Table A

### Table 10862

Compounds of the formula Ia.23 in which X is CH, —NR^{8b}R^{8c} is a ring A.98 and R⁵, R⁶ and R⁷ for a compound corresponds in each case to one row of Table A

### Table 10863

Compounds of the formula Ia.23 in which X is CH, —NR^{8b}R^{8c} is a ring A.99 and R⁵, R⁶ and R⁷ for a compound corresponds in each case to one row of Table A

### Table 10864

Compounds of the formula Ia.23 in which X is CH, —NR^{8b}R^{8c} is a ring A.100 and R⁵, R⁶ and R⁷ for a compound corresponds in each case to one row of Table A

### Table 10865

Compounds of the formula Ia.23 in which X is CH, —NR^{8b}R^{8c} is a ring A.101 and R⁵, R⁶ and R⁷ for a compound corresponds in each case to one row of Table A

### Table 10866

Compounds of the formula Ia.23 in which X is CH, —NR^{8b}R^{8c} is a ring A.102 and R⁵, R⁶ and R⁷ for a compound corresponds in each case to one row of Table A

### Table 10867

Compounds of the formula Ia.23 in which X is CH, —NR^{8b}R^{8c} is a ring A.103 and R⁵, R⁶ and R⁷ for a compound corresponds in each case to one row of Table A

### Table 10868

Compounds of the formula Ia.23 in which X is CH, —NR^{8b}R^{8c} is a ring A.104 and R⁵, R⁶ and R⁷ for a compound corresponds in each case to one row of Table A

### Table 10869

Compounds of the formula Ia.23 in which X is CH, —NR^{8b}R^{8c} is a ring A.105 and R⁵, R⁶ and R⁷ for a compound corresponds in each case to one row of Table A

### Table 10870

Compounds of the formula Ia.23 in which X is CH, —NR^{8b}R^{8c} is a ring A.106 and R⁵, R⁶ and R⁷ for a compound corresponds in each case to one row of Table A

### Table 10871

Compounds of the formula Ia.23 in which X is CH, —NR^{8b}R^{8c} is a ring A.107 and R⁵, R⁶ and R⁷ for a compound corresponds in each case to one row of Table A

### Table 10872

Compounds of the formula Ia.23 in which X is CH, —NR^{8b}R^{8c} is a ring A.108 and R⁵, R⁶ and R⁷ for a compound corresponds in each case to one row of Table A

### Table 10873

Compounds of the formula Ia.23 in which X is CH, —NR^{8b}R^{8c} is a ring A.109 and R⁵, R⁶ and R⁷ for a compound corresponds in each case to one row of Table A

### Table 10874

Compounds of the formula Ia.23 in which X is CH, —NR^{8b}R^{8c} is a ring A.110 and R⁵, R⁶ and R⁷ for a compound corresponds in each case to one row of Table A

### Table 10875

Compounds of the formula Ia.23 in which X is CH, —NR^{8b}R^{8c} is a ring A.111 and R⁵, R⁶ and R⁷ for a compound corresponds in each case to one row of Table A

### Table 10876

Compounds of the formula Ia.23 in which X is CH, —NR^{8b}R^{8c} is a ring A.112 and R⁵, R⁶ and R⁷ for a compound corresponds in each case to one row of Table A

### Table 10877

Compounds of the formula Ia.23 in which X is CH, —NR^{8b}R^{8c} is a ring A.113 and R⁵, R⁶ and R⁷ for a compound corresponds in each case to one row of Table A

### Table 10878

Compounds of the formula Ia.23 in which X is CH, —NR^{8b}R^{8c} is a ring A.114 and R⁵, R⁶ and R⁷ for a compound corresponds in each case to one row of Table A

### Table 10879

Compounds of the formula Ia.23 in which X is CH, —NR^{8b}R^{8c} is a ring A.115 and R⁵, R⁶ and R⁷ for a compound corresponds in each case to one row of Table A

### Table 10880

Compounds of the formula Ia.23 in which X is CH, —NR^{8b}R^{8c} is a ring A.116 and R⁵, R⁶ and R⁷ for a compound corresponds in each case to one row of Table A

### Table 10881

Compounds of the formula Ia.23 in which X is CH, —NR^{8b}R^{8c} is a ring A.117 and R⁵, R⁶ and R⁷ for a compound corresponds in each case to one row of Table A

### Table 10882

Compounds of the formula Ia.23 in which X is CH, —NR^{8b}R^{8c} is a ring A.118 and R⁵, R⁶ and R⁷ for a compound corresponds in each case to one row of Table A

### Table 10883

Compounds of the formula Ia.23 in which X is CH, —NR^{8b}R^{8c} is a ring A.119 and R⁵, R⁶ and R⁷ for a compound corresponds in each case to one row of Table A

### Table 10884

Compounds of the formula Ia.23 in which X is CH, —NR^{8b}R^{8c} is a ring A.120 and R⁵, R⁶ and R⁷ for a compound corresponds in each case to one row of Table A

### Table 10885

Compounds of the formula Ia.23 in which X is CH, —NR^{8b}R^{8c} is a ring A.121 and R⁵, R⁶ and R⁷ for a compound corresponds in each case to one row of Table A

### Table 10886

Compounds of the formula Ia.23 in which X is CH, —NR^{8b}R^{8c} is a ring A.122 and R⁵, R⁶ and R⁷ for a compound corresponds in each case to one row of Table A

### Table 10887

Compounds of the formula Ia.23 in which X is CH, —NR^{8b}R^{8c} is a ring A.123 and R⁵, R⁶ and R⁷ for a compound corresponds in each case to one row of Table A

### Table 10888

Compounds of the formula Ia.23 in which X is CH, —NR^{8b}R^{8c} is a ring A.124 and R⁵, R⁶ and R⁷ for a compound corresponds in each case to one row of Table A

### Table 10889

Compounds of the formula Ia.23 in which X is CH, —NR^{8b}R^{8c} is a ring A.125 and R⁵, R⁶ and R⁷ for a compound corresponds in each case to one row of Table A

### Table 10890

Compounds of the formula Ia.23 in which X is CH, —NR^{8b}R^{8c} is a ring A.126 and R⁵, R⁶ and R⁷ for a compound corresponds in each case to one row of Table A

### Table 10891

Compounds of the formula Ia.23 in which X is CH, —NR^{8b}R^{8c} is a ring A.127 and R⁵, R⁶ and R⁷ for a compound corresponds in each case to one row of Table A

### Table 10892

Compounds of the formula Ia.23 in which X is CH, —NR^{8b}R^{8c} is a ring A.128 and R⁵, R⁶ and R⁷ for a compound corresponds in each case to one row of Table A

### Table 10893

Compounds of the formula Ia.23 in which X is CH, —NR^{8b}R^{8c} is a ring A.129 and R⁵, R⁶ and R⁷ for a compound corresponds in each case to one row of Table A

### Table 10894

Compounds of the formula Ia.23 in which X is CH, —NR^{8b}R^{8c} is a ring A.130 and R⁵, R⁶ and R⁷ for a compound corresponds in each case to one row of Table A

### Table 10895

Compounds of the formula Ia.23 in which X is CH, —NR^{8b}R^{8c} is a ring A.131 and R⁵, R⁶ and R⁷ for a compound corresponds in each case to one row of Table A

### Table 10896

Compounds of the formula Ia.23 in which X is CH, —NR^{8b}R^{8c} is a ring A.132 and R⁵, R⁶ and R⁷ for a compound corresponds in each case to one row of Table A

### Table 10897

Compounds of the formula Ia.23 in which X is CH, —NR^{8b}R^{8c} is a ring A.133 and R⁵, R⁶ and R⁷ for a compound corresponds in each case to one row of Table A

### Table 10898

Compounds of the formula Ia.23 in which X is CH, —NR^{8b}R^{8c} is a ring A.134 and R⁵, R⁶ and R⁷ for a compound corresponds in each case to one row of Table A

### Table 10899

Compounds of the formula Ia.23 in which X is CH, —NR^{8b}R^{8c} is a ring A.135 and R⁵, R⁶ and R⁷ for a compound corresponds in each case to one row of Table A

### Table 10900

Compounds of the formula Ia.23 in which X is CH, —NR^{8b}R^{8c} is a ring A.136 and R⁵, R⁶ and R⁷ for a compound corresponds in each case to one row of Table A

### Table 10901

Compounds of the formula Ia.23 in which X is CH, —NR^{8b}R^{8c} is a ring A.137 and R⁵, R⁶ and R⁷ for a compound corresponds in each case to one row of Table A

### Table 10902

Compounds of the formula Ia.23 in which X is CH, —NR^{8b}R^{8c} is a ring A.138 and R⁵, R⁶ and R⁷ for a compound corresponds in each case to one row of Table A

### Table 10903

Compounds of the formula Ia.23 in which X is CH, —NR^{8b}R^{8c} is a ring A.139 and R⁵, R⁶ and R⁷ for a compound corresponds in each case to one row of Table A

### Table 10904

Compounds of the formula Ia.23 in which X is CH, —NR^{8b}R^{8c} is a ring A.140 and R⁵, R⁶ and R⁷ for a compound corresponds in each case to one row of Table A

### Table 10905

Compounds of the formula Ia.23 in which X is CH, —NR^{8b}R^{8c} is a ring A.141 and R⁵, R⁶ and R⁷ for a compound corresponds in each case to one row of Table A

### Tables 10906 to 10950

Compounds of the formula Ia.23 in which X is N, —NR^{8b}R^{8c} is as defined in any of tables 10861 to 10905 and R⁵, R⁶ and R⁷ for a compound corresponds in each case to one row of Table A

### Table 10951

Compounds of the formula Ia.24 in which X is CH, —R^{8d} is A.1 and R⁵, R⁶ and R⁷ for a compound corresponds in each case to one row of Table A

### Table 10952

Compounds of the formula Ia.24 in which X is CH, —R^{8d} is A.2 and R⁵, R⁶ and R⁷ for a compound corresponds in each case to one row of Table A

### Table 10953

Compounds of the formula Ia.24 in which X is CH, —R^{8d} is A.3 and R⁵, R⁶ and R⁷ for a compound corresponds in each case to one row of Table A

### Table 10954

Compounds of the formula Ia.24 in which X is CH, —R^{8d} is A.4 and R⁵, R⁶ and R⁷ for a compound corresponds in each case to one row of Table A

### Table 10955

Compounds of the formula Ia.24 in which X is CH, —R^{8d} is A.5 and R⁵, R⁶ and R⁷ for a compound corresponds in each case to one row of Table A

### Table 10956

Compounds of the formula Ia.24 in which X is CH, —R^{8d} is A.6 and R⁵, R⁶ and R⁷ for a compound corresponds in each case to one row of Table A

### Table 10957

Compounds of the formula Ia.24 in which X is CH, —R^{8d} is A.7 and R⁵, R⁶ and R⁷ for a compound corresponds in each case to one row of Table A

### Table 10958

Compounds of the formula Ia.24 in which X is CH, —R^{8d} is A.8 and R⁵, R⁶ and R⁷ for a compound corresponds in each case to one row of Table A

### Table 10959

Compounds of the formula Ia.24 in which X is CH, —R^{8d} is A.9 and R⁵, R⁶ and R⁷ for a compound corresponds in each case to one row of Table A

### Table 10960

Compounds of the formula Ia.24 in which X is CH, —R^{8d} is A.10 and R⁵, R⁶ and R⁷ for a compound corresponds in each case to one row of Table A

### Table 10961

Compounds of the formula Ia.24 in which X is CH, —R^{8d} is A.11 and R⁵, R⁶ and R⁷ for a compound corresponds in each case to one row of Table A

### Table 10962

Compounds of the formula Ia.24 in which X is CH, —R^{8d} is A.12 and R⁵, R⁶ and R⁷ for a compound corresponds in each case to one row of Table A

### Table 10963

Compounds of the formula Ia.24 in which X is CH, —R^{8d} is A.13 and R⁵, R⁶ and R⁷ for a compound corresponds in each case to one row of Table A

### Table 10964

Compounds of the formula Ia.24 in which X is CH, —R^{8d} is A.14 and R⁵, R⁶ and R⁷ for a compound corresponds in each case to one row of Table A

### Table 10965

Compounds of the formula Ia.24 in which X is CH, —R^{8d} is A.15 and R⁵, R⁶ and R⁷ for a compound corresponds in each case to one row of Table A

### Table 10966

Compounds of the formula Ia.24 in which X is CH, —R^{8d} is A.16 and R⁵, R⁶ and R⁷ for a compound corresponds in each case to one row of Table A

### Table 10967

Compounds of the formula Ia.24 in which X is CH, —R^{8d} is A.17 and R⁵, R⁶ and R⁷ for a compound corresponds in each case to one row of Table A

### Table 10968

Compounds of the formula Ia.24 in which X is CH, —R^{8d} is A.18 and R⁵, R⁶ and R⁷ for a compound corresponds in each case to one row of Table A

### Table 10969

Compounds of the formula Ia.24 in which X is CH, —R^{8d} is A.19 and R⁵, R⁶ and R⁷ for a compound corresponds in each case to one row of Table A

### Table 10970

Compounds of the formula Ia.24 in which X is CH, —R^{8d} is A.20 and R⁵, R⁶ and R⁷ for a compound corresponds in each case to one row of Table A

### Table 10971

Compounds of the formula Ia.24 in which X is CH, —R^{8d} is A.21 and R⁵, R⁶ and R⁷ for a compound corresponds in each case to one row of Table A

### Table 10972

Compounds of the formula Ia.24 in which X is CH, —R^{8d} is A.22 and R⁵, R⁶ and R⁷ for a compound corresponds in each case to one row of Table A

### Table 10973

Compounds of the formula Ia.24 in which X is CH, —R^{8d} is A.23 and R⁵, R⁶ and R⁷ for a compound corresponds in each case to one row of Table A

### Table 10974

Compounds of the formula Ia.24 in which X is CH, —R^{8d} is A.24 and R⁵, R⁶ and R⁷ for a compound corresponds in each case to one row of Table A

### Table 10975

Compounds of the formula Ia.24 in which X is CH, —R^{8d} is A.25 and R⁵, R⁶ and R⁷ for a compound corresponds in each case to one row of Table A

### Table 10976

Compounds of the formula Ia.24 in which X is CH, —R^{8d} is A.26 and R⁵, R⁶ and R⁷ for a compound corresponds in each case to one row of Table A

### Table 10977

Compounds of the formula Ia.24 in which X is CH, —R^{8d} is A.27 and R⁵, R⁶ and R⁷ for a compound corresponds in each case to one row of Table A

### Table 10978

Compounds of the formula Ia.24 in which X is CH, —R^{8d} is A.28 and R⁵, R⁶ and R⁷ for a compound corresponds in each case to one row of Table A

### Table 10979

Compounds of the formula Ia.24 in which X is CH, —R^{8d} is A.29 and R⁵, R⁶ and R⁷ for a compound corresponds in each case to one row of Table A

### Table 10980

Compounds of the formula Ia.24 in which X is CH, —R^{8d} is A.30 and R⁵, R⁶ and R⁷ for a compound corresponds in each case to one row of Table A

### Table 10981

Compounds of the formula Ia.24 in which X is CH, —R^{8d} is A.31 and R⁵, R⁶ and R⁷ for a compound corresponds in each case to one row of Table A

### Table 10982

Compounds of the formula Ia.24 in which X is CH, —R^{8d} is A.32 and R⁵, R⁶ and R⁷ for a compound corresponds in each case to one row of Table A

### Table 10983

Compounds of the formula Ia.24 in which X is CH, —R^{8d} is A.33 and R⁵, R⁶ and R⁷ for a compound corresponds in each case to one row of Table A

### Table 10984

Compounds of the formula Ia.24 in which X is CH, —R^{8d} is A.34 and R⁵, R⁶ and R⁷ for a compound corresponds in each case to one row of Table A

### Table 10985

Compounds of the formula Ia.24 in which X is CH, —R^{8d} is A.35 and R⁵, R⁶ and R⁷ for a compound corresponds in each case to one row of Table A

### Table 10986

Compounds of the formula Ia.24 in which X is CH, —R^{8d} is A.36 and R⁵, R⁶ and R⁷ for a compound corresponds in each case to one row of Table A

### Table 10987

Compounds of the formula Ia.24 in which X is CH, —R^{8d} is A.37 and R⁵, R⁶ and R⁷ for a compound corresponds in each case to one row of Table A

### Table 10988

Compounds of the formula Ia.24 in which X is CH, —R^{8d} is A.38 and R⁵, R⁶ and R⁷ for a compound corresponds in each case to one row of Table A

### Table 10989

Compounds of the formula Ia.24 in which X is CH, —R^{8d} is A.39 and R⁵, R⁶ and R⁷ for a compound corresponds in each case to one row of Table A

### Table 10990

Compounds of the formula Ia.24 in which X is CH, —R^{8d} is A.40 and R⁵, R⁶ and R⁷ for a compound corresponds in each case to one row of Table A

### Table 10991

Compounds of the formula Ia.24 in which X is CH, —R^{8d} is A.41 and R⁵, R⁶ and R⁷ for a compound corresponds in each case to one row of Table A

### Table 10992

Compounds of the formula Ia.24 in which X is CH, —R^{8d} is A.42 and R⁵, R⁶ and R⁷ for a compound corresponds in each case to one row of Table A

### Table 10993

Compounds of the formula Ia.24 in which X is CH, —R^{8d} is A.43 and R⁵, R⁶ and R⁷ for a compound corresponds in each case to one row of Table A

### Table 10994

Compounds of the formula Ia.24 in which X is CH, —R^{8d} is A.44 and R⁵, R⁶ and R⁷ for a compound corresponds in each case to one row of Table A

### Table 10995

Compounds of the formula Ia.24 in which X is CH, —R^{8d} is A.45 and R⁵, R⁶ and R⁷ for a compound corresponds in each case to one row of Table A

### Table 10996

Compounds of the formula Ia.24 in which X is CH, —R^{8d} is A.46 and R⁵, R⁶ and R⁷ for a compound corresponds in each case to one row of Table A

### Table 10997

Compounds of the formula Ia.24 in which X is CH, —R^{8d} is A.47 and R⁵, R⁶ and R⁷ for a compound corresponds in each case to one row of Table A

### Table 10998

Compounds of the formula Ia.24 in which X is CH, —R^{8d} is A.48 and R⁵, R⁶ and R⁷ for a compound corresponds in each case to one row of Table A

### Table 10999

Compounds of the formula Ia.24 in which X is CH, —R^{8d} is A.49 and R⁵, R⁶ and R⁷ for a compound corresponds in each case to one row of Table A

### Table 11000

Compounds of the formula Ia.24 in which X is CH, —R^{8d} is A.50 and R⁵, R⁶ and R⁷ for a compound corresponds in each case to one row of Table A

### Table 11001

Compounds of the formula Ia.24 in which X is CH, —R^{8d} is A.51 and R⁵, R⁶ and R⁷ for a compound corresponds in each case to one row of Table A

### Table 11002

Compounds of the formula Ia.24 in which X is CH, —R^{8d} is A.52 and R⁵, R⁶ and R⁷ for a compound corresponds in each case to one row of Table A

### Table 11003

Compounds of the formula Ia.24 in which X is CH, —R^{8d} is A.53 and R⁵, R⁶ and R⁷ for a compound corresponds in each case to one row of Table A

### Table 11004

Compounds of the formula Ia.24 in which X is CH, —R^{8d} is A.54 and R⁵, R⁶ and R⁷ for a compound corresponds in each case to one row of Table A

### Table 11005

Compounds of the formula Ia.24 in which X is CH, —R^{8d} is A.55 and R⁵, R⁶ and R⁷ for a compound corresponds in each case to one row of Table A

### Table 11006

Compounds of the formula Ia.24 in which X is CH, —R^{8d} is A.56 and R⁵, R⁶ and R⁷ for a compound corresponds in each case to one row of Table A

### Table 11007

Compounds of the formula Ia.24 in which X is CH, —R^{8d} is A.57 and R⁵, R⁶ and R⁷ for a compound corresponds in each case to one row of Table A

### Table 11008

Compounds of the formula Ia.24 in which X is CH, —R^{8d} is A.58 and R⁵, R⁶ and R⁷ for a compound corresponds in each case to one row of Table A

### Table 11009

Compounds of the formula Ia.24 in which X is CH, —R^{8d} is A.59 and R⁵, R⁶ and R⁷ for a compound corresponds in each case to one row of Table A

### Table 11010

Compounds of the formula Ia.24 in which X is CH, —R^{8d} is A.60 and R⁵, R⁶ and R⁷ for a compound corresponds in each case to one row of Table A

### Table 11011

Compounds of the formula Ia.24 in which X is CH, —R^{8d} is A.61 and R⁵, R⁶ and R⁷ for a compound corresponds in each case to one row of Table A

### Table 11012

Compounds of the formula Ia.24 in which X is CH, —R^{8d} is A.62 and R⁵, R⁶ and R⁷ for a compound corresponds in each case to one row of Table A

### Table 11013

Compounds of the formula Ia.24 in which X is CH, —R^{8d} is A.63 and R⁵, R⁶ and R⁷ for a compound corresponds in each case to one row of Table A

### Table 11014

Compounds of the formula Ia.24 in which X is CH, —R^{8d} is A.64 and R⁵, R⁶ and R⁷ for a compound corresponds in each case to one row of Table A

### Table 11015

Compounds of the formula Ia.24 in which X is CH, —R^{8d} is A.65 and R⁵, R⁶ and R⁷ for a compound corresponds in each case to one row of Table A

### Table 11016

Compounds of the formula Ia.24 in which X is CH, —R^{8d} is A.66 and R⁵, R⁶ and R⁷ for a compound corresponds in each case to one row of Table A

### Table 11017

Compounds of the formula Ia.24 in which X is CH, —R^{8d} is A.67 and R⁵, R⁶ and R⁷ for a compound corresponds in each case to one row of Table A

### Table 11018

Compounds of the formula Ia.24 in which X is CH, —R^{8d} is A.68 and R⁵, R⁶ and R⁷ for a compound corresponds in each case to one row of Table A

### Table 11019

Compounds of the formula Ia.24 in which X is CH, —R^{8d} is A.92 and R⁵, R⁶ and R⁷ for a compound corresponds in each case to one row of Table A

### Table 11020

Compounds of the formula Ia.24 in which X is CH, —R^{8d} is A.93 and R⁵, R⁶ and R⁷ for a compound corresponds in each case to one row of Table A

### Table 11021

Compounds of the formula Ia.24 in which X is CH, —R^{8d} is A.94 and R⁵, R⁶ and R⁷ for a compound corresponds in each case to one row of Table A

### Table 11022

Compounds of the formula Ia.24 in which X is CH, —R^{8d} is A.95 and R⁵, R⁶ and R⁷ for a compound corresponds in each case to one row of Table A

### Table 11023

Compounds of the formula Ia.24 in which X is CH, —R^{8d} is A.96 and R⁵, R⁶ and R⁷ for a compound corresponds in each case to one row of Table A

### Table 11024

Compounds of the formula Ia.24 in which X is CH, —R^{8d} is —CH₂-A.1 and R⁵, R⁶ and R⁷ for a compound corresponds in each case to one row of Table A

### Table 11025

Compounds of the formula Ia.24 in which X is CH, —R^{8d} is —CH₂-A.2 and R⁵, R⁶ and R⁷ for a compound corresponds in each case to one row of Table A

### Table 11026

Compounds of the formula Ia.24 in which X is CH, —R^{8d} is —CH₂-A.3 and R⁵, R⁶ and R⁷ for a compound corresponds in each case to one row of Table A

### Table 11027

Compounds of the formula Ia.24 in which X is CH, —R^{8d} is —CH₂-A.4 and R⁵, R⁶ and R⁷ for a compound corresponds in each case to one row of Table A

### Table 11028

Compounds of the formula Ia.24 in which X is CH, —R^{8d} is —CH₂-A.5 and R⁵, R⁶ and R⁷ for a compound corresponds in each case to one row of Table A

### Table 11029

Compounds of the formula Ia.24 in which X is CH, —R^{8d} is —CH₂-A.6 and R⁵, R⁶ and R⁷ for a compound corresponds in each case to one row of Table A

### Table 11030

Compounds of the formula Ia.24 in which X is CH, —R^{8d} is —CH₂-A.7 and R⁵, R⁶ and R⁷ for a compound corresponds in each case to one row of Table A

### Table 11031

Compounds of the formula Ia.24 in which X is CH, —R^{8d} is —CH₂-A.8 and R⁵, R⁶ and R⁷ for a compound corresponds in each case to one row of Table A

### Table 11032

Compounds of the formula Ia.24 in which X is CH, —R^{8d} is —CH₂-A.9 and R⁵, R⁶ and R⁷ for a compound corresponds in each case to one row of Table A

### Table 11033

Compounds of the formula Ia.24 in which X is CH, —R^{8d} is —CH₂-A.10 and R⁵, R⁶ and R⁷ for a compound corresponds in each case to one row of Table A

### Table 11034

Compounds of the formula Ia.24 in which X is CH, —R^{8d} is —CH₂-A.11 and R⁵, R⁶ and R⁷ for a compound corresponds in each case to one row of Table A

### Table 11035

Compounds of the formula Ia.24 in which X is CH, —R^{8d} is —CH₂-A.12 and R⁵, R⁶ and R⁷ for a compound corresponds in each case to one row of Table A

### Table 11036

Compounds of the formula Ia.24 in which X is CH, —R^{8d} is —CH₂-A.13 and R⁵, R⁶ and R⁷ for a compound corresponds in each case to one row of Table A

### Table 11037

Compounds of the formula Ia.24 in which X is CH, —R^{8d} is —CH₂-A.14 and R⁵, R⁶ and R⁷ for a compound corresponds in each case to one row of Table A

### Table 11038

Compounds of the formula Ia.24 in which X is CH, —R^{8d} is —CH₂-A.15 and R⁵, R⁶ and R⁷ for a compound corresponds in each case to one row of Table A

### Table 11039

Compounds of the formula Ia.24 in which X is CH, —R^{8d} is —CH₂-A.16 and R⁵, R⁶ and R⁷ for a compound corresponds in each case to one row of Table A

### Table 11040

Compounds of the formula Ia.24 in which X is CH, —R^{8d} is —CH₂-A.17 and R⁵, R⁶ and R⁷ for a compound corresponds in each case to one row of Table A

### Table 11041

Compounds of the formula Ia.24 in which X is CH, —R^{8d} is —CH₂-A.18 and R⁵, R⁶ and R⁷ for a compound corresponds in each case to one row of Table A

### Table 11042

Compounds of the formula Ia.24 in which X is CH, —R^{8d} is —CH₂-A.19 and R⁵, R⁶ and R⁷ for a compound corresponds in each case to one row of Table A

### Table 11043

Compounds of the formula Ia.24 in which X is CH, —R^{8d} is —CH₂-A.20 and R⁵, R⁶ and R⁷ for a compound corresponds in each case to one row of Table A

### Table 11044

Compounds of the formula Ia.24 in which X is CH, —R^{8d} is —CH₂-A.21 and R⁵, R⁶ and R⁷ for a compound corresponds in each case to one row of Table A

### Table 11045

Compounds of the formula Ia.24 in which X is CH, —R^{8d} is —CH₂-A.22 and R⁵, R⁶ and R⁷ for a compound corresponds in each case to one row of Table A

### Table 11046

Compounds of the formula Ia.24 in which X is CH, —R^{8d} is —CH₂-A.23 and R⁵, R⁶ and R⁷ for a compound corresponds in each case to one row of Table A

### Table 11047

Compounds of the formula Ia.24 in which X is CH, —R^{8d} is —CH₂-A.24 and R⁵, R⁶ and R⁷ for a compound corresponds in each case to one row of Table A

### Table 11048

Compounds of the formula Ia.24 in which X is CH, —R^{8d} is —CH₂-A.25 and R⁵, R⁶ and R⁷ for a compound corresponds in each case to one row of Table A

### Table 11049

Compounds of the formula Ia.24 in which X is CH, —R^{8d} is —CH₂-A.26 and R⁵, R⁶ and R⁷ for a compound corresponds in each case to one row of Table A

### Table 11050

Compounds of the formula Ia.24 in which X is CH, —R^{8d} is —CH₂-A.27 and R⁵, R⁶ and R⁷ for a compound corresponds in each case to one row of Table A

### Table 11051

Compounds of the formula Ia.24 in which X is CH, —R^{8d} is —CH₂-A.28 and R⁵, R⁶ and R⁷ for a compound corresponds in each case to one row of Table A

### Table 11052

Compounds of the formula Ia.24 in which X is CH, —R^{8d} is —CH₂-A.29 and R⁵, R⁶ and R⁷ for a compound corresponds in each case to one row of Table A

### Table 11053

Compounds of the formula Ia.24 in which X is CH, —R^{8d} is —CH₂-A.30 and R⁵, R⁶ and R⁷ for a compound corresponds in each case to one row of Table A

### Table 11054

Compounds of the formula Ia.24 in which X is CH, —R^{8d} is —CH₂-A.31 and R⁵, R⁶ and R⁷ for a compound corresponds in each case to one row of Table A

### Table 11055

Compounds of the formula Ia.24 in which X is CH, —R^{8d} is —CH₂-A.32 and R⁵, R⁶ and R⁷ for a compound corresponds in each case to one row of Table A

### Table 11056

Compounds of the formula Ia.24 in which X is CH, —R^{8d} is —CH₂-A.33 and R⁵, R⁶ and R⁷ for a compound corresponds in each case to one row of Table A

### Table 11057

Compounds of the formula Ia.24 in which X is CH, —R^{8d} is —CH₂-A.34 and R⁵, R⁶ and R⁷ for a compound corresponds in each case to one row of Table A

### Table 11058

Compounds of the formula Ia.24 in which X is CH, —R^{8d} is —CH₂-A.35 and R⁵, R⁶ and R⁷ for a compound corresponds in each case to one row of Table A

### Table 11059

Compounds of the formula Ia.24 in which X is CH, —R^{8d} is —CH₂-A.36 and R⁵, R⁶ and R⁷ for a compound corresponds in each case to one row of Table A

### Table 11060

Compounds of the formula Ia.24 in which X is CH, —R^{8d} is —CH₂-A.37 and R⁵, R⁶ and R⁷ for a compound corresponds in each case to one row of Table A

### Table 11061

Compounds of the formula Ia.24 in which X is CH, —R^{8d} is —CH₂-A.38 and R⁵, R⁶ and R⁷ for a compound corresponds in each case to one row of Table A

### Table 11062

Compounds of the formula Ia.24 in which X is CH, —R^{8d} is —CH₂-A.39 and R⁵, R⁶ and R⁷ for a compound corresponds in each case to one row of Table A

### Table 11063

Compounds of the formula Ia.24 in which X is CH, —R^{8d} is —CH₂-A.40 and R⁵, R⁶ and R⁷ for a compound corresponds in each case to one row of Table A

### Table 11064

Compounds of the formula Ia.24 in which X is CH, —R^{8d} is —CH₂-A.41 and R⁵, R⁶ and R⁷ for a compound corresponds in each case to one row of Table A

### Table 11065

Compounds of the formula Ia.24 in which X is CH, —R^{8d} is —CH₂-A.42 and R⁵, R⁶ and R⁷ for a compound corresponds in each case to one row of Table A

### Table 11066

Compounds of the formula Ia.24 in which X is CH, —R^{8d} is —CH₂-A.43 and R⁵, R⁶ and R⁷ for a compound corresponds in each case to one row of Table A

### Table 11067

Compounds of the formula Ia.24 in which X is CH, —R^{8d} is —CH₂-A.44 and R⁵, R⁶ and R⁷ for a compound corresponds in each case to one row of Table A

### Table 11068

Compounds of the formula Ia.24 in which X is CH, —R^{8d} is —CH₂-A.45 and R⁵, R⁶ and R⁷ for a compound corresponds in each case to one row of Table A

### Table 11069

Compounds of the formula Ia.24 in which X is CH, —R^{8d} is —CH₂-A.46 and R⁵, R⁶ and R⁷ for a compound corresponds in each case to one row of Table A

### Table 11070

Compounds of the formula Ia.24 in which X is CH, —R^{8d} is —CH₂-A.47 and R⁵, R⁶ and R⁷ for a compound corresponds in each case to one row of Table A

### Table 11071

Compounds of the formula Ia.24 in which X is CH, —R^{8d} is —CH₂-A.48 and R⁵, R⁶ and R⁷ for a compound corresponds in each case to one row of Table A

### Table 11072

Compounds of the formula Ia.24 in which X is CH, —R^{8d} is —CH₂-A.49 and R⁵, R⁶ and R⁷ for a compound corresponds in each case to one row of Table A

### Table 11073

Compounds of the formula Ia.24 in which X is CH, —R^{8d} is —CH₂-A.50 and R⁵, R⁶ and R⁷ for a compound corresponds in each case to one row of Table A

### Table 11074

Compounds of the formula Ia.24 in which X is CH, —R^{8d} is —CH₂-A.51 and R⁵, R⁶ and R⁷ for a compound corresponds in each case to one row of Table A

### Table 11075

Compounds of the formula Ia.24 in which X is CH, —R^{8d} is —CH₂-A.52 and R⁵, R⁶ and R⁷ for a compound corresponds in each case to one row of Table A

### Table 11076

Compounds of the formula Ia.24 in which X is CH, —R^{8d} is —CH₂-A.53 and R⁵, R⁶ and R⁷ for a compound corresponds in each case to one row of Table A

### Table 11077

Compounds of the formula Ia.24 in which X is CH, —R^{8d} is —CH₂-A.54 and R⁵, R⁶ and R⁷ for a compound corresponds in each case to one row of Table A

### Table 11078

Compounds of the formula Ia.24 in which X is CH, —R^{8d} is —CH₂-A.55 and R⁵, R⁶ and R⁷ for a compound corresponds in each case to one row of Table A

### Table 11079

Compounds of the formula Ia.24 in which X is CH, —R^{8d} is —CH₂-A.56 and R⁵, R⁶ and R⁷ for a compound corresponds in each case to one row of Table A

### Table 11080

Compounds of the formula Ia.24 in which X is CH, —R^{8d} is —CH₂-A.57 and R⁵, R⁶ and R⁷ for a compound corresponds in each case to one row of Table A

### Table 11081

Compounds of the formula Ia.24 in which X is CH, —R^{8d} is —CH₂-A.58 and R⁵, R⁶ and R⁷ for a compound corresponds in each case to one row of Table A

### Table 11082

Compounds of the formula Ia.24 in which X is CH, —R^{8d} is —CH₂-A.59 and R⁵, R⁶ and R⁷ for a compound corresponds in each case to one row of Table A

### Table 11083

Compounds of the formula Ia.24 in which X is CH, —R^{8d} is —CH₂-A.60 and R⁵, R⁶ and R⁷ for a compound corresponds in each case to one row of Table A

### Table 11084

Compounds of the formula Ia.24 in which X is CH, —R^{8d} is —CH₂-A.61 and R⁵, R⁶ and R⁷ for a compound corresponds in each case to one row of Table A

### Table 11085

Compounds of the formula Ia.24 in which X is CH, —R^{8d} is —CH₂-A.62 and R⁵, R⁶ and R⁷ for a compound corresponds in each case to one row of Table A

### Table 11086

Compounds of the formula Ia.24 in which X is CH, —R^{8d} is —CH₂-A.63 and R⁵, R⁶ and R⁷ for a compound corresponds in each case to one row of Table A

### Table 11087

Compounds of the formula Ia.24 in which X is CH, —R^{8d} is —CH₂-A.64 and R⁵, R⁶ and R⁷ for a compound corresponds in each case to one row of Table A

### Table 11088

Compounds of the formula Ia.24 in which X is CH, —R^{8d} is —CH₂-A.65 and R⁵, R⁶ and R⁷ for a compound corresponds in each case to one row of Table A

### Table 11089

Compounds of the formula Ia.24 in which X is CH, —R^{8d} is —CH₂-A.66 and R⁵, R⁶ and R⁷ for a compound corresponds in each case to one row of Table A

### Table 11090

Compounds of the formula Ia.24 in which X is CH, —R^{8d} is —CH₂-A.67 and R⁵, R⁶ and R⁷ for a compound corresponds in each case to one row of Table A

### Table 11091

Compounds of the formula Ia.24 in which X is CH, —R^{8d} is —CH₂-A.68 and R⁵, R⁶ and R⁷ for a compound corresponds in each case to one row of Table A

### Table 11092

Compounds of the formula Ia.24 in which X is CH, —R^{8d} is —CH₂-A.92 and R⁵, R⁶ and R⁷ for a compound corresponds in each case to one row of Table A

### Table 11093

Compounds of the formula Ia.24 in which X is CH, —R^{8d} is —CH₂-A.93 and R⁵, R⁶ and R⁷ for a compound corresponds in each case to one row of Table A

### Table 11094

Compounds of the formula Ia.24 in which X is CH, —R^{8d} is —CH₂-A.94 and R⁵, R⁶ and R⁷ for a compound corresponds in each case to one row of Table A

### Table 11095

Compounds of the formula Ia.24 in which X is CH, —R^{8d} is —CH₂-A.95 and R⁵, R⁶ and R⁷ for a compound corresponds in each case to one row of Table A

### Table 11096

Compounds of the formula Ia.24 in which X is CH, —R^{8d} is —CH₂-A.96 and R⁵, R⁶ and R⁷ for a compound corresponds in each case to one row of Table A

### Table 11097

Compounds of the formula Ia.24 in which X is CH, —R^{8d} is —CH₂—CH₂-A.59 and R⁵, R⁶ and R⁷ for a compound corresponds in each case to one row of Table A

### Table 11098

Compounds of the formula Ia.24 in which X is CH, —R^{8d} is —CF₂—CH₂-A.59 and R⁵, R⁶ and R⁷ for a compound corresponds in each case to one row of Table A

### Table 11099

Compounds of the formula Ia.24 in which X is CH, —R^{8d} is —CF₂—CHCl-A.59 and R⁵, R⁶ and R⁷ for a compound corresponds in each case to one row of Table A

### Table 11100

Compounds of the formula Ia.24 in which X is CH, —R^{8d} is —CH₂—OCH₃ and R⁵, R⁶ and R⁷ for a compound corresponds in each case to one row of Table A

### Table 11101

Compounds of the formula Ia.24 in which X is CH, —R^{8d} is —(CH₂)₂—OCH₃ and R⁵, R⁶ and R⁷ for a compound corresponds in each case to one row of Table A

### Table 11102

Compounds of the formula Ia.24 in which X is CH, —R^{8d} is —(CH₂)₃—OCH₃ and R⁵, R⁶ and R⁷ for a compound corresponds in each case to one row of Table A

### Table 11103

Compounds of the formula Ia.24 in which X is CH, —R^{8d} is —(CH₂)₄—OCH₃ and R⁵, R⁶ and R⁷ for a compound corresponds in each case to one row of Table A

### Tables 11104 to 11256

Compounds of the formula Ia.24 in which X is N, —R^{8d} is as defined in any of tables 10951 to 11103 and R⁵, R⁶ and R⁷ for a compound corresponds in each case to one row of Table A

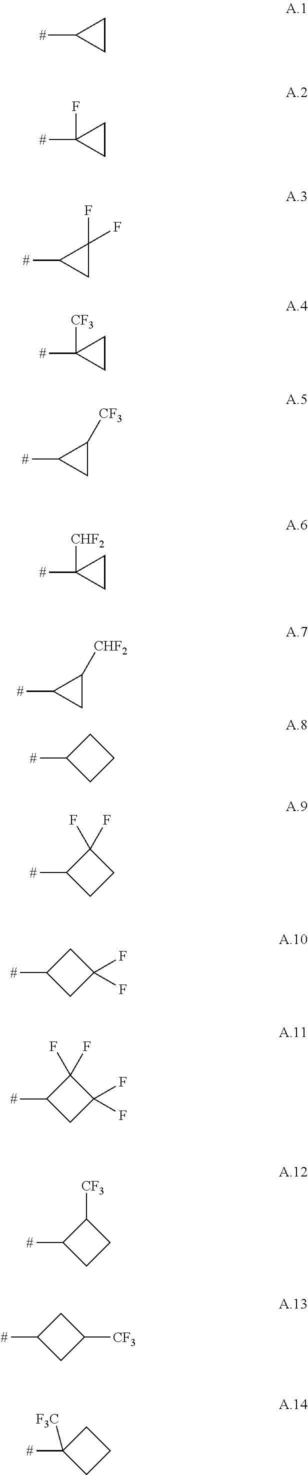

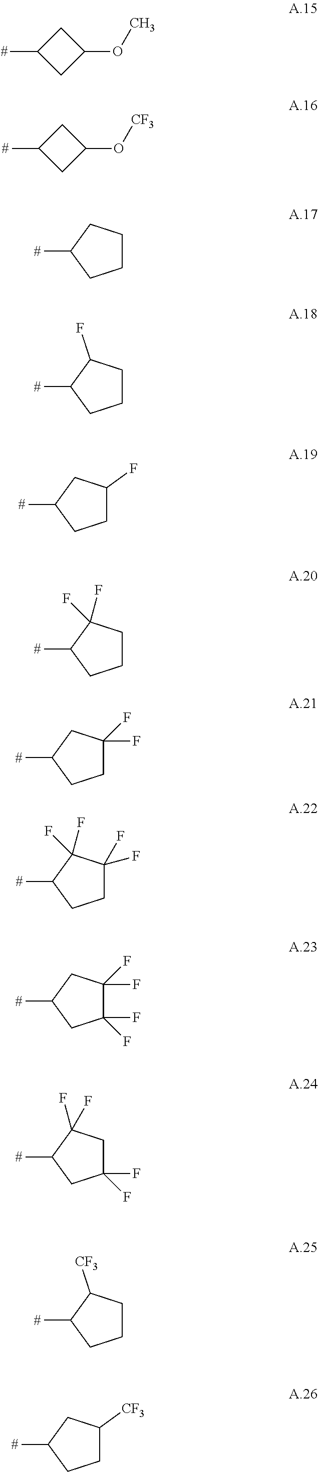

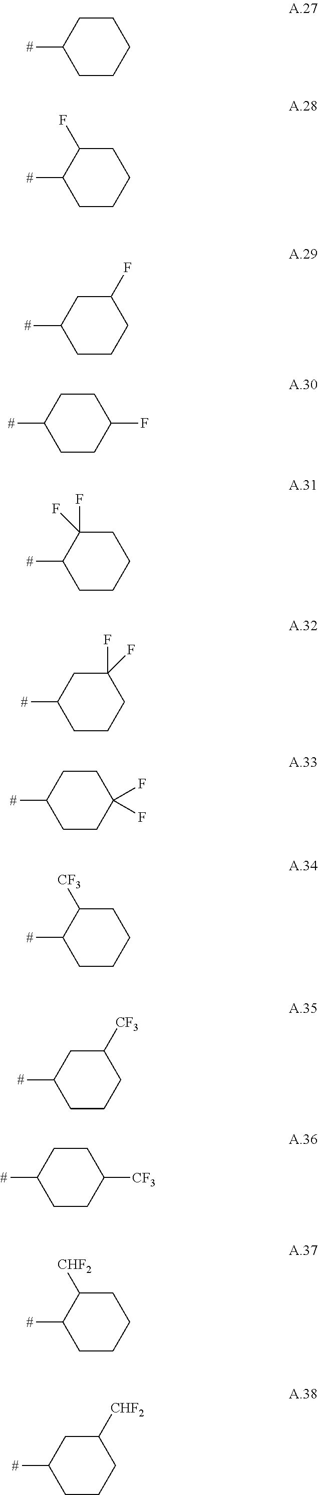

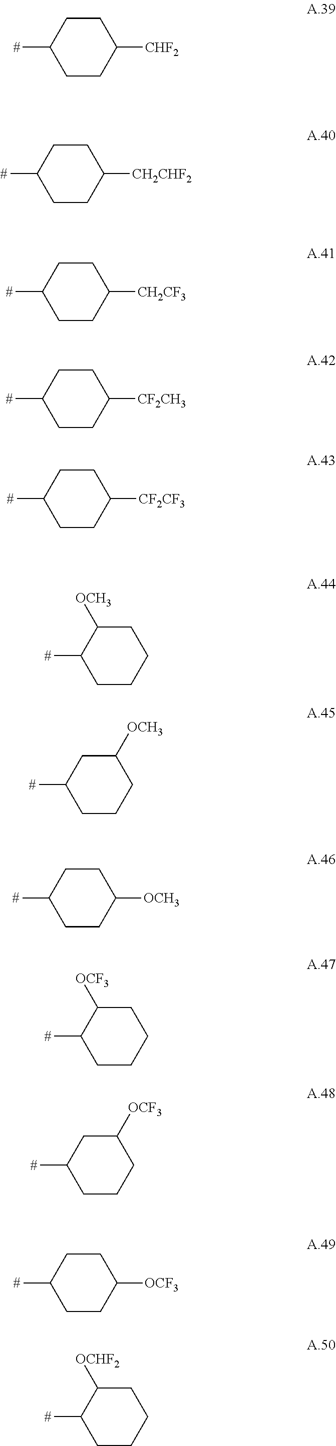

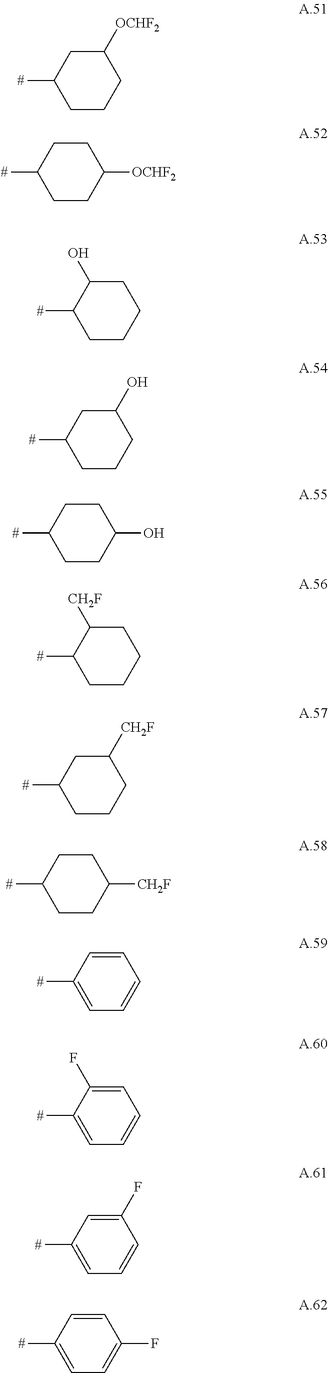

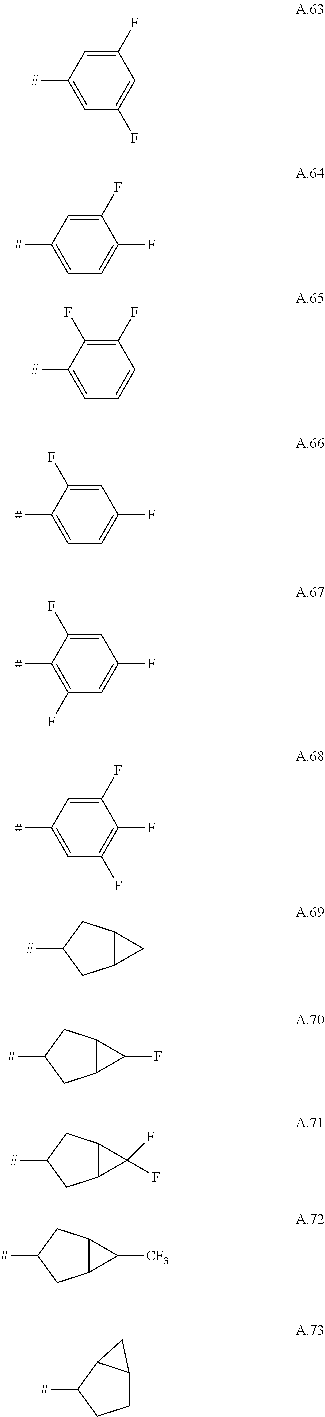

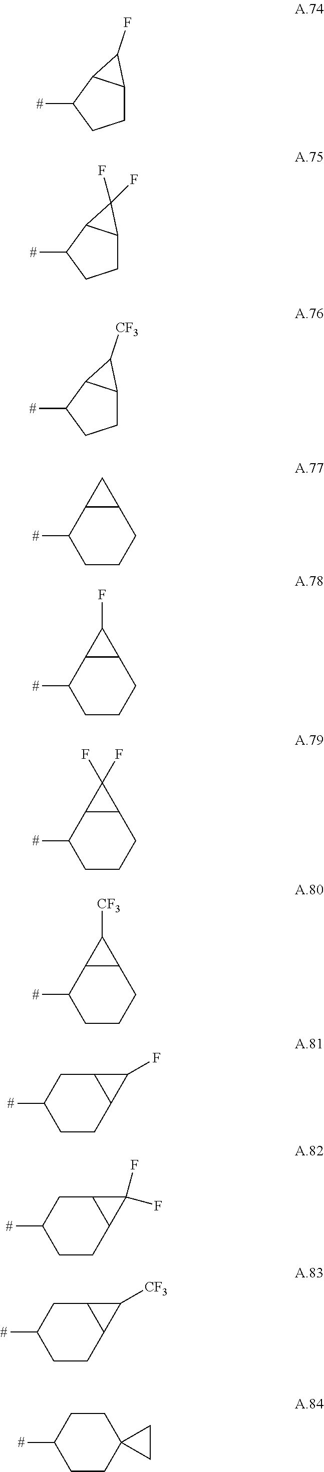

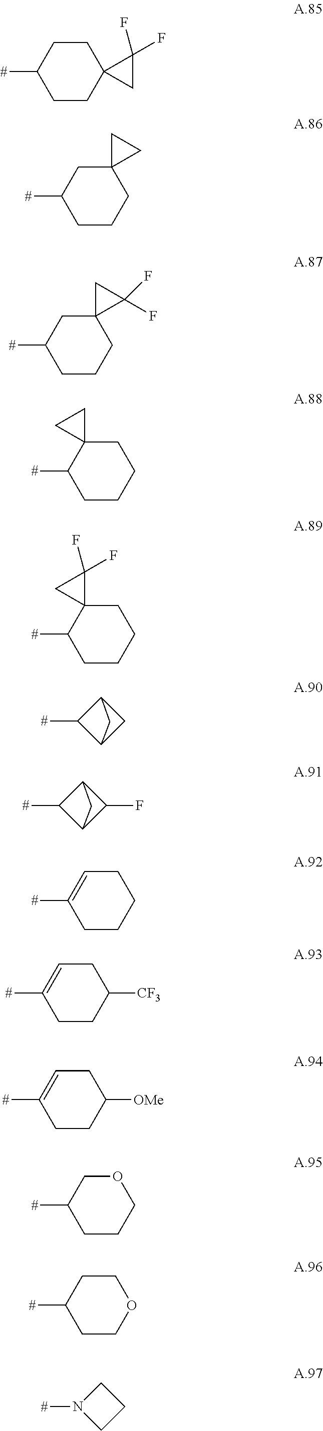

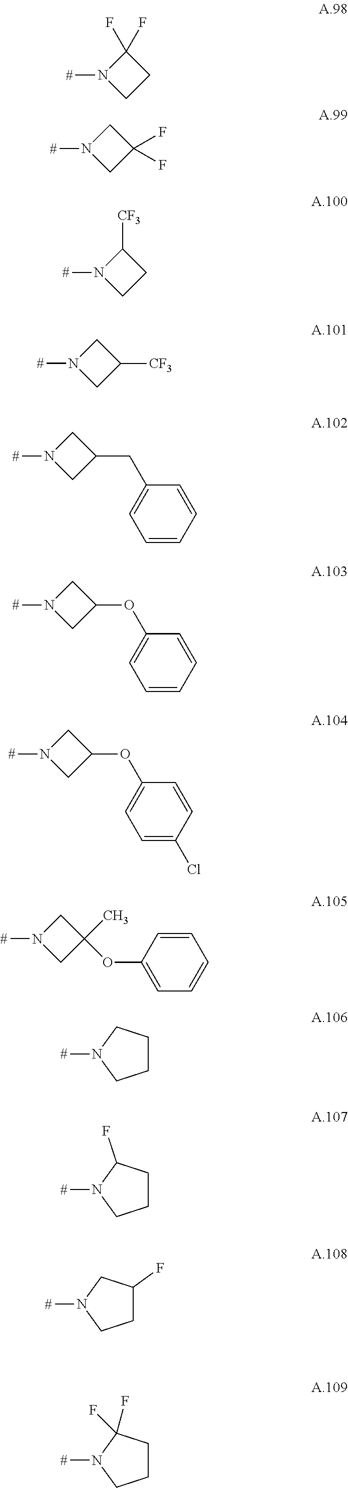

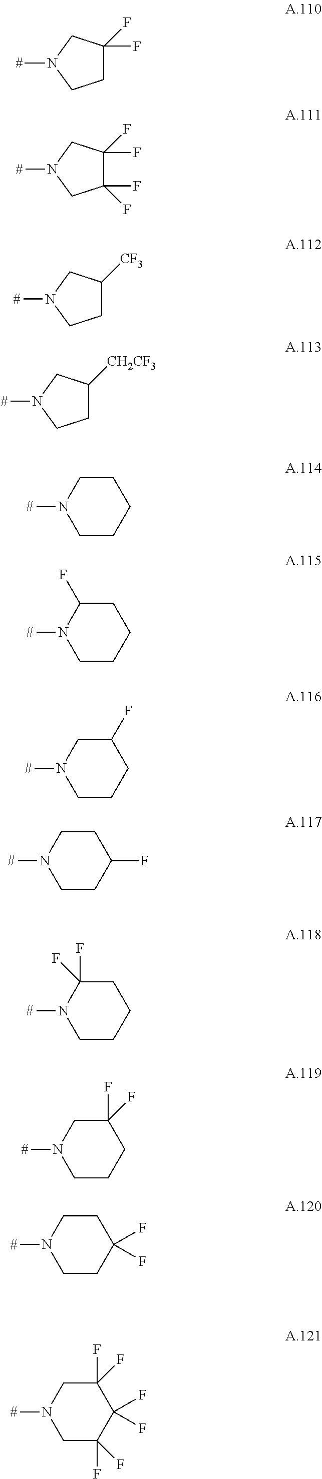

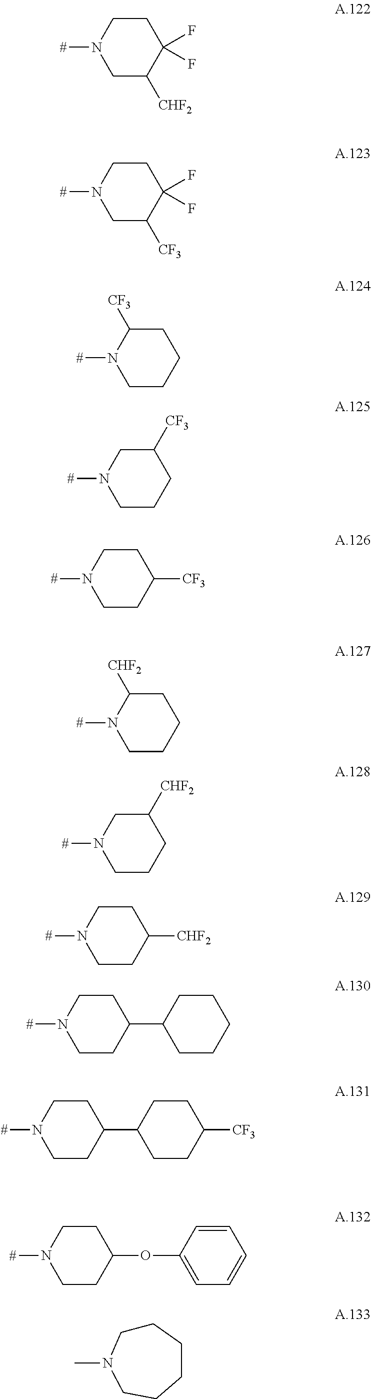

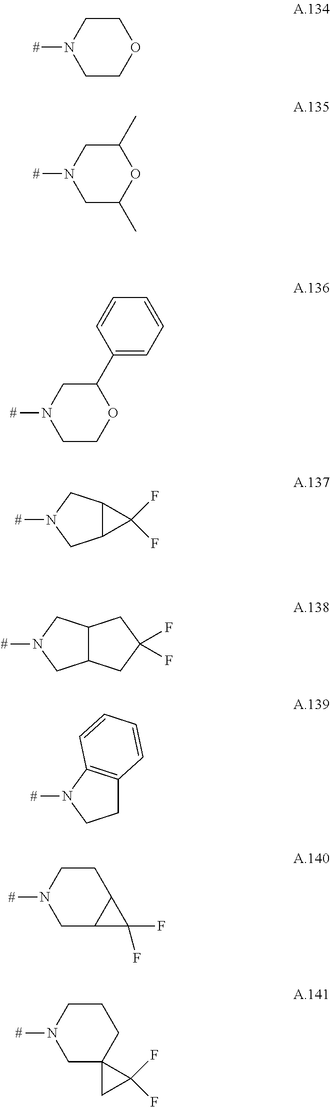

# is the attachment point to the remainder of the molecule

TABLE A

| No. | R⁵ | R⁶ | R⁷ |
|---|---|---|---|
| A-1. | H | H | OCH₃ |
| A-2. | H | H | OCH₂CH₃ |
| A-3. | H | H | OCH₂CH₂CH₃ |
| A-4. | H | H | OCH(CH₃)₂ |
| A-5. | H | H | OH |
| A-6. | H | H | CH₃ |
| A-7. | H | F | OCH₃ |
| A-8. | H | F | OCH₂CH₃ |
| A-9. | H | F | OCH₂CH₂CH₃ |
| A-10. | H | F | OCH(CH₃)₂ |
| A-11. | H | F | OH |
| A-12. | H | F | CH₃ |
| A-13. | H | Cl | OCH₃ |
| A-14. | H | Cl | OCH₂CH₃ |
| A-15. | H | Cl | OCH₂CH₂CH₃ |
| A-16. | H | Cl | OCH(CH₃)₂ |
| A-17. | H | Cl | OH |
| A-18. | H | Cl | CH₃ |
| A-19. | CH₃ | H | OCH₃ |
| A-20. | CH₃ | H | OCH₂CH₃ |
| A-21. | CH₃ | H | OCH₂CH₂CH₃ |
| A-22. | CH₃ | H | OCH(CH₃)₂ |
| A-23. | CH₃ | H | OH |
| A-24. | CH₃ | H | CH₃ |
| A-25. | CH₃ | F | OCH₃ |
| A-26. | CH₃ | F | OCH₂CH₃ |
| A-27. | CH₃ | F | OCH₂CH₂CH₃ |
| A-28. | CH₃ | F | OCH(CH₃)₂ |
| A-29. | CH₃ | F | OH |
| A-30. | CH₃ | F | CH₃ |
| A-31. | CH₃ | Cl | OCH₃ |
| A-32. | CH₃ | Cl | OCH₂CH₃ |
| A-33. | CH₃ | Cl | OCH₂CH₂CH₃ |
| A-34. | CH₃ | Cl | OCH(CH₃)₂ |
| A-35. | CH₃ | Cl | OH |
| A-36. | CH₃ | Cl | CH₃ |

Among the above compounds, preference is given to compounds Ia.1, Ia.2, Ia.23 and Ia.24.
In compounds Ia.1 to Ia.6, Ia.23 and Ia.24 the pyrrolidine ring is preferably derived from L-proline, i.e. it has preferably following configuration:

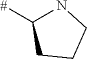

In a specific embodiment, the invention relates to compounds I selected from the compounds of the examples, either in form of free bases or of any pharmaceutically acceptable salt thereof or a stereoisomer, the racemate or any mixture of stereoisomers thereof or a tautomer or a tautomeric mixture or an N-oxide thereof.

The compounds of the present invention can be prepared by using routine techniques familiar to a skilled person. In particular, the compounds of the formula I can be prepared according to the following schemes, wherein the variables, if not stated otherwise, are as defined above.

Compounds of formula I wherein none of R¹ and R² is hydrogen (called hereinafter compounds I′) can be prepared as outlined in scheme 1 below. Amine 1 is coupled with amino acid derivative 2 under standard amidation conditions, wherein LG represents a suitable leaving group, such as Cl, Br, I, triflate or tosylate. The reaction is generally carried out under basic conditions. Alternatively, LG is OH and amidation is carried out in the presence of a coupling reagent. Suitable coupling reagent (activators) are well known and are for instance selected from carbodiimides, such as DCC (dicyclohexylcarbodiimide) and DCI (diisopropylcarbodiimide), benzotriazol derivatives, such as HATU (O-(7-azabenzotriazol-1-yl)-N,N,N′,N′-tetramethyluronium hexafluorophosphate), HBTU ((O-benzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate). Generally, the activator is used in excess.

The benzotriazol and phosphonium coupling reagents are generally used in a basic medium.

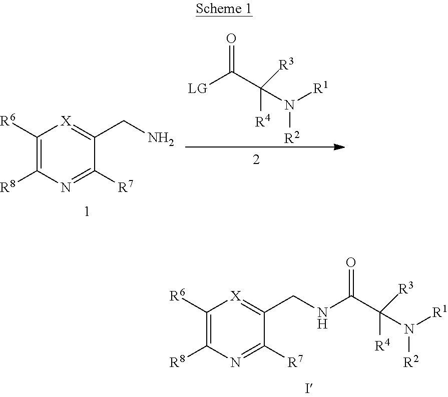

For preparing compounds I wherein R¹ or R¹ and R² are hydrogen (termed hereinafter compounds I″), protected amino acid derivatives 3 are suitably used (PG is a protective group; the below scheme 2 shows the case in which only R¹ is hydrogen). These are reacted with amine 1 as shown in scheme 2 below under amidation conditions as described above for the reaction in scheme 1. Suitable protective groups are for example C₁-C₄-alkylcarbonyl (e.g. acetyl), C₁-C₄-haloalkylcarbonyl (e.g. trifluoroacetyl), C₃-C₄-alkenylcarbonyl (e.g. allylcarbonyl), C₁-C₄-alkoxycarbonyl (e.g. Boc), C₁-C₄-haloalkoxycarbonyl, C₃-C₄-alkenyloxycarbonyl, C₁-C₄-alkylaminocarbonyl, di-(C₁-C₄-alkyl)-aminocarbonyl, C₁-C₄-alkylsulfonyl, C₁-C₄-haloalkylsulfonyl or benzyl. The choice of the protective group depends on the reaction conditions in the amidation reaction. The protective group is chosen so that it is not hydrolyzed during the amidation reaction. Deprotection of 4 yields I″. Deprotection conditions depend on the protective group used. If desired, compounds I″ can be converted into compounds I wherein R¹ or R¹ and R² are alkyl by subjecting compounds I″ to an alkylation reaction or to a reductive amination.

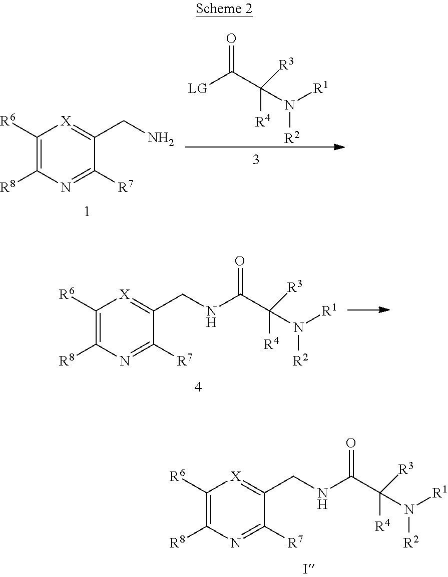

Alternatively, compounds I can be prepared as outlined in scheme 3 below. Carbonyl compounds 5 are reacted with amine 1 as shown in scheme 3 below under amidation conditions as described above for the reaction in scheme 1. LG¹ and LG² independently have one of the meanings given for LG in compounds 2 in scheme 1. Amination of compound 6 with the amine NHR¹R² is suitably carried out under basic conditions.

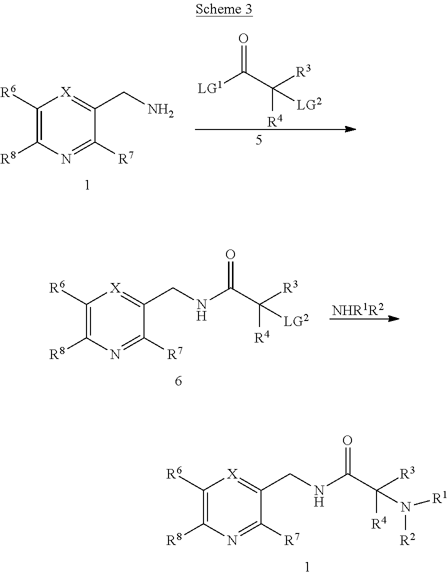

Compounds of formula I′ wherein none of R¹ and R² is hydrogen can alternatively be prepared by introducing the radical —R⁸ only after the introduction of the amino acid group, as shown in scheme 4 below. Amidation of amine 7 with the amino acid derivative 2 can be carried out in analogy to the amidation reaction in scheme 1. Compounds I′ wherein R⁸ is —OR^{8a} are e.g. obtained via Pd coupling of 8 with the alcohol HO—R^{8a}. The Pd catalyst is usually used with a phosphorus ligand, such as 2,2′-bis(diphenylphosphino)-1,1′-binaphthyl (BINAP), [1,1′-biphenyl]-2-diisopropyl phosphine, 1,1′-bis(diphenylphospino)ferrocene (dppf), X-phos, di-tert-butyl(2′,4′,6′-triisopropyl-[1,1′-biphenyl]-2-yl)phosphine (t-BuXPhos), 9,9-dimethyl-4,5-bis(diphenylphosphino)xanthene (Xantphos), 4,5-bis-(di-1-(3-methylindolyl)phosphoramidit)-2,7,9,9-tetramethylxanthene (MeSkatOX), triphenylphosphine, triphenylphosphite, tri-(2-(1,1-dimethylethyl)-4-methoxy-phenyl)-phosphite, tricyclohexylphosphine, butyldi-1-adamantylphosphine (cataCXium), 1,6-bis(diphenylphosphino)hexane (DPPH), 2,6-bis(2,5-dimethylphenyl)-1-octyl-4-phenylphosphacyclohexan (PCH), 2-dicyclohexylphosphino-2′,6′-diisopropoxybiphenyl (RuPhos) and the like. The reaction is generally carried out in the presence of a base, advantageously a non-nucleophilic base, e.g. a carbonate, such as lithium, sodium, potassium or caesium carbonate, DBU, DBN and the like, or a sterically hindered nucleophilic alcoholate, like sodium or potassium tert-butanolate. Sterically non-demanding nucleophilic bases can be used if they are first reacted with the alcohol HO—R^{8a} before compound 8 is added. Suitable bases for this purpose are e.g. methanolates, e.g. sodium or potassium methanolate, ethanolates, e.g. sodium or potassium ethanolate, hydroxides, such as sodium or potassium hydroxide, hydrides, such as sodium or potassium hydride, and LDA. Non-nucleophilic bases or sterically hindered nucleophilic alcoholates can of course also be used for first deprotonating the alcohol HO—R^{8a} before compound 8 is added, as long as they are strong enough for the deprotonation.

Compounds I′ wherein R⁸ is —NR^{8b}R^{8c} are e.g. obtained via a Buchwald-Hartwig reaction of 8 with the amine NHR^{8b}R^{8c}. The Buchwald-Hartwig reaction is transition metal-catalyzed, mostly Pd catalyzed, and is generally carried out in the presence of a base. The Pd catalyst is usually used with a phosphorus ligand, such as the phosphorus ligands listed above. Suitable bases are those listed above; advantageously a non-nucleophilic base is used.

Analogously, compounds I′ wherein R⁸ is —R^{8d} can be obtained via an S_{N} reaction on 8 if the hydrogen atom in H—R^{8d} is acidic enough to be removed under suitable conditions, such as basic medium and/or transition metal catalysis. This reaction path is for example viable for haloalkyl groups R^{8d}, especially for compounds H—R^{8d} in which the hydrogen atom shown is bound to a carbon atom carrying one or, better, two halogen atoms. The reaction proceeds even better if the carbon atom carrying said hydrogen atom and the one or two halogen atoms is further bound to an electron-withdrawing group, such as a carbonyl group. After completion of the coupling reaction this carbonyl group can be converted into a CH₂ or halogenated methylene group. As said, the reaction is carried out in the presence of a base and suitably also in the presence of a transition metal catalyst, especially of a Pd catalyst. Suitable bases are those listed above; advantageously a non-nucleophilic base is used. Suitable Pd catalysts and ligands therefor are also those mentioned above.

Alternatively, compounds I′ wherein R⁸ is —R^{8d} can be obtained via a Suzuki reaction (also called Suzuki coupling, Suzuki-Miyaura reaction or Suzuki-Miyaura coupling) (not shown in scheme 4) of 8 with an organoboron compound of R^{8d}. The organoboron compound is generally a compound of formula R^{8d}—BY₂, where Y is an alkyl group (e.g. C₁-C₄-alkyl), an O-alkyl group (e.g. C₁-C₄-alkoxy) or a hydroxyl group, or the two substituents Y form together with the boron atom they are bound to a mono-, bi- or polycyclic ring; or the organoboron compound is a compound of formula R^{8d}—BF₃M, where M is a metal equivalent. Examples of suitable organoboron compounds R^{8d}—BY₂ are R^{8d}—B(OH)₂, R^{8d}—B(O—C₁-C₄-alkyl)₂, R^{8d}—B(C₁-C₄-alkyl)₂, the pinacol ester of R^{8d}—B(OH)₂ (i.e. the two Y form together —O—C(CH₃)₂—C(CH₃)₂—O—), or the MIDA ester of R^{8d}—B(OH)₂ (MIDA=N-methyliminodiacetic acid; HO—C(═O)—CH₂—N(CH₃)—CH₂—C(═O)—OH; i.e. the two Y form together —O—C(═O)—CH₂—N(CH₃)—CH₂—C(═O)—O—). The Suzuki reaction is carried out in the presence of a transition metal catalyst, mostly a Pd or Ni catalyst, and generally also in the presence of a base. The Pd or Ni catalyst is usually used with a phosphorus ligand, such as the phosphorus ligands listed above. Suitable bases can be inorganic or organic. Examples for suitable inorganic bases are alkali metal carbonates, e.g. Li₂CO₃, Na₂CO₃, K₂CO₃ or Cs₂CO₃, alkali metal hydrogen carbonates, e.g. LiHCO₃, NaCO₃, KHCO₃ or CsHCO₃, alkali metal hydroxides, e.g. LiOH, NaOH or KOH, or phosphates, e.g. Li₃PO₄, Na₃PO₄, K₃PO₄ or Cs₃PO₄. Examples for suitable organic bases are open-chained amines, e.g. trimethylamine, triethylamine, tripropylamine, ethyldiisopropylamine and the like, basic N-heterocycles, such as morpoline, pyridine, lutidine, DABCO, DBU or DBN, alkoxylates, e.g. sodium or potassium methanolate, ethanolate, propanolate, isopropanolate, butanolate or tert-butanolate, especially sterically hindered alkoxylates, such as sodium or potassium tert-butanolate, silanolates, like sodium or potassium trimethylsilanolate ((CH₃)₃SiO⁻) or triisopropylsilanolate ((CH(CH₃)₂)₃SiO⁻), phosphazene bases (superbases), such as BEMP and t-Bu-P4, or sterically hindered phenolates.

Alternatively, compounds I′ wherein R⁸ is —R^{8d}, wherein R^{8d} is C₂-C₄-alkyl which carries a radical R^{9c}; C₂-C₄-haloalkyl which carries a radical R^{9c}; C₃-C₆-cycloalkyl which may carry one or more substituents R¹¹; or C₃-C₆-cycloalkenyl which may carry one or more substituents R¹¹, can be obtained via a Heck reaction (not shown in scheme 4) of 8 with an unsaturated compound of H—R^{8dd}, wherein R^{8dd} is an alkene precursor of the C₂-C₄-alkyl group R^{8d} which carries a radical R^{9c}; a haloalkene precursor of the C₂-C₄-haloalkyl group R^{8d} which carries a radical R^{9c}; a cyckloalkene precursor of the C₃-C₆-cycloalkyl group R^{8d} which may carry one or more substituents R¹¹; or is C₃-C₆-cycloalkenyl which may carry one or more substituents R¹¹. The Heck reaction is carried out in the presence of a transition metal catalyst, mostly a Pd catalyst, and generally also in the presence of a base. Suitable bases are those listed above; advantageously a non-nucleophilic base is used. Suitable Pd catalysts and ligands therefor are also those mentioned above. The resulting coupling product is then hydrogenated to the desired compound I′ in case that R^{8d} is to be C₂-C₄-alkyl which carries a radical R^{9c}; C₂-C₄-haloalkyl which carries a radical R^{9c}; or C₃-C₆-cycloalkyl which may carry one or more substituents R¹¹.

Alternatively, compounds I′ wherein R⁸ is —R^{8d}, wherein R^{8d} is C₂-C₄-alkyl which carries a radical R^{9c}; or is C₂-C₄-haloalkyl which carries a radical R^{9c}, can be obtained via a Sonogashira reaction (not shown in scheme 4) of 8 with a compound H—R^{8dd}, wherein R^{8dd} is an alkyne precursor of the C₂-C₄-alkyl group R^{8d} which carries a radical R^{9c} or is a haloalkyne precursor of the C₂-C₄-haloalkyl group R^{8d} which carries a radical R^{9c}, where the triple bond of the (halo)alkyne is in terminal position. The Sonogashira reaction is carried out in the presence of a transition metal catalyst, mostly a Pd catalyst, optionally of a copper(I) salt, and generally also in the presence of a base. Suitable bases are those listed above; advantageously a non-nucleophilic base is used. Suitable Pd catalysts and ligands therefor are also those mentioned above. The resulting coupling product is then hydrogenated.

Alternatively, compounds I′ wherein R⁸ is —R^{8d} can be obtained via a Negishi reaction (not shown in scheme 4) of 8 with an organozinc compound R^{8d}—ZnX wherein X is chloride, bromide, iodide, triflate or acetyloxy. Instead of organozinc compounds organoaluminum or organozirconium compounds can be used. If these are not reactive enough they can be transmetallated to the corresponding zinc compounds by addition of zinc salts (“double metal catalysis”). The Negishi reaction is carried out in the presence of a transition metal catalyst, mostly a Pd or Ni catalyst, where the Pd catalyst is often better suited. The reaction does not need the presence of a further booster, such as the base in the Suzuki coupling. Suitable Pd (inclusive ligands) are those mentioned above.

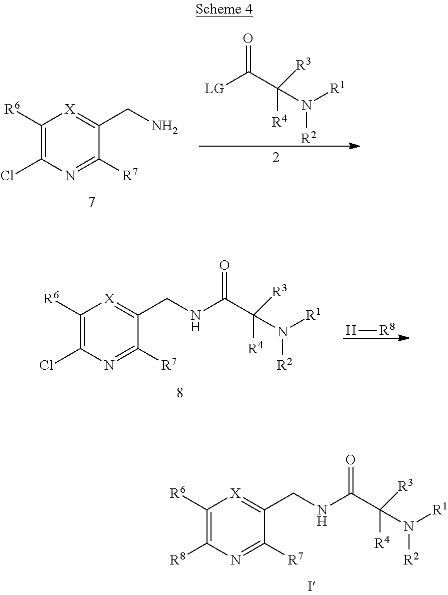

Also compounds I″ wherein R¹ or R¹ and R² are hydrogen can alternatively be prepared by introducing the radical —R⁸ only after the introduction of the amino acid group, as shown in scheme 5 below. Amidation of amine 7 with the protected amino acid derivative 3 can be carried out in analogy to the amidation reaction in scheme 2. Conversion of 9 to 4 can be carried out as described above for scheme 4. 4 can be deprotected to I″ as described in scheme 2 above.

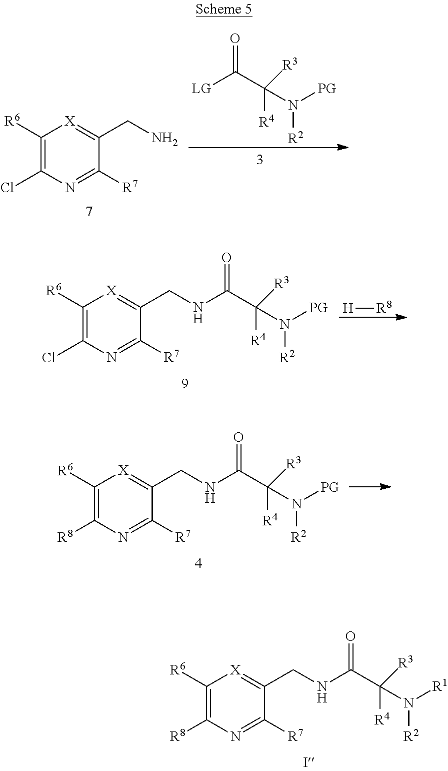

Yet another alternative for preparing compounds I′ by introducing the radical —R⁸ only after the introduction of the amino acid group is shown in scheme 6 below. Deprotection of 9 yields 10. Deprotection conditions depend on the protective group used. 10 is then converted into compounds 8 wherein R¹ or R¹ and R² are alkyl by subjecting compounds 10 to an alkylation reaction or to a reductive amination. Conversion of 8 into I′ can be carried out as described above for scheme 4.

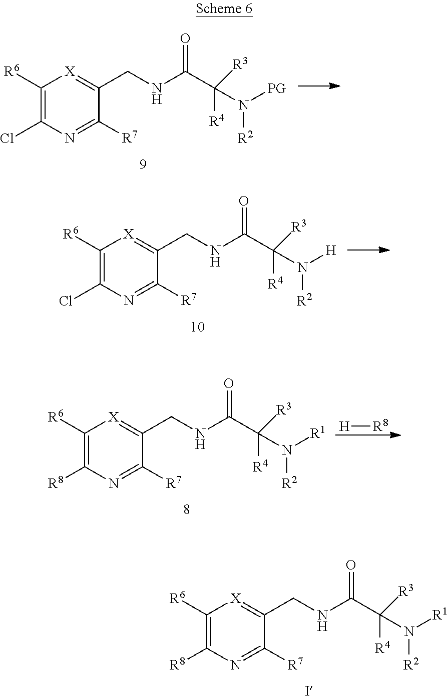

Compounds 1 wherein R⁷ is alkoxy (═R^{7′}; compounds termed hereinafter compounds 1′) can be prepared as outlined in scheme 7 below. This reaction path is suitable for compounds wherein R⁸ is —OR^{8a} or —NR^{8b}R^{8c}, but under certain conditions compounds wherein R⁸ is —R^{8d} can also be obtained analogously. Compound 11 is reacted with the compound H—R⁸ under conditions as described above for scheme 4. Due to the para-directing effect of CN the regioselectivity (as compared to the substitution of the fluorine substituent in ortho-position to CN and to the substitution of both fluorine atoms by —R⁸) is high if 11 and H—R⁸ are used in approximately stoichiometric amounts. Use of H—R⁸ in excess yields mixtures of the two regioisomers as well as compounds in which both fluorine atoms are replaced by —R⁸. In this case 12 has to be separated from the undesired side products by usual means, such as chromatography etc. Reaction of 12 with H—R^{7′} is carried out under analogous conditions as described above for scheme 4. Both the reaction of 11 with H—R⁸ as well as the reaction of 12 with H—R^{7′} are suitably carried out at low temperatures, e.g. at a temperature of at most 0° C., preferably of from −80 to 0° C. Hydrogenation of nitrile 13 yields 1′. Hydrogenation can be carried out under known conditions, e.g. using transition metal catalysts, such as Pd. Alternatively, reduction agents such as boranes, e.g. diborane, borane methylsulfide complex, borane THF complex and the like, or hydride complexes, e.g. lithium aluminum hydride, sodium borohydride and the like can be used for converting the nitrile into the aminomethyl compound 1′.

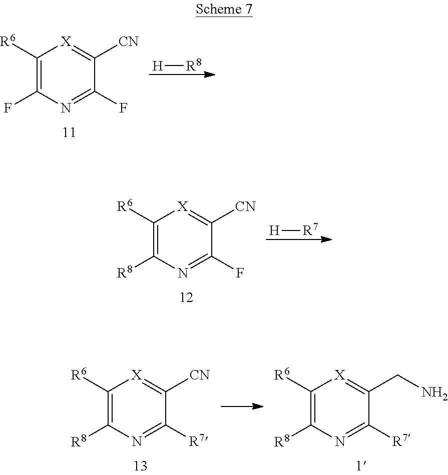

Compounds 1 wherein R⁷ is alkoxy (═R^{7′}; compounds termed hereinafter compounds 1′) can also be prepared as outlined in scheme 8 below. Compound 14 is reacted with the compound H—R⁸ under conditions as described above for scheme 4 or 7. Conversion of 13 into 1′ can be carried out as described above for scheme 7.

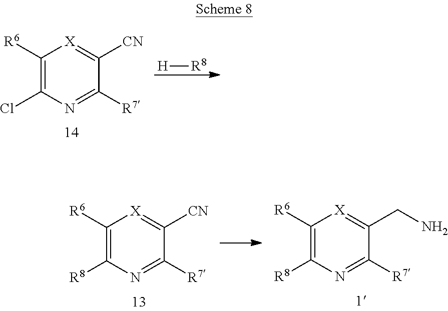

Compounds 1 wherein R⁷ is hydroxyl (compounds termed hereinafter compounds 1″) can be prepared as outlined in scheme 9 below. Compound 15 is reacted with the compound H—R⁸ under conditions as described above for scheme 4 or 7. 16 is then reacted with 2-methylsulfonylethanol in the presence of a strong non-nucleophilic base, such as sodium or potassium hydride. Acidic hydrolysis of the intermediate ether (not shown in scheme 9) yields the hydroxyl compound 17, the nitrile group of which is then reduced to an aminomethyl group as described above for scheme 7 to yield 1″.

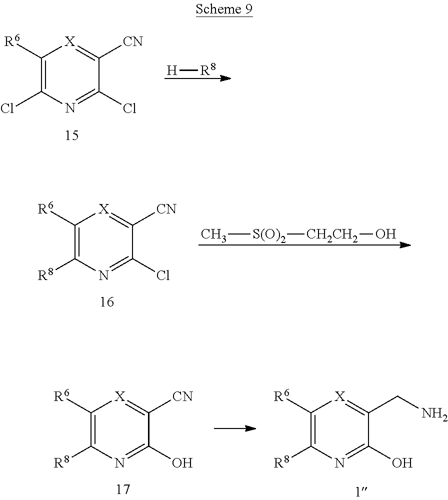

Compounds 7 wherein R⁷ is alkoxy (═R^{7′}; compounds termed hereinafter compounds 7′) can be prepared as outlined in scheme 10 below. Nitrile 18 is hydrolyzed with concentrated sulfuric acid to the amide 19. Compound 19 is then reacted with the alcohol H—R^{7′} under conditions as described above for scheme 4 to 7. 20 is formed rather regioselectively over the regioisomer (i.e. the compound wherein the chlorine atom ortho to R⁶ is substituted by 7′), probably because of the neighbouring effect of the amide group which facilitates the nucleophilic replacement of Cl in its ortho position. Conversion of 20 into 7′ can be carried in analogy to the reduction reaction described above for scheme 7.

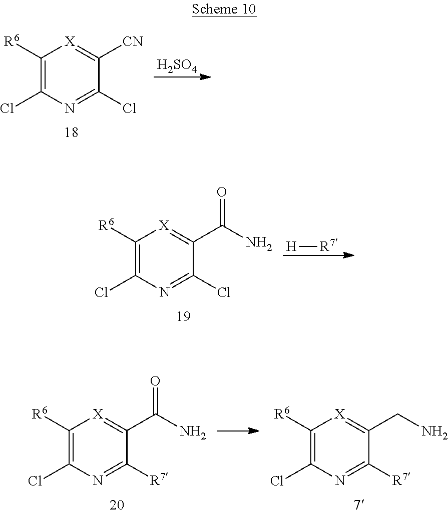

For obtaining compounds I wherein R⁵ is an alkyl group, compounds I, I′, 4, 6, 8 or 9 in which neither R¹ nor R² are hydrogen can be reacted with an alkylation agent, such as dimethylsulfate, methyliodide, triethyloxonium tetrafluoroborate and the like. Alternatively, the amino group in compounds 1′, 1″ or 7′ can be first protected, e.g. by reaction with Boc anhydride, so that just one free hydrogen atom is present on the amino group, and then the (mono)protected amino group can be reacted with an alkylation agent, such as dimethylsulfate, methyliodide, triethyloxonium tetrafluoroborate and the like.

If not otherwise indicated, the above-described reactions are generally carried out in a solvent at temperatures between room temperature and the boiling temperature of the solvent employed. Alternatively, the activation energy which is required for the reaction can be introduced into the reaction mixture using microwaves, something which has proved to be of value, in particular, in the case of the reactions catalyzed by transition metals (with regard to reactions using microwaves, see Tetrahedron 2001, 57, p. 9199 ff. p. 9225 ff. and also, in a general manner, “Microwaves in Organic Synthesis”, André Loupy (Ed.), Wiley-VCH 2002).

The acid addition salts of compounds I are prepared in a customary manner by mixing the free base with a corresponding acid, where appropriate in solution in an organic solvent, for example a lower alcohol, such as methanol, ethanol or propanol, an ether, such as methyl tert-butyl ether or diisopropyl ether, a ketone, such as acetone or methyl ethyl ketone, or an ester, such as ethyl acetate.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the preparation methods are within routine techniques.

Suitable protecting groups and the methods for protecting and deprotecting different substituents using such suitable protecting groups are well known to those skilled in the art; examples of which may be found in T. Greene and P. Wuts, Protective Groups in Organic Synthesis (3^{rd} ed.), John Wiley & Sons, NY (1999), which is herein incorporated by reference in its entirety. Synthesis of the compounds of the invention may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the invention is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the invention is required, it may be obtained by carrying out one of the above procedures using a pure geometric isomer as a starting material, or by resolution of a mixture of the geometric isomers of the compound or intermediates using a standard procedure such as chromatographic separation.

The present invention moreover relates to compounds of formula I as defined above, wherein at least one of the atoms has been replaced by its stable, non-radioactive isotope (e.g., hydrogen by deuterium, ¹²C by ¹³C, ¹⁴N by ¹⁵N, ¹⁶O by ¹⁸O) and preferably wherein at least one hydrogen atom has been replaced by a deuterium atom.

Of course, the unlabeled compounds according to the invention might naturally include certain amounts of these respective isotopes. Therefore, when referring to compounds I, wherein at least one of the atoms has been replaced by its stable, non-radioactive isotope, it will be understood that the isotope is present in a higher amount than would naturally occur.

Stable isotopes (e.g., deuterium, ¹³C, ¹⁵N, ¹⁸O) are nonradioactive isotopes which contain one additional neutron than the normally abundant isotope of the respective atom. Deuterated compounds have been used in pharmaceutical research to investigate the in vivo metabolic fate of the compounds by evaluation of the mechanism of action and metabolic pathway of the non deuterated parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

Incorporation of a heavy atom, particularly substitution of deuterium for hydrogen, can give rise to an isotope effect that could alter the pharmacokinetics of the drug.

Stable isotope labeling of a drug can alter its physico-chemical properties such as pKa and lipid solubility. These changes may influence the fate of the drug at different steps along its passage through the body. Absorption, distribution, metabolism or excretion can be changed. Absorption and distribution are processes that depend primarily on the molecular size and the lipophilicity of the substance. These effects and alterations can affect the pharmacodynamic response of the drug molecule if the isotopic substitution affects a region involved in a ligand-receptor interaction.

Drug metabolism can give rise to large isotopic effect if the breaking of a chemical bond to a deuterium atom is the rate limiting step in the process. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. In any reaction in which the breaking of this bond is the rate limiting step, the reaction will proceed slower for the molecule with the heavy isotope due to “kinetic isotope effect”. A reaction involving breaking a C-D bond can be up to 700 percent slower than a similar reaction involving breaking a C—H bond. If the C-D bond is not involved in any of the steps leading to the metabolite, there may not be any effect to alter the behavior of the drug. If a deuterium is placed at a site involved in the metabolism of a drug, an isotope effect will be observed only if breaking of the C-D bond is the rate limiting step. There is evidence to suggest that whenever cleavage of an aliphatic C—H bond occurs, usually by oxidation catalyzed by a mixed-function oxidase, replacement of the hydrogen by deuterium will lead to observable isotope effect. It is also important to understand that the incorporation of deuterium at the site of metabolism slows its rate to the point where another metabolite produced by attack at a carbon atom not substituted by deuterium becomes the major pathway a process called “metabolic switching”.

Deuterium tracers, such as deuterium-labeled drugs and doses, in some cases repeatedly, of thousands of milligrams of deuterated water, are also used in healthy humans of all ages, including neonates and pregnant women, without reported incident (e.g. Pons G and Rey E, Pediatrics 1999 104: 633; Coward W A et al., Lancet 1979 7: 13; Schwarcz H P, Control. Clin. Trials 1984 5(4 Suppl): 573; Rodewald L E et al., J. Pediatr. 1989 114: 885; Butte N F et al. Br. J. Nutr. 1991 65: 3; MacLennan A H et al. Am. J. Obstet Gynecol. 1981 139: 948). Thus, it is clear that any deuterium released, for instance, during the metabolism of compounds of this invention poses no health risk.

The weight percentage of hydrogen in a mammal (approximately 9%) and natural abundance of deuterium (approximately 0.015%) indicates that a 70 kg human normally contains nearly a gram of deuterium. Furthermore, replacement of up to about 15% of normal hydrogen with deuterium has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci 1960 84: 736; Czakja D M et al., Am. J. Physiol. 1961 201: 357). Higher deuterium concentrations, usually in excess of 20%, can be toxic in animals. However, acute replacement of as high as 15%-23% of the hydrogen in humans' fluids with deuterium was found not to cause toxicity (Blagojevic N et al. in “Dosimetry & Treatment Planning for Neutron Capture Therapy”, Zamenhof R, Solares G and Harling O Eds. 1994. Advanced Medical Publishing, Madison Wis. pp. 125-134; Diabetes Metab. 23: 251 (1997)).

Increasing the amount of deuterium present in a compound above its natural abundance is called enrichment or deuterium-enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %.

The hydrogens present on a particular organic compound have different capacities for exchange with deuterium. Certain hydrogen atoms are easily exchangeable under physiological conditions and, if replaced by deuterium atoms, it is expected that they will readily exchange for protons after administration to a patient. Certain hydrogen atoms may be exchanged for deuterium atoms by the action of a deuteric acid such as D2SO4/D2O. Alternatively, deuterium atoms may be incorporated in various combinations during the synthesis of compounds of the invention. Certain hydrogen atoms are not easily exchangeable for deuterium atoms. However, deuterium atoms at the remaining positions may be incorporated by the use of deuterated starting materials or intermediates during the construction of compounds of the invention.

Deuterated and deuterium-enriched compounds of the invention can be prepared by using known methods described in the literature. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; 20090082471, the methods are hereby incorporated by reference.

The present invention further relates to a pharmaceutical composition comprising a therapeutically effective amount of at least one compound I as defined above or an N-oxide, a tautomeric form, a stereoisomer or a pharmaceutically acceptable salt thereof, in combination with at least one pharmaceutically acceptable carrier and/or auxiliary substance; or comprising at least one compound I wherein at least one of the atoms has been replaced by its stable, non-radioactive isotope, preferably wherein at least one hydrogen atom has been replaced by a deuterium atom, in combination with at least one pharmaceutically acceptable carrier and/or auxiliary substance.

The present invention further relates to a compound I as defined above or an N-oxide, a tautomeric form, a stereoisomer or a pharmaceutically acceptable salt thereof for use as a medicament.

The present invention also relates to a compound I as defined above or an N-oxide, a tautomeric form, a stereoisomer or a pharmaceutically acceptable salt thereof for use in the treatment of disorders which respond to the modulation of the 5-HT_{2C} receptor.

The present invention also relates to the use of a compound I as defined above or of an N-oxide, a tautomeric form, a stereoisomer or a pharmaceutically acceptable salt thereof for the manufacture of a medicament for the treatment of disorders which respond to the modulation of the 5-HT_{2C} receptor, and to a method for treating disorders which respond to the modulation of the 5-HT_{2C} receptor, which method comprises administering to a subject in need thereof at least one compound I as defined above or an N-oxide, a tautomeric form, a stereoisomer or a pharmaceutically acceptable salt thereof.

The compounds of the present invention are modulators of the 5-HT_{2C} receptor. Specifically, the compounds of formula I are agonists or partial agonists of the 5-HT_{2C} receptor. Thus, in a specific embodiment, the invention relates to a compound I as defined above or an N-oxide, a tautomeric form, a stereoisomer or a pharmaceutically acceptable salt thereof for the treatment of disorders which respond to 5-HT_{2C} receptor agonists, further to the use of a compound I as defined above or of an N-oxide, a tautomeric form, a stereoisomer or a pharmaceutically acceptable salt thereof for the manufacture of a medicament for the treatment of disorders which respond to 5-HT_{2C} receptor agonists, and to a method for treating disorders which respond to 5-HT_{2C} receptor agonists, which method comprises administering to a subject in need thereof at least one compound I as defined above or an N-oxide, a tautomeric form, a stereoisomer or a pharmaceutically acceptable salt thereof.

Within the meaning of the invention, the term “disorder” denotes disturbances and/or anomalies which are as a rule regarded as being pathological conditions or functions and which can manifest themselves in the form of particular signs, symptoms and/or malfunctions. While the treatment according to the invention can be directed toward individual disorders, i.e. anomalies or pathological conditions, it is also possible for several anomalies, which may be causatively linked to each other, to be combined into patterns, i.e. syndromes, which can be treated in accordance with the invention.

In one aspect of the invention, the diseases to be treated are disorders are damage of the central nervous system, disorders of the central nervous system, eating disorders, ocular hypertension, cardiovascular disorders, gastrointestinal disorders and diabetes.

Disorders or diseases of the central nervous system are understood as meaning disorders which affect the spinal cord and, in particular, the brain. These are, for example, cognitive dysfunction, attention deficit disorder/hyperactivity syndrome and cognitive deficits related with schizophrenia, attention deficit/hyperactivity syndrome, personality disorders, affective disorders, motion or motor disorders, pain, migraine, sleep disorders (including disturbances of the Circadian rhythm), feeding disorders, diseases associated with neurodegeneration, addiction diseases, obesity or psoriasis.

Examples of cognitive dysfunction are deficits in memory, cognition, and learning, Alzheimer's disease, age-related cognitive decline, and mild cognitive impairment, or any combinations thereof. Examples of personality disorders are schizophrenia and cognitive deficits related to schizophrenia. Examples of affective disorders are depression, anxiety, bipolar disorder and obsessive compulsive disorders, or any combination thereof. Examples of motion or motor disorders are Parkinson's disease and epilepsy. Examples of feeding disorders are obesity, bulimia, weight loss and anorexia, especially anorexia nervosa. Examples of diseases associated with neurodegeneration are stroke, spinal or head trauma, and head injuries, such as hydrocephalus.

Pain condition includes nociceptive pain, neuropathic pain or a combination thereof. Such pain conditions or disorders can include, but are not limited to, postoperative pain, osteoarthritis pain, pain due to inflammation, rheumatoid arthritis pain, musculoskeletal pain, burn pain (including sunburn), ocular pain, the pain associated with dental conditions (such as dental caries and gingivitis), post-partum pain, bone fracture, herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, hyperalgesia and cancer.

In certain other embodiments, the disease condition is bladder dysfunction, including urinary incontinence.

Diabetes includes diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

The addiction diseases include psychiatric disorders and behavioral disturbances which are caused by the abuse of psychotropic substances, such as pharmaceuticals or narcotics, and also other addiction diseases, such as addiction to gaming (impulse control disorders not elsewhere classified). Examples of addictive substances are: opioids (e.g. morphine, heroin and codeine), cocaine; nicotine; alcohol; substances which interact with the GABA chloride channel complex, sedatives, hypnotics and tranquilizers, for example benzodiazepines; LSD; cannabinoids; psychomotor stimulants, such as 3,4-methylenedioxy-N-methylamphetamine (ecstasy); amphetamine and amphetamine-like substances such as methylphenidate, other stimulants including caffeine and nicotine. Addictive substances which come particularly into consideration are opioids, cocaine, amphetamine or amphetamine-like substances, nicotine and alcohol. Especially, addiction disorders include alcohol abuse, cocaine abuse, tobacco abuse and smoking cessation.

With regard to the treatment of addiction diseases, particular preference is given to those compounds according to the invention of the formula (I) which themselves do not possess any psychotropic effect. This can also be observed in a test using rats, which, after having been administered compounds which can be used in accordance with the invention, reduce their self administration of psychotropic substances, for example cocaine.

Examples of gastrointestinal disorders are irritable bowel syndrome.

Preferably, the disorders are selected from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease (e.g. aggression), behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, psychosis in Parkinson's disease, neuropsychiatric symptoms in Parkinson's disease (e.g. aggression), Lewy Body dementia, neuropsychiatric symptoms in Lewy Body dementia (e.g. aggression), spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance, and are specifically schizophrenia, depression, bipolar disorders, obesity, substance use disorders, neuropsychiatric symptoms in Alzheimer's disease (e.g. aggression) or neuropsychiatric symptoms in Parkinson's disease (e.g. aggression).

The compounds of the invention may be used for a preventive treatment (prophylaxis), in particular as relapse prophylaxis or phase prophylaxis, but are preferably used for a treatment in its proper sense (i.e. non-prophylactic), i.e. for the treatment of acute or chronic signs, symptoms and/or malfunctions. The treatment can be orientated symptomatically, for example as the suppression of symptoms. It can be effected over a short period, be orientated over the medium term or can be a long-term treatment, for example within the context of a maintenance therapy.

In another embodiment, the present invention relates to the use of a compound I as defined above or an N-oxide, a tautomeric form, a stereoisomer or a pharmaceutically acceptable salt thereof for preparing a medicament for preventing (the development of) a disease condition as described above and to a method for preventing (the development of) a disease condition as described above comprises administering to the subject in need of treatment thereof (e.g., a mammal, such as a human) a therapeutically effective amount of a compound I as defined above or an N-oxide, a tautomeric form, a stereoisomer or a pharmaceutically acceptable salt thereof. As used herein, the term “prevent” a disease condition by administration of any of the compounds described herein means that the detectable physical characteristics or symptoms of the disease or condition do not develop following the administration of the compound described herein. Alternatively, the method comprises administering to the subject a therapeutically effective amount of a compound I as defined above or an N-oxide, a tautomeric form, a stereoisomer or a pharmaceutically acceptable salt thereof, in combination with a therapeutically effective amount of at least one cognitive enhancing drug.

In yet another embodiment, the present invention relates to the use a compound I as defined above or an N-oxide, a tautomeric form, a stereoisomer or a pharmaceutically acceptable salt thereof for preparing a medicament for preventing the progression (e.g., worsening) of a disease condition and to a method for preventing the progression (e.g., worsening) of a disease condition, which method comprises administering to the subject in need of treatment thereof (e.g., a mammal, such as a human) a therapeutically effective amount of a compound I as defined above or an N-oxide, a tautomeric form, a stereoisomer or a pharmaceutically acceptable salt thereof.

There are several lines of evidence suggesting that 5-HT_{2C} agonists or partial agonists would have therapeutic use in a variety of diseases, disorders and conditions.

Knockout mice models lacking the 5-HT_{2C} receptor exhibit hyperphagia, obesity and are more prone to seizures and sudden death [Tecott L H, Sun L M, Akana S F, Strack A M, Lowenstein D H, Dallman M F, Julius D (1995) Eating disorder and epilepsy in mice lacking 5-HT_{2C} serotonin receptors. Nature 374:542-546]. They also exhibit compulsive-like behavior [Chou-Green J M, Holscher T D, Dallman M F, Akana S F (2003). Compulsive behavior in the 5-HT_{2C} receptor knockout mouse. Phys. Behav. 78:641-649], hyperresponsiveness to repeated stress [Chou-Green J M, Holscher T D, Dallman M F, Akana S F (2003). Repeated stress in young and old 5-HT_{2C} receptor knockout mouse. Phys. Behav. 79:217-226], wakefulness [Frank M G, Stryker M P, Tecott L H (2002). Sleep and sleep homeostasis in mice lacking the 5-HT_{2C} receptor. Neuropsychopharmacology 27:869-873], hyperactivity and drug dependence [Rocha B A, Goulding E H, O'Dell L E, Mead A N, Coufal N G, Parsons L H, Tecott L H (2002). Enhanced locomotor, reinforcing and neurochemical effects of cocaine in serotonin 5-hydroxytryptamine 2C receptor mutant mice. J. Neurosci. 22:10039-10045].

5-HT_{2C} is unique among other G-protein-coupled receptors (GPCRs) in that its pre-mRNA is a substrate for base modification via hydrolytic deamination of adenosines to yield inosines. Five adenosines, located within a sequence encoding the putative second intracellular domain can be converted to inosines. This editing can alter the coding potential of the triplet codons and allows for the generation of multiple different receptor isoforms. The edited receptor isoforms were shown to have reduced ability to interact with G-proteins in the absence of agonist stimulation [Werry, T D, Loiacono R, Sexton P A, Christopoulos A (2008). RNA editing of the serotonin 5-HT_{2C} receptor and its effects on cell signaling, pharmacology and brain function. Pharmac. Therap. 119:7-23].

Edited 5-HT_{2C} isoforms with reduced function are significantly expressed in the brains of depressed suicide victims [Schmauss C (2003) Serotonin 2C receptors: suicide, serotonin, and runaway RNA editing. Neuroscientist 9:237-242. Iwamoto K, Kato T (2003). RNA editing of serotonin 2C receptor in human postmortem brains of major mental disorders. Neurosci. Lett. 346:169-172] and in the learned helplessness rats (a well established animal model of depression) [Iwamotoa K, Nakatanib N, Bundoa M, Yoshikawab T, Katoa T (2005). Altered RNA editing of serotonin 2C receptor in a rat model of depression. Neurosci. Res. 53: 69-76] suggesting a link between 5-HT_{2C} function and depression. There are also implications of edited 5-HT_{2C} isoforms and spatial memory [Du Y, Stasko M, Costa A C, Davissone M T, Gardiner K J (2007). Editing of the serotonin 2C receptor pre-mRNA Effects of the Morris Water Maze. Gene 391:186-197]. In addition, fully edited isoforms of the human 5-HT_{2C} receptor display a striking reduction in sensitivity to lysergic acid diethylamide (LSD) and to atypical antipsychotic drugs clozapine and loxapine, suggesting a possible role of the receptor in the etiology and pharmacology of schizophrenia [Niswender C M, Herrick-Davis K., Dilley G E, Meltzer H Y, Overholser J C, Stockmeier C A, Emeson R B, Sanders-Bush E (2001). RNA Editing of the Human Serotonin 5-HT_{2C} Receptor: Alterations in Suicide and Implications for Serotonergic Pharmacotherapy. Neuropsychopharm. 24:478-491].

Recently, the availability of potent and selective 5-HT_{2C} receptor agonists made it possible to directly investigate the effects of 5-HT_{2C} agonists and their therapeutic potential. Thus recent studies demonstrated that selective 5-HT_{2C} agonists resulted in decreased food intake and body weight gain in normal and obese rats [Smith B M, et al. (2008). Discovery and structure-activity relationship of (1R)-8-chloro-2,3,4,5-tetrahydro-1-methyl-1H-3-benzazepine (Lorcaserin), a selective serotonin 5-HT_{2C} receptor agonist for the treatment of obesity. J Med Chem 51:305-313. Thomsen W J, Grottick A J, Menzaghi F, Reyes-Saldana H, Espitia S, Yuskin D, Whelan K, Martin M, Morgan M, Chen W, Al-Shama H, Smith B, Chalmers D, Behan D (2008) Lorcaserin, A Novel Selective Human 5-HT_{2C} Agonist: In Vitro and In Vivo Pharmacological Characterization. J Pharmacol Exp Ther. 325:577-587. Rosenzweig-Lipson S, Zhang J, Mazandarani H, Harrison B L, Sabb A, Sabalski J, Stack G, Welmaker G, Barrett J E, Dunlop J (2006) Antiobesity-like effects of the 5-HT_{2C} receptor agonist WAY-161503. Brain Res. 1073-1074:240-251. Dunlop J, Sabb A L, Mazandarani H, Zhang J, Kalgaonker S, Shukhina E, Sukoff S, Vogel R L, Stack G, Schechter L, Harrison B L, Rosenzweig-Lipson S (2005). WAY-163909 [97bR, 10aR)-1,2,3,4,8,9,10,10a-octahydro-7bH-cyclopenta-[b][1,4]diazepino[6,7,1hi]indole], a novel 5-hydroxytryptamine 2C receptor-selective agonist with anorectic activity. J Pharmacol Exp Ther. 313:862-869.].

Furthermore, selective 5-HT_{2C} receptor agonists produce antidepressant effects in animal models of depression comparable to those of SSRIs but with a much faster onset of action and a therapeutic window that avoids antidepressant-induced sexual dysfunction. These agonists were also effective in animal models of compulsive behavior such as scheduled induced polydipsia and they also exhibited decreased hyperactivity and aggression in rodents [Rosenzweig-Lipson S, Sabb A, Stack G, Mitchell P, Lucki I, Malberg J E, Grauer S, Brennan J, Cryan J F, Sukoff Rizzo S J, Dunlop J, Barrett J E, Marquis K L (2007) Antidepressant-like effects of the novel, selective, 5-HT_{2C} receptor agonist WAY-163909 in rodents. Psychopharmacology (Berlin) 192:159-170. Rosenzweig-Lipson S, Dunlop J, Marquis K L (2007) 5-HT_{2C} receptor agonists as an innovative approach for psychiatric disorders. Drug news Perspect, 20: 565-571. Cryan, J F, Lucki I (2000). Antidepressant-like behavioral effects mediated by 5-Hydroxytryptamine 2C receptors. J. Pharm. Exp. Ther. 295:1120-1126.].

Acute or chronic administration of 5-HT_{2C} agonists decreases the firing rate of ventral tegmental area dopamine neurons but not that of substantia nigra. In addition 5-HT_{2C} agonists reduce dopamine levels in the nucleus accumbens but not in the striatum (the region of the brain mostly associated with extrapyramidal side effects) [Di Matteo, V., Di Giovanni, G., Di Mascio, M., & Esposito, E. (1999). SB 242084, a selective serotonin 2C receptor antagonist, increases dopaminergic transmission in the mesolimbic system. Neuropharmacology 38, 1195-1205. Di Giovanni, G., Di Matteo, V., Di Mascio, M., & Esposito, E. (2000). Preferential modulation of mesolimbic vs. nigrostriatal dopaminergic function by serotonin2C/2B receptor agonists: a combined in vivo electrophysiological and microdialysis study. Synapse 35, 53-61. Marquis K L, Sabb A L, Logue S F, Brennan J A, Piesla M J, Comery T A, Grauer S M, Ashby C R, Jr., Nguyen H Q, Dawson L A, Barrett J E, Stack G, Meltzer H Y, Harrison B L, Rosenzweig-Lipson S (2007) WAY-163909 [(7bR,10aR)-1,2,3,4,8,9,10,10a-octahydro-7bH-cyclopenta-[b][1,4]diazepino[6,7,1hi]indole]: A novel 5-hydroxytryptamine 2C receptor-selective agonist with preclinical antipsychotic-like activity. J Pharmacol Exp Ther 320:486-496.]. Therefore it is expected that 5-HT_{2C} receptor agonists will selectively decrease mesolimbic dopamine levels without affecting the nigrostriatal pathway thus avoiding the EPS side effects of typical antipsychotics. Several 5-HT_{2C} receptor agonists have shown antipsychotic activity in animal models of schizophrenia without EPS based on the lack of effect in catalepsy [Marquis K L, Sabb A L, Logue S F, Brennan J A, Piesla M J, Comery T A, Grauer S M, Ashby C R, Jr., Nguyen H Q, Dawson L A, Barrett J E, Stack G, Meltzer H Y, Harrison B L, Rosenzweig-Lipson S (2007) WAY-163909 [(7bR,10aR)-1,2,3,4,8,9,10,10a-octahydro-7bH-cyclopenta-[b][1,4]diazepino[6,7,1hi]indole]: A novel 5-hydroxytryptamine 2C receptor-selective agonist with pre-clinical antipsychotic-like activity. J Pharmacol Exp Ther 320:486-496. Siuciak J A, Chapin D S, McCarthy S A, Guanowsky V, Brown J, Chiang P, Marala R, Patterson T, Seymour P A, Swick A, Iredale P A (2007) CP-809,101, a selective 5-HT_{2C} agonist, shows activity in animal models of antipsychotic activity. Neuropharmacology 52:279-290]. The antipsychotic activity of 5-HT_{2C} receptor agonists without EPS coupled with their beneficial effects in mood disorders and cognition and their antiobesity like effects render 5-HT_{2C} receptor agonists as unique agents to treat schizophrenia [Rosenzweig-Lipson S, Dunlop J, Marquis K L (2007) 5-HT_{2C} receptor agonists as an innovative approach for psychiatric disorders. Drug news Perspect, 20: 565-571. Dunlop J, Marquis K L, Lim H K, Leung L, Kao J, Cheesman C, Rosenzweig-Lipson S (2006). Pharmacological profile of the 5-HT_{2C} receptor agonist WAY-163909; therapeutic potential in multiple indications. CNS Dug Rev. 12:167-177.].

In addition 5-HT_{2C} modulation has been implicated in epilepsy [Isaac M (2005). Serotonergic 5-HT_{2C} receptors as a potential therapeutic target for the antiepileptic drugs. Curr. Topics Med. Chem. 5:59:67], psoriasis [Thorslund K, Nordlind K (2007). Serotonergic drugs—a possible role in the treatment of psoriasis? Drug News Perspect 20:521-525], Parkinson's disease and related motor disorders [Esposito E, Di Matteo V, Pierucci M, Benigno A, Di Giavanni, G (2007). Role of central 5-HT_{2C} receptor in the control of basal ganglia functions. The Basal Ganglia Pathophysiology: Recent Advances 97-127], behavioral deficits [Barr A M, Lahmann-Masten V, Paulus M, Gainetdinov R P, Caron M G, Geyer M A (2004). The selective serotonin-2A receptor antagonist M100907 reverses behavioral deficits in dopamine transporter knockout mice. Neuropsychopharmacology 29:221-228], anxiety [Dekeyne A, Mannoury la Cour C, Gobert A, Brocco M, Lejuene F, Serres F, Sharp T, Daszuta A, Soumier A, Papp M, Rivet J M, Flik G, Cremers T I, Muller O, Lavielle G, Millan M J (2208). S32006, a novel 5-HT_{2C} receptor antagonists displaying broad-based antidepressant and anxiolytic properties in rodent models. Psychopharmacology 199:549-568. Nunes-de-Souza V, Nunes-de-Souza R L, Rodgers R J, Canto-de-Souza A (2008). 5-HT2 receptor activation in the midbrain periaqueductal grey (PAG) reduces anxiety-like behavior in mice. Behav. Brain Res. 187:72-79.], migraine [Leone M, Rigamonti A, D'Amico D, Grazzi L, Usai S, Bussone G (2001). The serotonergic system in migraine. Journal of Headache and Pain 2(Suppl. 1):S43-S46], Alzheimer's disease [Arjona A A, Pooler A M, Lee R K, Wurtman R J (2002). Effect of a 5-HT_{2C} serotonin agonist, dexnorfenfluramine, on amyloid precursor protein metabolism in guinea pigs. Brain Res. 951:135-140], pain and spinal cord injury [Nakae A, Nakai K, Tanaka T, Hagihira S, Shibata M, Ueda K, Masimo T (2008). The role of RNA editing of the serotonin 2C receptor in a rat model of oro-facial neuropathic pain. The European Journal of Neuroscience 27:2373-2379. Nakae A, Nakai K, Tanaka T, Takashina M, Hagihira S, Shibata M, Ueda K, Mashimo T (2008). Serotonin 2C receptor mRNA editing in neuropathic pain model. Neurosci. Res. 60:228-231. Kao T, Shumsky J S, Jacob-Vadakot S, Timothy H B, Murray M, Moxon, K A (2006). Role of the 5-HT_{2C} receptor in improving weight-supported stepping in adult rats spinalized as neonates. Brain Res. 1112:159-168.], sexual dysfunction [Motofei I G (2008). A dual physiological character for sexual function: the role of serotonergic receptors. BJU International 101:531-534. Shimada I, Maeno K, Kondoh Y, Kaku H, Sugasawa K, Kimura Y, Hatanaka K, Naitou Y, Wanibuchi F, Sakamoto S, Tsukamoto S (2008). Synthesis and structure-activity relationships of a series of benzazepine derivatives as 5-HT_{2C} receptor agonists. Bioorg. Med. Chem. 16:3309-3320.], smoking cessation [Fletcher P J, Le A D, Higgins G A (2008). Serotonin receptors as potential targets for modulation of nicotine use and dependence. Progress Brain Res. 172:361-83], substance dependence [Bubar M J, Cunningham K A (2008). Prospects for serotonin 5-HT2R pharmacotherapy in psychostimulant abuse. Progress Brain Res. 172:319-46], and ocular hypertension [Sharif N A, McLaughlin M A, Kelly C R (2006). AL-34662: a potent, selective, and efficacious ocular hypotensive serotonin-2 receptor agonist. J Ocul Pharmacol Ther. 23:1-13].

Further, 5HT modulation can be useful in the treatment of pain, both neuropathic and nociceptive pain, see for example U.S. patent application publication US2007/0225277. Obata, Hideaki; Ito, Naomi; Sasaki, Masayuki; Saito, Shigeru; Goto, Fumio. Possible involvement of spinal noradrenergic mechanisms in the antiallodynic effect of intrathecally administered 5-HT2C receptor agonists in the rats with peripheral nerve injury. European Journal of Pharmacology (2007), 567(1-2), 89-94. Serotonin2C receptor mRNA editing in neuropathic pain model. Nakae, Aya; Nakai, Kunihiro; Tanaka, Tatsuya; Takashina, Masaki; Hagihira, Satoshi; Shibata, Masahiko; Ueda, Koichi; Mashimo, Takashi. Department of Anesthesiology & Intensive Care Medicine, Graduate School of Medicine, Osaka University, Neuroscience Research (Amsterdam, Netherlands) (2008), 60(2), 228-231. Antiallodynic effects of intrathecally administered 5-HT2C receptor agonists in rats with nerve injury. Obata, Hideaki; Saito, Shigeru; Sakurazawa, Shinobu; Sasaki, Masayuki; Usui, Tadashi; Goto, Fumio. Department of Anesthesiology, Gunma University Graduate School of Medicine, Maebashi, Gunma, Japan. Pain (2004), 108(1-2), 163-169. Influence of 5,7-dihydroxytryptamine (5,7-DHT) on the antinociceptive effect of serotonin (5-HT) 5 HT2C receptor agonist in male and female rats. Brus, Ryszard; Kasperska, Alicja; Oswiecimska, Joanna; Szkilnik, Ryszard. Department of Pharmacology, Silesian Medical University, Zabrze, Pol. Medical Science Monitor (1997), 3(5), 654-656.

Modulation of 5HT2 receptors may be beneficial in the treatment of conditions related to bladder function, in particular, urinary incontinence. [Discovery of a novel azepine series of potent and selective 5-HT2C agonists as potential treatments for urinary incontinence. Brennan, Paul E.; Whitlock, Gavin A.; Ho, Danny K. H.; Conlon, Kelly; McMurray, Gordon. Bioorganic & Medicinal Chemistry Letters (2009), 19(17), 4999-5003. Investigation of the role of 5-HT2 receptor subtypes in the control of the bladder and the urethra in the anesthetized female rat. Mbaki, Y.; Ramage, A. G. Department of Pharmacology, University College London, London, UK. British Journal of Pharmacology (2008), 155(3), 343-356.] In particular, compounds with agonist activity at 5-HT_{2C} have been shown to be useful in treating urinary incontinence, see for example U.S. Patent application publications US2008/0146583 and US 2007/0225274.

Further pre-clinical data suggest that 5-HT_{2C} agonists could be useful for the treatment of a number of psychiatric diseases, including schizophrenia, bipolar disorders, depression/anxiety, substance use disorders and especially disorders like neuropsychiatric symptoms in Alzheimer's disease: Aggression, psychosis/agitation represent key unmet medical needs. Clinical (Shen J H Q et al., A 6-week randomized, double-blind, placebo-controlled, comparator referenced trial of vabicaserin in acute schizophrenia. Journal of Psychiatric Research 53 (2014) 14-22; Liu J et al., Prediction of Efficacy of Vabicaserin, a 5-HT_{2C} Agonist, for the Treatment of Schizophrenia Using a Quantitative Systems Pharmacology Model. CPT Pharmacometrics Syst. Pharmacol. (2014) 3, e111;) and preclinical data (Dunlop J et al., Characterization of Vabicaserin (SCA-136), a Selective 5-Hydroxytryptamine 2C Receptor Agonist. J Pharmacol Exp Ther (2011) 337, 673-80; Siuciak J et al., CP-809,101, a selective 5-HT_{2C} agonist, shows activity in animal models of antipsychotic activity. Neuropharmacology 52 (2007) 279-290; Mosienko V et al., Exaggerated aggression and decreased anxiety in mice deficient in brain serotonin. Transl Psychiatry (2012) 2, e122; Del Guidice T et al., Stimulation of 5-HT_{2C} Receptors Improves Cognitive Deficits Induced by Human Tryptophan Hydroxylase2 Loss of Function Mutation. Neuropsychopharmacology (2014) 39, 1125-1134; Rosenzweig-Lipson et al., Antidepressant-like effects of the novel, selective, 5-HT_{2C} receptor agonist WAY-163909 in rodents. Psychopharmacology (2007) 192:159-170) suggest 5-HT_{2C} receptor stimulation to result in therapeutic efficacy in aggression, psychosis agitation and moderate pro-cognitive effects (Del Guidice T et al., Stimulation of 5-HT_{2C} Receptors Improves Cognitive Deficits Induced by Human Tryptophan Hydroxylase2 Loss of Function Mutation. Neuropsychopharmacology (2014) 39, 1125-1134; Siuciak J et al., CP-809,101, a selective 5-HT_{2C} agonist, shows activity in animal models of antipsychotic activity. Neuropharmacology 52 (2007) 279-290).

In the use and the method of the invention, an effective quantity of one or more compounds, as a rule formulated in accordance with pharmaceutical and veterinary practice, is administered to the individual to be treated, preferably a mammal, in particular a human being, productive animal or domestic animal. Whether such a treatment is indicated, and in which form it is to take place, depends on the individual case and is subject to medical assessment (diagnosis) which takes into consideration signs, symptoms and/or malfunctions which are present, the risks of developing particular signs, symptoms and/or malfunctions, and other factors.

Actual dosage levels of active ingredients in the pharmaceutical compositions of the present invention can be varied so as to obtain an amount of the active compound(s) that is effective to achieve the desired therapeutic response for a particular subject (e.g., a mammal, preferably, a human (patient)), compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

Compounds of the present invention can also be administered to a subject as a pharmaceutical composition comprising the compounds of interest in combination with at least one pharmaceutically acceptable carriers. The phrase “therapeutically effective amount” of the compound of the present invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a subject (namely, a mammal, such as a human) ranges from about 0.01 mg/kg body weight to about 100 mg/kg body weight. More preferable doses can be in the range of from about 0.01 mg/kg body weight to about 30 mg/kg body weight. If desired, the effective daily dose can be divided into multiple doses for purposes of administration. Consequently, single dose compositions may contain such amounts or submultiples thereof to make up the daily dose.

In one aspect, the present invention provides pharmaceutical compositions. The pharmaceutical compositions of the present invention comprise the compounds of the present invention or an N-oxide, a tautomeric form, a stereoisomer or a pharmaceutically acceptable salt or solvate thereof. The pharmaceutical compositions of the present invention comprise compounds of the present invention that can be formulated together with at least one non-toxic pharmaceutically acceptable carrier.

In yet another embodiment, the present invention provides a pharmaceutical composition comprising compounds of the present invention or an N-oxide, a tautomeric form, a stereoisomer or a pharmaceutically acceptable salt thereof, and one or more pharmaceutically acceptable carriers, alone or in combination with one or more compounds that are not the compounds of the present invention. Examples of one or more compounds that can be combined with the compounds of the present invention in pharmaceutical compositions, include, but are not limited to, one or more cognitive enhancing drugs.

The pharmaceutical compositions of this present invention can be administered to a subject (e.g., a mammal, such as a human) orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of the present invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable nonirritating carriers or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of the present invention include powders, sprays, ointments and inhalants. The active compound may be mixed under sterile conditions with a pharmaceutically acceptable carrier and any needed preservatives, buffers or propellants which may be required. Ophthalmic formulations, eye ointments, powders and solutions are also contemplated as being within the scope of this invention.

The compounds of the present invention can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well known in the art. For example, S. M. Berge et al. describe pharmaceutically acceptable salts in detail in (J. Pharmaceutical Sciences, 1977, 66: 1 et seq.). The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, ethylammonium and the like. Other representative organic amines useful for the formation of base addition salts include ethylenediamine, ethanolamine, diethanolamine, piperidine, piperazine and the like.

The compounds of the present invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The following examples serve to explain the invention without limiting it.

### EXAMPLES

### Abbreviations

EtOAc ethyl acetate
DMF dimethylformamide
DMSO dimethylsulfoxide
PE petrol ether
THF tetrahydrofuran
MeOH methanol
EtOH ethanol
iPrOH isopropanol
MeCN acetonitrile
Et₂O diethylether
MTBE methyl-tert-butylether
DCM dichloromethane
DCE dichloroethane
TFA trifluoroacetic acid
DIPEA diisopropylethyl amine
HATU O-(7-azabenzotriazol-1-yl)-N,N,N′,N′-tetramethyluronium hexafluorophosphate
HBTU 2-(1H-benzo[d][1,2,3]triazol-1-yl)-1,1,3,3-tetramethylisouronium hexafluorophosphate(V)
Boc/BOC tert-butoxycarbonyl
OAc acetate
KOtBu potassium tert-butanolate
TPGS-750-M a polyoxyethanyl-α-tocopheryl succinate derivative
sat saturated
aq aqueous
RT room temperature (20-25° C.)
h hour(s)
min minute(s)

The compounds were either characterized via proton-NMR in d₆-dimethylsulfoxide, d-chloroform or d₄-methanol on a 400 MHz, 500 MHz or 600 MHz NMR instrument (Bruker AVANCE), or by ¹³C-NMR at 125 MHz, or by ¹⁹F-NMR at 470 MHz, or by mass spectrometry, generally recorded via HPLC-MS in a fast gradient on C18-material (electrospray-ionisation (ESI) mode).

The magnetic nuclear resonance spectral properties (NMR) refer to the chemical shifts (δ) expressed in parts per million (ppm). The relative area of the shifts in the ¹H-NMR spectrum corresponds to the number of hydrogen atoms for a particular functional type in the molecule. The nature of the shift, as regards multiplicity, is indicated as singlet (s), broad singlet (br s), doublet (d), broad doublet (br d), triplet (t), broad triplet (br t), quartet (q), quintet (quint.), multiplet (m), doublet of doublets (dd), doublet of doublets of doublets (ddd), triplet of doublets (td), triplet of triplets (tt), doublet of triplets of doublets (dtd), doublet of triplets of triplets (dtt), doublet of triplets of quartets (dtq), quartet of doublets (qd), quartet of doublets of doublets (qdd) etc.

In the below examples the names of the synthesized final compounds are followed by a description of their structure. This description does however not include any information on the configuration/conformation of the compounds nor on their salt form; this information can be taken from the substances' names. Any discrepancy between name and structure is unintentional; in this case the name is decisive.

### I. Synthetic Examples

### Example 1

### (2S)—N-[[6-(4,4-Difluorocyclohexoxy)-5-fluoro-2-methoxy-3-pyridyl]methyl]-pyrrolidine-2-carboxamide; Fumaric Acid

(Compound of Formula Ia.1, Wherein X is CH, R⁵ is H, R⁶ is F, R⁷ is OCH₃ and R^{8a} is A.33)

### 1.1 6-((4,4-Difluorocyclohexyl)oxy)-2,5-difluoronicotinonitrile

### (Scheme 7 Step 1)

To a solution/suspension of potassium 2-methylpropan-2-olate (2.90 g, 25.8 mmol) in 30 ml of tetrahydrofuran at 0° C. was added 4,4-difluorocyclohexanol (3.79 g, 27.8 mmol) under nitrogen. After stirring for 30 minutes the reaction mixture was cooled to −70° C. and 2,5,6-trifluoronicotinonitrile (4.0 g, 25.3 mmol) in 15 ml of tetrahydrofuran was added slowly. The reaction mixture became orange and was stirred at −70° C. for 2 h and subsequently overnight at room temperature. After the addition of water, the aqueous layer was extracted three times with ethyl acetate, the combined organic layers were washed with saturated aqueous sodium chloride, dried over sodium sulphate, filtered and the solvent was evaporated. The raw material (7.21 g of a red-brown oil) was purified by column chromatography on silica gel (80 g column; heptane 100%→ethylacetate/heptane 80:20, 15 ml/min) to give 6-((4,4-difluorocyclohexyl)oxy)-2,5-difluoronicotinonitrile (6.94 g, yield 37%) as a white solid.

### 1.2 6-((4,4-Difluorocyclohexyl)oxy)-5-fluoro-2-methoxynicotinonitrile

### (Scheme 7 Step 2)

To a solution of potassium 2-methylpropan-2-olate (0.802 g, 7.15 mmol) in 24 ml of tetrahydrofuran at 0° C. was added methanol (0.229 ml, 7.15 mmol) under nitrogen. After stirring for 30 minutes the reaction mixture was cooled to −50° C. and 6-((4,4-difluorocyclohexyl)oxy)-2,5-difluoronicotinonitrile (1.40 ml, 5.11 mmol) in 24 ml of tetrahydrofuran was added slowly and the reaction temperature was kept at −50° C. for 2 h. Subsequently the cool bath was removed and the reaction warmed to room temperature. After the addition of water, the aqueous layer was extracted three times with ethyl acetate, the combined organic layers were washed with saturated aqueous sodium chloride, dried over sodium sulphate, filtered and the solvent was evaporated. The raw material (1.72 g of a white solid; yield: 100%) containing the desired product 6-((4,4-difluorocyclohexyl)oxy)-5-fluoro-2-methoxynicotinonitrile was used in the next step without further purification.

LCMS (ESI⁺) m/z [M+H]⁺: 287.1

### 1.3 (6-((4,4-Difluorocyclohexyl)oxy)-5-fluoro-2-methoxypyridin-3-yl)methanamine

### (Scheme 7 Step 3)

6-((4,4-Difluorocyclohexyl)oxy)-5-fluoro-2-methoxynicotinonitrile (1.55 g, 5.41 mmol) was dissolved in tetrahydrofuran (83 ml) under nitrogen. Boranemethylsulfide complex (5.41 ml, 10.83 mmol) was added at room temperature. The reaction mixture was stirred for 5 hours at 70° C. After cooling to 20° C. 14 ml of 2M HCl was added slowly (gassing, exothermic). 14 ml of methanol was added. The reaction mixture was stirred for 2 hours at 60° C. and subsequently at room temperature overnight. Water was added to the reaction mixture and the aqueous solution extracted twice with tert-butylmethylether. The pH of the aqueous solution was adjusted alkaline with 2M NaOH. The aqueous layer was extracted three times with dichloromethane. The dichloromethane phase was then washed with saturated aqueous sodium chloride, dried over sodium sulphate, filtered and the solvent evaporated. The raw material (1.06 g of a pale yellow oil) containing the desired product (6-((4,4-difluorocyclohexyl)oxy)-5-fluoro-2-methoxypyridin-3-yl)methanamine was used in the next step without further purification.

### 1.4 (S)-tert-Butyl 2-(((6-((4,4-difluorocyclohexyl)oxy)-5-fluoro-2-methoxypyridin-3-yl)methyl)carbamoyl)pyrrolidine-1-carboxylate

### (Scheme 2, Step 1)

The solution of (6-((4,4-difluorocyclohexyl)oxy)-5-fluoro-2-methoxypyridin-3-yl)methanamine (0.270 g, 0.931 mmol), N-ethyl-N-isopropylpropan-2-amine (0.325 ml, 1.862 mmol) and (S)-1-(tert-butoxycarbonyl)pyrrolidine-2-carboxylic acid (0.240 g, 1.117 mmol) in DMF (9.3 ml) was cooled to 0° C. 2-(1H-benzo[d][1,2,3]triazol-1-yl)-1,1,3,3-tetramethylisouronium hexafluorophosphate(V) (0.424 g, 1.117 mmol) was added to the reaction mixture and stirred 1 h at 0° C. and subsequently overnight at room temperature. After addition of water, the aqueous layer was extracted three times with ethylacetate, the combined organic layers were washed twice with 5% citric acid solution, once with water, with saturated aqueous sodium chloride, then dried over sodium sulphate, filtered and the solvent was evaporated. The raw material (0.526 g of a yellow oil), was purified by column chromatography on silica gel (12 g column; ethylacetate/heptane 20:80-80:20, 12 ml/min) to give (S)-tert-butyl 2-(((6-((4,4-difluorocyclohexyl)oxy)-5-fluoro-2-methoxypyridin-3-yl)methyl)carbamoyl)pyrrolidine-1-carboxylate (0.455 g, yield 100%) as a pale yellow oil.

LCMS (ESI⁺) m/z [M+H]⁺: 488.2

### 1.5 (S)—N-((6-((4,4-Difluorocyclohexyl)oxy)-5-fluoro-2-methoxypyridin-3-yl)methyl)pyrrolidine-2-carboxamide

### (Scheme 2, Step 2)

To a solution of (S)-tert-butyl 2-(((6-((4,4-difluorocyclohexyl)oxy)-5-fluoro-2-methoxypyridin-3-yl)methyl)carbamoyl)pyrrolidine-1-carboxylate (0.455 g, 0.933 mmol) in DCM (7.6 mL) was added TFA (0.719 ml, 9.33 mmol) dropwise at 0° C. The reaction solution was stirred overnight at room temperature and subsequently concentrated under reduced pressure. 1M NaOH was added to the residue, the aqueous layer was extracted three times with dichloromethane, the combined organic layers were washed with saturated aqueous sodium chloride, dried over sodium sulphate, filtered and the solvent was evaporated. The raw material (0.313 g, pale yellow oil), was purified by HPLC (column: xBridge prepMS C18, 19×150 mm, 5 μm; eluent: water with 0.1% TFA/MeOH with 0.1% TFA: 60/40→0/100; flow: 15 mL/min) to give the TFA salt of the desired compound, which was made alkaline to obtain the free base of (S)—N((6-((4,4-difluorocyclohexyl)oxy)-5-fluoro-2-methoxypyridin-3-yl)methyl)pyrrolidine-2-carboxamide (0.234 g, yield 65%) as a pale oil.

LCMS (ESI⁺) m/z [M+H]⁺: 388.2

### 1.6 (S)—N-((6-((4,4-Difluorocyclohexyl)oxy)-5-fluoro-2-methoxypyridin-3-yl)methyl)pyrrolidine-2-carboxamide; Fumaric Acid

Fumaric acid (0,069 g, 0,598 mmol) and (S)—N-((6-((4,4-difluorocyclohexyl)oxy)-5-fluoro-2-methoxypyridin-3-yl)methyl)pyrrolidine-2-carboxamide (0.234 g, 0.604 mmol) were dissolved in ethanol (2.6 ml), the solvent evaporated under reduced pressure and subsequently water added to the residue. Lyophilization of the aqueous solution gave (S)—N-((6-((4,4-difluorocyclohexyl)oxy)-5-fluoro-2-methoxypyridin-3-yl)methyl)pyrrolidine-2-carboxamide; fumaric acid (0.265 g, yield 87%) as white powder.

LCMS (ESI⁺) m/z [M+H]⁺: 388.2

¹H NMR (500 MHz, DMSO-d₆) δ ppm: 8.64 (t, J=5.9 Hz, 1H), 7.49 (d, J=10.4 Hz, 1H), 6.52 (s, 2H), 5.24-5.15 (m, 1H), 4.16 (dd, J=5.9, 1.6 Hz, 2H), 3.92 (dd, J=8.4, 5.7 Hz, 1H), 3.87 (s, 3H), 3.02 (tt, J=6.1, 3.0 Hz, 2H), 2.17-1.95 (m, 7H), 1.95-1.82 (m, 2H), 1.82-1.64 (m, 3H)

### Example 2

### (2S)—N-[(6-Benzyloxy-5-fluoro-2-methoxy-3-pyridyl)methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is CH₂-A.59)

### 2.1 2,6-Dichloro-5-fluoronicotinamide

### (Scheme 10 Step 1)

A solution of 2,6-dichloro-5-fluoronicotinonitrile (60 g, 314 mmol) in concentrated H₂SO₄ (600 mL) was stirred for 2 h at 60° C. After having cooled to 20° C., the reaction mixture was poured into ice-water, extracted with EtOAc (500 mL×3), and the organic phase was dried over Na₂SO₄ and concentrated under reduced pressure to give 2,6-dichloro-5-fluoronicotinamide (60 g, yield 91%) as a white solid.

¹H NMR (400 MHz, DMSO-d₆) δ ppm: 8.24-8.22 (d, J=8, 1H), 8.11 (s, 1H), 7.95 (s, 1H).

### 2.2 6-Chloro-5-fluoro-2-methoxynicotinamide

### (Scheme 10 Step 2)

To a solution of 2,6-dichloro-5-fluoronicotinamide (10 g, 47.8 mmol) in DMF (100 mL) was added sodium methanolate (7.75 g, 144 mmol) at 25° C. in portions and the mixture was stirred for 12 h at 25° C. Water (100 mL) was added and the reaction mixture was extracted with EtOAc (500 mL×3), and the organic phase was dried over Na₂SO₄ and concentrated under reduced pressure to give a residue, which was purified by column chromatography on silica gel (PE:EtOAc=5:1 to 4:1) to give the title compound 6-chloro-5-fluoro-2-methoxynicotinamide (2 g, yield 20%) as a white solid.

¹H NMR: (400 MHz, DMSO-d₆) δ ppm: 8.18-8.16 (d, J=8, 1H), 7.89 (s, 1H), 7.76 (s, 1H), 3.96 (s, 3H).

### 2.3 (6-Chloro-5-fluoro-2-methoxypyridin-3-yl)methanamine

### (Scheme 10 Step 3)

To a solution of 6-chloro-5-fluoro-2-methoxynicotinamide (5 g, 24.44 mmol) in THF (50 mL), a solution of BH₃.THF (98 mL, 98 mmol) was added dropwise at 20° C. Then the reaction mixture was stirred at 80° C. for 2 h. After cooled to 20° C., MeOH (20 mL) was added dropwise, and the resulting solution was stirred for 30 minutes. Then it was concentrated under reduced pressure and the residue was purified by preparative HPLC to give the title compound (6-chloro-5-fluoro-2-methoxypyridin-3-yl)methanamine (1.6 g, 34% yield).

¹H NMR: (400 MHz, DMSO-d₆) δ ppm: 8.29 (s, 2H), 8.00-7.98 (d, J=8, 1H), 3.99 (s, 2H), 3.91 (s, 3H).

LCMS (ESI⁺⁾ m/z [M+H]⁺: 191; RT: 1.857 min.

LC/MS: The gradient was 1-90% B in 3.4 min then 90-100% B to 3.85 min, finally changed to 1% B in 0.01 min under this condition for 0.65 min (0.8 mL/min flow rate. Mobile phase A was H₂O containing 0.0375% TFA, mobile phase B was acetonitrile containing 0.018% TFA. The column used for the chromatography is a 2.1×50 mm Venusil XBP-C18 column (5 μm particles). Detection methods are diode array (DAD) and evaporative light scattering (ELSD) detection as well as positive/negative electrospray ionization.)

### 2.4 (S)-tert-Butyl 2-(((6-chloro-5-fluoro-2-methoxypyridin-3-yl)methyl)carbamoyl)pyrrolidine-1-carboxylate

### (Scheme 5 Step 1)

To a solution of (S)-1-(tert-butoxycarbonyl)pyrrolidine-2-carboxylic acid (3.39 g, 15.74 mmol) in MeCN (40 mL) was added HATU (6.48 g, 17.05 mmol), and the mixture was stirred for 30 minutes at 25° C. Then (6-chloro-5-fluoro-2-methoxypyridin-3-yl)methanamine (2.5 g, 13.12 mmol) and DIPEA (4.58 mL, 26.2 mmol) were added at 25° C. The reaction mixture was stirred for 12 h at 25° C. Water (50 mL) was added, and the mixture was extracted with EtOAc (500 mL×3). The organic phase was dried and concentrated under reduced pressure and the residue was purified by preparative HPLC to give the title compound (S)-tert-butyl 2-(((6-chloro-5-fluoro-2-methoxypyridin-3-yl)methyl)carbamoyl)pyrrolidine-1-carboxylate (2.5 g, yield 49%) as a white solid.

¹H NMR: (400 MHz, CDCl₃) δ ppm: 7.37-7.35 (d, J=8, 1H), 4.29-4.23 (d, J=24, 3H), 3.88 (s, 3H), 3.36 (s, 2H), 2.28-1.83 (m, 4H), 1.39 (s, 9H).

LCMS (ESI⁺) m/z [M+H]⁺: 388.1; RT: 2.816 min.

LC/MS: The gradient was 10-100% B in 3.4 min then 100-100% B to 3.85 min, finally changed to 10% B in 0.01 min under this condition for 0.65 min (0.8 mL/min flow rate. Mobile phase A was H₂O containing 0.0375% TFA, mobile phase B was acetonitrile containing 0.018% TFA. The column used for the chromatography is a 2.1×50 mm Venusil XBP-C18 column (5 μm particles). Detection methods are diode array (DAD) and evaporative light scattering (ELSD) detection as well as positive/negative electrospray ionization.)

### 2.5 (S)-tert-Butyl 2-(((6-(benzyloxy)-5-fluoro-2-methoxypyridin-3-yl)methyl)carbamoyl)pyrrolidine-1-carboxylate

### (Scheme 5 Step 2)

To a solution of (S)-tert-butyl 2-(((6-chloro-5-fluoro-2-methoxypyridin-3-yl)methyl)carbamoyl)pyrrolidine-1-carboxylate (50 mg, 0.129 mmol) in toluene (2 mL) was added phenylmethanol (20.91 mg, 0.193 mmol), Cs₂CO₃ (84 mg, 0.258 mmol), [1,1′-biphenyl]-2-yldiisopropylphosphine (3.49 mg, 0.013 mmol) and Pd(OAc)₂ (2.89 mg, 0.013 mmol). The reaction mixture was stirred for 2 h at 100° C. The reaction mixture was concentrated under reduced pressure and the residue was purified by preparative HPLC to give the title compound (S)-tert-butyl 2-(((6-(benzyloxy)-5-fluoro-2-methoxypyridin-3-yl)methyl)carbamoyl)pyrrolidine-1-carboxylate (15 mg, yield 25%) as a yellow solid.

¹H NMR: (400 MHz, CDCl₃) δ ppm: 7.46-7.37 (m, 2H), 7.35-7.28 (m, 4H), 6.61 (s, 1H), 5.43 (s, 2H), 5.25 (s, 3H), 3.88-3.86 (d, J=8, 3H), 3.42 (s, 2H), 1.87 (s, 4H), 1.43-1.28 (d, J=60, 9H).

### 2.6 (S)—N-((6-(Benzyloxy)-5-fluoro-2-methoxypyridin-3-yl)methyl)pyrrolidine-2-carboxamide

To a solution of (S)-tert-butyl 2-(((6-(benzyloxy)-5-fluoro-2-methoxypyridin-3-yl)methyl)carbamoyl)pyrrolidine-1-carboxylate (100 mg, 0.218 mmol) in DCM (5 mL) was added TFA (0.5 mL) dropwise at 0° C. The reaction solution was stirred for 1 hour at 25° C. The reaction solution was concentrated under reduced pressure, and the residue was purified by preparative HPLC to give the title (S)—N-((6-(benzyloxy)-5-fluoro-2-methoxypyridin-3-yl)methyl)pyrrolidine-2-carboxamide (25.6 mg, yield 33%) as a yellow solid.

¹H NMR: (400 MHz, Methanol-d₄) δ ppm: 7.44-7.41 (m, 3H), 7.36-7.28 (m, 3H), 5.44 (s, 2H), 4.27-4.21 (m, 3H), 3.90 (s, 3H), 3.41-3.31 (m, 2H), 2.42-2.40 (m, 1H), 2.05-1.96 (m, 3H).

LCMS (ESI⁺) m/z [M+H]⁺: 360.2; RT: 2.652 min.

LC/MS: The gradient was 1-90% B in 3.4 min then 90-100% B to 3.85 min, finally changed to 1% B in 0.01 min under this condition for 0.65 min (0.8 mL/min flow rate. Mobile phase A was H₂O containing 0.0375% TFA, mobile phase B was acetonitrile containing 0.018% TFA. The column used for the chromatography is a 2.1×50 mm Venusil XBP-C18 column (5 μm particles). Detection methods are diode array (DAD) and evaporative light scattering (ELSD) detection as well as positive/negative electrospray ionization.)

### Example 3

### (2S)—N-[[6-(Cyclohexylmethoxy)-5-fluoro-2-methoxy-3-pyridyl]methyl]pyrrolidine-2-carboxamide; 2,2,2-trifluoroacetic acid

(Compound of Formula Ia.1, Wherein X is CH, R⁵ is H, R⁶ is F, R⁷ is OCH₃ and R^{8a} is CH₂-A.27)

The title compound was prepared using the procedure described in example 2, starting from cyclohexylmethanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 366.2

¹H NMR: (400 MHz, Methanol-d₄) δ ppm: 7.41-7.39 (d, J=8, 1H), 4.28-4.22 (m, 3H), 4.19-4.17 (d, J=8, 2H), 3.91 (s, 3H), 3.41-3.31 (m, 2H), 2.42-2.41 (m, 1H), 2.04-2.02 (m, 2H), 1.85-1.72 (m, 6H), 1.32-1.06 (m, 6H).

LCMS (ESI⁺) m/z [M+H]⁺: 366.2; RT: 2.580 min.

LC/MS: The gradient was 10-100% B in 3.4 min then 100-100% B to 3.85 min, finally changed to 10% B in 0.01 min under this condition for 0.65 min (0.8 mL/min flow rate. Mobile phase A was H₂O containing 0.0375% TFA, mobile phase B was acetonitrile containing 0.018% TFA. The column used for the chromatography is a 2.1×50 mm Venusil XBP-C18 column (5 μm particles). Detection methods are diode array (DAD) and evaporative light scattering (ELSD) detection as well as positive/negative electrospray ionization.)

### Example 4

### (2S)—N-[[6-[(4,4-Difluorocyclohexyl)methoxy]-5-fluoro-2-methoxy-3-pyridyl]-methyl]pyrrolidine-2-carboxamide; Fumaric Acid

(Compound of Formula Ia.1, Wherein X is CH, R⁵ is H, R⁶ is F, R⁷ is OCH₃ and R^{8a} is CH₂-A.33)

The title compound was prepared using the procedure described in example 2, starting from (4,4-difluorocyclohexyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the fumarate salt was formed by adding one equivalent fumaric acid.

LCMS (ESI+) m/z [M+H]+: 402.20

¹H NMR (500 MHz, Methanol-d₄) δ ppm 7.42 (d, J=10.0 Hz, 1H), 6.69 (s, 2H), 4.29 (d, J=2.4 Hz, 2H), 4.26 (d, J=6.2 Hz, 2H), 4.17 (dd, J=8.5, 6.8 Hz, 1H), 3.93 (s, 3H), 3.41-3.26 (m, 2H), 2.43-2.32 (m, 1H), 2.17-1.89 (m, 8H), 1.89-1.70 (m, 2H), 1.49-1.34 (m, 2H); one peak is under the MeOH signal.

### Example 5

### (S)—N-((6-((3,5-Difluorobenzyl)oxy)-5-fluoro-2-methoxypyridin-3-yl)methyl)pyrrolidine-2-carboxamide Hydrochloride

(Compound of Formula Ia.1, Wherein X is CH, R⁵ is H, R⁶ is F, R⁷ is OCH₃ and R^{8a} is CH₂-A.63)

The title compound was prepared using the procedure described in example 2, starting from (3,5-difluorophenyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

LCMS (ESI⁺) m/z [M+H]⁺: 396.3

¹H NMR (600 MHz, DMSO-d₆) δ ppm: 9.63 (s, 1H), 8.94 (t, J=5.7 Hz, 1H), 8.55 (s, 1H), 7.61 (d, J=10.3 Hz, 1H), 7.26-7.16 (m, 3H), 5.47 (s, 2H), 4.24-4.12 (m, 3H), 3.84 (s, 3H), 3.26-3.14 (m, 2H), 2.29 (ddt, J=12.7, 8.5, 6.3 Hz, 1H), 1.92-1.78 (m, 3H).

### Example 6

### (2S)—N-[[6-[(3,3-Difluorocyclopentyl)methoxy]-5-fluoro-2-methoxy-3-pyridyl]-methyl]pyrrolidine-2-carboxamide; Fumaric Acid

(Compound of Formula Ia.1, Wherein X is CH, R⁵ is H, R⁶ is F, R⁷ is OCH₃ and R^{8a} is CH₂-A.21)

The title compound was prepared using the procedure described in example 2, starting from (3,3-difluorocyclopentyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the fumarate salt was formed by adding one equivalent of fumaric acid.

LCMS (ESI⁺) m/z [M+H]⁺: 388.2

¹H NMR (500 MHz, DMSO-d6) δ ppm 8.63 (t, J=5.9 Hz, 1H), 7.49 (d, J=10.4 Hz, 1H), 6.52 (s, 2H), 4.32 (dd, J=6.7, 2.6 Hz, 2H), 4.16 (d, J=5.8 Hz, 2H), 3.97-3.90 (m, 1H), 3.87 (s, 3H), 3.05-2.99 (m, 2H), 2.69-2.54 (m, 1H), 2.36-2.22 (m, 1H), 2.22-2.02 (m, 3H), 2.02-1.85 (m, 2H), 1.83-1.69 (m, 3H), 1.69-1.53 (m, 1H)

### Example 7

### (2S)—N-[[6-[(3,3-Difluorocyclopentyl)methoxy]-5-fluoro-2-methoxy-3-pyridyl]-methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is CH₂-A.21)

The title compound was prepared from (2S)—N-[[6-[(3,3-difluorocyclopentyl)methoxy]-5-fluoro-2-methoxy-3-pyridyl]methyl]pyrrolidine-2-carboxamide (example 6) by reductive amination with formaldehyde.

(2S)—N-((6-((3,3-Difluorocyclopentyl)methoxy)-5-fluoro-2-methoxypyridin-3-yl)methyl)pyrrolidine-2-carboxamide (0.074 g, 0,191 mmol) was dissolved in DCE (5.8 ml) under nitrogen. Sodium triacetoxyborohydride (0.101 g, 0.478 mmol) and formaldehyde (1.442 ml, 19.10 mmol) were added. The reaction mixture was stirred overnight at room temperature. Saturated sodium bicarbonate solution was added to the reaction mixture. The aqueous layer was extracted three times with ethyl acetate, the combined organic layers were washed with saturated aqueous sodium chloride, dried over sodium sulphate, filtered and the solvent was evaporated. The raw material (0.102 g, colorless oil) was purified by column chromatography on silica gel (4 g column; dichloromethane/methanol: 0% methanol→10% methanol; 11 ml/min) to give the title compound (2S)—N-[[6-[(3,3-difluorocyclopentyl)methoxy]-5-fluoro-2-methoxy-3-pyridyl]methyl]-1-methyl-pyrrolidine-2-carboxamide (0.067 g, yield 87%) as a pale residue.

Finally the fumarate salt was formed by adding one equivalent of fumaric acid.

LCMS (ESI⁺) m/z [M+H]⁺: 402.2

¹H NMR (500 MHz, DMSO-d₆) δ ppm: 8.22 (t, J=6.1 Hz, 1H), 7.41 (d, J=10.4 Hz, 1H), 6.61 (s, 2H), 4.38-4.27 (m, 2H), 4.14 (dd, J=6.1, 3.1 Hz, 2H), 3.87 (s, 3H), 3.11-3.05 (m, 1H), 2.90 (dd, J=9.4, 5.7 Hz, 1H), 2.67-2.56 (m, 1H), 2.40-2.23 (m, 5H), 2.23-2.02 (m, 3H), 2.02-1.87 (m, 2H), 1.81-1.65 (m, 3H), 1.65-1.55 (m, 1H)

### Example 8

### (2S)—N-[[5-Fluoro-2-methoxy-6-[trans-4-(trifluoromethyl)cyclohexoxy]-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.36)

The title compound was prepared from (2S)—N-[[5-fluoro-2-methoxy-6-[4-(trifluoromethyl)cyclohexoxy]-3-pyridyl]methyl]pyrrolidine-2-carboxamide (example 11) by reductive amination with formaldehyde as described for example 7. Finally the fumarate salt was formed by adding one equivalent fumaric acid.

LCMS (ESI⁺) m/z [M+H]⁺: 434.2

¹H NMR (500 MHz, DMSO-d₆) δ ppm: 8.17 (t, J=6.1 Hz, 1H), 7.38 (d, J=10.5 Hz, 1H), 6.61 (s, 2H), 4.98-4.88 (m, 1H), 4.19-4.05 (m, 2H), 3.86 (s, 3H), 3.08-3.02 (m, 1H), 2.87-2.79 (m, 1H), 2.41-2.26 (m, 5H), 2.26-2.17 (m, 2H), 2.14-2.04 (m, 1H), 1.98-1.89 (m, 2H), 1.78-1.61 (m, 3H), 1.56-1.39 (m, 4H)

### Example 9

### (2S)—N-[[6-[(4,4-Difluorocyclohexyl)methoxy]-5-fluoro-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is CH₂-A.33)

The title compound was prepared from (2S)—N-[[6-[(4,4-difluorocyclohexyl)methoxy]-5-fluoro-2-methoxy-3-pyridyl]methyl]pyrrolidine-2-carboxamide (example 4) by reductive amination with formaldehyde as described for example 7. The fumarate salt was formed by adding one equivalent of fumaric acid.

LCMS (ESI⁺) m/z [M+H]⁺: 416.20

¹H NMR (500 MHz, Methanol-d₄) δ ppm: 7.41 (d, J=10.0 Hz, 1H), 6.70 (s, 2H), 4.29 (d, J=6.7 Hz, 2H), 4.26 (d, J=6.1 Hz, 2H), 3.93 (s, 3H), 3.74 (dd, J=9.1, 6.8 Hz, 1H), 3.61-3.51 (m, 1H), 3.04-2.93 (m, 1H), 2.77 (s, 3H), 2.53-2.37 (m, 1H), 2.15-2.02 (m, 3H), 2.02-1.89 (m, 5H), 1.89-1.70 (m, 2H), 1.50-1.34 (m, 2H)

### Example 10

### (2S)—N-[[6-(4,4-Difluorocyclohexoxy)-5-fluoro-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.33)

The title compound was prepared using the procedure described in example 5 starting from 4,4-difluorocyclohexanol, 2,5,6-trifluoronicotinonitrile, methanol and (S)-1-methylpyrrolidine-2-carboxylic acid. Finally the fumarate salt was formed by adding one equivalent fumaric acid.

LCMS (ESI⁺) m/z [M+H]⁺: 402.2

¹H NMR (500 MHz, DMSO-d₆) δ ppm: 8.27 (t, J=5.9 Hz, 1H), 7.42 (d, J=10.4 Hz, 1H), 6.60 (s, 2H), 5.22-5.17 (m, 1H), 4.15 (dd, J=6.1, 2.7 Hz, 2H), 3.87 (s, 3H), 3.14-3.08 (m, 1H), 2.96 (dd, J=9.4, 5.8 Hz, 1H), 2.44-2.32 (m, 4H), 2.17-2.08 (m, 1H), 2.08-1.96 (m, 6H), 1.96-1.83 (m, 2H), 1.81-1.65 (m, 3H)

### Example 11

### (2S)—N-[[5-Fluoro-2-methoxy-6-[trans-4-(trifluoromethyl)cyclohexoxy]-3-pyridyl]methyl]pyrrolidine-2-carboxamide; Fumaric Acid

(Compound of Formula Ia.1, Wherein X is CH, R⁵ is H, R⁶ is F, R⁷ is OCH₃ and R^{8a} is A.36)

The title compound was prepared using the procedure described in example 5 starting from trans-4-(trifluoromethyl)cyclohexanol, 2,5,6-trifluoronicotinonitrile, methanol and (S)-1-(tert-butoxycarbonyl)pyrrolidine-2-carboxylic acid, followed by BOC deprotection. Finally the fumarate salt was formed by adding one equivalent fumaric acid.

LCMS (ESI⁺) m/z [M+H]⁺: 420.2

¹H NMR (500 MHz, DMSO-d₆) δ ppm: 8.60 (t, J=5.8 Hz, 1H), 7.46 (d, J=10.4 Hz, 1H), 6.52 (s, 2H), 4.98-4.88 (m, 1H), 4.15 (d, J=5.9 Hz, 2H), 3.92-3.83 (m, 5H), 3.04-2.97 (m, 2H), 2.42-2.32 (m, 1H), 2.25-2.15 (m, 2H), 2.15-2.05 (m, 1H), 1.98-1.89 (m, 2H), 1.79-1.69 (m, 3H), 1.56-1.39 (m, 4H)

### Example 12

### (2S)—N-[[5-Fluoro-2-methoxy-6-(4-methoxycyclohexoxy)-3-pyridyl]methyl]-pyrrolidine-2-carboxamide; Fumaric Acid

(Compound of Formula Ia.1, Wherein X is CH, R⁵ is H, R⁶ is F, R⁷ is OCH₃ and R^{8a} is A.46)

The title compound was prepared using the procedure described in scheme 9 starting from 4-methoxycyclohexanol, 2,6-dichloro-5-fluoronicotinonitrile, 2-(methylsulfonyl)ethanol, iodomethane and (S)-1-(tert-butoxycarbonyl)pyrrolidine-2-carboxylic acid, followed by BOC deprotection. Finally the fumarate salt was formed by adding one equivalent fumaric acid.

### 12.1 2-Chloro-5-fluoro-6-((4-methoxycyclohexyl)oxy)nicotinonitrile

### (Scheme 9, Step 1)

In a 150 ml 3-necked flask 3.1 g of KOtBu (27.6 mmol) were suspended in tetrahydrofuran (10 ml), then a solution of 4-methoxycyclohexanol (3.8 g, 29.2 mmol) in 15 ml of tetrahydrofurane was added dropwise maintaining a temperature of 0° C. After stirring for 1h the mixture was cooled to −70° C., 2,6-dichloro-5-fluoror-3-pyridinecarbonitrile (4.7 g, 24.61 mmol) dissolved in 15 ml of tetrahydrofurane was added, and stirring was continued for 3 h at −70° C. and then overnight at room temperature. 100 ml of water were added, the mixture was concentrated, then 100 ml of 10% aqueous NaHCO₃ were added, the solution was extracted 4 times with 150 ml of ethyl acetate, the combined organic layers were washed with brine, dried over MgSO₄ and the solvent was removed. The crude product was then was subjected to flash chromatography (silica gel/dichloromethane) to yield 4.4 g of the title compound as an off-white solid.

### 12.2 5-Fluoro-2-hydroxy-6-((4-methoxycyclohexyl)oxy)nicotinonitrile

### (Scheme 9, Step 2 First Part)

NaH (0.9 g, 22.50 mmol) was added to a solution of 2-chloro-5-fluoro-6-((4-methoxycyclohexyl)oxy)nicotinonitrile (3.0 g, 10.54 mmol) and 2-(methylsulfonyl)ethanol (1.5 g, 12.08 mmol) in N,N-dimethylformamide (15 ml) at 5° C. and the mixture was stirred for 30 minutes at 5° C. A 2:1 mixture of water/N,N-dimethylformamide (20 ml) and then water (50 ml) was added, the mixture acidified to pH 1-2 using about 20 ml of 1N HCl, extracted 3 times with each 100 ml of ethyl acetate, the combined organic layers were washed with brine, dried over MgSO₄, filtered and evaporated. The obtained crude product was purified using flash chromatography (silica gel/gradient from 10-20% methanol in dichloromethane) to give 3.2 g of the title compound as yellow oil; MS (ESI+) m/z [M+H]+: 267.15.

### 12.3 5-Fluoro-2-methoxy-6-((4-methoxycyclohexyl)oxy)nicotinonitrile

### (Scheme 9, Step 2 Second Part)

A mixture of 5-fluoro-2-hydroxy-6-((4-methoxycyclohexyl)oxy)nicotinonitrile (720 mg, 2.70 mmol), silver carbonate (950 mg, 3.45 mmol) and iodomethane (190 μl, 3.05 mmol) in 18 ml of acetonitrile was heated to 105° C. for 65 minutes in a Biotage microwave. The mixture then was filtered, the filtrate concentrated and the remainder subjected to flash chromatography (silica gel/10% methanol in dichloromethane) to give 0.51 g of the title compound as an amorphous white solid; MS (ESI+) m/z [M+H]+: 281.2.

### 12.4 (5-Fluoro-2-methoxy-6-((4-methoxycyclohexyl)oxy)pyridin-3-yl)methanamine

### (Scheme 7 Step 3)

In a 150 3-necked flask cobalt chloride hexahydrate (1.1 g, 4.62 mmol) and sodium borohydride (0.31 g, 8.19 mmol) were added to 5-fluoro-2-methoxy-6-((4-methoxycyclohexyl)oxy)nicotinonitrile (0.8 g, 2.85 mmol) in methanol (10 ml) and the mixture was stirred for 2 h at room temperature. 10 ml of water and 45 ml of concentrated HCl were added, and after stirring for 5 minutes adjusted to pH 11 using NH₄OH (about 45 ml). The mixture was extracted 3 times with each 80 ml of dichloromethane, the combined organic layers were washed with brine, dried over MgSO₄, filtered and evaporated to give 700 mg of a brownish oil, which was used in the next reaction without further purification.

### 12.5 (S)-tert-Butyl 2-(((5-fluoro-2-methoxy-6-((4-methoxycyclohexyl)oxy)pyridin-3-yl)methyl)carbamoyl)pyrrolidine-1-carboxylate

### (Scheme 2, Step 1)

To a solution of (5-fluoro-2-methoxy-6-((4-methoxycyclohexyl)oxy)pyridin-3-yl)methanamine (660 mg, 2.089 mmol) and Boc-L-proline (550 mg, 2.56 mmol) in N,N-dimethylformamide (12 ml) at 5° C. was added COMU ((1-cyano-2-ethoxy-2-oxoethylidenaminooxy)dimethylamino-morpholino-carbenium hexafluorophosphate) (1200 mg, 2.80 mmol) and N,N-diisopropylethylamine (900 μl, 5.15 mmol) and the mixture was stirred overnight at room temperature. The mixture was concentrated, the remainder taken up in ethyl acetate (100 ml), washed with water, dried over MgSO₄, filtered and evaporated to give 1.9 g of a reddish oil which was subjected to flash chromatography (silica gel/gradient 10-20% methanol in dichloromethane) to give 0.99 g of the title compound as an amorphous yellow solid; LCMS (ESI+) m/z [M+H]+: 482.4.

### 12.6 (S)—N-((5-Fluoro-2-methoxy-6-((4-methoxycyclohexyl)oxy)pyridin-3-yl)methyl)pyrrolidine-2-carboxamide

### (Scheme 2, Step 2)

To a solution of (S)-tert-butyl 2-(((5-fluoro-2-methoxy-6-((4-methoxycyclohexyl)oxy)pyridin-3-yl)methyl)carbamoyl)pyrrolidine-1-carboxylate (590 mg, 1,103 mmol) in dichloromethane (15 ml) at 5° C. TFA (1.0 ml, 12.98 mmol) was added and then the mixture was stirred overnight at room temperature. Dichloromethane (20 ml) and saturated aqueous NaHCO₃ (10 ml) were added, and the organic layer was separated and concentrated to give 490 mg of a yellow oil, which was subjected to flash chromatography (silica gel/gradient 20-30% methanol in dichloromethane) to give 320 mg of the title compound as an oil.

LCMS (ESI⁺) m/z [M+H]⁺: 382.2.

### 12.7 (S)—N-((5-Fluoro-2-methoxy-6-((4-methoxycyclohexyl)oxy)pyridin-3-yl)methyl)pyrrolidine-2-carboxamide, Fumaric Acid

A solution of (S)—N-((5-fluoro-2-methoxy-6-((4-methoxycyclohexyl)oxy)pyridin-3-yl)methyl)pyrrolidine-2-carboxamide (19 mg, 0.050 mmol) and fumaric acid (6 mg, 0.052 mmol) in ethanol (10 ml) was stirred at room temperature for 30 minutes, filtered and the obtained solid dried to give 24 mg of the title compound as an amorphous white solid.

¹H NMR (500 MHz, DMSO-d₆) δ ppm: 8.57 (m, 1H), 7.45 (d, 1H), 6.54 (s, 2H), 5.07 and 4.97 (each m, together 1H-diasteromere), 4.14 (d, 2H), 3.85 (s, 3H), 3.24 (s, 3H), 3.00 (m, 2H), 2.15-2.05 (m, 2H), 1.97 (m, 1H), 1.81-1.65 (m, 9H), 1.53 (m, 1H), 1.36 (m, 1H).

### Example 13

### (2S)—N-[[5-Fluoro-2-methoxy-6-(4-methoxycyclohexoxy)-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.46)

The title compound was prepared from (S)—N-((5-fluoro-2-methoxy-6-((4-methoxycyclohexyl)oxy)pyridin-3-yl)methyl)pyrrolidine-2-carboxamide (example 12) by reductive amination with formaldehyde. Finally the fumarate salt was formed by adding one equivalent of fumaric acid.

To a solution of (S)—N-((5-fluoro-2-methoxy-6-((4-methoxycyclohexyl)oxy)pyridin-3-yl)methyl)pyrrolidine-2-carboxamide (95 mg, 0.249 mmol) in methanol (5 ml) were added formaldehyde (40 μL, 0.537 mmol), subsequently zinc chloride (60 mg, 0.440 mmol), and after 10 minutes sodium cyanoborohydride (30 mg, 0.477 mmol). The reaction mixture was stirred at room temperature for 1 h, then concentrated, treated with 10 ml of saturated aqueous NaHCO₃ and 20 ml of dichloromethane, and the different layers were separated via Chromabond PTS. Evaporation of the organic layer gave 80 mg of the crude product, which was subjected to flash chromatography (silica gel/gradient 10-20% methanol in dichloromethane) to give 60 mg as oil.

LC MS (ESI⁺) m/z [M+H]⁺: 396.2.

Conversion into the fumarate as described for example 12 gave 77 mg of the title compound as an off-white amorphous solid.

¹H NMR (500 MHz, DMSO-d₆) δ ppm: 8.19 (m, 1H), 7.37 (d, 1H), 6.61 (s, 2H), 5.06 and 4.97 (each m, together 1H-diasteromers), 4.13 (d, 2H), 3.84 (s, 3H), 3.24 (s, 3H), 3.04 (m, 1H), 2.82 (m, 1H), 2.29 (s, 3H), 2.15-1.95 (m, 3H), 1.90-1.60 (m, 9H), 1.51 (m, 1H), 1.38 (m, 1H).

### Example 14

### (2S)—N-[[5-Fluoro-2-methoxy-6-[[4-(trifluoromethyl)cyclohexyl]methoxy]-3-pyridyl]methyl]pyrrolidine-2-carboxamide; Fumaric Acid

(Compound of Formula Ia.1, Wherein X is CH, R⁵ is H, R⁶ is F, R⁷ is OCH₃ and R^{8a} is CH₂-A.36)

The title compound was prepared using the procedure described in example 12 starting from trans-4-(trifluoromethyl)cyclohexyl)methanol, 2,6-dichloro-5-fluoronicotinenitrile, 2-(methylsulfonyl)ethanol, iodomethane and (S)-1-(tert-butoxycarbonyl)pyrrolidine-2-carboxylic acid, followed by BOC deprotection. Finally the fumarate salt was formed by adding one equivalent fumaric acid.

LCMS (ESI⁺) m/z [M+H]⁺: 434.3 (free base)

¹H NMR (500 MHz, DMSO-d₆) δ ppm: 8.52 (m, 1H), 7.44 (d, 1H), 6.52 (s, 2H), 4.16 (d, 2H), 4.02 (d, 2H), 3.79 (m, 1H), 2.23 (m, 1H), 2.06 (m, 1H), 1.90 (m, 4H), 1.80 (m, 1H), 1.70 (m, 3H), 1.28 (m, 2H), 1.11. (m, 2H).

### Example 15

### (2S)—N-[[5-Fluoro-2-methoxy-6-[[4-(trifluoromethyl)cyclohexyl]methoxy]-3-pyridyl]methyl]-1-methyl-pyrrolidine-2-carboxamide

(Compound of Formula Ia.2, Wherein X is CH, R⁵ is H, R⁶ is F, R⁷ is OCH₃ and R^{8a} is CH₂-A.36)

The title compound was prepared from (2S)—N-[[5-fluoro-2-methoxy-6-[[4-(trifluoromethyl)cyclohexyl]methoxy]-3-pyridyl]methyl]pyrrolidine-2-carboxamide (example 14) by reductive amination with formaldehyde as described in example 13.

LCMS (ESI⁺) m/z [M+H]⁺: 448.2

¹H NMR (500 MHz, DMSO-d₆) δ ppm: 8.14 (m, 1H), 7.38 (d, 1H), 4.19 (d, 2H), 4.11 (d, 2H), 3.86 (s, 3H), 3.01 (m, 1H), 2.73 (m, 1H), 2.25 (m, 5H), 2.06 (m, 1H), 1.91 (m, 4H), 1.91 (m, 1H), 1.79 (m, 3H), 1.30 (m, 2H), 1.15 (m, 2H).

### Example 16

### (2S)—N-[[6-[[(1R)-3,3-Difluorocyclopentyl]methoxy]-5-fluoro-2-methoxy-3-pyridyl]methyl]-1-methyl-pyrrolidine-2-carboxamide

(Compound of Formula Ia.2, Wherein X is CH, R⁵ is H, R⁶ is F, R⁷ is OCH₃ and R^{8a} is CH₂-A.21)

The title compound was prepared by the separation of the two isomers of (2S)—N-[[6-[(3,3-difluorocyclopentyl)methoxy]-5-fluoro-2-methoxy-3-pyridyl]methyl]-1-methyl-pyrrolidine-2-carboxamide (example 7) on a chiral column via HPLC.

Analytic method: Agilent 1100 HPLC; column: Chiralpak® IA, 4.6×250 mm, 5 μm; eluent: 97% n-heptane, 3% isopropanol, 0.4% diethylamine; flow rate: 0.9 mL/min; Time: 19.5 min

Preparative method: Gilson 215/333 Prep-HPLC; column: Chiralpak® IA, 20×250 mm, 5 μm; eluent: 97% n-heptane, 3% isopropanol, 0.4% diethylamine; flow rate: 13 mL/min; Time: 25 min

LCMS (ESI⁺) m/z [M+H]⁺: 402.3

¹H NMR (600 MHz, CDCl₃) δ ppm: 7.67 (s, 1H), 7.30 (d, J=9.8 Hz, 1H), 4.34-4.25 (m, 4H), 3.90 (s, 3H), 3.08 (t, J=7.8 Hz, 1H), 2.90 (dd, J=10.2, 5.4 Hz, 1H), 2.72-2.63 (m, 1H), 2.38-2.29 (m, 5H), 2.27-2.16 (m, 2H), 2.16-1.92 (m, 3H), 1.86-1.64 (m, 4H)

### Example 17

### (2S)—N-[[6-[[(1S)-3,3-Difluorocyclopentyl]methoxy]-5-fluoro-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is CH₂-A.21)

The title compound was prepared by the separation of the two isomers of (2S)—N-[[6-[(3,3-difluorocyclopentyl)methoxy]-5-fluoro-2-methoxy-3-pyridyl]methyl]-1-methyl-pyrrolidine-2-carboxamide (example 7) on a chiral HPLC column.

Analytic method: Agilent 1100 HPLC; column: Chiralpak® IA, 4.6×250 mm, 5 μm; eluent: 97% n-heptane, 3% isopropanol, 0.4% diethylamine; flow rate: 0.9 mL/min; Time: 16.1 min

Preparative method: Gilson 215/333 Prep-HPLC; column: Chiralpak® IA, 20×250 mm, 5 μm; eluent: 97% n-heptane, 3% isopropanol, 0.4% diethylamine; flow rate: 13 mL/min; Time: 22 min

Finally the fumarate salt was formed by adding one equivalent fumaric acid.

LCMS (ESI⁺) m/z [M+H]⁺: 402.3

¹H NMR (500 MHz, DMSO-d₆) δ ppm: 8.18 (t, J=6.1 Hz, 1H), 7.44 (d, J=10.5 Hz, 1H), 6.61 (s, 2H), 4.30-4.25 (m, 1H), 4.25-4.19 (m, 1H), 4.19-4.12 (m, 2H), 3.92 (s, 3H), 3.08 (td, J=6.9, 3.6 Hz, 1H), 2.92 (dd, J=9.4, 5.9 Hz, 1H), 2.65-2.54 (m, 1H), 2.43-2.36 (m, 1H), 2.33 (s, 3H), 2.32-2.26 (m, 1H), 2.25-2.02 (m, 3H), 2.02-1.88 (m, 2H), 1.81-1.55 (m, 4H)

### Example 18

### (2S)—N-[[5-Fluoro-2-methoxy-6-(2,2,3,3,3-pentafluoropropoxy)-3-pyridyl]methyl]-pyrrolidine-2-carboxamide; Succinic Acid

(Compound of Formula Ia.1, Wherein X is CH, R⁵ is H, R⁶ is F, R⁷ is OCH₃ and R^{8a} is CH₂CF₂CF₃)

The title compound was prepared using the procedure described in example 2, starting from 2,2,3,3,3-pentafluoropropan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 402.20

¹H NMR (500 MHz, Methanol-d₄) δ ppm 7.52 (d, J=9.8 Hz, 1H), 5.04 (tt, J=13.1, 1.2 Hz, 2H), 4.31 (s, 2H), 4.12 (dd, J=8.6, 6.5 Hz, 1H), 3.95 (s, 3H), 3.33-3.19 (m, 2H), 2.52 (s, 4H), 2.41-2.26 (m, 1H), 2.04-1.87 (m, 3H); one peak is under the MeOH signal.

### Example 19

### (2S)—N-[[5-Fluoro-2-methoxy-6-[cis-4-(trifluoromethyl)cyclohexoxy]-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.36)

The title compound was prepared using the procedure described in example 1 starting from cis-4-(trifluoromethyl)cyclohexanol, 2,5,6-trifluoronicotinonitrile, methanol and (S)-1-methylpyrrolidine-2-carboxylic acid. Finally the fumarate salt was formed by adding one equivalent fumaric acid.

LCMS (ESI⁺) m/z [M+H]⁺: 434.3

¹H NMR (500 MHz, DMSO-d₆) δ ppm: 8.26 (t, J=5.9 Hz, 1H), 7.41 (d, J=10.3 Hz, 1H), 6.61 (s, 2H), 5.28-5.24 (m, 1H), 4.15 (dd, J=6.1, 2.4 Hz, 2H), 3.85 (s, 3H), 3.14-3.07 (m, 1H), 2.95 (dd, J=9.4, 5.7 Hz, 1H), 2.46-2.37 (m, 2H), 2.35 (s, 3H), 2.18-2.04 (m, 3H), 1.80-1.64 (m, 7H), 1.64-1.51 (m, 2H)

### Example 20

### (2S)—N-[[5-Fluoro-2-methoxy-6-[cis-4-(trifluoromethyl)cyclohexoxy]-3-pyridyl]methyl]pyrrolidine-2-carboxamide; Fumaric Acid

(Compound of Formula Ia.1, Wherein X is CH, R⁵ is H, R⁶ is F, R⁷ is OCH₃ and R^{8a} is A.36)

The title compound was prepared using the procedure described in example 1 starting from cis-4-(trifluoromethyl)cyclohexanol, 2,5,6-trifluoronicotinonitrile, methanol and (S)-1-(tert-butoxycarbonyl)pyrrolidine-2-carboxylic acid, followed by BOC deprotection. Finally the fumarate salt was formed by adding one equivalent fumaric acid.

LCMS (ESI⁺) m/z [M+H]⁺: 420.3

¹H NMR (500 MHz, DMSO-d₆) δ ppm: 8.68 (t, J=5.9 Hz, 1H), 7.49 (d, J=10.3 Hz, 1H), 6.52 (s, 2H), 5.30-5.22 (m, 1H), 4.16 (d, J=5.8 Hz, 2H), 3.97 (dd, J=8.3, 6.0 Hz, 1H), 3.85 (s, 3H), 3.06 (t, J=6.8 Hz, 2H), 2.47-2.34 (m, 1H), 2.19-2.11 (m, 1H), 2.11-2.04 (m, 2H), 1.85-1.64 (m, 7H), 1.64-1.50 (m, 2H)

### Example 21

### (2S)—N-[[6-(4,4-Difluorocyclohexoxy)-5-fluoro-2-methoxy-3-pyridyl]methyl]-1-methyl-2,5-dihydropyrrole-2-carboxamide

(Compound of Formula Ia.8, Wherein X is CH, R⁵ is H, R⁶ is F, R⁷ is OCH₃ and R^{8a} is A.33)

The title compound was prepared using the procedure described in example 1 starting from 4,4-difluorocyclohexanol, 2,5,6-trifluoronicotinonitrile, methanol and (S)-1-(tert-butoxycarbonyl)-2,5-dihydro-1H-pyrrole-2-carboxylic acid, followed by BOC deprotection and subsequent reductive amination with formaldehyde as described in example 7.

LCMS (ESI⁺) m/z [M+H]⁺: 400.4

¹H NMR (600 MHz, CDCl₃) δ ppm: 7.88 (s, 1H), 7.29 (d, J=9.6 Hz, 1H), 5.83 (s, 2H), 5.21 (tt, J=6.0, 3.1 Hz, 1H), 4.92-4.88 (m, 1H), 4.37-4.24 (m, 2H), 4.05-3.89 (m, 2H), 3.88 (s, 3H), 2.54 (s, 3H), 2.24-2.03 (m, 4H), 2.03-1.84 (m, 4H)

### Example 22

### (S)—N-((6-(Bicyclo[1.1.1]pentan-1-ylmethoxy)-5-fluoro-2-methoxypyridin-3-yl)methyl)pyrrolidine-2-carboxamide Hydrochloride

(Compound of Formula Ia.1, Wherein X is CH, R⁵ is H, R⁶ is F, R⁷ is OCH₃ and R^{8a} is CH₂-A.90)

The title compound was prepared using the procedure described in example 2, starting from bicyclo[1.1.1]pentan-1-ylmethanol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent of hydrochloric acid.

LCMS (ESI⁺) m/z [M+H]⁺: 350.3

¹H NMR (500 MHz, methanol-d₄) δ ppm: 7.40 (d, J=10.1 Hz, 1H), 4.33 (s, 2H), 4.27 (s, 2H), 4.09 (dd, J=8.5, 6.6 Hz, 1H), 3.92 (s, 3H), 3.35-3.27 (m, 1H; overlap with MeOD peak), 3.22 (dt, J=11.3, 6.9 Hz, 1H), 2.50 (s, 1H), 2.39-2.28 (m, 1H), 2.03-1.87 (m, 3H), 1.81 (s, 6H).

### Example 23

### (2S)—N-[[6-(3,3-Difluorocyclopentoxy)-5-fluoro-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.21)

The title compound was prepared using the procedure described in example 1 starting from 3,3-difluorocyclopentanol, 2,5,6-trifluoronicotinonitrile, methanol and (S)-1-methylpyrrolidine-2-carboxylic acid. Finally the fumarate salt was formed by adding one equivalent fumaric acid.

LCMS (ESI⁺) m/z [M+H]⁺: 388.30

¹H NMR (500 MHz, Methanol-d₄) δ ppm: 7.42 (d, J=9.8 Hz, 1H), 6.70 (s, 2H), 5.52-5.44 (m, 1H), 4.37-4.23 (m, 2H), 3.93 (s, 3H), 3.74-3.65 (m, 1H), 3.53 (ddd, J=10.8, 7.7, 3.7 Hz, 1H), 3.01-2.90 (m, 1H), 2.75 (s, 3H), 2.72-2.57 (m, 1H), 2.50-2.39 (m, 1H), 2.39-2.23 (m, 3H), 2.23-2.02 (m, 3H), 2.02-1.87 (m, 2H)

### Example 24

### (2S)—N-[[6-(3,3-Difluorocyclopentoxy)-5-fluoro-2-methoxy-3-pyridyl]methyl]-pyrrolidine-2-carboxamide; Fumaric Acid

(Compound of Formula Ia.1, Wherein X is CH, R⁵ is H, R⁶ is F, R⁷ is OCH₃ and R^{8a} is A.21)

The title compound was prepared using the procedure described in example 1 starting from 3,3-difluorocyclopentanol, 2,5,6-trifluoronicotinonitrile, methanol and (S)-1-(tertbutoxycarbonyl)pyrrolidine-2-carboxylic acid, followed by BOC deprotection. Finally the fumarate salt was formed by adding one equivalent fumaric acid.

LCMS (ESI⁺) m/z [M+H]⁺: 374.30

¹H NMR (500 MHz, Methanol-d₄) δ ppm: 7.44 (d, J=10.0 Hz, 1H), 6.68 (s, 2H), 5.52-5.45 (m, 1H), 4.29 (d, J=2.1 Hz, 2H), 4.22 (dd, J=8.5, 6.8 Hz, 1H), 3.93 (s, 3H), 3.41-3.27 (m, 2H), 2.73-2.61 (m, 1H), 2.43-2.25 (m, 4H), 2.22-1.93 (m, 5H); one peak is under the MeOH signal.

### Example 25

### (2R)—N-[[6-[[(1R)-3,3-Difluorocyclopentyl]methoxy]-5-fluoro-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is CH₂-A.21)

The title compound was prepared using the procedure described in example 1 starting from (R)-(3,3-difluorocyclopentyl)methanol, 2,5,6-trifluoronicotinonitrile, methanol and racemic 1-methylpyrrolidine-2-carboxylic acid. The separation of the two isomers was performed on a chiral column via HPLC.

Analytic method: Agilent 1100 HPLC; column: Chiralpak® IA, 4.6×250 mm, 5 μm; eluent: 97% n-heptane, 3% isopropanol, 0.4% diethylamine; flow rate: 0.9 mL/min, Time: 18.8 min

Preparative method: Gilson 215/333 Prep-HPLC; column: Chiralpak® IA, 20×250 mm, 5 μm; eluent: 97% n-heptane, 3% isopropanol, 0.4% diethylamine; flow rate: 13 mL/min; Time: 25 min

Finally the fumarate salt was formed by adding one equivalent fumaric acid.

LCMS (ESI⁺) m/z [M+H]⁺: 402.30

¹H NMR (500 MHz, Methanol-d₄) δ ppm: 7.41 (d, J=9.8 Hz, 1H), 6.70 (s, 2H), 4.41-4.31 (m, 2H), 4.31-4.25 (m, 2H), 3.93 (s, 3H), 3.77-3.69 (m, 1H), 3.54 (ddd, J=11.0, 7.7, 3.9 Hz, 1H), 3.01-2.91 (m, 1H), 2.76 (s, 3H), 2.72-2.58 (m, 1H), 2.50-2.36 (m, 1H), 2.36-2.23 (m, 1H), 2.23-2.14 (m, 1H), 2.14-1.89 (m, 6H), 1.74-1.61 (m, 1H)

### Example 26

### (2S)—N-[[6-[(1S)-3,3-Difluorocyclopentoxy]-5-fluoro-2-methoxy-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is A.21)

The title compound was prepared using the procedure described in example 1 starting from 3,3-difluorocyclopentanol, 2,5,6-trifluoronicotinonitrile, methanol and (S)-1-methylpyrrolidine-2-carboxylic acid. The separation of the two isomers was performed with SFC

Analytic method: SFC (Agilent 1260 Infinity Hybrid SFC); column: Chiralpak® IA for SFC, 4.6×100 mm, 5 μm; eluent: 75% CO₂; 25% (acetonitrile with 0.1% diethylamine); flow rate: 3.5 mL/min; time: 3.98 min

Preparative method: SFC (Waters Prep 100q SFC); column: Chiralpak® IA for SFC, 20×250 mm, 5 μm eluent: 65% CO₂; 35% (70% acetonitrile, 30% dichlormethane with 0.1% diethylamine); flow rate: 60 mL/min: time: 7.14 min

Finally the hydrochloride salt was formed by adding one equivalent hydrochloric acid.

LCMS (ESI⁺) m/z [M+H]⁺: 388.3

¹H NMR (600 MHz, DMSO-d₆) δ ppm: 9.71 (s, 1H), 9.13 (t, J=5.6 Hz, 1H), 7.60 (d, J=10.4 Hz, 1H), 5.46 (dd, J=6.8, 3.6 Hz, 1H), 4.30-4.16 (m, 2H), 4.10 (q, J=8.0 Hz, 1H), 3.88 (s, 3H), 3.61-3.52 (m, 1H), 3.22-3.10 (m, 1H), 2.94-2.84 (m, 1H), 2.81 (d, J=4.7 Hz, 3H), 2.77-2.67 (m, 1H), 2.40-2.24 (m, 3H), 2.24-2.13 (m, 1H), 2.13-1.94 (m, 2H), 1.94-1.76 (m, 2H)

### Example 27

### (2S)—N-[[6-[(1R)-3,3-Difluorocyclopentoxy]-5-fluoro-2-methoxy-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is A.21)

The title compound was prepared using the procedure described in example 1 starting from 3,3-difluorocyclopentanol, 2,5,6-trifluoronicotinonitrile, methanol and (S)-1-methylpyrrolidine-2-carboxylic acid. The separation of the two isomers was performed with SFC using the same method as described in example 26. Finally the hydrochloride salt was formed by adding one equivalent hydrochloric acid.

Analytic method: SFC (Agilent 1260 Infinity Hybrid SFC); column: Chiralpak® IA for SFC, 4.6×100 mm, 5 μm; eluent: 75% CO₂; 25% (acetonitrile with 0.1% diethylamine); flow rate: 3.5 mL/min; time: 4.57 min

Preparative method: SFC (Waters Prep 100q SFC); column: Chiralpak® IA for SFC, 20×250 mm, 5 μm eluent: 65% CO₂; 35% (70% acetonitrile, 30% dichlormethane with 0.1% diethylamine); flow rate: 60 mL/min: time: 8.21 min

LCMS (ESI⁺) m/z [M+H]⁺: 388.2

¹H NMR (500 MHz, DMSO-d₆) δ ppm: 9.75 (s, 1H), 9.21 (t, J=5.6 Hz, 1H), 8.87 (s, 7H), 7.61 (d, J=10.4 Hz, 1H), 5.46 (tt, J=6.5, 3.8 Hz, 1H), 4.22 (dd, J=5.6, 3.5 Hz, 2H), 4.19-4.08 (m, 1H), 3.88 (s, 3H), 3.60-3.54 (m, 1H), 3.19-3.09 (m, 1H), 2.93-2.84 (m, 15H), 2.82 (d, J=4.7 Hz, 3H), 2.77-2.64 (m, 1H), 2.54-2.47 (m, 1H, under dmso peak), 2.39-2.23 (m, 3H), 2.23-2.12 (m, 1H), 2.12-1.94 (m, 2H), 1.94-1.77 (m, 2H), 1.19 (t, J=7.3 Hz, 22H); one peak is under the DMSO signal.

### Example 28

### (2S)—N-[[5-Fluoro-6-(trans-4-fluorocyclohexoxy)-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.30)

The title compound was prepared using the procedure described in example 1 starting from 4-fluorocyclohexanol, 2,5,6-trifluoronicotinonitrile, methanol and (S)-1-methylpyrrolidine-2-carboxylic acid. The intermediate 5-fluoro-6-((4-fluorocyclohexyl)oxy)-2-methoxynicotinonitrile was separated via column chromatography on silica gel (40 g column; heptane 100%→80% ethylacetate, 14 ml/min) to 5-fluoro-6-(((1s,4s)-4-fluorocyclohexyl)oxy)-2-methoxynicotinonitrile and 5-fluoro-6-(((1r,4r)-4-fluorocyclohexyl)oxy)-2-methoxynicotinonitrile, which were processed further according to example 5. Finally the fumarate salt was formed by adding one equivalent fumaric acid.

LCMS (ESI⁺) m/z [M+H]⁺: 384.30

¹H NMR (500 MHz, Methanol-d₄) δ ppm: 7.40 (d, J=10.0 Hz, 1H), 6.70 (s, 2H), 5.23-5.14 (m, 1H), 4.74 (dtt, J=48.0, 7.0, 3.0 Hz, 1H), 4.34-4.23 (m, 2H), 3.91 (s, 3H), 3.76 (dd, J=9.3, 6.8 Hz, 1H), 3.61-3.52 (m, 1H), 3.04-2.96 (m, 1H), 2.77 (s, 3H), 2.51-2.39 (m, 1H), 2.16-1.91 (m, 7H), 1.83-1.69 (m, 4H)

### Example 29

### (2S)—N-[[5-Fluoro-6-(cis-4-fluorocyclohexoxy)-2-methoxy-3-pyridyl]methyl]-1-methyl-pyrrolidine-2-carboxamide; Fumaric Acid

(Compound of Formula Ia.2, Wherein X is CH, R⁵ is H, R⁶ is F, R⁷ is OCH₃ and R^{8a} is A.30)

The title compound was prepared using the procedure described in example 1 starting from 4-fluorocyclohexanol, 2,5,6-trifluoronicotinonitrile, methanol and (S)-1-methylpyrrolidine-2-carboxylic acid. The intermediate 5-fluoro-6-((4-fluorocyclohexyl)oxy)-2-methoxynicotinonitrile was separated via column chromatography on silica gel (40 g column; heptane 100%-80% ethylacetate, 14 ml/min) to 5-fluoro-6-(((1s,4s)-4-fluorocyclohexyl)oxy)-2-methoxynicotinonitrile and 5-fluoro-6-(((1r,4r)-4-fluorocyclohexyl)oxy)-2-methoxynicotinonitrile, which were processed further according to example 5. Finally the fumarate salt was formed by adding one equivalent fumaric acid.

LCMS (ESI⁺) m/z [M+H]⁺: 384.30

¹H NMR (500 MHz, Methanol-d₄) δ ppm: 7.41 (d, J=10.0 Hz, 1H), 6.70 (s, 2H), 5.15-5.08 (m, 1H), 4.69 (dtt, J=48.7, 6.5, 3.1 Hz, 1H), 4.34-4.21 (m, 2H), 3.91 (s, 3H), 3.75 (dd, J=9.1, 6.8 Hz, 1H), 3.61-3.52 (m, 1H), 2.99 (dt, J=10.8, 8.2 Hz, 1H), 2.77 (s, 3H), 2.55-2.36 (m, 1H), 2.19-2.04 (m, 1H), 2.04-1.71 (m, 10H)

### Example 30

### (2S)—N-[[5-Fluoro-6-(cis-4-fluorocyclohexoxy)-2-methoxy-3-pyridyl]methyl]-pyrrolidine-2-carboxamide; Fumaric Acid

(Compound of Formula Ia.1, Wherein X is CH, R⁵ is H, R⁶ is F, R⁷ is OCH₃ and R^{8a} is A.30)

The title compound was prepared using the procedure described in example 1 starting from 4-fluorocyclohexanol, 2,5,6-trifluoronicotinonitrile, methanol and (S)-1-(tertbutoxycarbonyl)pyrrolidine-2-carboxylic acid, followed by BOC deprotection. The intermediate 5-fluoro-6-((4-fluorocyclohexyl)oxy)-2-methoxynicotinonitrile was separated via column chromatography on silica gel (40 g column; heptane 100%->80% ethylacetate, 14 ml/min) to 5-fluoro-6-(((1s,4s)-4-fluorocyclohexyl)oxy)-2-methoxynicotinonitrile and 5-fluoro-6-(((1r,4r)-4-fluorocyclohexyl)oxy)-2-methoxynicotinonitrile, which were processed further according to example 5. Finally the fumarate salt was formed by adding one equivalent fumaric acid.

LCMS (ESI⁺) m/z [M+H]⁺: 370.30

¹H NMR (500 MHz, Methanol-d₄) δ ppm: 7.42 (d, J=10.0 Hz, 1H), 6.68 (s, 2H), 5.12 (tt, J=7.3, 3.6 Hz, 1H), 4.69 (dtt, J=48.7, 6.6, 3.2 Hz, 1H), 4.29 (d, J=2.5 Hz, 2H), 4.22 (dd, J=8.5, 6.8 Hz, 1H), 3.90 (s, 3H), 3.41-3.26 (m, 2H), 2.45-2.33 (m, 1H), 2.08-1.72 (m, 11H); one peak is under the MeOH signal.

### Example 31

### (2S)—N-[[5-Fluoro-6-(trans-4-fluorocyclohexoxy)-2-methoxy-3-pyridyl]methyl]-pyrrolidine-2-carboxamide; Fumaric Acid

(Compound of Formula Ia.1, Wherein X is CH, R⁵ is H, R⁶ is F, R⁷ is OCH₃ and R^{8a} is A.30)
The title compound was prepared using the procedure described in example 1 starting from trans-4-fluorocyclohexanol, 2,5,6-trifluoronicotinonitrile, methanol and (S)-1-(tert-butoxycarbonyl)pyrrolidine-2-carboxylic acid, followed by BOC deprotection. The intermediate 5-fluoro-6-((4-fluorocyclohexyl)oxy)-2-methoxynicotinonitrile was separated via column chromatography on silica gel (40 g column; heptane 100%→80% ethylacetate, 14 ml/min) to 5-fluoro-6-(((1s,4s)-4-fluorocyclohexyl)oxy)-2-methoxynicotinonitrile and 5-fluoro-6-(((1r,4r)-4-fluorocyclohexyl)oxy)-2-methoxynicotinonitrile, which were processed further according to example 5. Finally the fumarate salt was formed by adding one equivalent fumaric acid.

LCMS (ESI+) m/z [M+H]+: 370.30

¹H NMR (500 MHz, Methanol-d₄) δ ppm: 7.42 (d, J=10.1 Hz, 1H), 6.68 (s, 2H), 5.22-5.14 (m, 1H), 4.73 (br d, J=47.1 Hz, 1H), 4.29 (d, J=2.8 Hz, 2H), 4.22 (dd, J=8.4, 6.8 Hz, 1H), 3.91 (s, 3H), 3.41-3.27 (m, 2H), 2.44-2.30 (m, 1H), 2.15-1.92 (m, 7H), 1.81-1.67 (m, 4H); one peak is under the MeOH signal.

### Example 32

### (2S)—N-[[6-[[(1S)-3,3-Difluorocyclopentyl]methoxy]-5-fluoro-2-methoxy-3-pyridyl]methyl]pyrrolidine-2-carboxamide; Fumaric Acid

(Compound of Formula Ia.1, Wherein X is CH, R⁵ is H, R⁶ is F, R⁷ is OCH₃ and R^{8a} is CH₂-A.21)

The title compound was prepared using the procedure described in example 1 starting from (S)-(3,3-difluorocyclopentyl)methanol, 2,5,6-trifluoronicotinonitrile, methanol and (S)-1-(tert-butoxycarbonyl)pyrrolidine-2-carboxylic acid, followed by BOC deprotection. Finally the fumarate salt was formed by adding one equivalent fumaric acid.

LCMS (ESI⁺) m/z [M+H]⁺: 388.3

¹H NMR (600 MHz, DMSO-d₆) δ ppm: 8.65 (t, J=5.9 Hz, 1H), 7.49 (d, J=10.4 Hz, 1H), 6.51 (s, 2H), 4.32 (dd, J=6.7, 3.4 Hz, 2H), 4.15 (dd, J=5.9, 1.9 Hz, 2H), 3.92 (dd, J=8.2, 5.6 Hz, 1H), 3.87 (s, 3H), 3.05-2.96 (m, 2H), 2.66-2.57 (m, 1H), 2.33-2.23 (m, 1H), 2.23-2.02 (m, 3H), 2.02-1.86 (m, 2H), 1.81-1.70 (m, 3H), 1.67-1.54 (m, 1H)

### Example 33

### (2S)—N-[[6-[[(1R)-3,3-Difluorocyclopentyl]methoxy]-5-fluoro-2-methoxy-3-pyridyl]methyl]pyrrolidine-2-carboxamide; Fumaric Acid

(Compound of Formula Ia.1, Wherein X is CH, R⁵ is H, R⁶ is F, R⁷ is OCH₃ and R^{8a} is CH₂-A.21)

The title compound was prepared using the procedure described in example 1 starting from (R)-(3,3-difluorocyclopentyl)methanol, 2,5,6-trifluoronicotinonitrile, methanol and (S)-1-(tert-butoxycarbonyl)pyrrolidine-2-carboxylic acid, followed by BOC deprotection. Finally the fumarate salt was formed by adding one equivalent fumaric acid.

LCMS (ESI⁺) m/z [M+H]⁺: 388.4

¹H NMR (500 MHz, DMSO-d₆) δ ppm: 8.61 (t, J=5.9 Hz, 1H), 7.48 (d, J=10.4 Hz, 1H), 6.52 (s, 2H), 4.37-4.24 (m, 2H), 4.16 (s, 2H), 3.93-3.83 (m, 4H), 3.00 (tt, J=7.8, 3.9 Hz, 2H), 2.67-2.54 (m, 1H), 2.39-2.22 (m, 1H), 2.22-2.00 (m, 3H), 2.00-1.87 (m, 2H), 1.82-1.66 (m, 3H), 1.66-1.51 (m, 1H)

### Example 34

### (2S,4R)—N-[[6-(4,4-Difluorocyclohexoxy)-5-fluoro-2-methoxy-3-pyridyl]methyl]-4-fluoro-pyrrolidine-2-carboxamide; Fumaric Acid

(Compound of Formula Ia.5, Wherein X is CH, R⁵ is H, R⁶ is F, R⁷ is OCH₃ and R^{8a} is A.33)

The title compound was prepared using the procedure described in example 1 starting from (6-((4,4-difluorocyclohexyl)oxy)-5-fluoro-2-methoxypyridin-3-yl)methanamine and (2S,4R)-1-(tert-butoxycarbonyl)-4-fluoropyrrolidine-2-carboxylic acid, followed by BOC deprotection. Finally the fumarate salt was formed by adding one equivalent fumaric acid.

LCMS (ESI⁺) m/z [M+H]⁺: 406.4

¹H NMR (500 MHz, DMSO-d₆) δ ppm: 8.44 (t, J=6.1 Hz, 1H), 7.44 (d, J=10.3 Hz, 1H), 6.60 (s, 2H), 5.33-5.18 (m, 1H), 5.23-5.15 (m, 1H), 4.19-4.08 (m, 2H), 3.91-3.82 (m, 4H), 3.14 (ddd, J=23.5, 13.4, 2.2 Hz, 1H), 2.96 (ddd, J=38.5, 13.4, 3.4 Hz, 1H), 2.28 (dddd, J=22.7, 14.6, 7.9, 2.1 Hz, 1H), 2.12-1.95 (m, 6H), 1.95-1.79 (m, 3H)

### Example 35

### (2S,4R)-4-Fluoro-N-[[5-fluoro-2-methoxy-6-[4-(trifluoromethyl)cyclohexoxy]-3-pyridyl]methyl]pyrrolidine-2-carboxamide; Fumaric Acid

(Compound of Formula Ia.5, Wherein X is CH, R⁵ is H, R⁶ is F, R⁷ is OCH₃ and R^{8a} is A.36)

The title compound was prepared using the procedure described in example 1 starting from trans-4-(trifluoromethyl)cyclohexanol, 2,5,6-trifluoronicotinonitrile, methanol and (2S,4R)-1-(tert-butoxycarbonyl)-4-fluoropyrrolidine-2-carboxylic acid, followed by BOC deprotection. Finally the fumarate salt was formed by adding one equivalent fumaric acid.

LCMS (ESI⁺) m/z [M+H]⁺: 438.3

¹H NMR (500 MHz, DMSO-d₆) δ ppm: 8.43 (t, J=6.0 Hz, 1H), 7.42 (d, J=10.4 Hz, 1H), 6.60 (s, 2H), 5.25 (dt, J=54.4, 3.7 Hz, 1H), 4.99-4.87 (m, 1H), 4.13 (dd, J=5.9, 1.7 Hz, 2H), 3.90-3.82 (m, 4H), 3.13 (ddd, J=23.5, 13.4, 2.1 Hz, 1H), 2.96 (ddd, J=38.5, 13.4, 3.3 Hz, 1H), 2.43-2.34 (m, 1H), 2.32-2.16 (m, 3H), 1.99-1.79 (m, 3H), 1.55-1.39 (m, 4H)

### Example 36

### (2S)—N-[[5-Fluoro-2-methoxy-6-[4-(trifluoromethyl)cyclohexoxy]-3-pyridyl]-methyl]azetidine-2-carboxamide; Succinic Acid

(Compound of Formula Ia.9, Wherein X is CH, R⁵ is H, R⁶ is F, R⁷ is OCH₃ and R^{8a} is A.36)

The title compound was prepared using the procedure described in example 1 starting from trans-4-(trifluoromethyl)cyclohexanol, 2,5,6-trifluoronicotinonitrile, methanol and (S)-1-(tert-butoxycarbonyl)azetidine-2-carboxylic acid, followed by BOC deprotection. Finally the succinate salt was formed by adding one equivalent succinic acid.

LCMS (ESI⁺) m/z [M+H]⁺: 406.3

¹H NMR (500 MHz, DMSO-d₆) δ ppm 8.38 (t, J=6.0 Hz, 1H), 7.47 (d, J=10.5 Hz, 1H), 5.01-4.87 (m, 1H), 4.36 (t, J=8.4 Hz, 1H), 4.17 (d, J=5.9 Hz, 2H), 3.87 (s, 3H), 3.66 (dt, J=8.3 Hz, 1H), 3.34 (td, J=8.5, 4.7 Hz, 1H), 2.54-2.45 (m, 1H), 2.41-2.30 (m, 5H), 2.28-2.14 (m, 3H), 2.01-1.85 (m, 2H), 1.57-1.38 (m, 4H); one peak is under the water and one under the DMSO signal.

### Example 37

### (2S)—N-[[6-(4,4-Difluorocyclohexoxy)-5-fluoro-2-methoxy-3-pyridyl]methyl]-azetidine-2-carboxamide; Succinic Acid

(Compound of Formula Ia.9, Wherein X is CH, R⁵ is H, R⁶ is F, R⁷ is OCH₃ and R^{8a} is A.33)

The title compound was prepared using the procedure described in example 1 starting from (6-((4,4-difluorocyclohexyl)oxy)-5-fluoro-2-methoxypyridin-3-yl)methanamine and (S)-1-(tert-butoxycarbonyl)azetidine-2-carboxylic acid, followed by BOC deprotection. Finally the succinate salt was formed by adding one equivalent succinic acid.

LCMS (ESI⁺) m/z [M+H]⁺: 374.3

¹H NMR (500 MHz, DMSO-d₆) δ ppm 8.39 (t, J=6.0 Hz, 1H), 7.49 (d, J=10.3 Hz, 1H), 5.20 (tt, J=6.9, 3.2 Hz, 1H), 4.38 (t, J=8.3 Hz, 1H), 4.18 (d, J=6.0 Hz, 2H), 3.88 (s, 3H), 3.67 (q, J=8.3 Hz, 1H), 3.34 (td, J=8.6, 4.8 Hz, 1H), (2.46-2.53, m, 1H), 2.36 (s, 4H), 2.26-2.15 (m, 1H), 2.14-1.95 (m, 6H), 1.95-1.84 (m, 2H); one peak is under the DMSO signal.

### Example 38

### (2S)—N-[[5-Fluoro-2-methoxy-6-[4-(trifluoromethyl)cyclohexoxy]-3-pyridyl]methyl]-1-methyl-azetidine-2-carboxamide; Succinic Acid

(Compound of Formula Ia.10, Wherein X is CH, R⁵ is H, R⁶ is F, R⁷ is OCH₃ and R^{8a} is A.36)

The title compound was prepared from (2S)—N-[[5-fluoro-2-methoxy-6-[4-(trifluoromethyl)cyclohexoxy]-3-pyridyl]methyl]azetidine-2-carboxamide (example 36) by reductive amination with formaldehyde as described for example 7. Finally the succinate salt was formed by adding one equivalent succinic acid.

LCMS (ESI⁺) m/z [M+H]⁺: 420.4

¹H NMR (500 MHz, DMSO-d₆) δ ppm: 12.25 (br s, 2H), 8.18 (t, J=6.1 Hz, 1H), 7.39 (d, J=10.4 Hz, 1H), 5.03-4.86 (m, 1H), 4.14 (d, J=6.1 Hz, 2H), 3.87 (s, 3H), 3.45 (t, J=8.4 Hz, 1H), 3.29 (d, J=6.7 Hz, 3H), 3.00-2.77 (m, 1H), 2.41 (s, 4H), 2.39-2.34 (m, 1H), 2.28 (s, 3H), 2.25-2.13 (m, 1H), 2.02-1.90 (m, 3H), 1.56-1.37 (m, 4H); one peak is under the DMSO signal.

### Example 39

### (2S)—N-[[6-(4,4-Difluorocyclohexoxy)-5-fluoro-2-methoxy-3-pyridyl]methyl]-1-methyl-azetidine-2-carboxamide; Succinic Acid

(Compound of Formula Ia.10, Wherein X is CH, R⁵ is H, R⁶ is F, R⁷ is OCH₃ and R^{8a} is A.33)

The title compound was prepared from (2S)—N-[[6-(4,4-difluorocyclohexoxy)-5-fluoro-2-methoxy-3-pyridyl]methyl]azetidine-2-carboxamide (example 37) by reductive amination with formaldehyde as described for example 7. Finally the succinate salt was formed by adding one equivalent succinic acid.

LCMS (ESI⁺) m/z [M+H]⁺: 388.3

¹H NMR (500 MHz, DMSO-d₆) δ ppm: 12.27 (br s, 1H), 8.18 (t, J=6.2 Hz, 1H), 7.41 (d, J=10.3 Hz, 1H), 5.20 (tt, J=5.7, 3.0 Hz, 1H), 4.15 (d, J=6.3 Hz, 2H), 3.87 (s, 3H), 3.44 (dd, J=8.4 Hz, 1H), 3.30-3.28 (m, 1H), 2.89 (td, J=8.5, 6.7 Hz, 1H), 2.40 (s, 4H), 2.28 (s, 3H), 2.21 (dtd, J=10.7, 8.2, 2.5 Hz, 1H), 2.12-1.95 (m, 7H), 1.95-1.81 (m, 2H)

### Example 40

### (2S)—N-[[5-Fluoro-2-methoxy-6-(4,4,4-trifluorobutoxy)-3-pyridyl]methyl]-pyrrolidine-2-carboxamide; Succinic Acid

(Compound of Formula Ia.1, Wherein X is CH, R⁵ is H, R⁶ is F, R⁷ is OCH₃ and R^{8a} is (CH₂)₃CF₃)

The title compound was prepared using the procedure described in example 2, starting from 4,4,4-trifluorobutan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 380.4

¹H NMR (500 MHz, DMSO-d₆) δ ppm: 8.47 (t, J=6.0 Hz, 1H), 7.45 (d, J=10.3 Hz, 1H), 4.42 (t, J=6.4 Hz, 2H), 4.15 (d, J=6.0 Hz, 2H), 3.87 (s, 3H), 3.76 (dd, J=8.7, 5.4 Hz, 1H), 2.99-2.91 (m, 2H), 2.45-2.38 (m, 2H), 2.36 (s, 4H), 2.11-2.02 (m, 1H), 2.02-1.94 (m, 2H), 1.76-1.63 (m, 3H)

### Example 41

### (2S)—N-[[6-[(2,2-Difluorocyclopropyl)methoxy]-5-fluoro-2-methoxy-3-pyridyl]methyl]pyrrolidine-2-carboxamide; Succinic Acid

(Compound of Formula Ia.1, Wherein X is CH, R⁵ is H, R⁶ is F, R⁷ is OCH₃ and R^{8a} is CH₂-A.3)

The title compound was prepared using the procedure described in example 2, starting from (2,2-difluorocyclopropyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 360.4

¹H NMR (500 MHz, DMSO-d₆) δ ppm: 8.47 (t, J=6.1 Hz, 1H), 7.47 (d, J=10.4 Hz, 1H), 4.54 (ddd, J=10.2, 6.8, 2.9 Hz, 1H), 4.32 (ddd, J=11.9, 8.4, 1.8 Hz, 1H), 4.15 (d, J=6.0 Hz, 2H), 3.88 (s, 3H), 3.78-3.72 (m, 1H), 3.00-2.87 (m, 2H), 2.35 (s, 4H), 2.34-2.22 (m, 1H), 2.10-1.99 (m, 1H), 1.77-1.65 (m, 4H), 1.56-1.47 (m, 1H)

### Example 42

### (2S)—N-[[6-[(3,3-Difluorocyclobutyl)methoxy]-5-fluoro-2-methoxy-3-pyridyl]-methyl]pyrrolidine-2-carboxamide; Succinic Acid

(Compound of Formula Ia.1, Wherein X is CH, R⁵ is H, R⁶ is F, R⁷ is OCH₃ and R^{8a} is CH₂-A.10)

The title compound was prepared using the procedure described in example 2, starting from (3,3-difluorocyclobutyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 374.40

¹H NMR (500 MHz, Methanol-d₄) δ ppm 7.42 (d, J=10.0 Hz, 1H), 4.44 (d, J=5.8 Hz, 2H), 4.28 (d, J=2.1 Hz, 2H), 4.12 (dd, J=8.5, 6.6 Hz, 1H), 3.93 (s, 3H), 3.34-3.20 (m, 2H), 2.76-2.60 (m, 3H), 2.52 (s, 4H), 2.50-2.41 (m, 2H), 2.38-2.30 (m, 1H), 2.04-1.85 (m, 3H); one peak is under the MeOH signal.

### Example 43

### 2-(Azetidin-1-yl)-N-[[6-(4,4-difluorocyclohexoxy)-5-fluoro-2-methoxy-3-pyridyl]-methyl]propanamide; Succinic Acid

(Compound of Formula Ia.20, Wherein X is CH, R⁵ is H, R⁶ is F, R⁷ is OCH₃ and R^{8a} is A.33)

The title compound was prepared according to scheme 3 using the intermediate (6-((4,4-difluorocyclohexyl)oxy)-5-fluoro-2-methoxypyridin-3-yl)methanamine (example 5) and 2-bromopropanoyl chloride, followed by nucleophilic displacement of the bromine atoms by the azetidine residue.

### 43.1 2-Bromo-N-((6-((4,4-difluorocyclohexyl)oxy)-5-fluoro-2-methoxypyridin-3-yl)methyl)propanamide

(6-((4,4-Difluorocyclohexyl)oxy)-5-fluoro-2-methoxypyridin-3-yl)methanamine (0.250 g, 0.861 mmol) was dissolved in dichloromethane (8.6 ml) at room temperature under nitrogen to give a yellow solution. 2-Bromopropanoyl chloride (0.106 ml, 1.033 mmol) and diisopropylethylamine (0.331 ml, 1.895 mmol) were added. The reaction mixture was stirred at room temperature for 18 hours. Water was added to the reaction mixture. The pH was adjusted alkaline with 2M NaOH. The aqueous layer was extracted three times with dichloromethane; the combined organic layers were washed with saturated aqueous sodium chloride, dried over sodium sulphate, filtered and the solvent evaporated. The crude product (498 mg, brown oil) was purified by column chromatography on silica gel (12 g-column, ethyl acetate/heptane, 10% ethyl acetate→90% ethyl acetate, flow: 13 ml/min.) to give 2-bromo-N-((6-((4,4-difluorocyclohexyl)oxy)-5-fluoro-2-methoxypyridin-3-yl)methyl)propanamide (182 mg, yield: 60%) as yellow oil. LCMS (ESI+) m/z [M+H]+: 425.0.

### 43.2 2-(Azetidin-1-yl)-N-[[6-(4,4-difluorocyclohexoxy)-5-fluoro-2-methoxy-3-pyridyl]methyl]propanamide

2-(Azetidin-1-yl)-N-((6-((4,4-difluorocyclohexyl)oxy)-5-fluoro-2-methoxypyridin-3-yl)methyl)propanamide (0.170 g, 0.400 mmol) and azetidine (0.228 g, 4.0 mmol) were dissolved in dioxane and the mixture was heated in the microwave at 120° C. for 1 h. Water was added to the reaction mixture. The pH was adjusted alkaline with 2M NaOH. The aqueous layer was extracted three times with ethyl acetate, the combined organic layers were washed with saturated aqueous sodium chloride, dried over sodium sulphate, filtered and the solvent was evaporated. The crude product (210 mg, yellow oil) was purified by column chromatography on silica gel (4 g-column; dichloromethane/methanol, 100% dichloromethane→50% methanol, flow: 11 ml/min) to give 2-(azetidin-1-yl)-N-[[6-(4,4-difluorocyclohexoxy)-5-fluoro-2-methoxy-3-pyridyl]methyl]propanamide (89 mg, yield: 56%) as colorless oil. Finally the succinate salt was formed by adding one equivalent succinic acid.

LCMS (ESI⁺) m/z [M+H]⁺: 402.3

¹H NMR (500 MHz, DMSO-d₆) δ ppm: 12.39 (br s, 1H), 8.09 (t, J=6.0 Hz, 1H), 7.37 (d, J=10.6 Hz, 1H), 5.20 (tt, J=7.0, 3.2 Hz, 1H), 4.21-4.06 (m, 2H), 3.86 (s, 3H), 3.28 (ddd, J=7.0 Hz, 2H), 3.21 (ddd, J=7.0 Hz, 2H), 2.93 (q, J=6.8 Hz, 1H), 2.40 (s, 4H), 2.11-1.94 (m, 8H), 1.94-1.82 (m, 2H), 1.04 (d, J=6.8 Hz, 3H)

### Example 44

### (2S)—N-[[6-(3,3-Difluorocyclobutoxy)-5-fluoro-2-methoxy-3-pyridyl]methyl]-pyrrolidine-2-carboxamide; Succinic Acid

(Compound of Formula Ia.1, Wherein X is CH, R⁵ is H, R⁶ is F, R⁷ is OCH₃ and R^{8a} is A.10)

The title compound was prepared using the procedure described in example 2, starting from 3,3-difluorocyclobutanol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 360.3

¹H NMR (600 MHz, DMSO-d₆) δ ppm: 8.59 (t, J=5.9 Hz, 1H), 7.51 (d, J=10.4 Hz, 1H), 5.18-5.06 (m, 1H), 4.16 (d, J=5.9 Hz, 2H), 3.87 (s, 4H), 3.21 (ddt, J=15.1, 11.8, 7.3 Hz, 2H), 3.03-2.97 (m, 2H), 2.86-2.74 (m, 2H), 2.37 (s, 8H), 2.17-2.05 (m, 1H), 1.78-1.68 (m, 3H); three peaks overlap with water.

### Example 45

### (2S)—N-[[5-Fluoro-2-methoxy-6-(3,3,4,4,4-pentafluorobutoxy)-3-pyridyl]methyl]-pyrrolidine-2-carboxamide; Succinic Acid

(Compound of Formula Ia.1, Wherein X is CH, R⁵ is H, R⁶ is F, R⁷ is OCH₃ and R^{8a} is (CH₂)₂CF₂CF₃)

The title compound was prepared using the procedure described in example 2, starting from 3,3,4,4,4-pentafluorobutan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 416.25

¹H NMR (500 MHz, Methanol-d₄) δ ppm 7.45 (d, J=9.9 Hz, 1H), 4.69 (t, J=6.4 Hz, 2H), 4.29 (d, J=1.2 Hz, 2H), 4.13 (dd, J=8.5, 6.5 Hz, 1H), 3.94 (s, 3H), 3.35-3.21 (m, 2H), 2.78-2.63 (m, 2H), 2.52 (s, 4H), 2.39-2.31 (m, 1H), 2.05-1.84 (m, 3H)

### Example 46

### (2S)—N-[[5-Fluoro-2-methoxy-6-(2,2,3,3-tetrafluoropropoxy)-3-pyridyl]methyl]-pyrrolidine-2-carboxamide; Succinic Acid

(Compound of Formula Ia.1, Wherein X is CH, R⁵ is H, R⁶ is F, R⁷ is OCH₃ and R^{8a} is CH₂CF₂CHF₂)

The title compound was prepared using the procedure described in example 2, starting from 2,2,3,3-tetrafluoropropan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 384.20

¹H NMR (600 MHz, Methanol-d₄) δ ppm: 7.52 (d, J=9.8 Hz, 1H), 6.27 (tt, J=52.6, 4.8 Hz, 1H), 4.88-4.80 (m, 2H), 4.30 (s, 2H), 4.15 (dd, J=8.5, 6.6 Hz, 1H), 3.95 (s, 3H), 3.35-3.21 (m, 2H), 2.51 (s, 4H), 2.39-2.31 (m, 1H), 2.02-1.89 (m, 3H); one peak is under the MeOH signal.

### Example 47

### (2S)—N-[[5-Fluoro-2-methoxy-6-[(2,2,3,3-tetrafluorocyclobutyl)methoxy]-3-pyridyl]methyl]pyrrolidine-2-carboxamide; Succinic Acid

(Compound of Formula Ia.1, Wherein X is CH, R⁵ is H, R⁶ is F, R⁷ is OCH₃ and R^{8a} is CH₂-A.11)

The title compound was prepared using the procedure described in example 2, starting from (2,2,3,3-tetrafluorocyclobutyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 410.2

¹H NMR (500 MHz, DMSO-d₆) δ ppm: 8.49 (t, J=6.0 Hz, 1H), 7.47 (d, J=10.3 Hz, 1H), 4.67-4.47 (m, 2H), 4.15 (d, J=6.1 Hz, 2H), 3.89 (s, 3H), 3.79-3.72 (m, 1H), 3.48-3.36 (m, 1H), 3.01-2.91 (m, 2H), 2.91-2.79 (m, 1H), 2.66-2.53 (m, 1H), 2.36 (s, 4H), 2.12-1.99 (m, 1H), 1.80-1.61 (m, 3H); one peak is under the water signal.

### Example 48

### (2S)—N-[[5-Fluoro-2-methoxy-6-[[1-(trifluoromethyl)cyclopropyl]methoxy]-3-pyridyl]methyl]pyrrolidine-2-carboxamide; Succinic Acid

(Compound of Formula Ia.1, Wherein X is CH, R⁵ is H, R⁶ is F, R⁷ is OCH₃ and R^{8a} is CH₂-A.4)

The title compound was prepared using the procedure described in example 2, starting from (1-(trifluoromethyl)cyclopropyl)methanol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 392.3

¹H NMR (600 MHz, DMSO-d₆) δ ppm: 8.52 (t, J=6.0 Hz, 1H), 7.47 (d, J=10.3 Hz, 1H), 4.54 (s, 2H), 4.15 (d, J=5.9 Hz, 2H), 3.87 (s, 3H), 3.81-3.77 (m, 1H), 3.00-2.92 (m, 2H), 2.35 (s, 4H), 2.15-2.01 (m, 1H), 1.78-1.62 (m, 3H), 1.15-1.08 (m, 2H), 1.08-1.01 (m, 2H)

### Example 49

### (2S)—N-[[5-Fluoro-2-methoxy-6-(4,4,4-trifluoro-2-methyl-butoxy)-3-pyridyl]-methyl]pyrrolidine-2-carboxamide; Succinic Acid

(Compound of Formula Ia.1, Wherein X is CH, R⁵ is H, R⁶ is F, R⁷ is OCH₃ and R^{8a} is CH₂CH(CH₃)CH₂CF₃)

The title compound was prepared using the procedure described in example 2, starting from 4,4,4-trifluoro-2-methylbutan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 394.3

¹H NMR (600 MHz, DMSO-d₆) δ ppm: 8.51 (t, J=6.0 Hz, 1H), 7.46 (d, J=10.4 Hz, 1H), 4.31 (dd, J=10.5, 5.9 Hz, 1H), 4.22 (dd, J=10.6, 6.1 Hz, 1H), 4.15 (d, J=5.9 Hz, 2H), 3.87 (s, 3H), 3.81-3.75 (m, 1H), 3.00-2.89 (m, 2H), 2.49-2.40 (m, 1H), 2.35 (s, 4H), 2.34-2.20 (m, 2H), 2.11-2.02 (m, 1H), 1.75-1.66 (m, 3H), 1.08 (d, J=6.4 Hz, 3H)

### Example 50

### (2S)—N-[[5-Fluoro-2-methoxy-6-(2,2,3,3,3-pentafluoro-1-methyl-propoxy)-3-pyridyl]methyl]pyrrolidine-2-carboxamide; Succinic Acid

(Compound of Formula Ia.1, Wherein X is CH, R⁵ is H, R⁶ is F, R⁷ is OCH₃ and R^{8a} is CH(CH₃)CF₂CF₃)

The title compound was prepared using the procedure described in example 2, starting from 3,3,4,4,4-pentafluorobutan-2-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 416.2

¹H NMR (600 MHz, DMSO-d₆) δ ppm 8.56 (t, J=6.0 Hz, 1H), 7.54 (d, J=10.1 Hz, 1H), 5.97 (dt, J=17.5, 6.5 Hz, 1H), 4.16 (d, J=6.0 Hz, 2H), 3.90 (s, 3H), 3.82-3.76 (m, 1H), 3.00-2.91 (m, 2H), 2.36 (s, 4H), 2.11-2.01 (m, 1H), 1.77-1.66 (m, 3H), 1.54 (d, J=6.4 Hz, 3H)

### Example 51

### (2R)—N-[[6-(4,4-Difluorocyclohexoxy)-5-fluoro-2-methoxy-3-pyridyl]methyl]-pyrrolidine-2-carboxamide Hydrochloride

(Compound of Formula Ia.1, Wherein X is CH, R⁵ is H, R⁶ is F, R⁷ is OCH₃ and R^{8a} is A.33)

The title compound was prepared using the procedure described in example 1 starting from (6-((4,4-difluorocyclohexyl)oxy)-5-fluoro-2-methoxypyridin-3-yl)methanamine and (R)-1-(tert-butoxycarbonyl)pyrrolidine-2-carboxylic acid, followed by BOC deprotection. Finally the hydrochloride salt was formed by adding one equivalent hydrochloric acid.

LCMS (ESI+) m/z [M+H]+: 388.2

¹H NMR (600 MHz, DMSO-d₆) δ ppm: 9.61 (s, 1H), 8.93 (t, J=5.7 Hz, 1H), 8.55 (s, 1H), 7.58 (d, J=10.3 Hz, 1H), 5.28-5.14 (m, 1H), 4.24-4.14 (m, 3H), 3.88 (s, 3H), 3.26-3.13 (m, 2H), 2.29 (ddt, J=12.6, 8.4, 6.2 Hz, 1H), 2.10-1.96 (m, 6H), 1.93-1.79 (m, 5H)

### Example 52

### (2R)—N-[[6-(4,4-Difluorocyclohexoxy)-5-fluoro-2-methoxy-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is A.33)

The title compound was prepared from (2R)—N-[[6-(4,4-difluorocyclohexoxy)-5-fluoro-2-methoxy-3-pyridyl]methyl]pyrrolidine-2-carboxamide (example 51) by reductive amination with formaldehyde as described for example 7. Finally the hydrochloride salt was formed by adding one equivalent hydrochloric acid.

LCMS (ESI⁺) m/z [M+H]⁺: 402.2

¹H NMR (600 MHz, DMSO-d₆) δ ppm: 9.70 (s, 1H), 9.09 (t, J=5.6 Hz, 1H), 7.60 (d, J=10.3 Hz, 1H), 5.27-5.11 (m, 1H), 4.31-4.14 (m, 2H), 4.14-4.03 (m, 1H), 3.87 (s, 3H), 3.61-3.50 (m, 1H), 3.20-3.11 (m, 1H), 2.81 (d, J=4.4 Hz, 3H), 2.49-2.42 (m, 1H), 2.10-1.96 (m, 7H), 1.96-1.78 (m, 4H)

### Example 53

### (2S)—N-[[5-Fluoro-2-methoxy-6-(2,2,3,3-tetrafluoro-1-methyl-propoxy)-3-pyridyl]-methyl]pyrrolidine-2-carboxamide; Succinic Acid

(Compound of Formula Ia.1, Wherein X is CH, R⁵ is H, R⁶ is F, R⁷ is OCH₃ and R^{8a} is CH(CH₃)CF₂CHF₂)

The title compound was prepared using the procedure described in example 2, starting from 3,3,4,4-tetrafluorobutan-2-ol and (S)-tert-butyl 2-(((6-chloro-5-fluoro-2-methoxypyridin-3-yl)methyl)carbamoyl)pyrrolidine-1-carboxylate, followed by BOC deprotection. Finally the succinate salt was formed by adding one equivalent succinic acid.

LCMS (ESI⁺) m/z [M+H]⁺: 398.20

¹H NMR (500 MHz, Methanol-d₄) δ ppm: 7.51 (d, J=9.9 Hz, 1H), 6.23 (tdd, J=52.6, 7.9, 2.6 Hz, 1H), 5.76-5.60 (m, 1H), 4.30 (s, 2H), 4.14 (dd, J=8.5, 6.4 Hz, 1H), 3.94 (s, 3H), 3.36-3.18 (m, 2H), 2.51 (s, 4H), 2.45-2.28 (m, 1H), 2.07-1.85 (m, 3H), 1.52 (d, J=6.5 Hz, 3H); one peak is under the MeOH signal.

### Example 54

### (2S)—N-[[6-[(6,6-Difluoro-3-bicyclo[3.1.0]hexanyl)oxy]-5-fluoro-2-methoxy-3-pyridyl]methyl]pyrrolidine-2-carboxamide 2,2,2-trifluoroacetic Acid

(Compound of Formula Ia.1, Wherein X is CH, R⁵ is H, R⁶ is F, R⁷ is OCH₃ and R^{8a} is A.71)

The title compound was prepared using the procedure described in example 2, starting from 6,6-difluorobicyclo[3.1.0]hexan-3-ol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 386.20

¹H NMR (500 MHz, Methanol-d₄) δ ppm: 7.42 (d, J=10.0 Hz, 1H), 5.30 (q, J=5.3 Hz, 1H), 4.29 (d, J=5.3 Hz, 2H), 4.21 (dd, J=8.4, 6.9 Hz, 1H), 3.92 (s, 3H), 3.46-3.36 (m, 1H), 3.31-3.23 (m, 1H), 2.58-2.48 (m, 2H), 2.45-2.34 (m, 1H), 2.24-2.11 (m, 4H), 2.10-1.90 (m, 3H).

### Example 55

### (2S)—N-[[5-Fluoro-6-[(1R,2R)-2-fluorocyclohexoxy]-2-methoxy-3-pyridyl]methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.28)

The title compound was prepared using the procedure described in example 2, starting from trans-2-cyclohexanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 370.30

¹H NMR (500 MHz, Methanol-d₄) δ ppm: 7.43 (d, J=9.9 Hz, 1H), 5.27-5.07 (m, 1H), 4.69-4.61 (m, 0.5H), 4.59-4.51 (m, 0.5H), 4.29 (s, 2H), 4.25-4.19 (m, 1H), 3.92 (s, 3H), 3.49-3.22 (m, 2H), 2.43-2.37 (m, 1H), 2.24-2.10 (m, 2H), 2.09-1.93 (m, 3H), 1.79-1.73 (m, 2H), 1.68-1.57 (m, 1H), 1.56-1.46 (m, 1H), 1.45-1.38 (m, 2H).

### Example 56

### (2S)—N-[[6-[(6,6-Difluoro-3-bicyclo[3.1.0]hexanyl)methoxy]-5-fluoro-2-methoxy-3-pyridyl]methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is CH₂-A.71)

The title compound was prepared using the procedure described in example 2, starting from (6,6-difluorobicyclo[3.1.0]hexan-3-yl)methanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 400.30

¹H NMR (500 MHz, Methanol-d₄) δ ppm: 7.42 (d, J=10.0 Hz, 1H), 4.35-4.17 (m, 5H), 3.92 (s, 3H), 3.46-3.26 (m, 2H), 2.55-2.44 (m, 1H), 2.43-2.35 (m, 1H), 2.20-2.12 (m, 1H), 2.08-1.94 (m, 6H), 1.91-1.85 (m, 2H).

### Example 57

### (2S)—N-[[5-Fluoro-2-methoxy-6-[2-(trifluoromethyl)cyclohexoxy]-3-pyridyl]methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.34)

The title compound was prepared using the procedure described in example 2, starting from 2-(trifluoromethyl)cyclohexanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 420.30

¹H NMR (500 MHz, Methanol-d₄) δ ppm: 7.44 (d, J=9.9 Hz, 1H), 5.72 (q, J=2.2 Hz, 1H), 4.29 (s, 2H), 4.25-4.19 (m, 1H), 3.90 (s, 3H), 3.45-3.32 (m, 2H), 2.54-2.48 (m, 1H), 2.45-2.35 (m, 1H), 2.19-2.13 (m, 1H), 2.12-1.90 (m, 5H), 1.86-1.80 (m, 1H), 1.71-1.52 (m, 3H), 1.51-1.39 (m, 1H).

### Example 58

### (2S)—N-[[6-(3,3-Difluorocyclohexoxy)-5-fluoro-2-methoxy-3-pyridyl]methyl]-pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.32)

The title compound was prepared using the procedure described in example 2, starting from 3,3-difluorocyclohexanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 388.20

¹H NMR (500 MHz, Methanol-d₄) δ ppm: 7.44 (d, J=9.8 Hz, 1H), 5.24-5.18 (m, 1H), 4.30 (s, 2H), 4.25-4.19 (m, 1H), 3.92 (s, 3H), 3.46-3.36 (m, 2H), 2.63-2.50 (m, 1H), 2.43-2.37 (m, 1H), 2.16-2.13 (m, 1H), 2.12-1.74 (m, 7H), 1.70-1.58 (m, 2H).

### Example 59

### (2 S,4R)—N-[[6-(4,4-Difluorocyclohexoxy)-5-fluoro-2-methoxy-3-pyridyl]methyl]-4-methyl-pyrrolidine-2-carboxamide Hydrochloride

(Compound of Formula Ia.3, Wherein X is CH, R⁵ is H, R⁶ is F, R⁷ is OCH₃ and R^{8a} is A.33)

The title compound was prepared using the procedure described in example 1 starting from (6-((4,4-difluorocyclohexyl)oxy)-5-fluoro-2-methoxypyridin-3-yl)methanamine and (2S,4R)-1-(tert-butoxycarbonyl)-4-methylpyrrolidine-2-carboxylic acid, followed by BOC deprotection. Finally the hydrochloride salt was formed by adding one equivalent hydrochloric acid.

LCMS (ESI⁺) m/z [M+H]⁺: 402.2

¹H NMR (600 MHz, DMSO-d₆) δ ppm: 9.88 (s, 1H), 8.94 (t, J=5.6 Hz, 1H), 8.54 (s, 1H), 7.58 (d, J=10.3 Hz, 1H), 5.21 (tt, J=6.8, 3.2 Hz, 1H), 4.36-4.27 (m, 1H), 4.27-4.11 (m, 2H), 3.87 (s, 3H), 3.44-3.37 (m, 1H), 2.81-2.69 (m, 1H), 2.34-2.23 (m, J=7.1 Hz, 1H), 2.11-1.95 (m, 7H), 1.95-1.83 (m, 3H), 1.02 (d, J=6.8 Hz, 3H)

### Example 60

### (2S,4R)—N-[[5-Fluoro-2-methoxy-6-[4-(trifluoromethyl)cyclohexoxy]-3-pyridyl]methyl]-4-methyl-pyrrolidine-2-carboxamide Hydrochloride

(Compound of Formula Ia.3, Wherein X is CH, R⁵ is H, R⁶ is F, R⁷ is OCH₃ and R^{8a} is A.36)

The title compound was prepared using the procedure described in example 1 starting from trans-4-(trifluoromethyl)cyclohexanol, 2,5,6-trifluoronicotinonitrile, methanol and (2S,4R)-1-(tert-butoxycarbonyl)-4-methylpyrrolidine-2-carboxylic acid, followed by BOC deprotection. Finally the hydrochloride salt was formed by adding one equivalent hydrochloric acid.

LCMS (ESI⁺) m/z [M+H]⁺: 434.2

¹H NMR (600 MHz, DMSO-d₆) δ ppm: 9.84 (s, 1H), 8.92 (t, J=5.6 Hz, 1H), 8.61-8.45 (m, 1H), 7.56 (d, J=10.3 Hz, 1H), 4.95 (tt, J=10.6, 4.2 Hz, 1H), 4.33-4.27 (m, 1H), 4.24-4.14 (m, 2H), 3.87 (s, 3H), 2.74 (ddd, J=14.3, 11.4, 6.4 Hz, 1H), 2.44-2.34 (m, 1H), 2.34-2.24 (m, 1H), 2.24-2.17 (m, 2H), 2.09-2.00 (m, 1H), 2.00-1.84 (m, 3H), 1.56-1.40 (m, 4H), 1.02 (d, J=6.8 Hz, 3H)

### Example 61

### (2S,4R)—N-[[6-(4,4-Difluorocyclohexoxy)-5-fluoro-2-methoxy-3-pyridyl]methyl]-1,4-dimethyl-pyrrolidine-2-carboxamide Hydrochloride

(Compound of Formula Ia.4, Wherein X is CH, R⁵ is H, R⁶ is F, R⁷ is OCH₃ and R^{8a} is A.33)

The title compound was prepared from ((2S,4R)—N-[[6-(4,4-difluorocyclohexoxy)-5-fluoro-2-methoxy-3-pyridyl]methyl]-4-methyl-pyrrolidine-2-carboxamide (example 59) by reductive amination with formaldehyde as described for example 7. Finally the hydrochloride salt was formed by adding one equivalent hydrochloric acid.

LCMS (ESI+) m/z [M+H]+: 416.2

¹H NMR (500 MHz, DMSO-d₆) δ ppm: 9.77 (s, 1H), 9.24 (t, J=5.6 Hz, 1H), 7.61 (d, J=10.4 Hz, 1H), 5.21 (tt, J=5.9, 3.1 Hz, 1H), 4.30-4.15 (m, 3H), 3.88 (s, 3H), 3.63 (ddd, J=11.0, 6.8, 3.8 Hz, 1H), 2.90-2.74 (m, 4H), 2.30 (tdd, J=14.6, 9.6, 7.1 Hz, 1H), 2.21-1.96 (m, 8H), 1.89 (tt, J=7.4, 4.0 Hz, 2H), 1.12-0.97 (m, 3H)

### Example 62

### (2S,4R)—N-[[5-Fluoro-2-methoxy-6-[4-(trifluoromethyl)cyclohexoxy]-3-pyridyl]methyl]-1,4-dimethyl-pyrrolidine-2-carboxamide Hydrochloride

(Compound of Formula Ia.4, Wherein X is CH, R⁵ is H, R⁶ is F, R⁷ is OCH₃ and R^{8a} is A.36)

The title compound was prepared from (2S,4R)—N-[[5-fluoro-2-methoxy-6-[4-(trifluoromethyl)cyclohexoxy]-3-pyridyl]methyl]-4-methyl-pyrrolidine-2-carboxamide (example 60) by reductive amination with formaldehyde as described for example 7. Finally the hydrochloride salt was formed by adding one equivalent hydrochloric acid.

LCMS (ESI⁺) m/z [M+H]⁺: 448.4

¹H NMR (600 MHz, DMSO-d₆) δ ppm: 9.69 (s, 1H), 9.01 (s, 1H), 7.57 (d, J=10.3 Hz, 1H), 4.95 (tt, J=10.6, 4.3 Hz, 1H), 4.27-4.10 (m, 3H), 3.87 (s, 3H), 3.61 (dd, J=10.8, 6.9 Hz, 1H), 2.86-2.72 (m, 4H), 2.44-2.33 (m, 1H), 2.33-2.24 (m, 1H), 2.24-2.15 (m, 2H), 2.13-2.01 (m, 2H), 2.01-1.89 (m, 2H), 1.57-1.38 (m, 4H), 1.04 (d, J=6.7 Hz, 3H)

### Example 63

### (2S)—N-[[5-Fluoro-2-methoxy-6-[[1-(trifluoromethyl)cyclopropyl]methoxy]-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is CH₂-A.4)

The title compound was prepared using the procedure described in scheme 4 starting from (1-(trifluoromethyl)cyclopropyl)methanol and (S)—N-((6-chloro-5-fluoro-2-methoxypyridin-3-yl)methyl)-1-methylpyrrolidine-2-carboxamide. Finally the hydrochloride salt was formed by adding one equivalent hydrochloric acid.

### 63.1 (S)—N-((6-chloro-5-fluoro-2-methoxypyridin-3-yl)methyl)-1-methylpyrrolidine-2-carboxamide

### (Scheme 4 Step 1)

To a solution of (S)-1-methylpyrrolidine-2-carboxylic acid (3.77 g, 29.2 mmol) in acetonitrile (100 mL) was added 2-(7-Aza-1H-benzotriazole-1-yl)-1,1,3,3-tetramethyluronium hexafluorophosphate (12.12 g, 31.9 mmol) at 0° C. The mixture was stirred at 0° C. for 2 h. Then (6-chloro-5-fluoro-2-methoxypyridin-3-yl)methanamine hydrochloride (see example 2; 7 g, 26.6 mmol) and ethyl diisopropylamine (18.56 mL, 106 mmol) were added. The mixture was stirred at 23° C. for 12 h. Two additional vials were set up as described above. All three reaction mixtures were combined and the solvent was removed under reduced pressure. The residue was treated with water (300 mL) and extracted with ethyl acetate (3×200 mL). The organic layer was washed with brine (200 mL) and dried over Na₂SO₄, filtered and concentrated under reduced pressure. The residue was purified by preparative HPLC to obtain (S)—N((6-chloro-5-fluoro-2-methoxypyridin-3-yl)methyl)-1-methylpyrrolidine-2-carboxamide (12.5 g, 52.0%) as white solid.

¹H NMR (400 MHz, DMSO-d₆) δ ppm: 8.30 (br. s., 1H), 7.52 (d, J=8.4 Hz, 1H), 4.19 (d, J=5.7 Hz, 2H), 3.89 (s, 3H), 3.04 (d, J=3.5 Hz, 1H), 2.86-2.74 (m, 1H), 2.38-2.22 (m, 4H), 2.16-2.02 (m, 1H), 1.79-1.60 (m, 3H)

### 63.2 (2S)—N-[[5-Fluoro-2-methoxy-6-[[1-(trifluoromethyl)cyclopropyl]methoxy]-3-pyridyl]methyl]-1-methyl-pyrrolidine-2-carboxamide

### (Scheme 4 Step 2)

To a solution of (S)—N-((6-chloro-5-fluoro-2-methoxypyridin-3-yl)methyl)-1-methylpyrrolidine-2-carboxamide (104 mg, 0.346 mmol) in toluene (3.5 mL) was added (1-(trifluoromethyl)cyclopropyl)methanol (97 mg, 0.692 mmol), Cs₂CO₃ (225 mg, 0.692 mmol), [1,1′-biphenyl]-2-yldi-tert-butylphosphine (10.32 mg, 0.035 mmol) and diacetoxypalladium (7.77 mg, 0.035 mmol). The reaction mixture was stirred for 7 h at 140° C. in a pressure vial (Q-Tube). The reaction mixture was concentrated in vacuo. Water was added to the reaction mixture. The pH was adjusted alkaline with 2M NaOH. The aqueous layer was extracted three times with ethyl acetate, the combined organic layers were washed with saturated aqueous sodium chloride, dried over sodium sulphate, filtered and the solvent was evaporated. The residue was purified by preparative HPLC to give (2S)—N-[[5-fluoro-2-methoxy-6-[[1-(trifluoromethyl)cyclopropyl]-methoxy]-3-pyridyl]methyl]-1-methyl-pyrrolidine-2-carboxamide (20 mg, yield 14%) as a colorless oil. Finally the hydrochloride salt was formed by adding one equivalent hydrochloric acid.

LCMS (ESI⁺) m/z [M+H]⁺: 406.2

¹H NMR (600 MHz, DMSO-d₆) δ ppm: 9.70 (s, 1H), 9.09 (t, J=5.7 Hz, 1H), 7.61 (d, J=10.3 Hz, 1H), 4.55 (s, 2H), 4.26-4.17 (m, 2H), 4.12-4.05 (m, 1H), 3.88 (s, 3H), 3.60-3.50 (m, 1H), 3.19-3.09 (m, 1H), 2.81 (s, 3H), 2.47 (s, 1H), 2.05 (s, 1H), 1.92-1.80 (m, 2H), 1.13-1.09 (m, 2H), 1.09-1.04 (m, 2H)

### Example 64

### N-[[6-[(6,6-Difluoro-3-bicyclo[3.1.0]hexanyl)oxy]-5-fluoro-2-methoxy-3-pyridyl]methyl]-1-methyl-pyrrolidine-2-carboxamide Dihydrochloride

(Compound of Formula Ia.2, Wherein X is CH, R⁵ is H, R⁶ is F, R⁷ is OCH₃ and R^{8a} is A.71)

The title compound was prepared from (2S)—N-[[6-[(6,6-difluoro-3-bicyclo[3.1.0]-hexanyl)oxy]-5-fluoro-2-methoxy-3-pyridyl]methyl]pyrrolidine-2-carboxamide (example 54) by reductive amination with formaldehyde as described for example 7. Finally the hydrochloride salt was formed by adding one equivalent hydrochloric acid.

LCMS (ESI⁺) m/z [M+H]⁺: 400.3

### Example 65

### (S)—N-((2-Methyl-6-(((1R,4S)-4-(trifluoromethyl)cyclohexyl)oxy)pyridin-3-yl)methyl)pyrrolidine-2-carboxamide

(Compound of Formula Ia.1, Wherein X is CH, R⁵ is H, R⁶ is H, R⁷ is CH₃ and R^{8a} is A.36)

The title compound was prepared using the procedure described in example 1, starting from (1R,4R)-4-(trifluoromethyl)cyclohexanol and 6-chloro-2-methylnicotinonitrile, followed by the reduction of the nitrile group to (2-methyl-6-(((1s,4s)-4-(trifluoromethyl)cyclohexyl)oxy)pyridin-3-yl)methanamine. Amide coupling with (S)-1-(tert-butoxycarbonyl)pyrrolidine-2-carboxylic acid followed by BOC resulted in the desired compound (S)—N-((2-Methyl-6-(((1R,4S)-4-(trifluoromethyl)cyclohexyl)oxy)pyridin-3-yl)methyl)pyrrolidine-2-carboxamide

### 65.1 2-Methyl-6-(((1R,4R)-4-(trifluoromethyl)cyclohexyl)oxy)nicotinonitrile

(1R,4R)-4-(Trifluoromethyl)cyclohexanol (152 mg, 0.90 mmol) was added to THF (10 ml) under argon. The reaction mixture was cooled to 0° C. Sodium hydride (52.1 mg, 1.09 mmol) was added in one portion and stirred at 0° C. for 1 h. 6-Chloro-2-methylnicotinonitrile (138 mg, 0.90 mmol) was added and the reaction mixture was stirred at room temperature overnight. The reaction mixture was quenched with water and extracted 4× with dichloromethane, dried over magnesium sulfate, filtered and concentrated to yield 300 mg of a residue, which was used without any further purification in the next reaction.

LCMS (ESI⁺) m/z [M+H]⁺: 285.20

### 65.2 (2-Methyl-6-(((1R,4R)-4-(trifluoromethyl)cyclohexyl)oxy)pyridin-3-yl)methanamine

2-Methyl-6-(((1R,4R)-4-(trifluoromethyl)cyclohexyl)oxy)nicotinonitrile (300 mg, 1.06 mmol) was added to THF (15 ml) under argon. Borane dimethyl sulfide complex (1.06 ml, 2.11 mmol) was added. The reaction mixture was heated to 70° C. for 6 h. The reaction mixture was cooled to room temperature. Hydrogen chloride (2.72 ml, 5.43 mmol) was slowly added to the reaction mixture. Subsequently methanol (2.72 ml, 67.2 mmol) was added and the reaction mixture was heated to 60° C. for 1 h. After cooling to room temperature, the reaction mixture was adjusted to an alkaline pH value, extracted 4× with dichloromethane, dried over magnesium sulfate, filtered and concentrated in vacuo to yield 280 mg. The residue was used without any further purification in the next reaction.

LCMS (ESI⁺) m/z [M+H-NH₃]⁺: 272.20

### 65.3 (S)-tert-Butyl 2-(((2-methyl-6-(((1R,4S)-4-(trifluoromethyl)cyclohexyl)oxy)pyridin-3-yl)methyl)carbamoyl)pyrrolidine-1-carboxylate

(2-Methyl-6-(((1R,4R)-4-(trifluoromethyl)cyclohexyl)oxy)pyridin-3-yl)methanamine (280 mg, 0.97 mmol) was dissolved in DMF (8 ml). (S)-1-(tert-butoxycarbonyl)pyrrolidine-2-carboxylic acid (251 mg, 1.17 mmol) was added. After cooling to 0° C., N-ethyl-N-isopropylpropan-2-amine (0.34 ml, 1.94 mmol 1) and 2-(1H-benzo[d][1,2,3]-triazol-1-yl)-1,1,3,3-tetramethylisouronium hexafluorophosphate (442 mg, 1.17 mmol) were added. The reaction mixture was stirred at room temperature for 3 days. The reaction mixture was diluted with water, extracted 3× with ethyl acetate. The combined organic layer was washed with: 2× with citric acid 10%, 1× with water, 2× with aqueous sodium bicarbonate solution, 1× with water and 1× with brine, dried over MgSO₄, filtered and concentrated in vacuo. The residue was purified via column chromatography to yield 310 mg of the desired product (66% yield).

LCMS (ESI⁺) m/z [M+H]⁺: 486.30

### 65.4 (S)—N-((2-Methyl-6-(((1R,4S)-4-(trifluoromethyl)cyclohexyl)oxy)pyridin-3-yl)methyl)pyrrolidine-2-carboxamide

(S)-tert-Butyl 2-(((2-methyl-6-(((1R,4S)-4-(trifluoromethyl)cyclohexyl)oxy)pyridin-3-yl)methyl)carbamoyl)pyrrolidine-1-carboxylate (309 mg, 0.64 mmol) was dissolved in dichloromethane (10 ml). 2,2,2-Trifluoroacetic acid (0.49 ml, 6.40 mmol) was added and the reaction mixture was stirred at room temperature overnight. The reaction mixture was diluted with water. The aqueous phase was adjusted to an alkaline pH value and extracted 4× with dichloromethane, dried over magnesium sulfate, filtered and concentrated in vacuo. The residue was purified via column chromatography to yield 245 mg of the desired product (95% yield).

LCMS (ESI⁺) m/z [M+H]⁺: 386.20

¹H NMR (500 MHz, d₆-DMSO): δ 8.25 (m, 1H), 7.45 (d, 1H), 6.55 (d, 1H), 4.90 (m, 1H), 4.20 (m, 2H), 3.55 (m, 1H), 2.80 (m, 2H), 2.35 (s, 3H), 2.15 (m, 2H), 2.00-1.90 (m, 4H), 1.70-1.55 (m, 3H), 1.45 (m, 4H).

### Example 66

### (2S)—N-[[6-[4-(Difluoromethyl)cyclohexoxy]-5-fluoro-2-methoxy-3-pyridyl]methyl]-pyrrolidine-2-carboxamide hydrochloride

(Compound of Formula Ia.1, Wherein X is CH, R⁵ is H, R⁶ is F, R⁷ is OCH₃ and R^{8a} is A.39)

The title compound was prepared using the procedure described in example 2, starting from 4-(difluoromethyl)cyclohexanol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

LCMS (ESI⁺) m/z [M+H]⁺: 402.30

¹H NMR (500 MHz, Methanol-d₄) δ ppm: 7.36 (d, J=10.0 Hz, 1H), 5.69 (td, J=56.9, 4.4 Hz, 1H), 4.96 (tt, J=10.8, 4.3 Hz, 1H), 4.25 (s, 2H), 3.91 (s, 3H), 3.87-3.75 (m, 1H), 3.17-3.07 (m, 1H), 3.07-2.98 (m, 1H), 2.30-2.12 (m, 3H), 1.99-1.90 (m, 2H), 1.90-1.74 (m, 4H), 1.56-1.44 (m, 2H), 1.44-1.33 (m, 2H)

### Example 67

### (2S)—N-[[6-[4-(Difluoromethoxy)cyclohexoxy]-5-fluoro-2-methoxy-3-pyridyl]-methyl]pyrrolidine-2-carboxamide

(Compound of Formula Ia.1, Wherein X is CH, R⁵ is H, R⁶ is F, R⁷ is OCH₃ and R^{8a} is A.52)

The title compound was prepared using the procedure described in example 2, starting from 4-(difluoromethoxy)cyclohexanol and (S)-tert-butyl 2-(((6-chloro-5-fluoro-2-methoxypyridin-3-yl)methyl)carbamoyl)pyrrolidine-1-carboxylate, followed by BOC deprotection.

¹H NMR (500 MHz, Methanol-d₄) δ ppm: 7.41 (d, J=10.0 Hz, 1H), 6.41 (t, J=76.1 Hz, 1H), 5.15-5.09 (m, 1H), 4.40-4.12 (m, 4H), 3.92 (s, 3H), 3.51-3.25 (m, 1H), 2.43-2.37 (m, 1H), 2.21-2.11 (m, 2H), 2.10-1.91 (m, 6H), 1.80-1.61 (m, 4H).

### Example 68

### (2S)—N-[[6-[(2,2-Difluorocyclopropyl)methoxy]-5-fluoro-2-methoxy-3-pyridyl]-methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is CH₂-A.3)

The title compound was prepared from (2S)—N-[[6-[(2,2-difluorocyclopropyl)methoxy]-5-fluoro-2-methoxy-3-pyridyl]methyl]pyrrolidine-2-carboxamide (example 41) by reductive amination with formaldehyde as described for example 7. Finally the hydrochloride salt was formed by adding one equivalent hydrochloric acid.

LCMS (ESI⁺) m/z [M+H]⁺: 374.3

¹H NMR (600 MHz, DMSO-d₆) δ ppm: 9.66 (s, 1H), 8.98 (s, 1H), 7.60 (d, J=10.3 Hz, 1H), 4.55 (ddd, J=11.6, 6.8, 2.8 Hz, 1H), 4.32 (ddd, J=11.7, 8.5, 1.6 Hz, 1H), 4.27-4.16 (m, 2H), 4.09-3.97 (m, 1H), 3.89 (s, 3H), 3.60-3.51 (m, 1H), 3.16-3.09 (m, 1H), 2.80 (s, 3H), 2.47-2.42 (m, 1H), 2.35-2.25 (m, 1H), 2.09-1.99 (m, 1H), 1.94-1.79 (m, 2H), 1.79-1.68 (m, 1H), 1.58-1.47 (m, 1H)

### Example 69

### (S)—N-((5-Fluoro-2-methoxy-6-(2,3,3,3-tetrafluoro-2-(trifluoromethyl)propoxy)pyridin-3-yl)methyl)pyrrolidine-2-carboxamide Hydrochloride

(Compound of Formula Ia.1, Wherein X is CH, R⁵ is H, R⁶ is F, R⁷ is OCH₃ and R^{8a} is CH₂CF(CF₃)₂)

The title compound was prepared using the procedure described in example 2, starting from 2,3,3,3-tetrafluoro-2-(trifluoromethyl)propan-1-ol and (S)-tert-butyl 2-(((6-chloro-5-fluoro-2-methoxypyridin-3-yl)methyl)carbamoyl)pyrrolidine-1-carboxylate, followed by BOC deprotection.

One slight modification was made with respect to the Pd coupling conditions:

### 69.1 (S)-tert-butyl 2-(((5-fluoro-2-methoxy-6-(2,3,3,3-tetrafluoro-2-(trifluoromethyl)propoxy)pyridin-3-yl)methyl)carbamoyl)pyrrolidine-1-carboxylate

2,3,3,3-Tetrafluoro-2-(trifluoromethyl)propan-1-ol (413 mg, 2.063 mmol) was dissolved under nitrogen in 2 mL of toluene, sodium hydride (53.6 mg, 1.341 mmol) was added and the mixture was stirred at 50-80° C. for 15 min. Then (S)-tert-butyl 2-(((6-chloro-5-fluoro-2-methoxypyridin-3-yl)methyl)carbamoyl)pyrrolidine-1-carboxylate (200 mg, 0.516 mmol), 2,2′-bis(diphenylphosphino)-1,1′-binaphthalene/BINAP (21.19 mg, 0.034 mmol) and tris(dibenzylideneacetone)dipalladium(0) (15.58 mg, 0,017 mmol) were added as solids at once. The reaction mixture was diluted with 3 mL of toluene and stirred at 85° C. for 3.25 h. Stirring was continued at room temperature (RT) overnight. Since the reaction was only very poor an additional amount of alcoholate, prepared by stirring 1.3 eq NaH and 2 eq of 2,3,3,3-tetrafluoro-2-(trifluoromethyl)propan-1-ol in 3 mL of toluene for 10 min, was added to the reaction mixture with 1 spatula catalyst and subsequently heated at 85° C. for 3.25 h followed by stirring at RT overnight. Since the reaction was still not complete one spatula RuPhos and cat. were added and the reaction mixture was heated at 85° C. for 7.5 followed by stirring overnight. Water was added and extracted with ethyl acetate. The water-layer was twice extracted with ethyl acetate. The ethyl acetate layer was dried with sodium sulfate, filtered and evaporated. The raw material (369 mg) was purified by column chromatography on silica gel (12 g column; heptane 100%→ethylacetate/heptane 50:50, 30 ml/min) to give (S)-tert-butyl 2-(((5-fluoro-2-methoxy-6-(2,3,3,3-tetrafluoro-2-(trifluoromethyl)propoxy)pyridin-3-yl)methyl)carbamoyl)pyrrolidine-1-carboxylate (159 mg, yield 30%) as a white foam.

After BOC deprotection the hydrochloride salt was formed by adding one equivalent of hydrochloric acid.

LCMS (ESI+) m/z [M+H]⁺: 452.20

¹H NMR (500 MHz, Methanol-d₄) δ ppm: 7.54 (d, J=9.7 Hz, 1H), 5.25-5.09 (m, 2H), 4.32 (s, 2H), 4.21 (dd, J=8.4, 6.8 Hz, 1H), 3.96 (s, 3H), 3.41-3.35 (m, 1H), 3.32-3.27 (m, 1H), 2.44-2.36 (m, 1H), 2.08-1.92 (m, 3H).

### Example 70

### (2S)—N-[[6-(1-Adamantylmethoxy)-5-fluoro-2-methoxy-3-pyridyl]methyl]-pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is CH₂-adamantan-1-yl)

The title compound was prepared using the procedure described in example 2, starting from (3r,5r,7r)-adamantan-1-ylmethanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI+) m/z [M+H]+: 418.30

¹H NMR (500 MHz, Methanol-d₄): δ 7.40 (d, J=10.0 Hz, 1H), 4.28 (m, 2H), 4.18 (m, 1H), 3.97 (s, 2H), 3.92 (s, 3H), 3.38-3.28 (m, 2H), 2.38 (m, 1H), 2.15-1.95 (m, 6H), 1.85-1.65 (m, 12H).

### Example 71

### (2S)—N-[[6-(3,3-Difluorocyclobutoxy)-5-fluoro-2-methoxy-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is A.10)

The title compound was prepared using the procedure described in example 1 starting 3,3-difluorocyclobutanol, 2,5,6-trifluoronicotinonitrile, methanol and (2S,4R)-1-(tert-butoxycarbonyl)-4-methylpyrrolidine-2-carboxylic acid, followed by BOC deprotection. Finally the hydrochloride salt was formed by adding one equivalent hydrochloric acid.

LCMS (ESI⁺) m/z [M+H]⁺: 374.3

¹H NMR (500 MHz, DMSO-d₆) δ ppm: 9.71 (s, 1H), 9.12 (d, J=5.9 Hz, 1H), 7.63 (d, J=10.2 Hz, 1H), 5.18-5.09 (m, 1H), 4.28-4.16 (m, 2H), 4.10 (t, J=8.3 Hz, 1H), 3.87 (s, 3H), 3.56 (s, 1H), 3.28-3.08 (m, 3H), 2.89-2.71 (m, 5H), 2.50-2.44 (m, 1H), 2.13-1.98 (m, 1H), 1.97-1.80 (m, 2H).

### Example 72

### (2S)—N-((6-((4-(1,1-Difluoroethyl)cyclohexyl)oxy)-5-fluoro-2-methoxypyridin-3-yl)methyl)pyrrolidine-2-carboxamide 2,2,2-trifluoroacetate

(Compound of Formula Ia.1, Wherein X is CH, R⁵ is H, R⁶ is F, R⁷ is OCH₃ and R^{8a} is A.42)

The title compound was prepared using the procedure described in example 2, starting from 4-(1,1-difluoroethyl)cyclohexanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 416.30

¹H NMR (500 MHz, Methanol-d₄): δ 7.40 (d, J=9.9 Hz, 1H), 4.95 (m, 1H), 4.29 (m, 2H), 4.21 (m, 1H), 3.92 (s, 3H), 3.40 (m, 2H), 2.40 (m, 1H), 2.26 (m, 2H), 2.05-1.95 (m, 5H), 1.85 (m, 1H), 1.60-1.35 (m, 7H).

### Example 73

### (2S)—N-[[6-[4-(1,1-Difluoroethyl)cyclohexoxy]-5-fluoro-2-methoxy-3-pyridyl]-methyl]pyrrolidine-2-carboxamide, Isomer 1

(Compound of Formula Ia.1, Wherein X is CH, R⁵ is H, R⁶ is F, R⁷ is OCH₃ and R^{8a} is A.42)

The title compound was prepared as described in example 72. The separation of the two diastereomers was accomplished via SFC (see general description of Preparative SFC).

### Isomer 1:

LCMS (ESI⁺) m/z [M+H]⁺: 416.30

### General Description of Preparative SFC:

Preparative separations were carried out on a Waters Prep 100q SFC System, controlled by Waters MassLynx Software. The system consists of an open bed injector/collector, a heated column compartment including a switch for 6 columns, a CO₂-booster pump, a pump module for modifier flow and an UV-detector. To enable quantitative collection, the gas liquid separator was driven with a make-up flow of 30 mL/min Methanol. The backpressure regulator was set to 120 bar and heated to 60° C. If not stated otherwise, the columns were 250 mm in length, 20 mm in diameter and packed with 5 μm material. They were kept at 30° C. during the separation. As mobile phase, a mixture of liquefied CO₂ and organic modifier with additive was used as indicated for each sample. The flow rate was kept at 100 g/min.

### Example 74

### (2S)—N-[[6-[4-(1,1-Difluoroethyl)cyclohexoxy]-5-fluoro-2-methoxy-3-pyridyl]-methyl]pyrrolidine-2-carboxamide, Isomer 2

(Compound of Formula Ia.1, Wherein X is CH, R⁵ is H, R⁶ is F, R⁷ is OCH₃ and R^{8a} is A.42)

The title compound was prepared as described in example 72. The separation of the two diastereomers was accomplished via SFC (see general description of Preparative SFC).

### Isomer 2:

LCMS (ESI+) m/z [M+H]⁺: 416.30

### Example 75

### (2S)—N-[[5-fluoro-6-[4-(fluoromethyl)cyclohexoxy]-2-methoxy-3-pyridyl]methyl]-pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.58)

The title compound was prepared using the procedure described in example 2, starting from 4-(((tert-butyldimethylsilyl)oxy)methyl)cyclohexanol and (S)-tert-butyl 2-(((6-chloro-5-fluoro-2-methoxypyridin-3-yl)methyl)carbamoyl)pyrrolidine-1-carboxylate, followed by cleavage of the TBS protection group, transformation of the hydroxymethyl into the fluoromethyl group via the mesylate leaving group and final BOC deprotection of the proline amide moiety.

### 75.1 (S)-tert-Butyl 2-(((6-((4-(((tert-butyldimethylsilyl)oxy)methyl)cyclohexyl)oxy)-5-fluoro-2-methoxypyridin-3-yl)methyl)carbamoyl)pyrrolidine-1-carboxylate

Under an argon atmosphere a solution of 250 mg of (S)-tert-butyl 2-(((6-chloro-5-fluoro-2-methoxypyridin-3-1)methyl)carbamoyl)pyrrolidine carboxylate (0.645 mmol, 1.00 eq), 205 mg of 4-(((tert-butyldimethylsilyl)oxy)methyl)cyclohexanol (0.838 mmol, 1.30 eq), 7.24 mg of palladium(II) acetate (0.032 mmol, 0.050 eq), 19.2 mg of JohnPhos (0.064 mmol, 0.10 eq) and 420 mg cesium carbonate (1.29 mmol, 2.00 eq) in toluene (4.5 mL) was heated in a microwave at 120° C. for 16h. Then the reaction mixture was concentrated under reduced pressure and water and NaOH_{aq} (1M) was added to the residue. The aqueous layer was extracted three times with ethyl acetate and the combined organic phases were dried over MgSO₄, filtrated and the solvent was evaporated under reduced pressure. The crude product was purified by column chromatography on silica (eluent: 0-10% methanol in dichloromethane) to yield the title compound (78%, 0.504 mmol).

ESI-MS: m/z (%): 618.40 (100, [M+Na]⁺).

### 75.2 (S)-tert-butyl 2-(((5-fluoro-6-((4-(hydroxymethyl)cyclohexyl)oxy)-2-methoxypyridin-3-yl)methyl)carbamoyl)pyrrolidine-1-carboxylate

To a solution of 375 mg (S)-tert-butyl 2-(((6-((4-(((tert-butyldimethylsilyl)oxy)methyl)cyclohexyl)oxy)-5-fluoro-2-methoxypyridin-3-yl)methyl)carbamoyl)pyrrolidine-1 carboxylate (0.441 mmol, 1.00 eq) in THF (15 mL) was added 0.881 mL of tetrabutylammonium fluoride (1M in THF, 0.881 mmol, 2.00 eq). The mixture was stirred at room temperature for 18 h. Then a saturated aqueous NH₄Cl solution was added and the mixture was extracted with ethyl acetate. The organic phase was dried over MgSO₄, filtrated and the solvent was evaporated. The crude product was purified by column chromatography on silica (eluent: 0-10% methanol in dichloromethane) to yield the title compound (26%, 0.114 mmol).

ESI-MS: m/z (%): 382.30 (100, [M-Boc+H]⁺), 482.30 (10, [M+H]⁺), 504.30 (60, [M+Na]⁺).

### 75.3 (S)-tert-butyl 2-(((5-fluoro-2-methoxy-6-((4-(((methylsulfonyl)oxy)methyl)cyclohexyl)oxy)pyridin-3-yl)methyl)carbamoyl)pyrrolidine-1-carboxylate

A solution of 55 mg (S)-tert-butyl 2-(((5-fluoro-6-((4-(hydroxymethyl)cyclohexyl)oxy)-2-methoxypyridin-3-yl)methyl)carbamoyl)pyrrolidine-1-carboxylate (0.114 mmol, 1.00 eq) and 0.032 mL triethylamine (0.228 mmol, 2.00 eq) in dichloromethane (2 ml) was cooled to 0° C. and 9.79 μl methanesulfonyl chloride (0.126 mmol, 1.10 eq) was slowly added. Then the mixture was warmed to room temperature and stirred for 2 h. Water was added and the solution was extracted with dichloromethane. The organic phase was dried over MgSO₄, filtrated, the solvent was evaporated. The crude product was purified by column chromatography on silica (eluent: 0-10% methanol in dichloromethane) to yield the title compound (53%, 0.061 mmol).

ESI-MS: m/z (%): 460.30 (100, [M-Boc+H]⁺), 582.30 (90, [M+Na]⁺).

### 75.4 (S)-tert-Butyl 2-(((5-fluoro-6-((4-(fluoromethyl)cyclohexyl)oxy)-2-methoxypyridin-3-yl)methyl)carbamoyl)pyrrolidine-1-carboxylate

A solution of 34 mg (S)-tert-butyl 2-(((5-fluoro-2-methoxy-6-((4-(((methylsulfonyl)oxy)methyl)cyclohexyl)oxy)pyridin-3-yl)methyl)carbamoyl)pyrrolidine-1-carboxylate (0.061 mmol, 1.00 eq) and 36.9 mg cesium fluoride (0.243 mmol, 4.00 eq) in t-butanol (2 ml) was heated in the microwave at 90° C. for 50 h. Then a saturated aqueous NaHCO₃ solution was added and the mixture was extracted with dichloromethane. The combined organic phases were dried over MgSO₄ and the solvent was evaporated. The crude product was purified by column chromatography on silica (eluent: 0-10% methanol in dichloromethane) to yield the title compound (89%, 0.054 mmol).

ESI-MS: m/z (%): 384.30 (60, [M-Boc+H]⁺), 484.40 (5, [M+H]⁺), 506.40 (100, [M+Na]⁺).

### 75.5 (S)—N-((5-Fluoro-6-((4-(fluoromethyl)cyclohexyl)oxy)-2-methoxypyridin-3-yl)methyl)pyrrolidine-2-carboxamide

A solution of 26 mg (S)-tert-butyl 2-(((5-fluoro-6-((4-(fluoromethyl)cyclohexyl)oxy)-2-methoxypyridin-3-yl)methyl)carbamoyl)pyrrolidine-1-carboxylate (0.054 mmol, 1.00 eq) and 0.021 mL trifluoroacetic acid (0.269, 5.00 eq) in dichloromethane (2 mL) was stirred at room temperature for 24 h. Then the solvent was evaporated and the crude product was purified via preparative HPLC to yield the title compound as TFA salt (16%, 0.008 mmol).

LC ESI-MS: m/z (%): 384.20 (100, [M+H]⁺).

¹H NMR (600 MHz, CDCl₃): δ ppm: 8.00 (s, 1H), 7.25 (s, 1H), 4.93 (tt, J=10.9, 4.2 Hz, 1H), 4.66 (dd, J=8.4, 5.7 Hz, 1H), 4.33 (d, J=5.9 Hz, 1H), 4.29 (d, J=5.6 Hz, 2H), 4.25 (d, J=5.9 Hz, 1H), 3.87 (d, J=1.6 Hz, 3H), 3.39 (t, J=6.6 Hz, 2H), 2.42 (d, J=10.1 Hz, 1H), 2.28-2.20 (m, 2H), 2.09-1.96 (m, 3H), 1.91 (dq, J=13.5, 3.3 Hz, 2H), 1.80-1.70 (m, 2H), 1.54 (tp, J=12.3, 3.8 Hz, 2H), 1.28-1.16 (m, 2H).

### Example 76

### (2S)—N-[[5-Fluoro-2-methoxy-6-[[1-(trifluoromethyl)cyclopropyl]methoxy]-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is CH₂-A.4)

The title compound was prepared using the procedure described in example 2, starting from 4(1-(trifluoromethyl)cyclopropyl)methanol and (S)-tert-butyl 2-(((6-chloro-5-fluoro-2-methoxypyridin-3-yl)methyl)(methyl)carbamoyl)pyrrolidine-1-carboxylate, followed by BOC deprotection. Finally the hydrochloride salt was formed by adding one equivalent hydrochloric acid.

### 76.1 (S)-tert-Butyl 2-(((6-chloro-5-fluoro-2-methoxypyridin-3-yl)methyl)(methyl)carbamoyl)pyrrolidine-1-carboxylate

(S)-tert-butyl 2-(((6-chloro-5-fluoro-2-methoxypyridin-3-yl)methyl)carbamoyl)pyrrolidine-1-carboxylate (1 g, 2.58 mmol) was dissolved in DMF (25 mL) at 0° C. under nitrogen to give a light yellow solution. Sodium hydride (0.155 g, 3.87 mmol) was added slowly. The reaction mixture was stirred at 0° C. for 45 min. Then iodomethane (0.242 mL, 3.87 mmol) was added. The reaction mixture was allowed to warm to room temperature (RT) and was stirred for 18 h. The color of the reaction mixture turned from light red to yellow overnight. The reaction mixture was cooled down to 5° C. and water was added. The pH was basified with 2M NaOH and then extracted 3× with MTBE. The combined organic layers were dried with sodium sulfate, filtered and concentrated. The crude product (744 mg) was purified by column chromatography on silica gel (40 g-column, ethyl acetate/heptane, 0% ethyl acetate→70% ethyl acetate, flow: 40 ml/min.) to give (S)-tert-butyl 2-(((6-chloro-5-fluoro-2-methoxypyridin-3-yl)methyl)(methyl)carbamoyl)pyrrolidine-1-carboxylate (559 mg, yield: 54%) as yellow oil. After BOC deprotection the hydrochloride salt was formed by adding one equivalent of hydrochloric acid.

LCMS (ESI⁺) m/z [M+H]⁺: 406.25

¹H NMR (500 MHz, Methanol-d₄) δ ppm: 7.43 (d, J=9.8 Hz, 1H, A), 7.39 (d, J=9.9 Hz, 1H, B), 4.67-4.40 (m, 4H, A and B), 4.34-4.28 (m, 1H, A and B), 3.94 (s, 3H, A), 3.92 (s, 3H, B), 3.27-3.33 (m, 1H, A and B, under MeOH peak), 3.09-3.01 (m, 1H, A and B), 3.05 (s, 3H, B), 2.86 (s, 3H, A), 2.43-2.28 (m, 1H, A and B), 2.03 1.71 (m, 3H, A and B), 1.15-1.08 (m, 2H, A and B), 1.02-0.96 (m, 2H, A and B).

The NMR shows two sets of signals based on rotameres A and B.

### Example 77

### (2S)—N-[[6-[(2,2-Difluorocyclopropyl)methoxy]-5-fluoro-2-methoxy-3-pyridyl]-methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is CH₂-A.33)

The title compound was prepared using the procedure described in example 2 and 76, starting from 4(1-(trifluoromethyl)cyclopropyl)methanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 374.20

¹H NMR (500 MHz, Methanol-d₄) δ ppm: 7.43 (d, J=9.8 Hz, 1H, A), 7.39 (d, J=10.0 Hz, 1H, B), 4.68-4.23 (m, 5H, A and B), 3.95 (s, 3H, A), 3.93 (s, 3H, B), 3.30-3.24 (m, 1H, A and B), 3.05 (s, 3H, B), 3.04-2.99 (m, 1H, A and B), 2.86 (s, 3H, A), 2.40-2.27 (m, 1H, A and B), 2.26-2.14 (m, 1H, A and B), 2.00-1.72 (m, 3H, A and B), 1.66-1.56 (m, 1H, A and B), 1.41-1.32 (m, 1H, A and B). The NMR shows two sets of signals based on rotameres A and B.

### Example 78

### (2S)—N-[[6-(3,3-Difluorocyclobutoxy)-5-fluoro-2-methoxy-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is A.10)

The title compound was prepared using the procedure described in example 2, starting from 4(1-(trifluoromethyl)cyclopropyl)methanol and (S)-tert-butyl 2-(((6-chloro-5-fluoro-2-methoxypyridin-3-yl)methyl)(methyl)carbamoyl)pyrrolidine-1-carboxylate (example 77), followed by BOC deprotection. Finally the hydrochloride salt was formed by adding one equivalent hydrochloric acid.

LCMS (ESI⁺) m/z [M+H]⁺: 374.20

¹H NMR (500 MHz, Methanol-d₄) δ ppm: 7.48 (d, J=9.8 Hz, 1H, A), 7.43 (d, J=10.1 Hz, 1H, B), 5.23-5.08 (m, 1H, A and B), 4.68-4.60 (m, 1H, B), 4.55-4.43 (m, 2H, A and B), 4.30 (d, J=16.0 Hz, 1H, A), 3.94 (s, 3H, A), 3.92 (s, 3H, B), 3.42-3.34 (m, 1H, A and B), 3.25-3.09 (m, 3H, A and B), 3.05 (s, 3H, B), 2.85 (s, 3H, A), 2.84 2.70 (m, 2H, A and B), 2.51-2.38 (m, 1H, A and B), 2.08-1.81 (m, 3H, A and B). The NMR shows two sets of signals based on rotameres A and B.

### Example 79

### (2S,3R)—N-[[5-Fluoro-2-methoxy-6-[4-(trifluoromethyl)cyclohexoxy]-3-pyridyl]-methyl]-3-methyl-azetidine-2-carboxamide; Fumaric Acid

(Compound of Formula Ia.11, Wherein X is CH, R⁵ is H, R⁶ is F, R⁷ is OCH₃ and R^{8a} is A.36)

The title compound was prepared using the procedure described in example 1 starting from trans-4-(trifluoromethyl)cyclohexanol, 2,5,6-trifluoronicotinonitrile, methanol and (2S,3R)-1-(tert-butoxycarbonyl)-3-methylazetidine-2-carboxylic acid, followed by BOC deprotection. Finally the fumarate salt was formed by adding one equivalent fumaric acid.

### 79.1 5-Fluoro-2-methoxy-6-(((1 r,4r)-4-(trifluoromethyl)cyclohexyl)oxy)nicotinonitrile

Step 1: To a solution of potassium 2-methylpropan-2-olate (3.95 g, 35.2 mmol) in THF (25 ml) at 0° C. was added trans-4-(trifluoromethyl)cyclohexanol (6.20 g, 36.9 mmol) under nitrogen. After stirring for 30 minutes the reaction mixture was added slowly to a solution of 2,5,6-trifluoronicotinonitrile (5.3 g, 33.5 mmol) in THF (25 ml) at −60° C. The reaction mixture was stirred for 1 hour at −60° C. and 1 hour at 0° C.

Step 2: To a solution of potassium 2-methylpropan-2-olate (4.51 g, 40.2 mmol) in THF (25 ml) at 0° C. was added methanol (1.343 g, 41.9 smmol) under nitrogen. After stirring for 30 minutes the reaction mixture was added slowly to the solution of step 1 at −60° C. The reaction mixture was stirred for 2 hour at −60° C. NH₄Cl (sat. aq 30 mL) was added to the reaction mixture. The pH was adjusted to 8 with 2M NaOH. The aqueous layer was extracted three times with ethyl acetate, the combined organic layers were washed with saturated aqueous sodium chloride, dried over sodium sulphate, filtered and the solvent evaporated to give 5-fluoro-2-methoxy-6-(((1r,4r)-4-(trifluoromethyl)cyclohexyl)oxy)nicotinonitrile (10.3 g, 32.4 mmol, 97% yield) as a gray solid. The crude product was used directly for the next step without further purification. Reduction of the nitrile to the amine was performed according to example 5. (2S,3R)-methyl 3-methyl-1-picolinoylazetidine-2-carboxylate was prepared as described in Journal of the American Chemical Society (2012), 134(1), 3-6, followed by acid hydrolysis and subsequent BOC protection of the amine group to yield (2S,3R)-1-(tert-butoxycarbonyl)-3-methylazetidine-2-carboxylic acid, which was used in the peptide coupling with (5-fluoro-2-methoxy-6-(((1r,4r)-4-(trifluoromethyl)cyclohexyl)oxy)pyridin-3-yl)methanamine according to the procedure described in example 1.

LCMS (ESI⁺) m/z [M+H]⁺: 420.30

¹H NMR (500 MHz, Methanol-d₄) δ ppm: 7.42 (d, J=10.0 Hz, 1H), 6.69 (s, 2H), 5.04-4.92 (m, 1H), 4.52 (d, J=7.3 Hz, 1H), 4.30 (s, 2H), 4.02-3.95 (m, 1H), 3.92 (s, 3H), 3.68 (dd, J=10.1, 7.9 Hz, 1H), 2.96-2.83 (m, 1H), 2.33-2.14 (m, 3H), 2.09-1.99 (m, 2H), 1.59-1.44 (m, 4H), 1.35 (d, J=6.8 Hz, 3H).

### Example 80

### (2S,3 S)—N-[[5-Fluoro-2-methoxy-6-[4-(trifluoromethyl)cyclohexoxy]-3-pyridyl]methyl]-3-methyl-azetidine-2-carboxamide; Fumaric Acid

(Compound of Formula Ia.11, Wherein X is CH, R⁵ is H, R⁶ is F, R⁷ is OCH₃ and R^{8a} is A.36)

The title compound was prepared using the procedure described in example 1, 5 and 79 starting from trans-4-(trifluoromethyl)cyclohexanol, 2,5,6-trifluoronicotinonitrile, methanol and (2S,3S)-1-(tert-butoxycarbonyl)-3-methylazetidine-2-carboxylic acid, followed by BOC deprotection. Finally the fumarate salt was formed by adding one equivalent fumaric acid.

LCMS (ESI⁺) m/z [M+H]⁺: 420.30

¹H NMR (500 MHz, Methanol-d₄) δ ppm: 7.47 (d, J=10.0 Hz, 1H), 6.69 (s, 2H), 5.01-4.94 (m, 1H), 4.93 (d, J=9.2 Hz, 1H), 4.36 (d, J=14.6 Hz, 1H, AB signal), 4.27 (d, J=14.6 Hz, 1H, AB signal), 4.14 (dd, J=10.2, 8.7 Hz, 1H), 3.92 (s, 3H), 3.56 (dd, J=10.3, 6.8 Hz, 1H), 3.24-3.12 (m, 1H), 2.34-2.15 (m, 3H), 2.08-2.00 (m, 2H), 1.60-1.45 (m, 4H), 1.06 (d, J=7.1 Hz, 3H).

### Example 81

### (2S)—N-[[5-Fluoro-6-(4-hydroxycyclohexoxy)-2-methoxy-3-pyridyl]methyl]-pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.55)

The title compound was obtained as side product by BOC cleavage of (S)-tert-butyl 2-(((6-((4-(difluoromethoxy)cyclohexyl)oxy)-5-fluoro-2-methoxypyridin-3-yl)methyl)carbamoyl)pyrrolidine-1-carboxylate (precursor described in example 67) and purified by RP HPLC.

LCMS (ESI⁺) m/z [M+H]⁺: 368.30

¹H NMR (500 MHz, Methanol-d₄): δ 7.41 (d, J=10.0 Hz, 1H), 6.41 (t, J=76.1 Hz, 1H), 5.10 (m, 1H), 4.30-4.20 (m, 4H), 3.92 (s, 3H), 3.39 (m, 2H), 2.40 (m, 1H), 2.15 (m, 2H), 2.05-1.95 (m, 5H), 1.70-1.60 (m, 4H).

### Example 82

### (1R,3 S,5R)—N-[[5-Fluoro-2-methoxy-6-[4-(trifluoromethyl)cyclohexoxy]-3-pyridyl]methyl]-4-azabicyclo[3.1.0]hexane-3-carboxamide

(Compound of Formula Ia.13, Wherein X is CH, R⁵ is H, R⁶ is F, R⁷ is OCH₃ and R^{8a} is A.36)

The title compound was prepared using the procedure described in example 1, 5 and 79 starting from trans-4-(trifluoromethyl)cyclohexanol, 2,5,6-trifluoronicotinonitrile, methanol and (1R,3S,5R)-2-(tert-butoxycarbonyl)-2-azabicyclo[3.1.0]hexane-3-carboxylic acid, followed by BOC deprotection.

LCMS (ESI⁺) m/z [M+H]⁺: 432.30

¹H NMR (500 MHz, CDCl₃): δ 8.26 (bs, 1H), 7.24 (d, J=9.8 Hz, 1H), 4.91 (m, 1H), 4.48 (m, 1H), 4.28 (m, 2H), 3.86 (s, 3H), 3.32 (m, 1H), 2.49 (m, 1H), 2.29 (m, 2H), 2.18 (m, 1H), 2.10-2.00 (m, 3H), 1.80 (m, 1H), 1.57-1.47 (m, 4H), 0.98 (m, 1H), 0.80 (m, 1H).

### Example 83

### N-[[5-Fluoro-2-methoxy-6-[4-(trifluoromethyl)cyclohexoxy]-3-pyridyl]methyl]-4-azabicyclo[3.1.0]hexane-5-carboxamide

(Compound of Formula Ia.17, Wherein X is CH, R⁵ is H, R⁶ is F, R⁷ is OCH₃ and R^{8a} is A.36)

The title compound was prepared using the procedure described in example 1, 5 and 79 starting from trans-4-(trifluoromethyl)cyclohexanol, 2,5,6-trifluoronicotinonitrile, methanol and 2-(tert-butoxycarbonyl)-2-azabicyclo[3.1.0]hexane-1-carboxylic acid, followed by BOC deprotection.

LCMS (ESI⁺) m/z [M+H]⁺: 432.25

¹H NMR (500 MHz, CDCl₃): δ 7.36 (bs, 1H), 7.28 (d, J=8.7 Hz, 1H), 4.90 (m, 1H), 4.27 (m, 2H), 3.85 (s, 3H), 3.59 (m, 1H), 3.03 (m, 1H), 2.37 (m, 1H), 2.28 (m, 3H), 2.15-2.05 (m, 4H), 1.61 (m, 1H), 1.55-1.45 (m, 5H).

### Example 84

### (1S,4S,5R)—N-[[5-Fluoro-2-methoxy-6-[4-(trifluoromethyl)cyclohexoxy]-3-pyridyl]methyl]-3-azabicyclo[3.1.0]hexane-4-carboxamide Hydrochloride

(Compound of Formula Ia.15, Wherein X is CH, R⁵ is H, R⁶ is F, R⁷ is OCH₃ and R^{8a} is A.36)

The title compound was prepared using the procedure described in example 1, 5 and 79 starting from trans-4-(trifluoromethyl)cyclohexanol, 2,5,6-trifluoronicotinonitrile, methanol and (1R,2S,5S)-3-(tert-butoxycarbonyl)-3-azabicyclo[3.1.0]hexane-2-carboxylic acid, followed by BOC deprotection. Finally the hydrochloride salt was formed by adding one equivalent hydrochloric acid.

LCMS (ESI⁺) m/z [M+H]⁺: 432.4

¹H NMR (500 MHz, DMSO-d₆) δ ppm: 10.00 (s, 1H), 9.07 (t, J=5.6 Hz, 1H), 8.34 (s, 1H), 7.53 (d, J=10.4 Hz, 1H), 4.99-4.89 (m, 1H), 4.39 (s, 1H), 4.22 (d, J=5.7 Hz, 2H), 3.88 (s, 3H), 2.44-2.30 (m, 1H), 3.47-3.17 (m, 2H, under water signal), 2.27-2.14 (m, 2H), 2.07 (tt, J=8.2, 4.3 Hz, 1H), 2.01-1.88 (m, 2H), 1.76 (tt, J=8.0, 4.1 Hz, 1H), 1.57-1.38 (m, 4H), 0.69-0.60 (m, 1H), 0.60-0.51 (m, 1H).

### Example 85

### (1R,4S,5S)—N-[[5-Fluoro-2-methoxy-6-[4-(trifluoromethyl)cyclohexoxy]-3-pyridyl]methyl]-3-azabicyclo[3.1.0]hexane-4-carboxamide Hydrochloride

(Compound of Formula Ia.15, Wherein X is CH, R⁵ is H, R⁶ is F, R⁷ is OCH₃ and R^{8a} is A.36)

The title compound was prepared using the procedure described in example 1, 5 and 79 starting from trans-4-(trifluoromethyl)cyclohexanol, 2,5,6-trifluoronicotinonitrile, methanol and (1S,2S,5R)-3-(tert-butoxycarbonyl)-3-azabicyclo[3.1.0]hexane-2-carboxylic acid, followed by BOC deprotection. Finally the hydrochloride salt was formed by adding one equivalent hydrochloric acid.

LCMS (ESI⁺) m/z [M+H]⁺: 432.4

¹H NMR (500 MHz, DMSO-d₆) δ ppm: 9.56 (s, 1H), 9.01 (t, J=5.7 Hz, 2H), 7.59 (d, J=10.4 Hz, 1H), 5.01-4.85 (m, 1H), 4.25 (s, 1H), 4.22 (dd, J=15.3, 5.5 Hz, 1H), 4.16 (dd, J=15.3, 5.5 Hz, 1H), 3.87 (s, 3H), 3.45-3.37 (m, 1H), 3.27 (d, J=11.0 Hz, 1H), 2.45-2.30 (m, 1H), 2.24-2.17 (m, 2H), 1.95 (dt, J=10.0, 3.2 Hz, 2H), 1.84-1.73 (m, 2H), 1.58-1.38 (m, 4H), 0.81-0.72 (m, 2H).

### Example 86

### (2S)—N-[[6-(2,2-Difluoroethoxy)-5-fluoro-2-methoxy-3-pyridyl]methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is CH₂CHF₂)

The title compound was prepared using the procedure described in example 2, starting from 2,2-difluoroethanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 334.20

¹H NMR (500 MHz, Methanol-d₄): δ 7.50 (d, J=9.8 Hz, 1H), 6.22 (t, J=55.2 Hz, 1H), 4.61 (m, 2H), 4.31 (m, 2H), 4.23 (m, 1H), 3.95 (s, 3H), 3.37 (m, 2H), 2.40 (m, 1H), 2.02 (m, 3H).

### Example 87

### (2S)—N-[[5-Fluoro-2-methoxy-6-(2,2,2-trifluoroethoxy)-3-pyridyl]methyl]-pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is CH₂CF₃)

The title compound was prepared using the procedure described in example 2, starting from 2,2,2-trifluoroethanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 352.20

¹H NMR (500 MHz, Methanol-d₄): δ 7.53 (d, J=9.8 Hz, 1H), 4.94 (m, 2H), 4.32 (m, 2H), 4.24 (m, 1H), 3.95 (s, 3H), 3.37 (m, 2H), 2.40 (m, 1H), 2.03 (m, 3H).

### Example 88

### (2S)—N-[[5-Fluoro-2-methoxy-6-[(1S,3R)-3-(trifluoromethyl)cyclohexoxy]-3-pyridyl]methyl]pyrrolidine-2-carboxamide Hydrochloride

(Compound of Formula Ia.1, Wherein X is CH, R⁵ is H, R⁶ is F, R⁷ is OCH₃ and R^{8a} is A.35)

The title compound was prepared using the procedure described in example 2, starting from 3-(trifluoromethyl)cyclohexanol and (S)-tert-butyl 2-(((6-chloro-5-fluoro-2-methoxypyridin-3-yl)methyl)carbamoyl)pyrrolidine-1-carboxylate, followed by BOC deprotection. The separation of the isomers was performed with chiral SFC.

Analytic method: Agilent 1260 Infinity SFC; column: Chiralpak® IC, 4.6×100 mm, 5 μm; eluent: 85% CO₂; 15% methanol with 0.2% aqueous ammonium hydroxide; flow rate: 3.5 mL/min: Time: 1.29, 1.61 min.

Preparative method: SFC (Waters Prep 100q SFC); column: Chiralpak® IC for SFC, 20×250 mm, 5 m eluent: 80% CO₂; 20% methanol with 0.2% aqueous ammonium hydroxide; flow rate: 100 g/min: time: 2.38 min.

The structure assigned to the 3-(trifluoromethyl)cyclohexanoyl moiety is based on NMR and is only relative and not absolute. Finally the hydrochloride salt was formed by adding one equivalent hydrochloric acid.

LCMS (ESI⁺) m/z [M+H]⁺: 420.2

¹H NMR (500 MHz, DMSO-d₆) δ ppm: 9.63 (s, 1H), 8.92 (t, J=5.7 Hz, 1H), 8.54 (s, 1H), 7.57 (d, J=10.4 Hz, 1H), 5.01 (tt, J=11.0, 4.2 Hz, 1H), 4.26-4.12 (m, 3H), 3.87 (s, 3H), 3.28-3.12 (m, 2H), 2.68-2.52 (m, 1H), 2.40-2.33 (m, 1H), 2.33-2.25 (m, 1H), 2.15 (d, J=12.2 Hz, 1H), 1.93-1.78 (m, 5H), 1.55-1.32 (m, 3H), 1.30-1.20 (m, 1H).

### Example 89

### (2S)—N-[[5-Fluoro-2-methoxy-6-[(1S,3S)-3-(trifluoromethyl)cyclohexoxy]-3-pyridyl]methyl]pyrrolidine-2-carboxamide Hydrochloride

(Compound of Formula Ia.1, Wherein X is CH, R⁵ is H, R⁶ is F, R⁷ is OCH₃ and R^{8a} is A.35)

The title compound was prepared as described in example 88. The separation of the isomers was performed with chiral SFC.

Preparative method: SFC (Waters Prep 100q SFC); column: Chiralpak® IC for SFC, 20×250 mm, 5 μm eluent: 80% CO₂; 20% methanol with 0.2% aqueous ammonium hydroxide; flow rate: 100 g/min: time: 1.99 min.

The structure assigned to the 3-(trifluoromethyl)cyclohexanoyl moiety is based on NMR and is only relative and not absolute. Finally the hydrochloride salt was formed by adding one equivalent hydrochloric acid.

LCMS (ESI⁺) m/z [M+H]⁺: 420.2

¹H NMR (500 MHz, DMSO-d₆) δ ppm: 9.62 (s, 1H), 8.92 (t, J=5.6 Hz, 1H), 8.55 (s, 1H), 7.58 (d, J=10.2 Hz, 1H), 5.41 (s, 1H), 4.29-4.12 (m, 3H), 3.85 (s, 3H), 3.28-3.11 (m, 2H), 2.58-2.41 (m, 1H), 2.34-2.25 (m, 1H), 2.14 (d, J=14.0 Hz, 1H), 1.98 (d, J=8.1 Hz, 1H), 1.93-1.77 (m, 4H), 1.75-1.54 (m, 4H), 1.42-1.30 (m, 1H).

### Example 90

### (2S)—N-[[5-Fluoro-2-methoxy-6-[(2,2,3,3-tetrafluorocyclobutyl)methoxy]-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is CH₂-A. 11)

The title compound was prepared using the procedure described in example 63 starting from 2,2,3,3-tetrafluorocyclobutanol and (S)—N-((6-chloro-5-fluoro-2-methoxypyridin-3-yl)methyl)-1-methylpyrrolidine-2-carboxamide. Finally the hydrochloride salt was formed by adding one equivalent hydrochloric acid.

LCMS (ESI⁺) m/z [M+H]⁺: 424.3

¹H NMR (600 MHz, DMSO-d₆) δ ppm: 9.79 (s, 1H), 9.32 (t, J=5.5 Hz, 1H), 7.64 (d, J=10.2 Hz, 1H), 4.60 (dd, J=11.4, 5.4 Hz, 1H), 4.54 (dd, J=11.5, 9.0 Hz, 1H), 4.27-4.19 (m, 2H), 4.19-4.12 (m, 1H), 3.90 (s, 3H), 3.57 (ddt, J=11.6, 7.7, 4.1 Hz, 1H), 3.42-3.36 (m, 1H), 3.21-3.11 (m, 1H), 2.92-2.83 (m, 1H), 2.82 (d, J=3.6 Hz, 3H), 2.65-2.55 (m, 1H), 2.55-2.47 (m, 1H), 2.10-2.02 (m, 1H), 1.93-1.81 (m, 2H).

### Example 91

### (2S)—N-[[5-Fluoro-2-methoxy-6-(2,2,3,3-tetrafluoropropoxy)-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is CH₂CF₂CHF₂)

The title compound was prepared using the procedure described in example 63 starting from 2,2,3,3-tetrafluoropropan-1-ol and (S)—N-((6-chloro-5-fluoro-2-methoxypyridin-3-yl)methyl)-1-methylpyrrolidine-2-carboxamide. Finally the hydrochloride salt was formed by adding one equivalent hydrochloric acid.

LCMS (ESI⁺) m/z [M+H]⁺: 398.2

¹H NMR (600 MHz, Methanol-d₄) δ ppm: 7.54 (d, J=9.8 Hz, 1H), 6.28 (tt, J=52.6, 4.8 Hz, 1H), 4.88 (t, J=13.0 Hz, 2H, overlaps with water peak), 4.35 (d, J=15.0 Hz, 1H, AB signal), 4.31 (d, J=15.0 Hz, 1H, AB signal), 4.14-4.04 (m, 1H), 3.96 (s, 3H), 3.76-3.66 (m, 1H), 3.25-3.17 (m, 1H), 2.92 (s, 3H), 2.62-2.50 (m, 1H), 2.26-2.15 (m, 1H), 2.08-1.97 (m, 2H).

### Example 92

### (2S)—N-[[6-[(3,3-Difluorocyclobutyl)methoxy]-5-fluoro-2-methoxy-3-pyridyl]-methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is CH₂-A.10)

The title compound was prepared using the procedure described in example 63 starting from (3,3-difluorocyclobutyl)methanol and (S)—N-((6-chloro-5-fluoro-2-methoxypyridin-3-yl)methyl)-1-methylpyrrolidine-2-carboxamide. Finally the hydrochloride salt was formed by adding one equivalent hydrochloric acid.

LCMS (ESI⁺) m/z [M+H]⁺: 388.3

¹H NMR (600 MHz, Methanol-d₄) δ ppm: 7.45 (d, J=10.0 Hz, 1H), 4.44 (d, J=6.3 Hz, 2H), 4.33 (d, J=14.8 Hz, 1H), 4.29 (d, J=14.9 Hz, 1H), 4.09-4.04 (m, 1H), 3.93 (s, 3H), 3.71 (ddd, J=11.4, 7.6, 4.2 Hz, 1H), 3.21 (dt, J=11.2, 8.2 Hz, 1H), 2.91 (s, 3H), 2.76-2.60 (m, 3H), 2.59-2.52 (m, 1H), 2.52-2.42 (m, 2H), 2.24-2.15 (m, 1H), 2.08-1.96 (m, 2H).

### Example 93

### (2S)—N-[[5-Fluoro-6-(2-fluorocyclopentoxy)-2-methoxy-3-pyridyl]methyl]-pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.18)

The title compound was prepared using the procedure described in example 2, starting from 2-fluorocyclopentan-1-ol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 356.30

¹H NMR (500 MHz, Methanol-d₄): δ 7.44 (d, J=10.1 Hz, 1H), 5.41 (m, 1H), 5.05 (m, 1H), 4.30-4.20 (m, 3H), 3.95 (s, 3H), 3.37 (m, 2H), 2.40 (m, 1H), 2.28 (m, 1H), 2.10-1.80 (m, 8H).

### Example 94

### (2S,4R)—N-[[6-(3,3-Difluorocyclobutoxy)-5-fluoro-2-methoxy-3-pyridyl]methyl]-4-fluoro-pyrrolidine-2-carboxamide Hydrochloride

(Compound of Formula Ia.5, Wherein X is CH, R⁵ is H, R⁶ is F, R⁷ is OCH₃ and R^{8a} is A.10)

The title compound was prepared using the procedure described in example 1 starting 3,3-difluorocyclobutanol, 2,5,6-trifluoronicotinonitrile, methanol and (2S,4R)-1-(tert-butoxycarbonyl)-4-fluoropyrrolidine-2-carboxylic acid, followed by BOC deprotection. Finally the hydrochloride salt was formed by adding one equivalent hydrochloric acid.

LCMS (ESI⁺) m/z [M+H]⁺: 378.3

### Example 95

### (2S)—N-[[6-(7,7-Difluoronorcaran-2-yl)oxy-5-fluoro-2-methoxy-3-pyridyl]methyl]-pyrrolidine-2-carboxamide

(Compound of Formula Ia.1, Wherein X is CH, R⁵ is H, R⁶ is F, R⁷ is OCH₃ and R^{8a} is A.79)

The title compound was prepared using the procedure described in example 2, starting from 7,7-difluorobicyclo[4.1.0]heptan-2-ol and (S)-tert-butyl 2-(((6-chloro-5-fluoro-2-methoxypyridin-3-yl)methyl)carbamoyl)pyrrolidine-1-carboxylate, followed by BOC deprotection.

LCMS (ESI⁺) m/z [M+H]⁺: 400.30

¹H NMR (500 MHz, Methanol-d₄): δ 7.46 (d, J=10.0 Hz, 1H), 5.18 (m, 1H), 4.30 (m, 2H), 4.23 (m, 1H), 3.95 (s, 3H), 3.37 (m, 2H), 2.40 (m, 1H), 2.05-1.55 (m, 9H), 1.32 (m, 2H).

### Example 96

### (2S)—N-[[6-[[1-(Difluoromethyl)cyclopropyl]methoxy]-5-fluoro-2-methoxy-3-pyridyl]methyl]-1-methyl-pyrrolidine-2-carboxamide; (2S,3S)-2,3-dihydroxybutanedioic Acid

(Compound of Formula Ia.2, Wherein X is CH, R⁵ is H, R⁶ is F, R⁷ is OCH₃ and R^{8a} is CH₂-A.6)

The title compound was prepared using the procedure described in example 63 starting from (1-(difluoromethyl)cyclopropyl)methanol and (S)—N-((6-chloro-5-fluoro-2-methoxypyridin-3-yl)methyl)-1-methylpyrrolidine-2-carboxamide.

### 96.1 (1-(Hydroxymethyl)cyclopropyl)methyl Benzoate

21.89 g of cyclopropane-1,1-diyldimethanol (0.214 mol) were dissolved in DCM (400 ml) and 29.8 ml triethylamine (1.0 eq.) was added. Then, 30.1 g of benzoyl chloride (1.0 eq.) was slowly added to the vigorously stirred mixture. The reaction temperature was adjusted to room temperature and stirring was continued overnight. The crude mixture was washed with saturated sodium bicarbonate solution, the organic layer was separated, dried with MgSO₄ and evaporated to dryness. The oily residue was purified by flash chromatography on silica with petrolether/ether. Monobenzoylated alcohol (1-(hydroxymethyl)cyclopropyl)methyl benzoate was obtained as a colorless oil (22.38 g, 51%).

LCMS (ESI⁺) m/z: [M⁺H]⁺ 207.10, [M⁺Na]⁺ 229.15

¹H NMR (600 MHz, CDCl₃) δ ppm: 0.58-0.70 (m, 4H), 2.18 (s, 1H, OH), 3.53 (s, 2H), 4.32 (s, 2H), 7.42-7.49 (m, 2H), 7.55-7.60 (m, 1H), 8.04-8.09 (m, 2H).

### 96.2 (1-Formylcyclopropyl)methyl Benzoate

21.88 g of Dess-Martin periodinane (1.4 eq.) was added at 0° C. to a solution of 7.6 g (1-(hydroxymethyl)cyclopropyl)methyl benzoate (36.9 mmol) in DCM (100 ml). The reaction mixture was allowed to reach room temperature and stirred overnight. Then, the crude mixture was poured onto a mixture of 0.1 M sodium thiosulfate and sodium bicarbonate solution until the gas evolution has finished. The organic layer was separated, washed twice with water, dried with MgSO₄ and evaporated to dryness. The crude oily residue was purified by flash chromatography on silica with petrolether/ether. (1-Formylcyclopropyl)methyl benzoate was obtained as colorless resin (5.84 g, 78%) which slowly started crystallizing.

LCMS (ESI⁺) m/z [M⁺H]⁺ 205.30, [M+Na]+ 227.30.

¹H NMR (500 MHz, DMSO-d₆) δ ppm: 1.27-1.41 (m, 4H), 4.45 (s, 2H), 7.49-7.56 (m, 2H), 7.63-7.70 (m, 1H), 7.90-7.96 (m, 2H), 8.87 (s, 1H, CHO).

### 96.3 (1-(Difluoromethyl)cyclopropyl)methyl Benzoate

5.52 g of (1-formylcyclopropyl)methyl benzoate (27.0 mmol) were dissolved in DCM (75 ml) under argon atmosphere and cooled with ice while 23.0 ml Deoxofluor solution (50% in toluene, 2.0 eq.) was added dropwise via syringe. The reaction mixture was stirred at 0° C. for 2 h, allowed to reach room temperature and stirring was continued for another 2 h. The crude mixture was diluted with DCM and washed with saturated sodium bicarbonate solution. The organic layer was separated, dried with MgSO₄ and evaporated to dryness. The yellowish oil was purified by flash chromatography on silica with petrolether/ether. (1-(Difluoromethyl)cyclopropyl)methyl benzoate was obtained as a colorless oil (4.35 g, 71%).

LCMS (ESI⁺) m/z [M⁺H]⁺ 227.15, [M+Na]+ 249.15 Weak ESI-MS ionization!

### 96.4 (1-(Difluoromethyl)cyclopropyl)methanol

4.35 g of benzoate (19.22 mmol) was dissolved in a mixture of MeOH (75 ml) and 2M NaOH (11 ml) at room temperature and stirred overnight. The reaction mixture was diluted with Et₂O and washed twice with sodium bicarbonate solution. The organic layer was separated, dried with MgSO₄ and carefully evaporated to dryness. (1-(Difluoromethyl)cyclopropyl)methanol was obtained as slowly crystallizing colorless oil (2.00 g, 85%).

LCMS (ESI⁺) m/z [M⁺H]⁺ 123.20

¹H NMR (500 MHz, DMSO-d₆) δ ppm: 0.52-0.73 (m, 4H), 3.46 (d, J=5.7 Hz, 2H), 4.76 (t, J=5.8 Hz, 1H, OH), 5.97 (t, J=57.5 Hz, 1H).

¹⁹F NMR (471 MHz, DMSO-d₆): δ −121.42 (d, J=57.5 Hz).

### 96.5 (2S)—N-[[6-[[1-(Difluoromethyl)cyclopropyl]methoxy]-5-fluoro-2-methoxy-3-pyridyl]methyl]-1-methyl-pyrrolidine-2-carboxamide

Amide formation of (1-(difluoromethyl)cyclopropyl)methano 1 and (S)—N-((6-chloro-5-fluoro-2-methoxypyridin-3-yl)methyl)-1-methylpyrrolidine-2-carboxamide was performed according to example 63. Finally the (2S,3S)-2,3-dihydroxybutanedioic acid salt was formed by adding one equivalent of the corresponding acid.

LCMS (ESI⁺) m/z [M⁺H]⁺ 388.4

¹H NMR (600 MHz, Methanol-d₄) δ ppm: 0.82 (ddd, J=5.5, 4.2, 2.5 Hz, 2H), 0.91-0.97 (m, 2H), 1.97-2.09 (m, 2H), 2.12-2.23 (m, 1H), 2.47-2.58 (m, 1H), 2.90 (s, 3H), 3.14-3.21 (m, 1H), 3.69 (ddd, J=11.3, 7.4, 4.1 Hz, 1H), 3.93 (s, 3H), 4.02 (t, J=8.0 Hz, 1H), 4.29 (d, J=14.9 Hz, 1H), 4.33 (d, J=14.8 Hz, 1H), 4.47 (s, 2H), 4.51 (s, 2H), 5.91 (t, J=57.4 Hz, 1H), 7.45 (d, J=9.9 Hz, 1H).

### Example 97

### (2S)—N-[[6-[[1-(Difluoromethyl)cyclopropyl]methoxy]-5-fluoro-2-methoxy-3-pyridyl]methyl]pyrrolidine-2-carboxamide; (2S,3S)-2,3-dihydroxybutanedioic Acid

(Compound of Formula Ia.1, Wherein X is CH, R⁵ is H, R⁶ is F, R⁷ is OCH₃ and R^{8a} is CH₂-A.6)

The title compound was prepared using the procedure described in example 2, starting from (1-(difluoromethyl)cyclopropyl)methanol (example 96) and (S)-tert-butyl 2-(((6-chloro-5-fluoro-2-methoxypyridin-3-yl)methyl)carbamoyl)pyrrolidine-1-carboxylate, followed by BOC deprotection. Finally the (2S,3S)-2,3-dihydroxybutanedioic acid salt was formed by adding one equivalent of the corresponding acid.

LCMS (ESI⁺) m/z [M+H]⁺: 374.35

¹H NMR (600 MHz, Methanol-d₄) δ ppm: 0.82 (dp, J=4.8, 2.4 Hz, 2H), 0.91-0.96 (m, 2H), 2.00 (dp, J=35.2, 7.3 Hz, 3H), 2.40 (dd, J=13.1, 7.0 Hz, 1H), 3.31-3.35 (m, 1H), 3.38 (dt, J=11.1, 7.0 Hz, 1H), 3.92 (s, 3H), 4.20-4.34 (m, 3H), 4.42 (s, 2H), 4.47 (d, J=1.3 Hz, 2H), 5.91 (t, J=57.4 Hz, 1H), 7.45 (d, J=10.0 Hz, 1H).

### Example 98

### (2S)—N-[[5-Fluoro-2-methoxy-6-[[1-(trifluoromethyl)cyclopropyl]methoxy]-3-pyridyl]methyl]-N,1-dimethyl-pyrrolidine-2-carboxamide Hydrochloride

(Compound of Formula Ia.2, Wherein X is CH, R⁵ is CH₃, R⁶ is F, R⁷ is OCH₃ and R^{8a} is CH₂-A.4)

The title compound was prepared using the procedure described in example 2 and 63 starting from (1-(trifluoromethyl)cyclopropyl)methanol and (S)—N-((6-chloro-5-fluoro-2-methoxypyridin-3-yl)methyl)-N,1-dimethylpyrrolidine-2-carboxamide, which was synthesized by N-methylation of (S)—N-((6-chloro-5-fluoro-2-methoxypyridin-3-yl)methyl)-1-methylpyrrolidine-2-carboxamide.

### 98.1 (S)—N-((6-chloro-5-fluoro-2-methoxypyridin-3-yl)methyl)-N,1-dimethylpyrrolidine-2-carboxamide

(S)—N-((6-Chloro-5-fluoro-2-methoxypyridin-3-yl)methyl)-1-methylpyrrolidine-2-carboxamide (1.5 g, 4.97 mmol) was dissolved in DMF (40 ml) at 0° C. under nitrogen to give a colorless solution. NaH (0.249 g, 6.21 mmol) was added slowly. The reaction mixture was stirred at 0° C. for 30 minutes. Iodomethane (0.389 ml, 6.21 mmol) was added. The reaction mixture was stirred at 0° C. for 1 hour. The reaction mixture was stirred at room temperature for 17 hours. The reaction mixture was cooled to 5° C. Water was added to the reaction mixture. The pH was adjusted alkaline with 2M NaOH. The aqueous layer was extracted three times with tert-butylmethylether, the combined organic layers were washed with saturated aqueous sodium chloride, dried over sodium sulphate, filtered and the solvent evaporated to obtain (S)—N-((6-chloro-5-fluoro-2-methoxypyridin-3-yl)methyl)-N,1-dimethylpyrrolidine-2-carboxamide (1.5 g) as yellow oil.

LCMS (ESI⁺) m/z [M+H]⁺: 316.2

Coupling of (1-(trifluoromethyl)cyclopropyl)methanol and (S)—N-((6-chloro-5-fluoro-2-methoxypyridin-3-yl)methyl)-N,1-dimethylpyrrolidine-2-carboxamide was performed according to example 2 and 63. Finally the hydrochloride salt was formed by adding one equivalent hydrochloric acid.

LCMS (ESI⁺) m/z [M+H]⁺: 420.3

¹H NMR (600 MHz, DMSO-d₆) δ ppm: δ 9.60 (s, 1H, A and B), 7.73 (d, J=10.2 Hz, 1H, A), 7.56 (d, J=10.2 Hz, 1H, B), 4.74-4.59 (m, 1H, A and B), 4.57 (s, 2H, A), 4.55 (s, 2H, B), 4.49-4.35 (m, 2H, B and 1H A), 4.27 (d, J=15.9 Hz, 1H, A), 3.89 (s, 3H, A), 3.87 (s, 3H, B), 3.67-3.54 (m, 1H, A and B), 3.20-3.05 (m, 1H, A and B), 2.94 (s, 3H, B), 2.83 (d, J=4.2 Hz, 3H, A), 2.81 (d, J=4.2 Hz, 3H, B), 2.78 (s, 3H, A), 2.66-2.48 (m, 1H. A and B), 2.14-2.02 (m, 1H, A and B), 1.94-1.73 (m, 2H, A and B), 1.14-1.01 (m, 4H, A and B); Rotamers ratio A:B 1:2.

### Example 99

### (2S)—N-[[6-(3,3-Difluorocyclobutoxy)-5-fluoro-2-methoxy-3-pyridyl]methyl]-N,1-dimethyl-pyrrolidine-2-carboxamide Hydrochloride

(Compound of Formula Ia.2, Wherein X is CH, R⁵ is CH₃, R⁶ is F, R⁷ is OCH₃ and R^{8a} is A.10)

The title compound was prepared using the procedure described in example 2, 63 and 98 starting from 3,3-difluorocyclobutanol and (S)—N-((6-chloro-5-fluoro-2-methoxypyridin-3-yl)methyl)-N,1-dimethylpyrrolidine-2-carboxamide. Finally the hydrochloride salt was formed by adding one equivalent hydrochloric acid.

LCMS (ESI⁺) m/z [M+H]⁺: 388.2

¹H NMR (500 MHz, DMSO-d₆) δ ppm: 9.59 (s, 1H, A and B), 7.74 (d, J=10.2 Hz, 1H, A), 7.57 (d, J=10.3 Hz, 1H, B), 5.22-5.07 (m, 1H, A and B), 4.73-4.56 (m, 1H, A and B), 4.53-4.37 (m, 2H, B and 1H, A), 4.27 (d, J=16.1 Hz, 1H, A), 3.89 (s, 3H, A), 3.86 (s, 3H, B), 3.65-3.53 (m, 1H, A and B), 3.28-3.07 (m, 3H, A and B), 2.94 (s, 3H, B), 2.88-2.74 (m, 5H, B and 8H, A), 2.66-2.48 (m, 1H, A and B), 2.15-2.02 (m, 1H, A and B), 1.94-1.74 (m, 2H, A and B). Rotamers: ratio A:B 1:2.

### Example 100

### (2S)—N-[[5-Fluoro-2-methoxy-6-(2,2,2-trifluoroethoxy)-3-pyridyl]methyl]-1-methyl-pyrrolidine-2-carboxamide Hydrochloride

(Compound of Formula Ia.2, Wherein X is CH, R⁵ is H, R⁶ is F, R⁷ is OCH₃ and R^{8a} is CH₂CF₃)

The title compound was prepared using the procedure described in example 63 starting from 2,2,2-trifluoroethanol and (S)—N-((6-chloro-5-fluoro-2-methoxypyridin-3-yl)methyl)-1-methylpyrrolidine-2-carboxamide. Finally the hydrochloride salt was formed by adding one equivalent hydrochloric acid.

LCMS (ESI⁺) m/z [M+H]⁺: 366.4

¹H NMR (500 MHz, DMSO-d₆) δ ppm: 9.70 (s, 1H), 9.13 (t, J=5.7 Hz, 1H), 7.70 (d, J=10.2 Hz, 1H), 5.13 (d, J=8.9 Hz, 1H, AB signal), 5.09 (d, J=9.0 Hz, 1H, AB signal), 4.25 (dd, J=5.5, 1.8 Hz, 2H), 4.11 (d, J=8.7 Hz, 1H), 3.91 (s, 3H), 3.60-3.52 (m, 1H), 3.18-3.12 (m, 1H), 2.82 (s, 3H), 2.49-2.43 (m, 1H), 2.12-2.01 (m, 1H), 1.95-1.81 (m, 2H).

### Example 101

### (2S)-2-(Azetidin-1-yl)-N-[[6-(3,3-difluorocyclobutoxy)-5-fluoro-2-methoxy-3-pyridyl]methyl]propanamide Hydrochloride

(Compound of Formula Ia.20, Wherein X is CH, R⁵ is H, R⁶ is F, R⁷ is OCH₃ and R^{8a} is A.10)

The title compound was prepared using the procedure described in example 1 starting from 3,3-difluorocyclobutanol, 2,5,6-trifluoronicotinonitrile, methanol and (S)-2-(azetidin-1-yl)propanoic acid.

### 101.1 (S)-Benzyl 2-(azetidin-1-yl)propanoate

(S)-Benzyl 2-aminopropanoate hydrochloride (1.0 g, 4.64 mmol) was dissolved in acetonitrile (35 ml) at room temperature under nitrogen to give a white suspension. Diisopropylethylamine (3.64 ml, 20.86 mmol) and 1,3-dibromopropane (0.518 ml, 5.10 mmol) were added. The reaction mixture gave a colorless solution. The reaction mixture was stirred at RT for 5 hours. Stirring was continued for 14 hours at room temperature. Since the reaction was not complete, additional diisopropylethylamine (1.14 ml) and 1,3-dibromopropane (0.162 ml) were added. Stirring was continued for 5 hours at reflux. Despite of still 15% of the starting material the reaction mixture was cooled to room temperature. Water was added to the reaction mixture. The pH was adjusted alkaline with 2M NaOH. The aqueous layer was extracted three times with ethyl acetate, the combined organic layers were washed with saturated aqueous sodium chloride, dried over sodium sulphate, filtered and the solvent evaporated. The crude product (1.47 g of a yellow oil) was purified by column chromatography on silica gel (24 g-column, dichloromethane/methanol, dichloromethane 100%→methanol 25%, 24 g-column, flow: 13 ml/min) to give (S)-benzyl 2-(azetidin-1-yl)propanoate (431 mg, yield: 44%) as pale yellow oil.

### 101.2 (S)-2-(Azetidin-1-yl)propanoic Acid

(S)-Benzyl 2-(azetidin-1-yl)propanoate (140 mg, 0.638 mmol) was dissolved in THF (13 ml) and hydrogenated in the H-cube at 50° C. and 70 bar H₂ by continuous circulation until no starting material could be detected any more. After evaporation of the solvent, the raw material of (S)-2-(azetidin-1-yl)propanoic acid (54 mg, yield 66%) was used in the next step without further purification.

Amide formation of (6-(3,3-difluorocyclobutoxy)-5-fluoro-2-methoxypyridin-3-yl)methanamine and ((S)-2-(azetidin-1-yl)propanoic acid was performed according to example 1 followed by BOC deprotection. Finally the hydrochloride salt was formed by adding one equivalent hydrochloric acid.

LCMS (ESI⁺) m/z [M+H]⁺: 374.3

¹H NMR (600 MHz, DMSO-d₆) δ ppm: 10.43 (br s, 1H), 8.92 (s, 1H), 7.58 (d, J=10.3 Hz, 1H), 5.18-5.08 (m, 1H), 4.23-4.13 (m, 2H), 4.12-3.88 (m, 5H), 3.86 (s, 3H), 3.26-3.13 (m, 2H), 2.81 (tdd, J=15.3, 13.8, 5.4 Hz, 2H), 2.35 (br s, 2H), 1.28 (d, J=6.9 Hz, 3H).

### Example 102

### (2S)—N-[[5-Fluoro-2-methoxy-6-(2,2,3,3-tetrafluoropropoxy)-3-pyridyl]methyl]-N,1-dimethyl-pyrrolidine-2-carboxamide Hydrochloride

(Compound of Formula Ia.2, Wherein X is CH, R⁵ is CH₃, R⁶ is F, R⁷ is OCH₃ and R^{8a} is CH₂CF₂CHF₂)

The title compound was prepared using the procedure described in example 2, 63 and 98 starting from 2,2,3,3-tetrafluoropropan-1-ol and (S)—N-((6-chloro-5-fluoro-2-methoxypyridin-3-yl)methyl)-N,1-dimethylpyrrolidine-2-carboxamide. Finally the hydrochloride salt was formed by adding one equivalent hydrochloric acid.

LCMS (ESI⁺) m/z [M+H]⁺: 412.2

¹H NMR (600 MHz, DMSO-d₆) δ ppm: 9.64 (br s, 1H, A), 9.60 (br s, 1H, B), 7.82 (d, J=10.0 Hz, 1H, A), 7.64 (d, J=10.1 Hz, 1H, B), 6.68 (q, J=50.8, 5.0 Hz, 1H, A and B), 5.05-4.91 (m, 2H, A and B), 4.71-4.59 (m, 1H, A and B), 4.53-4.39 (m, 2H, B and 1H, A), 4.30 (d, J=16.2 Hz, 1H, A), 3.92 (s, 3H, A), 3.90 (s, 3H, B), 3.65-3.55 (m, 1H, A and B), 3.21-3.07 (m, 1H, A and B), 2.95 (s, 3H, B), 2.83 (d, J=4.8 Hz, 3H, A), 2.81 (d, J=4.9 Hz, 3H, B), 2.79 (s, 3H, A), 2.63-2.47 (m, 1H, A and B), 2.15 2.02 (m, 1H, A and B), 1.94-1.74 (m, 2H, A and B). Rotamers: ratio A:B 1:2

### Example 103

### (2S)—N-[[5-Fluoro-2-methoxy-6-[(2,2,3,3-tetrafluorocyclobutyl)methoxy]-3-pyridyl]methyl]-N,1-dimethyl-pyrrolidine-2-carboxamide Hydrochloride

(Compound of Formula Ia.2, Wherein X is CH, R⁵ is CH₃, R⁶ is F, R⁷ is OCH₃ and R^{8a} is CH₂-A.11)

The title compound was prepared using the procedure described in example 2, 63 and 98 starting from (2,2,3,3-tetrafluorocyclobutyl)methanol and (S)—N-((6-chloro-5-fluoro-2-methoxypyridin-3-yl)methyl)-N,1-dimethylpyrrolidine-2-carboxamide. Finally the hydrochloride salt was formed by adding one equivalent hydrochloric acid.

LCMS (ESI⁺) m/z [M+H]⁺: 438.3

¹H NMR (500 MHz, DMSO-d₆) δ ppm: 9.59 (br s, 1H, A and B), 7.74 (d, J=10.1 Hz, 1H, A), 7.57 (dd, J=10.2, 1.3 Hz, 1H, B), 4.70-4.51 (m, 3H, A and B), 4.50-4.37 (m, 2H, B and 1H, A), 4.28 (d, J=16.0 Hz, 1H, A), 3.92 (s, 3H, A), 3.89 (s, 3H, B), 3.65-3.55 (m, 1H, A and B), 3.48-3.37 (m, 1H, A and B), 3.18-3.06 (m, 1H, A and B), 2.94 (s, 3H, B), 2.92-2.75 (m, 4H, B and 7H, A), 2.67-2.51 (m, 2H, A and B), 2.15-2.03 (m, 1H, A and B), 1.96-1.75 (m, 2H, A and B). Rotamers: ratio A:B 1:2

### Example 104

### (2S)—N-[[5-Fluoro-2-methoxy-6-(3-methoxycyclobutoxy)-3-pyridyl]methyl]-pyrrolidine-2-carboxamide Hydrochloride

(Compound of Formula Ia.1, Wherein X is CH, R⁵ is H, R⁶ is F, R⁷ is OCH₃ and R^{8a} is A.15)

The title compound was prepared using the procedure described in example 2, starting from cis-3-methoxycyclobutanol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

LCMS (ESI⁺) m/z [M+H]⁺: 354.3

¹H NMR (600 MHz, Methanol-d₄) δ: 7.37 (d, J=10.0 Hz, 1H), 4.88-4.81 (m, 2H), 4.62 (s, 0.4H impurity), 4.29-4.20 (m, 1H), 3.91 (s, 3H), 3.86-3.80 (m, 1H), 3.72 (p, J=6.9 Hz, 1H), 3.26 (s, 3H), 3.12 (dt, J=10.6, 6.4 Hz, 1H), 3.04 (dt, J=10.7, 6.6 Hz, 1H), 2.95-2.87 (m, 2H), 2.24-2.16 (m, 1H), 2.08-2.00 (m, 2H), 1.88-1.77 (m, 3H). Signal at 4.88-4.81 overlaps with water peak.

### Example 105

### (2S)—N-[[5-Fluoro-2-methoxy-6-[3-(trifluoromethyl)cyclobutoxy]-3-pyridyl]-methyl]pyrrolidine-2-carboxamide Hydrochloride

(Compound of Formula Ia.1, Wherein X is CH, R⁵ is H, R⁶ is F, R⁷ is OCH₃ and R^{8a} is A.13)

The title compound was prepared using the procedure described in example 2, starting from trans-3-(trifluoromethyl)cyclobutanol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

LCMS (ESI⁺) m/z [M+H]⁺: 392.2

¹H NMR (600 MHz, Methanol-d₄) δ: 7.42 (d, J=10.0 Hz, 1H), 5.33 (p, J=6.9 Hz, 1H), 4.62 (s, 1.4H impurity), 4.27 (d, J=1.6 Hz, 2H), 3.98 (t, J=7.5 Hz, 1H), 3.90 (s, 3H), 3.25-3.20 (m, 1H), 3.18-3.04 (m, 2H), 2.69 (ddt, J=11.6, 7.4, 3.9 Hz, 2H), 2.59-2.50 (m, 2H), 2.32-2.22 (m, 1H), 1.95-1.82 (m, 3H).

### Example 106

### (2S)—N-[[6-[(3,3-Difluorocyclobutyl)methoxy]-5-fluoro-2-methoxy-3-pyridyl]-methyl]-N,1-dimethyl-pyrrolidine-2-carboxamide Hydrochloride

(Compound of Formula Ia.2, Wherein X is CH, R⁵ is CH₃, R⁶ is F, R⁷ is OCH₃ and R^{8a} is CH₂-A.10)

The title compound was prepared using the procedure described in example 2, 63 and 98 starting from (3,3-difluorocyclobutyl)methanol and (S)—N-((6-chloro-5-fluoro-2-methoxypyridin-3-yl)methyl)-N,1-dimethylpyrrolidine-2-carboxamide. Finally the hydrochloride salt was formed by adding one equivalent hydrochloric acid.

LCMS (ESI⁺) m/z [M+H]⁺: 402.4

¹H NMR (500 MHz, DMSO-d₆) δ: 9.59 (s, 1H, A and B),7.71 (d, J=10.2 Hz, 1H, A), 7.54 (d, J=10.4 Hz, 1H, B), 4.64 (dq, J=24.9, 8.2 Hz, 1H, A and B), 4.52-4.36 (m, 4H, B and 3H, A), 4.26 (d, J=16.0 Hz, 1H, A), 3.90 (s, 3H, A), 3.88 (s, 3H, B), 3.65 3.55 (m, 1H, A and B), 3.18-3.06 (m, 1H, A and B), 2.94 (s, 3H, B), 2.83 (d, J=4.6 Hz, 3H, A), 2.81 (d, J=4.6 Hz, 3H, B), 2.78 (s, 3H, A), 2.76-2.67 (m, 2H, A and B), 2.67-2.54 (m, 2H, A and B), 2.49-2.40 (m, 2H, A and B; overlap with DMSO peak), 2.16-2.04 (m, 1H, A and B), 1.94-1.75 (m, 2H, A and B). Rotamers: ratio A:B 1:2

### Example 107

### (2S)—N-[[5-(3,3-Difluorocyclobutoxy)-3-methoxy-pyrazin-2-yl]methyl]pyrrolidine-2-carboxamide; 2,2,2-trifluoroacetic Acid

(Compound of Formula Ia.1, Wherein X is N, R⁵ is H, R⁶ is H, R⁷ is OCH₃ and R^{8a} is A.10)

The title compound was prepared using the procedure described in scheme 2 and 7 starting from 3,3-difluorocyclobutanol, 3,5-difluoropyrazine-2-carbonitrile and methanol forming 5-(3,3-difluorocyclobutoxy)-3-methoxypyrazine-2-carbonitrile. The reduction of the nitrile function was performed with BH₃ according to example 5.

### 107.1 3-Chloro-5-(3,3-difluorocyclobutoxy)pyrazine-2-carbonitrile

### (Scheme 8 Step 1 First Part)

Potassium tert-butoxide (0.658 g, 5.86 mmol) and tetrahydrofuran (8 ml) were mixed to give a colorless suspension. The reaction mixture was cooled to 0° C. and 3,3-difluorocyclobutanol (0,683 g, 6.32 mmol) diluted in tetrahydrofuran (3 ml) were added by a syringe and the reaction mixture was stirred for 30 min at 0° C. while the color changed to orange. Then, the reaction mixture was cooled to −78° C. and a solution of 3,5-dichloropyrazine-2-carbonitrile (1 g, 5.75 mmol) in tetrahydrofuran (4 ml) was added over 10 min by syringe. The cooling bath was removed and the reaction mixture was stirred for 2 h at 25° C. The reaction mixture was diluted with ethyl acetate and 1M sodium hydroxide was added. The aqueous layer was extracted with ethyl acetate (3×). The organic layer was dried over sodium sulfate and concentrated. The residue was purified by flash chromatography to give 3-chloro-5-(3,3-difluorocyclobutoxy)pyrazine-2-carbonitrile (1.11 g, 4.54 mmol).

LCMS (ESI⁺) m/z [M+H]⁺: 246

### 107.2 5-(3,3-Difluorocyclobutoxy)-3-methoxypyrazine-2-carbonitrile

### (Scheme 8 Step 1 Second Part)

A suspension of potassium tert-butoxide (0.713 g, 6.35 mmol) in tetrahydrofuran (2 ml) was cooled to 0° C. Then, methanol (0.26 ml, 6.35 mmol) was added via syringe to the reaction mixture and stirred for 30 minutes at 0° C. The mixture was cooled to −40° C. and a solution of 3-chloro-5-(3,3-difluorocyclobutoxy)pyrazine-2-carbonitrile (1.11 g, 4.54 mmol) in tetrahydrofuran (10 ml) was added over 10 minutes via syringe to the reaction mixture. After 1 h the cooling bath was removed and the reaction mixture allowed to warm to room temperature and stirred for additional 2 hours. The reaction mixture was diluted with water and extracted with ethyl acetate (5×). The organic phase was dried over sodium sulfate and concentrated. The residue was absorbed on bulk isolute sorbent and purified by flash chromatography to give 5-(3,3-difluorocyclobutoxy)-3-methoxypyrazine-2-carbonitrile (0.68 mg, 1.41 mmol).

LCMS (ESI⁺) m/z [M+H]⁺: 242

### 107.3 (5-(3,3-Difluorocyclobutoxy)-3-methoxypyrazin-2-yl)methanamine

### (Scheme 8 Step 2)

5-(3,3-Difluorocyclobutoxy)-3-methoxypyrazine-2-carbonitrile (678 mg, 2.81 mmol) and borane dimethyl sulfide (1406 μl, 2.81 mmol) complex were added to a microwave vial and the mixture was stirred until gas evolution stopped. The reaction mixture was heated in the microwave for 30 minutes at 90° C. Additional borane dimethyl sulfide (4217 μl, 8.43 mmol) was added and the mixture was heated in the microwave at 90° C. for another 30 minutes. The reaction mixture was slowly added to a diluted aqueous solution of hydrogen chloride and stirred for 1 h. Sodium hydroxide solution was added to the reaction mixture until pH 9. The aqueous layer was extracted with methylenedichloride (5×). The organic phase was dried over sodium sulfate and concentrated to give (5-(3,3-difluorocyclobutoxy)-3-methoxypyrazin-2-yl)methanamine (90 mg, 0.19 mmol).

LCMS (ESI⁺) m/z [M+H]⁺: 246

Peptide coupling of (5-(3,3-difluorocyclobutoxy)-3-methoxypyrazin-2-yl)methanamine with (S)-1-(tert-butoxycarbonyl)pyrrolidine-2-carboxylic acid, followed by BOC deprotection, was performed according to example 1. The trifluoroacetate salt was obtained by HPLC chromatography on a reversed phase column.

LCMS (ESI⁺) m/z [M+H]⁺: 343

¹H NMR (600 MHz, DMSO-d₆) δ ppm: 9.20 (s, 1H), 8.87 (t, J=5.6 Hz, 1H), 8.56 (s, 1H), 7.90-7.76 (m, 1H), 5.23-5.00 (m, 1H), 4.53-4.28 (m, 2H), 4.18 (ddd, J=10.7, 6.4, 4.1 Hz, 1H), 4.02-3.85 (m, 5H), 3.29-3.10 (m, 2H), 2.90-2.71 (m, 2H), 2.33-2.22 (m, 1H), 1.99-1.76 (m, 3H).

### Example 108

### (2S)—N-[[5-(4,4-Difluorocyclohexoxy)-3-methoxy-pyrazin-2-yl]methyl]pyrrolidine-2-carboxamide; 2,2,2-trifluoroacetic Acid

(Compound of Formula Ia.1, Wherein X is N, R⁵ is H, R⁶ is H, R⁷ is OCH₃ and R^{8a} is A.33)

The title compound was prepared using the procedure described in scheme 2 and 7 and example 107 starting from 4,4-difluorocyclohexanol, 3,5-difluoropyrazine-2-carbonitrile, methanol and (S)-1-(tert-butoxycarbonyl)pyrrolidine-2-carboxylic acid, followed by BOC deprotection. The trifluoroacetate salt was obtained by HPLC chromatography on a reversed phase column.

LCMS (ESI⁺) m/z [M+H]⁺:371.2

¹H NMR (500 MHz, Methanol-d₄) δ: 7.69 (s, 1H), 5.26-5.18 (m, 1H), 4.51 (d, J=15.9 Hz, 1H), 4.46 (d, J=15.8 Hz, 1H), 4.29 (dd, J=8.5, 6.5 Hz, 1H), 3.99 (s, 3H), 3.40 (td, J=7.0, 3.4 Hz, 1H), 3.35-3.31 (m, 1H), 2.48-2.35 (m, 1H), 2.21-1.89 (m, 11H).9.20

### Example 109

### (2S)—N-[[5-Fluoro-2-methoxy-6-[3,3,3-trifluoro-2-methyl-2-(trifluoromethyl)propoxy]-3-pyridyl]methyl]pyrrolidine-2-carboxamide Hydrochloride

(Compound of Formula Ia.1, Wherein X is CH, R⁵ is H, R⁶ is F, R⁷ is OCH₃ and R^{8a} is CH₂C(CH₃)(CF₃)₂)

The title compound was prepared using the procedure described in example 2, starting from 3,3,3-trifluoro-2-methyl-2-(trifluoromethyl)propan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

LCMS (ESI⁺) m/z [M+H]⁺: 392.2

¹H NMR (500 MHz, Methanol-d₄) δ: 7.48 (d, J=9.8 Hz, 1H), 4.78 (s, 2H), 4.30 (s, 2H), 4.07 (dd, J=8.5, 6.4 Hz, 1H), 3.95 (s, 3H), 3.30-3.24 (m, 1H), 3.20 (dt, J=11.3, 6.9 Hz, 1H), 2.40-2.28 (m, 1H), 2.05-1.86 (m, 3H), 1.54 (s, 3H).

### Example 110

### (2S)—N-[[5-Fluoro-6-(2,2,3,3,4,4,4-heptafluorobutoxy)-2-methoxy-3-pyridyl]-methyl]pyrrolidine-2-carboxamide Hydrochloride

(Compound of Formula Ia.1, Wherein X is CH, R⁵ is H, R⁶ is F, R⁷ is OCH₃ and R^{8a} is CH₂CF₂CF₂CF₃)

The title compound was prepared using the procedure described in example 2, starting from 2,2,3,3,4,4,4-heptafluorobutan-1-ol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

LCMS (ESI⁺) m/z [M+H]⁺: 452.20

¹H NMR (500 MHz, Methanol-d₄) δ: 7.53 (d, J=9.8 Hz, 1H), 5.09 (tq, J=13.6, 1.7 Hz, 2H), 4.31 (s, 2H), 4.13 (dd, J=8.5, 6.6 Hz, 1H), 3.96 (s, 3H), 3.36-3.27 (m, 1H, overlap with water peak), 3.24 (dt, J=11.4, 7.0 Hz, 1H), 2.40-2.30 (m, 1H), 2.04-1.89 (m, 3H).

### Example 111

### (2S)—N-[[5-Fluoro-2-methoxy-6-[2,2,2-trifluoro-1-(trifluoromethyl)ethoxy]-3-pyridyl]methyl]pyrrolidine-2-carboxamide Hydrochloride

(Compound of Formula Ia.1, Wherein X is CH, R⁵ is H, R⁶ is F, R⁷ is OCH₃ and R^{8a} is CH(CF₃)₂)

The title compound was prepared using the procedure described in example 2, starting from 1,1,1,3,3,3-hexafluoropropan-2-ol and (S)-tert-butyl 2-(((6-chloro-5-fluoro-2-methoxypyridin-3-yl)methyl)carbamoyl)pyrrolidine-1-carboxylate, followed by BOC deprotection. Finally the hydrochloride salt was formed by adding one equivalent hydrochloric acid.

Instead of [1,1′-biphenyl]-2-yldiisopropylphosphine and Pd(OAc)₂ as catalyst, dicyclohexyl(2′,6′-diisopropoxy-[1,1′-biphenyl]-2-yl)phosphine/RuPhos and tris[dibenzylidenacetone)dipalladium(0) were used as catalyst.

LCMS (ESI⁺) m/z [M+H]⁺: 420.1

¹H NMR (500 MHz, Methanol-d₄) δ: 6 7.61 (d, J=9.6 Hz, 1H), 6.64 (h, J=6.1 Hz, 1H), 4.32 (s, 2H), 4.02 (dd, J=8.5, 6.4 Hz, 1H), 3.97 (s, 3H), 3.23 (dt, J=10.9, 6.8 Hz, 1H), 3.16 (dt, J=11.0, 6.8 Hz, 1H), 2.33-2.25 (m, 1H), 1.98-1.84 (m, 3H).

### Example 112

### (2S)—N-[[5-Fluoro-2-methoxy-6-(2,2,3,3-tetrafluorocyclobutoxy)-3-pyridyl]methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.11)

The title compound was prepared using the procedure described in example 2, starting from 2,2,3,3-tetrafluorocyclobutanol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

Instead of [1,1′-biphenyl]-2-yldiisopropylphosphine and Pd(OAc)₂ as catalyst, dicyclohexyl(2′,6′-diisopropoxy-[1,1′-biphenyl]-2-yl)phosphine/RuPhos and tris[dibenzylidenacetone)dipalladium(0) were used as catalyst.

LCMS (ESI⁺) m/z [M+H]⁺: 396.1

¹H NMR (600 MHz, Methanol-d₄) δ: 7.54 (dd, J=9.9, 4.5 Hz, 1H), 5.58-5.47 (m, 1H), 4.35-4.28 (m, 2H), 4.22 (ddd, J=8.2, 7.0, 2.7 Hz, 1H), 3.95 (d, J=0.9 Hz, 3H), 3.43-3.37 (m, 1H; under water peak), 3.34-3.25 (m, 1H), 3.24-3.12 (m, 1H), 2.88-2.76 (m, 1H), 2.44-2.37 (m, 1H), 2.08-2.02 (m, 2H), 2.02-1.94 (m, 1H).

### Example 113

### (2S)—N-[[5-Fluoro-2-methoxy-6-(2,2,2-trifluoroethoxy)-3-pyridyl]methyl]-N,1-dimethyl-pyrrolidine-2-carboxamide Hydrochloride

(Compound of Formula Ia.2, Wherein X is CH, R⁵ is CH₃, R⁶ is F, R⁷ is OCH₃ and R^{8a} is CH₂CF₃)

The title compound was prepared using the procedure described in example 2, 63 and 98 starting from 2,2,2-trifluoroethanol and (S)—N-((6-chloro-5-fluoro-2-methoxypyridin-3-yl)methyl)-N,1-dimethylpyrrolidine-2-carboxamide. Finally the hydrochloride salt was formed by adding one equivalent hydrochloric acid.

LCMS (ESI⁺) m/z [M+H]⁺: 380.3

¹H NMR (600 MHz, DMSO-d₆) δ ppm: 9.64 (d, J=27.2 Hz, 1H, A and B), 7.84 (d, J=10.0 Hz, 1H, A), 7.66 (d, J=10.1 Hz, 1H, B), 5.17-5.07 (m, 2H, A and B), 4.71-4.62 (m, 1H, A and B), 4.51-4.41 (m, 2H, B and 1H, A), 4.31 (d, J=16.1 Hz, 1H, A), 3.93 (s, 3H, A), 3.90 (s, 3H, B), 3.64-3.56 (m, 1H, A and B), 3.18-3.09 (m, 1H, A and B), 2.96 (s, 3H, B), 2.83 (d, J=4.7 Hz, 3H, A), 2.81 (d, J=4.8 Hz, 3H, B), 2.79 (s, 3H, A), 2.63-2.55 (m, 1H, A and B), 2.14-2.05 (m, 1H, A and B), 1.93-1.77 (m, 2H, A and B). Rotamers: A and B

### Example 114

### (2S)—N-[[6-(4,4-Difluorocyclohexoxy)-5-fluoro-2-methoxy-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is A.33)

The title compound was prepared using the procedure described in example 2, starting from 4,4 difluorocyclohexanol and (S)-tert-butyl 2-(((6-chloro-5-fluoro-2-methoxypyridin-3-yl)methyl)(methyl)carbamoyl)pyrrolidine-1-carboxylate

### 114.1 tert-Butyl ((6-chloro-5-fluoro-2-methoxypyridin-3-yl)methyl)carbamate

A solution of (6-chloro-5-fluoro-2-methoxypyridin-3-yl)methanamine hydrochloride (2.5 g, 11 mmol; example 2 and scheme 10, step 3) in tetrahydrofuran (25 mL) was adjusted to pH=9 with aqueous Na₂CO₃ (3 mL). Di-tert-butyl dicarbonate (3.83 mL, 16.5 mmol) was added at 0° C. and the mixture was stirred at 25° C. for 2 h. The mixture was diluted with water (20 mL) and extracted with ethyl acetate (3×20 mL). The organic layer was washed with brine (10 mL), dried over Na₂SO₄, filtered and concentrated under reduced pressure. The residue was purified by column chromatography on silica gel (eluted with petroleum ether/ethyl acetate=30/1) to obtain tert-butyl ((6-chloro-5-fluoro-2-methoxypyridin-3-yl)methyl)carbamate (3.1 g, 10.7 mmol, 97% yield) as a white solid.

¹H NMR (400 MHz, DMSO-d₆) δ ppm: 7.58 (d, J=8.4 Hz, 1H), 7.36 (br. s., 1H), 4.05 (d, J=5.7 Hz, 2H), 3.88 (s, 3H), 1.45-1.31 (m, 9H)

### 114.2 tert-Butyl ((6-chloro-5-fluoro-2-methoxypyridin-3-yl)methyl)(methyl)carbamate

To a solution of tert-butyl ((6-chloro-5-fluoro-2-methoxypyridin-3-yl)methyl)carbamate (3.0 g, 10.3 mmol) in tetrahydrofuran (30 mL) was added NaH (0.83 g, 20.6 mmol) at 0° C. After stirring at 0° C. for 0.5 h, iodomethane (2.2 g, 15.5 mmol) was added and the mixture was stirred at 25° C. for 12 h. The mixture was quenched with aqueous NH₄Cl (30 mL) and extracted with ethyl acetate (3×20 mL). The organic layer was washed with brine (20 mL), dried over Na₂SO₄, filtered and concentrated under reduced pressure. The residue was purified by column chromatography (eluted with petroleum ether/ethyl acetate=30/1) to obtain tert-butyl ((6-chloro-5-fluoro-2-methoxypyridin-3-yl)methyl)(methyl)carbamate (2.5 g, 14.2 mmol) as a yellow oil.

¹H NMR (400 MHz, DMSO-d₆) δ ppm: 7.52 (d, J=10.1 Hz, 1H), 4.28 (s, 2H), 3.88 (s, 3H), 2.87-2.78 (m, 3H), 1.47-1.26 (m, 9H)

### 114.3 1-(6-Chloro-5-fluoro-2-methoxypyridin-3-yl)-N-methylmethanamine

To a solution of tert-butyl ((6-chloro-5-fluoro-2-methoxypyridin-3-yl)methyl)(methyl)carbamate (2.5 g, 8.2 mmol) in ethyl acetate (25 mL) was added HCl (g)/ethyl acetate (4M, 10 mL) at 0° C. After the addition, the mixture was stirred at 25° C. for 4 h. The solvent was removed under reduced pressure to obtain 1-(6-chloro-5-fluoro-2-methoxypyridin-3-yl)-N-methylmethanamine hydrochloride (1.77 g, 8.6 mmol, 89% yield) as a white solid.

¹H NMR (400 MHz, DMSO-d₆) δ 9.48 (br. s., 2H), 8.20 (d, J=8.4 Hz, 1H), 4.06 (s, 2H), 3.91 (s, 3H), 2.54 (s, 3H)

(S)-tert-butyl 2-(((6-chloro-5-fluoro-2-methoxypyridin-3-yl)methyl)(methyl)carbamoyl)pyrrolidine-1-carboxylate was prepared as described in example 2 and in scheme 5 step 1 by peptide coupling of (S)-1-(tert-butoxycarbonyl)pyrrolidine-2-carboxylic acid with -(6-chloro-5-fluoro-2-methoxypyridin-3-yl)-N-methylmethanamine. The subsequent Pd-catalyzed coupling reaction of 4,4 difluorocyclohexanol with (S)-tert-butyl 2-(((6-chloro-5-fluoro-2-methoxypyridin-3-yl)methyl)(methyl)carbamoyl)pyrrolidine-1-carboxylate was performed using the procedure described in example 2, which was followed by the BOC deprotection. Finally the hydrochloride salt was formed by adding one equivalent hydrochloric acid.

LCMS (ESI⁺) m/z [M+H]⁺: 402.3

¹H NMR (500 MHz, Methanol-d₄) δ ppm: 7.47 (d, J=9.9 Hz, 1H, A), 7.41 (d, J=9.8 Hz, 1H, B), 5.32-5.24 (m, 1H, A and B), 4.74 (dd, J=8.7, 7.0 Hz, 1H, A), 4.64 (d, J=15.9 Hz, 1H, A, AB signal), 4.57 (dd, J=8.8, 7.1 Hz, 1H, B), 4.53 (d, J=14.8 Hz, 1H, B, AB signal), 4.46 (d, J=14.9 Hz, 1H, B, AB signal), 4.29 (d, J=15.8 Hz, 1H, A, AB signal), 3.94 (s, 3H, A), 3.91 (s, 3H, B), 3.45-3.37 (m, 1H, A and B), 3.29-3.22 (m, 1H, A and B), 3.05 (s, 3H, B), 2.86 (s, 3H, A), 2.49 (dtd, J=13.5, 8.4, 6.4 Hz, 1H, A and B), 2.19-1.83 (m, 11H, A and B). Rotamers: ratio A:B 1:2

### Example 115

### (2S)—N-[[6-[(3,3-Difluorocyclobutyl)methoxy]-5-fluoro-2-methoxy-3-pyridyl]-methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is CH₂-A.10)

The title compound was prepared using the procedure described in example 2 and 114, starting from (3,3-difluorocyclobutyl)methanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 388.3

¹H NMR (500 MHz, Methanol-d₄) δ ppm: 7.46 (d, J=9.8 Hz, 1H, A), 7.41 (d, J=10.1 Hz, 1H, B), 4.73 (dd, J=8.7, 7.0 Hz, 1H, A), 4.64 (d, J=15.9 Hz, 1H, A, AB signal), 4.58-4.43 (m, 5H, B and 2H, A), 4.29 (d, J=15.9 Hz, 1H, A, AB signal), 3.96 (s, 3H, A), 3.93 (s, 3H, B), 3.46-3.34 (m, 1H, A and B), 3.29-3.20 (m, 1H, A and B), 3.05 (s, 3H, B), 2.85 (s, 3H, A), 2.76-2.59 (m, 3H, A and B), 2.53-2.41 (m, 3H, A and B), 2.10-1.83 (m, 3H, A and B). Rotamers: ratio A:B 1:2

### Example 116

### (2S)—N-[[5-Fluoro-2-methoxy-6-[trans-4-(trifluoromethyl)cyclohexoxy]-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is A.36)

The title compound was prepared using the procedure described in example 2 and 114, starting from trans-4-(trifluoromethyl)cyclohexanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 434.3

¹H NMR (500 MHz, DMSO-d₆) δ ppm: 9.79 (br s, 1H, A), 9.58 (br s, 1H, B), 8.49 (br s, 1H, A and B), 7.65 (d, J=10.3 Hz, 1H, A), 7.47 (d, J=10.3 Hz, 1H, B), 5.01-4.91 (m, 1H, A and B), 4.74-4.66 (m, 1H, A), 4.65-4.59 (m, 1H, B), 4.57 (d, J=15.8 Hz, 1H, A, AB signal), 4.45-4.34 (m, 2H, B), 4.26 (d, J=16.0 Hz, 1H, A, AB signal), 3.90 (s, 3H, A), 3.87 (s, 3H, B), 3.29-3.22 (m, 1H, A and B), 3.22-3.13 (m, 1H, A and B), 2.99 (s, 3H, B), 2.76 (s, 3H, A), 2.45-2.32 (m, 2H, A and B), 2.25-2.18 (m, 2H, A and B), 1.99-1.82 (m, 4H, A and B), 1.82-1.73 (m, 1H, A and B), 1.60-1.41 (m, 4H, A and B). Rotamers: ratio A:B 1:2

### Example 117

### (2S)—N-[[5-Fluoro-6-(2,2,3,3,4,4,4-heptafluorobutoxy)-2-methoxy-3-pyridyl]-methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is CH₂CF₂CF₂CF₃)

The title compound was prepared using the procedure described in example 2 and 114, starting from 2,2,3,3,4,4,4-heptafluorobutan-1-ol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 466.2

¹H NMR (500 MHz, DMSO-d₆) δ ppm: 9.63 (br s, 1H, A and B), 8.56 (br s, 1H, A and B), 7.78 (d, J=10.1 Hz, 1H, A), 7.61 (d, J=10.1 Hz, 1H, B), 5.31-5.19 (m, 2H, A and B), 4.68 (t, J=8.0 Hz, 1H, A), 4.66-4.57 (m, 1H, A and 1H, B), 4.45 (d, J=15.5 Hz, 1H, B, AB signal), 4.39 (d, J=15.4 Hz, 1H, B, AB signal), 4.31 (d, J=16.0 Hz, 1H, A, AB signal), 3.94 (s, 3H, A), 3.91 (s, 3H, B), 3.30-3.21 (m, 1H, A and B), 3.21-3.13 (m, 1H, A and B), 3.01 (s, 3H, B), 2.77 (s, 3H, A), 2.44-2.36 (m, 1H, A and B), 2.00 1.85 (m, 2H, A and B), 1.85-1.74 (m, 1H, A and B). Rotamers: ratio A:B 1:2

### Example 118

### (2S)—N-[[6-(2,2-Difluorospiro[2.5]octan-6-yl)oxy-5-fluoro-2-methoxy-3-pyridyl]methyl]pyrrolidine-2-carboxamide; 2,2,2-trifluoroacetic Acid

(Compound of Formula Ia.1, Wherein X is CH, R⁵ is H, R⁶ is F, R⁷ is OCH₃ and R^{8a} is A.85)

The title compound was prepared using the procedure described in example 2, starting from 1,1-difluorospiro[2.5]octan-6-ol and (S)-tert-butyl 2-(((6-chloro-5-fluoro-2-methoxypyridin-3-yl)methyl)carbamoyl)pyrrolidine-1-carboxylate, followed by BOC deprotection. The trifluoroacetate salt was obtained by HPLC chromatography on a reversed phase column.

LCMS (ESI⁺) m/z [M+H]⁺: 414.3

### Example 119

### (2S)—N-[[5-Fluoro-2-methoxy-6-[cis-4-(trifluoromethyl)cyclohexoxy]-3-pyridyl]-methyl]-N-methyl-pyrrolidine-2-carboxamide hydrochloride

(Compound of Formula Ia.1, Wherein X is CH, R⁵ is CH₃, R⁶ is F, R⁷ is OCH₃ and R^{8a} is A.36)

The title compound was prepared using the procedure described in example 2 and 114, starting from cis-4-(trifluoromethyl)cyclohexanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 434.3

¹H NMR (500 MHz, DMSO-d₆) δ ppm: δ 9.98-9.92 (m, 1H, A and B), 9.74-9.67 (m, 1H, C and D), 8.53-8.46 (m, 1H, A, B, C and D), 7.67 (d, J=10.2 Hz, 1H, A), 7.67 (d, J=10.2 Hz, 1H, B), 7.49 (d, J=10.2 Hz, 1H, C), 7.48 (d, J=10.3 Hz, 1H, D), 5.31 5.27 (m, 1H, A and C), 4.99-4.91 (m, 1H, B and D), 4.73-4.67 (m, 1H, A and B), 4.66-4.59 (m, 1H, C and D), 4.58 (d, J=15.7 Hz, 1H, B, AB signal), 4.57 (d, J=15.9 Hz, 1H, A, AB signal), 4.42 (d, J=15.3 Hz, 1H, C, AB signal), 4.41 (d, J=15.3 Hz, 1H, D, AB signal), 4.37 (d, J=15.2 Hz, 1H, D, AB signal), 4.37 (d, J=15.2 Hz, 1H, C, AB signal), 4.26 (d, J=16.0 Hz, 1H, A, AB signal), 4.25 (d, J=15.9 Hz, 1H, B, AB signal), 3.90 (s, 3H, B), 3.88 (s, 3H, A), 3.87 (s, 3H, D), 3.85 (s, 3H, C), 3.31-3.21 (m, 1H, A, B, C and D), 3.21-3.15 (m, 1H, A, B, C and D), 2.99 (s, 3H, C), 2.99 (s, 3H, D), 2.76 (s, 3H, A), 2.75 (s, 3H, B), 2.45-2.37 (m, 2H, A, B, C and D), 2.24-2.19 (m, 2H, B and D), 2.12-2.05 (m, 2H, A and C), 1.98-1.67 (m, 6H, A, B, C and D), 1.62 1.53 (m, 2H, A, B, C and D), 1.52-1.43 (m, 1H, A, B, C and D). Extremely complex NMR: 2 Rotamers due to N-Me (1:2) and 2 conformations (1:3) of 6-ring A:B:C:D ratio 3:1:6:2.

### Example 120

### (2S)—N-[[5-Fluoro-2-methoxy-6-[trans-3-(trifluoromethyl)cyclobutoxy]-3-pyridyl]-methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is A.13)

The title compound was prepared using the procedure described in example 2 and 114, starting from 3-(trifluoromethyl)cyclobutanol and (S)-tert-butyl 2-(((6-chloro-5-fluoro-2-methoxypyridin-3-yl)methyl)(methyl)carbamoyl)pyrrolidine-1-carboxylate. The palladium coupling was performed under slightly modified conditions:

### 120.1 (S)-tert-Butyl 2-(((5-fluoro-2-methoxy-6-(3-(trifluoromethyl)cyclobutoxy)pyridin-3-yl)methyl)(methyl)carbamoyl)pyrrolidine-1-carboxylate

3-(Trifluoromethyl)cyclobutanol (139 mg, 0.995 mmol) was dissolved under nitrogen in 2 mL of toluene; sodium hydride (51.8 mg, 1.294 mmol) was added and the mixture stirred at 50-80° C. for 15 min. Then (S)-tert-butyl 2-(((6-chloro-5-fluoro-2-methoxypyridin-3-yl)methyl)(methyl)carbamoyl)pyrrolidine-1-carboxylate (200 mg, 0.498 mmol), 2,2′-bis(diphenylphosphino)-1,1′-binaphthalene/BINAP (20.45 mg, 0.033 mmol) and tris(dibenzylideneacetone)dipalladium(0) (15.04 mg, 0.016 mmol) were added as solids at once. The reaction mixture was diluted with 3 mL toluene and stirred at 100° C. for 8 h. Water was added and the mixture was extracted with ethyl acetate. The aqueous layer was twice extracted with ethyl acetate. The ethyl acetate layer was dried with sodium sulfate, filtered and evaporated. The residue was purified by column chromatography on silica gel (0-70% ethylacetate in n-heptane) to obtain the two diasteromers (S)-tert-butyl 2-(((5-fluoro-2-methoxy-6-(trans-3-(trifluoromethyl)cyclobutoxy)pyridin-3-yl)methyl)(methyl)carbamoyl)pyrrolidine-1-carboxylate (77 mg, 28% yield; example 120) and (S)-tert-butyl 2-(((5-fluoro-2-methoxy-6-((cis-3-(trifluoromethyl)cyclobutoxy)pyridin-3-yl)methyl)(methyl)carbamoyl)pyrrolidine-1-carboxylate (63 mg, 20% yield; example 121) as a pale yellow oil.

After BOC deprotection the hydrochloride salt was formed by adding one equivalent hydrochloric acid.

LCMS (ESI⁺) m/z [M+H]⁺: 406.2

¹H NMR (500 MHz, Methanol-d₄) δ ppm: 7.45 (d, J=9.8 Hz, 1H, A), 7.40 (d, J=10.0 Hz, 1H, B), 5.34 (p, J=7.1 Hz, 1H, A and B), 4.63 (d, J=16.1 Hz, 1H, A, AB signal), 4.60-4.39 (m, 2H, B and 1H, A and B), 4.29 (d, J=16.0 Hz, 1H, A, AB signal), 3.92 (s, 3H, A), 3.90 (s, 3H, B), 3.39-3.33 (m, 1H, A and B; overlap with water peak), 3.20-3.06 (m, 2H, A and B), 3.04 (s, 3H, B), 2.85 (s, 3H, A), 2.70 (ddt, J=11.7, 7.7, 4.1 Hz, 2H, A and B), 2.61-2.50 (m, 2H, A and B), 2.47-2.35 (m, 1H, A and B), 2.04 1.78 (m, 3H, A and B). 2 Rotamers A:B=1:2.

### Example 121

### (2S)—N-[[5-Fluoro-2-methoxy-6-[cis-3-(trifluoromethyl)cyclobutoxy]-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is A.13)

The title compound was obtained as second diasteromer from column chromatography of (S)-tert-butyl 2-(((5-fluoro-2-methoxy-6-(3-(trifluoromethyl)cyclobutoxy)pyridin-3-yl)methyl)(methyl)carbamoyl)pyrrolidine-1-carboxylate (example 120), followed by BOC deprotection and hydrochloride formation by adding one equivalent hydrochloric acid.

LCMS (ESI⁺) m/z [M+H]⁺: 406.2

¹H NMR (500 MHz, Methanol-d₄) δ ppm: 7.42 (d, J=9.8 Hz, 1H, A), 7.38 (d, J=10.1 Hz, 1H, B), 5.18 (p, J=7.4 Hz, 1H, A and B), 4.63 (d, J=16.2 Hz, 1H, A, AB signal), 4.59-4.40 (m, 2H, B and 1H, A), 4.34-4.26 (m, 1H, A and B), 3.93 (s, 3H, A), 3.91 (s, 3H, B), 3.35-3.24 (m, 1H, A and B; overlap with water peak), 3.09-3.01 (m, 3H, B and 1H, A and B), 2.92-2.81 (m, 3H, A and 1H, A and B), 2.80-2.71 (m, 2H, A and B), 2.41-2.24 (m, 3H, A and B), 2.01-1.73 (m, 3H, A and B). Rotamers A:B=1:2.

### Example 122

### (2S)—N-[[6-[(3,5-Difluorophenyl)methoxy]-5-fluoro-2-methoxy-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is —CH₂-A.63)

The title compound was prepared using the procedure described in example 2, 114 and 120, starting from (3,5-difluorophenyl)methanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 410.20

¹H NMR (500 MHz, Methanol-d₄) δ ppm: 7.46 (d, J=9.8 Hz, 1H, A), 7.42 (d, J=9.8 Hz, 1H, B), 7.10-7.02 (m, 2H, A and B), 6.88 (tq, J=9.1, 2.4 Hz, 1H, A and B), 5.48 (s, 2H, A), 5.46 (s, 2H, B), 4.63 (d, J=16.1 Hz, 1H, A, AB signal), 4.54-4.47 (m, 1H, A and 1H, B), 4.45 (d, J=15.0 Hz, 1H, B, AB signal), 4.35 (dd, J=8.7, 7.0 Hz, 1H, B), 4.30 (d, J=16.1 Hz, 1H, A, AB signal), 3.90 (s, 3H, A), 3.88 (s, 3H, B), 3.35-3.28 (m, 1H, A and B), 3.09 (dt, J=11.1, 7.2 Hz, 1H, A and B), 3.04 (s, 3H, B), 2.86 (s, 3H, A), 2.44-2.28 (m, 1H, A and B), 2.04-1.74 (m, 3H, A and B). Rotamers A:B=1:2.

### Example 123

### (2S)—N-[[6-(3,3-Difluorocyclopentoxy)-5-fluoro-2-methoxy-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is A.21)

The title compound was prepared using the procedure described in example 2, 114 and 120, starting from 3,3-difluorocyclopentanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 388.2

¹H NMR (500 MHz, DMSO-d₆) δ ppm: 9.89 (br s, 1H, A), 9.65 (br s, 1H, B), 8.49 (br s, 1H, A and B), 7.68 (d, J=10.1 Hz, 1H, A), 7.50 (d, J=10.2 Hz, 1H, B), 5.51-5.40 (m, 1H, A and B), 4.73-4.66 (m, 1H, A), 4.66-4.59 (m, 1H, B), 4.58 (d, J=16.1 Hz, 1H, A, AB signal), 4.42 (d, J=15.3 Hz, 1H, B, AB signal), 4.37 (d, J=15.3 Hz, 1H, B, AB signal), 4.27 (d, J=15.9 Hz, 1H, A, AB signal), 3.91 (s, 3H, A), 3.88 (s, 3H, B), 3.29-3.22 (m, 1H, A and B), 3.22-3.13 (m, 1H, A and B), 2.99 (s, 3H, B), 2.76 (s, 3H, A), 2.79-2.65 (m, 1H, A and B), 2.45-2.37 (m, 1H, A and B), 2.37-2.24 (m, 3H, A and B), 2.24-2.12 (m, 1H, A and B), 2.06-1.97 (m, 1H, A and B), 1.97-1.85 (m, 2H, A and B), 1.85-1.74 (m, 1H, A and B). Rotamers A:B=1:2.

### Example 124

### (2S)—N-[[5-Fluoro-6-[(4-fluorophenyl)methoxy]-2-methoxy-3-pyridyl]methyl]-N-methyl-pyrrolidine-2-carboxamide Hydrochloride

(Compound of Formula Ia.1, Wherein X is CH, R⁵ is CH₃, R⁶ is F, R⁷ is OCH₃ and R^{8a} is —CH₂-A.62)

The title compound was prepared using the procedure described in example 2, 114 and 120, starting from (4-fluorophenyl)methanol and (S)-tert-butyl 2-(((6-chloro-5-fluoro-2-methoxypyridin-3-yl)methyl)(methyl)carbamoyl)pyrrolidine-1-carboxylate followed by BOC deprotection. Finally the hydrochloride salt was formed by adding one equivalent hydrochloric acid.

LCMS (ESI⁺) m/z [M+H]⁺: 392.3

¹H NMR (500 MHz, DMSO-d₆) δ ppm: 9.88 (br s, 1H, A), 9.63 (br s, 1H, B), 8.48 (br s, 1H, A and B), 7.67 (d, J=10.2 Hz, 1H, A), 7.56-7.51 (m, 2H, A and B), 7.50 (d, J=10.3 Hz, 1H, B), 7.26-7.17 (m, 2H, A and B), 5.45 (s, 2H, A), 5.43 (s, 2H, B), 4.73 4.66 (m, 1H, A), 4.65-4.58 (m, 1H, B), 4.57 (d, J=15.9 Hz, 1H, A, AB signal), 4.41 (d, J=15.3 Hz, 1H, B, AB signal), 4.37 (d, J=15.2 Hz, 1H, B, AB signal), 4.26 (d, J=16.1 Hz, 1H, A, AB signal), 3.92 (s, 3H, A), 3.88 (s, 3H, B), 3.28-3.21 (m, 1H, A and B), 3.21-3.12 (m, 1H, A and B), 2.99 (s, 3H, B), 2.75 (s, 3H, A), 2.45-2.34 (m, 1H, A and B), 1.99-1.84 (m, 2H, A and B), 1.84-1.73 (m, 1H, A and B). Rotamers A:B=1:2.

### Example 125

### (2S)—N-[(5-Fluoro-2-methoxy-6-phenyl-3-pyridyl)methyl]pyrrolidine-2-carboxamide; 2,2,2-trifluoroacetic Acid

(Compound of Formula Ia.24, Wherein X is CH, R⁵ is H, R⁶ is F, R⁷ is OCH₃ and R^{8d} is A.59)

(S)-tert-Butyl 2-(((6-chloro-5-fluoro-2-methoxypyridin-3-yl)methyl)carbamoyl)pyrrolidine-1-carboxylate (50 mg, 0.129 mmol; example 2, step 2.4), phenylboronic acid (20.95 mg, 0.172 mmol), sodium hydrogencarbonate (344 μL, 0.516 mmol, 1.5M) and tetrakis(triphenylphosphine)palladium(0) (10.43 mg, 9.02 μmol) were suspended in DMF (1.5 mL) and flushed with argon. The reaction mixture stirred in the Microwave at 100° C. for 30 min. After addition of water, the aqueous layer was extracted three times with ethyl acetate, then dried over sodium sulfate, filtered and the solvent was evaporated. The raw material (83 mg), was purified by column chromatography on silica gel (12 g column; cyclohexane 100%→cyclohexane: ethyl acetate 70:30, 30 ml/min) to give (2S)—N-[(5-fluoro-2-methoxy-6-phenyl-3-pyridyl)methyl]pyrrolidine-2-carboxamide (42 mg, yield 76%). To a solution of (2S)—N-[(5-fluoro-2-methoxy-6-phenyl-3-pyridyl)methyl]pyrrolidine-2-carboxamide (42 mg, 0.098 mmol) in DCM (2 ml) was added TFA (188 μl, 2.445 mmol). The reaction mixture was stirred at room temperature overnight and concentrated under reduced pressure, and subsequently water added to the residue. Lyophilization of the aqueous solution gave the title compound (2S)—N-[(5-fluoro-2-methoxy-6-phenyl-3-pyridyl)methyl]pyrrolidine-2-carboxamide; 2,2,2-trifluoroacetic acid (38 mg, yield 86%, purity 98%).

LCMS (ESI⁺) m/z [M+H]⁺: 330.20

¹H NMR (500 MHz, methanol-4) δ ppm: 8.03-7.98 (m, 2H), 7.51 (d, J=10.8 Hz, 1H), 7.48-7.43 (m, 2H), 7.43-7.37 (m, 1H), 4.42 (s, 2H), 4.33-4.27 (m, 1H), 4.05 (s, 3H), 3.47-3.38 (m, 1H), 3.38-3.32 (m, 1H), 2.51-2.38 (m, 11H), 2.13-1.99 (m, 3H)

### Example 126

### (2S)—N-[[6-[(4,4-Difluorocyclohexyl)amino]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is A.33)

(S)-tert-Butyl 2-(((6-chloro-5-fluoro-2-methoxypyridin-3-yl)methyl)carbamoyl)pyrrolidine-1-carboxylate (200 mg, 0.516 mmol), 4,4-difluorocyclohexane amine (84 mg, 0.619 mmol), 2,2′-bis(diphenylphosphino)-1,1′-binaphthalene (BINAP) (32.1 mg, 0.052 mmol), cesium carbonate (420 mg, 1.289 mmol) and bis(dibenzylideneacetone)palladium(0) (29.7 mg, 0.052 mmol) were suspended in toluene (3 ml) and flushed with argon. The reaction mixture stirred in the Microwave at 140° C. for 12 h. The reaction mixture was concentrated and the obtained crude product (401 mg) was dissolved in DCM. Bulk Isolute Sorbent was added and the product was purified using flash chromatography (4 g column; DCM 100%→DCM:MeOH 30:70, 5 ml/min) to give (2S)—N-[[6-[(4,4-difluorocyclohexyl)amino]-5-fluoro-2-methoxy-3-pyridyl]methyl]pyrrolidine-2-carboxamide (253.9 mg, yield 101%). The material was used as crude product without further purification. To a solution of (2S)—N-[[6-[(4,4-difluorocyclohexyl)amino]-5-fluoro-2-methoxy-3-pyridyl]methyl]pyrrolidine-2-carboxamide (253 mg, 0.520 mmol) in DCM (5 ml) was added TFA (801 μl, 10.40 mmol). The reaction mixture was stirred at room temperature overnight and subsequently concentrated under reduced pressure. The obtained crude product (568 mg) was dissolved in DCM and Bulk Isolute Sorbent was added. The product was purified twice using flash chromatography (4 g column; DCM 100%→DCM:MeOH 0:100, 18 ml/min) to give (S)—N-((6-((4,4-difluorocyclohexyl)amino)-5-fluoro-2-methoxypyridin-3-yl)methyl)pyrrolidine-2-carboxamide (180.6 mg) as crude product. Preparative HPLC chromatography on a reversed phase column (eluents contained 0.1% TFA) yielded the title compound (S)—N((6-((4,4-difluorocyclohexyl)amino)-5-fluoro-2-methoxypyridin-3-yl)methyl)pyrrolidine-2-carboxamide 2,2,2-trifluoroacetate (102.1 mg, yield 39%)

LCMS (ESI⁺) m/z [M+H]⁺: 387.30

¹H NMR (500 MHz, methanol-d4) δ ppm: 7.17 (d, J=10.8 Hz, 1H), 4.29-4.15 (m, 3H), 4.09-4.00 (m, 1H), 3.88 (s, 3H), 3.46-3.30 (m, 2H), 2.41-2.33 (m, 1H), 2.16-1.82 (m, 10H), 1.75-1.62 (m, 1H).

### Example 127

### (2S)—N-[(6-Cyclopropyl-5-fluoro-2-methoxy-3-pyridyl)methyl]pyrrolidine-2-carboxamide; 2,2,2-trifluoroacetic Acid

(Compound of Formula Ia.24, Wherein X is CH, R⁵ is H, R⁶ is F, R⁷ is OCH₃ and R^{8d} is A.1)

(S)-tert-Butyl 2-(((6-chloro-5-fluoro-2-methoxypyridin-3-yl)methyl)carbamoyl)pyrrolidine-1-carboxylate (200 mg, 0.516 mmol) and cyclopropylboronic acid (44.3 mg, 0.516 mmol) were dissolved in dioxane (2.5 ml) to give a yellow solution. Cesium carbonate (420 mg, 1.289 mmol) and 1,1′-bis(diphenylphospino)ferrocene-palladium(II)dichloride dichloromethane complex (39 mg, 0.048 mmol) and water (0.6 ml) were added and the mixture was flushed with argon. The reaction mixture was stirred in the Microwave at 80° C. for 10 h. After addition of water, the aqueous layer was extracted three times with ethyl acetate, then dried over sodium sulfate, filtered and the solvent was evaporated. The raw material was taken onto Bulk Isolute Sorbent and purified using flash chromatography (4 g column; DCM 100%→DCM:MeOH 80:20, 18 ml/min) to give (S)-tert-butyl 2-((4-cyclopropyl-2-methoxybenzyl)carbamoyl)pyrrolidine-1-carboxylate (173.3 mg, yield 57.4%, purity 60%). The material was used as crude product without further purification. To a solution of (S)-tert-butyl 2-((4-cyclopropyl-2-methoxybenzyl)carbamoyl)pyrrolidine-1-carboxylate (173.3 mg, 0.463 mmol) in DCM (10 ml) was added TFA (0.713 ml, 9.26 mmol). The reaction mixture was stirred at room temperature for 2 h. The reaction mixture was diluted with DCM and was washed once with 2M NaOH. The basified aqueous layer was extracted 5× with DCM. The combined organic layers were dried with sodium sulfate, filtered and concentrated. Preparative HPLC chromatography on a reversed phase column yielded the title compound (S)—N-(4-cyclopropyl-2-methoxybenzyl)pyrrolidine-2-carboxamide 2,2,2-trifluoroacetate (56.6 mg, yield 31.5%).

LCMS (ESI⁺) m/z [M+H]⁺: 294.20

¹H NMR (600 MHz, CDCl₃) δ ppm: 7.75 (m, 1H), 7.15 (d, 1H), 4.65 (m, 1H), 4.35-4.30 (m, 2H), 3.85 (s, 3H), 3.40 (m, 2H), 2.50-2.30 (m, 2H), 2.25-2.20 (m, 1H), 2.10-2.00 (m, 3H), 1.05 (m, 2H), 0.95 (m, 2H).

### Example 128

### (2S)—N-[[5-Fluoro-2-methoxy-6-(1-piperidyl)-3-pyridyl]methyl]pyrrolidine-2-carboxamide

(Compound of Formula Ia.23, Wherein X is CH, R⁵ is H, R⁶ is F, R⁷ is OCH₃, and NR^{8b}R^{8c} is A.114)

(S)-tert-Butyl 2-(((6-chloro-5-fluoro-2-methoxypyridin-3-yl)methyl)carbamoyl)pyrrolidine-1-carboxylate (200 mg, 0.516 mmol), piperidine (61 μl, 0.619 mmol), 2,2′-bis(diphenylphosphino)-1,1′-binaphthalene (BINAP) (32.1 mg, 0.052 mmol), cesium carbonate (420 mg, 1.289 mmol) and bis(dibenzylideneacetone)palladium(0) (29.7 mg, 0.052 mmol) were suspended in toluene (3 ml) and flushed with argon. The reaction mixture was stirred in the Microwave at 140° C. for 2 h. The reaction mixture was concentrated and the obtained crude product (721 mg) was twice purified using flash chromatography (4 g column; DCM 100%→DCM:MeOH 80:20, 18 ml/min) to give (S)-tert-butyl 2-(((5-fluoro-2-methoxy-6-(piperidin-1-yl)pyridin-3-yl)methyl)carbamoyl)pyrrolidine-1-carboxylate (135 mg, yield 60%). To a solution of (S)-tert-butyl 2-(((5-fluoro-2-methoxy-6-(piperidin-1-yl)pyridin-3-yl)methyl)carbamoyl)pyrrolidine-1 carboxylate (135 mg, 0.309 mmol) in DCM (5 ml) was added TFA (477 μl, 6.19 mmol). The reaction mixture was stirred at room temperature overnight and subsequently concentrated under reduced pressure. The obtained crude product (162 mg) was dissolved in ethyl acetate. Bulk Isolute Sorbent was added and the product was purified using flash chromatography (4 g column; DCM 100%→DCM:MeOH 60:40, 18 ml/min) to give the title compound ((S)—N-((5-fluoro-2-methoxy-6-(piperidin-1-yl)pyridin-3-yl)methyl)pyrrolidine-2-carboxamide (87 mg, yield 83.7%).

LCMS (ESI⁺) m/z [M+H]⁺: 337.20

¹H NMR (500 MHz, CDCl₃) δ ppm: 7.86 (m, 1H), 7.10 (d, J=12.5 Hz, 1H), 4.60 (m, 1H), 4.30-4.20 (m, 2H), 3.85 (s, 3H), 3.50-3.35 (m, 5H), 2.45-2.35 (m, 1H), 2.10-1.80 (m, 5H), 1.65-1.60 (m, 6H).

### Example 129

### (2S)—N-[[6-(Cyclohexylmethyl)-5-fluoro-2-methoxy-3-pyridyl]methyl]pyrrolidine-2-carboxamide

(Compound of Formula Ia.24, Wherein X is CH, R⁵ is H, R⁶ is F, R⁷ is OCH₃ and R^{8d} is —CH₂-A.27)

(S)-tert-Butyl 2-(((6-chloro-5-fluoro-2-methoxypyridin-3-yl)methyl)carbamoyl)pyrrolidine-1-carboxylate (250 mg, 0.645 mmol), cyclohexylmethylboronic acid (101 mg, 0.709 mmol), cesium carbonate (525 mg, 1,612 mmol) and bis(diphenylphospino)ferrocene-palladium(II)dichloride dichloromethane complex (52.6 mg, 0.064 mmol) were dissolved in dioxane (12 ml) and water (2 ml) and flushed 3× with argon. The reaction mixture was stirred in the microwave at 80° C. for 10 h, then 130° C. for 2 h and finally at 140° C. for 12 h. The reaction mixture was filtered under vacuum over diatomaceous earth, washed and concentrated. The crude product (773.7 mg) was dissolved in DCM. Bulk Isolute Sorbent was added and the product was purified using flash chromatography (4 g column; DCM 100%→DCM:MeOH 0:100, 18 ml/min) to give (S)-tert-butyl 2-(((6-(cyclohexylmethyl)-5-fluoro-2-methoxypyridin-3-yl)methyl)carbamoyl)pyrrolidine-1-carboxylate (317.2 mg). The material was used as crude product without further purification. To a solution of (S)-tert-butyl 2-(((6-(cyclohexylmethyl)-5-fluoro-2-methoxypyridin-3-yl)methyl)carbamoyl)pyrrolidine-1-carboxylate (317 mg, 0.705 mmol) in DCM (5 ml) was added TFA (1,087 ml, 14.10 mmol). The reaction mixture was stirred at room temperature overnight and subsequently concentrated under reduced pressure. The obtained crude product (544 mg) was dissolved in DCM. Bulk Isolute Sorbent was added and the product was purified using flash chromatography (4 g column; DCM 100%→DCM:MeOH 50:50, 18 ml/min) to give 265.7 mg crude product. The title product was obtained by preparative HPLC chromatography on a reversed phase column (26.3 mg, yield 11%)

LCMS (ESI⁺) m/z [M+H]⁺: 350.40

¹H NMR (500 MHz, methanol-d4) δ ppm: 7.34 (d, J=9.1 Hz, 1H), 4.34 (s, 2H), 4.30-4.25 (m, 1H), 3.93 (s, 3H), 3.45-3.35 (m, 1H), 2.60-2.55 (m, 2H), 2.45-2.35 (m, 1H), 2.10-1.95 (m, 3H), 1.80-1.75 (m, 1H), 1.70-1.60 (m, 6H), 1.30-1.20 (m, 4H), 1.10-1.00 (m, 2H).

### Example 130

### (2S)—N-[[5-Fluoro-2-methoxy-6-[[4-(trifluoromethyl)cyclohexyl]amino]-3-pyridyl]methyl]pyrrolidine-2-carboxamide

(Compound of Formula Ia.23, Wherein X is CH, R⁵ is H, R⁶ is F, R⁷ is OCH₃, R^{8b} is H and R^{8c} is A.36)

(S)-tert-Butyl 2-(((6-chloro-5-fluoro-2-methoxypyridin-3-yl)methyl)carbamoyl)pyrrolidine-1-carboxylate (200 mg, 0.516 mmol), 4-(trifluoromethyl)cyclohexanamine (103 mg, 0.619 mmol), 2,2′-bis(diphenylphosphino)-1,1′-binaphthalene (BINAP) (32.1 mg, 0.052 mmol), cesium carbonate (420 mg, 1.289 mmol) and bis(dibenzylideneacetone)palladium(0) (29.7 mg, 0.052 mmol) were suspended in toluene (4 ml) and flushed with argon to give a brown suspension. The reaction mixture was stirred in the Microwave at 140° C. for 4 h. The reaction mixture was concentrated and the obtained crude product was taken onto Bulk Isolute Sorbent and purified using flash chromatography (12 g column; DCM 100%→DCM:MeOH 80:20, 30 ml/min) to give (2S)-tert-butyl 2-(((5-fluoro-2-methoxy-6-((4-(trifluoromethyl)cyclohexyl)amino)pyridin-3-yl)methyl)carbamoyl)pyrrolidine-1-carboxylate (182.8 mg, yield 41%, purity 60%). To a solution of (2S)-tert-butyl 2-(((5-fluoro-2-methoxy-6-((4-(trifluoromethyl)cyclohexyl)amino)pyridin-3-yl)methyl)carbamoyl)pyrrolidine-1-carboxylate (182.8 mg, 0.282 mmol) in DCM (10 ml) was added TFA (0.453 ml, 5.64 mmol). The reaction mixture was stirred at room temperature for 2 h. The reaction mixture was diluted with DCM and was washed once with 2M NaOH. The basified aqueous layer was extracted 5× with DCM. The combined organic layers were dried with sodium sulfate, filtered and concentrated. The crude product was taken onto Bulk Isolute Sorbent and purified using flash chromatography (12 g column; n-heptane 100%→n-heptane: (ethyl acetate/EtOH 3:1) 0:100, 30 ml/min) to give the title compound (2S)—N-((5-fluoro-2-methoxy-6-((4-(trifluoromethyl)cyclohexyl)amino)pyridin-3-yl)methyl)pyrrolidine-2-carboxamide (125 mg, yield 101%, purity 95%).

LCMS (ESI⁺) m/z [M+H]⁺: 419.20

¹H NMR (600 MHz, CDCl₃) δ ppm: 12.47 (s, 1H), 7.45 (q, J=5.8 Hz, 1H), 7.08 (dd, J=10.4, 8.3 Hz, 1H), 4.62 (ddd, J=8.4, 6.1, 2.2 Hz, 1H), 4.29-4.18 (m, 3H), 3.86 (d, J=5.8 Hz, 3H), 3.40 (tdd, J=18.0, 11.7, 6.7 Hz, 2H), 2.42 (ddd, J=12.8, 8.7, 5.9 Hz, 1H), 2.15-1.93 (m, 6H), 1.84-1.78 (m, 1H), 1.71-1.54 (m, 5H).

### Example 131

### (2S)—N-[[6-(Cyclohexylamino)-5-fluoro-2-methoxy-3-pyridyl]methyl]pyrrolidine-2-carboxamide

(Compound of Formula Ia.23, Wherein X is CH, R⁵ is H, R⁶ is F, R⁷ is OCH₃, R^{8b} is H and R^{8c} is A.27)

(S)-tert-Butyl 2-(((6-chloro-5-fluoro-2-methoxypyridin-3-yl)methyl)carbamoyl)pyrrolidine-1-carboxylate (200 mg, 0.516 mmol), cyclohexane amine (71 μl, 0.619 mmol) and cesium carbonate (420 mg, 1.289 mmol) were suspended in toluene (4 ml) to give a brown suspension. Then 2,2′-bis(diphenylphosphino)-1,1′-binaphthalene (BINAP) (32.1 mg, 0.052 mmol) and bis(dibenzylideneacetone)palladium(0) (29.7 mg, 0.052 mmol) were added and the reaction mixture was flushed with argon. The reaction mixture was stirred in the Microwave at 140° C. for 4 h. The reaction mixture was concentrated and the obtained crude product was taken onto Bulk Isolute Sorbent and purified using flash chromatography (12 g column; DCM 100%→DCM:MeOH 80:20, 30 ml/min) to give (S)-tert-butyl 2-(((6-(cyclohexylamino)-5-fluoro-2-methoxypyridin-3-yl)methyl)carbamoyl)pyrrolidine-1-carboxylate (271.3 mg, yield 82%, purity 70%). To a solution of (S)-tert-butyl 2-(((6-(cyclohexylamino)-5-fluoro-2-methoxypyridin-3-yl)methyl)carbamoyl)pyrrolidine-1-carboxylate (271.3 mg, 0.422 mmol) in DCM (10 ml) was added TFA (0.649 ml, 8.43 mmol). The reaction mixture was stirred at room temperature for 2 h. The reaction mixture was diluted with DCM and was washed once with 2M NaOH. The basified aqueous layer was extracted 5× with DCM. The combined organic layers were dried with sodium sulfate, filtered and concentrated. The raw material was taken onto Bulk Isolute Sorbent and purified using flash chromatography (12 g column; n-heptane 100%→n-heptane: (ethyl acetate/EtOH 3:1) 0:100, 30 ml/min) to give the tile compound (S)—N-((6-(cyclohexylamino)-5-fluoro-2-methoxypyridin-3-yl)methyl)pyrrolidine-2-carboxamide (134.9 mg, yield 87%, purity 95%).

LCMS (ESI⁺) m/z [M+H]⁺: 351.30

¹H NMR (600 MHz, CDCl₃) δ ppm: 12.32 (s, 1H), 7.50 (t, J=5.8 Hz, 1H), 7.05 (d, J=10.5 Hz, 1H), 4.60 (dd, J=8.6, 5.8 Hz, 1H), 4.28-4.17 (m, 2H), 3.85 (s, 3H), 3.90-3.79 (m, 1H), 3.44-3.33 (m, 2H), 2.39 (dddd, J=12.0, 10.5, 7.2, 4.9 Hz, 1H), 2.10-1.92 (m, 6H), 1.79-1.72 (m, 2H), 1.65 (dt, J=13.0, 3.9 Hz, 1H),1.45-1.34 (m, 2H), 1.31-1.12 (m, 2H).

### Example 132

### (2S)—N-[(6-Cyclohexyl-5-fluoro-2-methoxy-3-pyridyl)methyl]pyrrolidine-2-carboxamide; 2,2,2-trifluoroacetic Acid

(Compound of Formula Ia.24, Wherein X is CH, R⁵ is H, R⁶ is F, R⁷ is OCH₃ and R^{8d} is A.27)

(S)-tert-Butyl 2-(((6-chloro-5-fluoro-2-methoxypyridin-3-yl)methyl)carbamoyl)pyrrolidine-1-carboxylate (127 mg, 0.327 mmol) and cyclohex-1-en-1-ylboronic acid (41.2 mg, 0.327 mmol) were dissolved in dioxane (2.5 ml) to give a yellow solution. Cesium carbonate (267 mg, 0.819 mmol) and 1,1′-bis(diphenylphospino)ferrocene-palladium(II)dichloride dichloromethane complex (26.7 mg, 0.033 mmol) and water (0.6 ml) were added and the mixture was flushed with argon. The reaction mixture was stirred in the Microwave at 80° C. for 10 h and then at 100° C. for 2 h. The reaction mixture was concentrated and the obtained crude product was taken onto Bulk Isolute Sorbent and purified using flash chromatography (12 g column; DCM 100%→DCM:MeOH 80:20, 30 ml/min) to give (S)-tert-butyl 2-(((6-(cyclohex-1-en-1-yl)-5-fluoro-2-methoxypyridin-3-yl)methyl)carbamoyl)pyrrolidine-1-carboxylate (88.5 mg, yield 31.2%, purity 50%). The material was used as crude product without further purification. (S)-tert-Butyl 2-(((6-(cyclohex-1-en-1-yl)-5-fluoro-2-methoxypyridin-3-yl)methyl)carbamoyl)pyrrolidine-1-carboxylate (88.5 mg, 0,102 mmol) was dissolved in ethyl acetate (10 ml) to give a yellow solution. The reaction mixture was flushed with nitrogen, and then palladium on carbon (10.86 mg, 0.102 mmol) was added. The reaction mixture was flushed 3× with hydrogen and stirred at room temperature overnight. Celite was added to the reaction mixture and filtered over Celite under vacuum and washed with ethyl acetate. The organic mother liquor was concentrated to give (S)-tert-butyl 2-(((6-cyclohexyl-5-fluoro-2-methoxypyridin-3-yl)methyl)carbamoyl)pyrrolidine-1-carboxylate (95 mg, yield 107%, purity 50%). The material was used as crude product without further purification. To a solution of (S)-tert-butyl 2-(((6-cyclohexyl-5-fluoro-2-methoxypyridin-3-yl)methyl)carbamoyl)pyrrolidine-1-carboxylate (95 mg, 0.218 mmol) in DCM (10 ml) was added TFA (0.336 ml, 4.36 mmol). The reaction mixture was stirred at room temperature for 2 h and then concentrated. Preparative HPLC chromatography on a reversed phase column yielded the title compound (S)—N-((6-cyclohexyl-5-fluoro-2-methoxypyridin-3-yl)methyl)pyrrolidine-2-carboxamide 2,2,2-trifluoroacetate (4.4 mg, yield 4.49%).

LCMS (ESI+) m/z [M+H]⁺: 336.30

¹H NMR (600 MHz, CDCl₃) δ ppm: 12.18 (s, 1H), 7.57 (s, 1H), 7.17 (d, J=9.0 Hz, 1H), 4.71 (s, 1H), 4.50-4.24 (m, 2H), 3.93 (s, 3H), 3.43 (dd, J=15.3, 7.3 Hz, 1H), 2.91 (dddd, J=13.4, 7.1, 5.6, 3.3 Hz, 1H), 2.44 (t, J=7.8 Hz, 1H), 1.91-1.54 (m, 9H), 1.52-1.14 (m, 4H).

### Example 133

### (2S)—N-[[5-Fluoro-2-methoxy-6-[4-(trifluoromethyl)-1-piperidyl]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.126)

(S)-tert-Butyl 2-(((6-chloro-5-fluoro-2-methoxypyridin-3-yl)methyl)carbamoyl)pyrrolidine-1-carboxylate (100 mg, 0.258 mmol), 4-(trifluoromethyl)cyclohexane amine (103 mg, 0.619 mmol) and cesium carbonate (210 mg, 0.645 mmol) were suspended in toluene (4 ml) and flushed with argon to give a brown suspension. Then 2,2′-bis(diphenylphosphino)-1,1′-binaphthalene (BINAP) (16.06 mg, 0.026 mmol) and bis(dibenzylideneacetone)palladium(0) (14.83 mg, 0.026 mmol) were added and the reaction mixture was flushed with argon. The reaction mixture was stirred in the Microwave at 140° C. for 4 h. The reaction mixture was concentrated and the obtained crude product was taken onto Bulk Isolute Sorbent and purified using flash chromatography (12 g column; n-heptane 100%→n-heptane: (ethyl acetate/EtOH 3:1) 0:100, 30 ml/min) to give (S)-tert-butyl 2-(((5-fluoro-2-methoxy-6-(4-(trifluoromethyl)piperidin-1-yl)pyridin-3-yl)methyl)carbamoyl)pyrrolidine-1-carboxylate (91 mg, yield 70%). To a solution of (S)-tert-butyl 2-(((5-fluoro-2-methoxy-6-(4-(trifluoromethyl)piperidin-1-yl)pyridin-3-yl)methyl)carbamoyl)pyrrolidine-1-carboxylate (91 mg, 0.180 mmol) in DCM (10 ml) was added TFA (0.278 ml, 3.61 mmol). The reaction mixture was stirred at room temperature for 2 h. The reaction mixture was diluted with DCM and was washed once with 2M NaOH. The basified aqueous layer was extracted 3× with DCM. The combined organic layers were dried with sodium sulfate, filtered and concentrated. The raw material was taken onto Bulk Isolute Sorbent and purified using flash chromatography (4 g column; n-heptane 100%→n-heptane: (ethyl acetate/EtOH 3:1) 0:100, 18 ml/min) to give (S)—N-((5-fluoro-2-methoxy-6-(4-(trifluoromethyl)piperidin-1-yl)pyridin-3-yl)methyl)pyrrolidine-2-carboxamide (purity 75%). Preparative HPLC chromatography on a reversed phase column (eluents contained 0.1% TFA) gave the title compound (S)—N-((5-fluoro-2-methoxy-6-(4-(trifluoromethyl)piperidin-1-yl)pyridin-3-yl)methyl)pyrrolidine-2-carboxamide 2,2,2-trifluoroacetate (54 mg, yield 54.9%, purity 95%).

LCMS (ESI⁺) m/z [M+H]⁺: 405.30

¹H NMR (600 MHz, CDCl₃) δ ppm: 11.73 (s, 1H), 7.40 (t, J=5.8 Hz, 1H), 7.15 (d, J=12.2 Hz, 1H), 4.68 (s, 1H), 4.33-4.19 (m, 4H), 3.87 (s, 3H), 3.45-3.39 (m, 2H), 2.84 (tt, J=13.0, 2.5 Hz, 3H), 2.42 (s, 1H), 2.25 (dtq, J=16.6, 8.3, 4.1 Hz, 1H), 2.13-1.96 (m, 3H), 1.91 (ddd, J=11.7, 3.9, 2.0 Hz, 2H), 1.69 (qd, J=12.6, 4.1 Hz, 2H).

### Example 134

### (2S)—N-[[6-(4,4-Difluoro-1-piperidyl)-5-fluoro-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.120)

(S)-tert-Butyl 2-(((6-chloro-5-fluoro-2-methoxypyridin-3-yl)methyl)carbamoyl)pyrrolidine-1-carboxylate (100 mg, 0.258 mmol), 4,4-difluoropiperidine (37.5 mg, 0.309 mmol) and cesium carbonate (210 mg, 0.645 mmol) were suspended in toluene (4 ml) to give a brown suspension. Then 2,2′-bis(diphenylphosphino)-1,1′-binaphthalene (BINAP) (16.06 mg, 0.026 mmol) and bis(dibenzylideneacetone)palladium(0) (14.83 mg, 0.026 mmol) were added and the reaction mixture was flushed with argon. The reaction mixture was stirred in the Microwave at 140° C. for 4 h. The reaction mixture was concentrated and the obtained crude product was taken onto Bulk Isolute Sorbent and purified using flash chromatography (12 g column; n-heptane 100%→n-heptane: (ethyl acetate/EtOH 3:1) 0:100, 30 ml/min) to give (S)-tert-butyl 2-(((6-(4,4-difluoropiperidin-1-yl)-5-fluoro-2-methoxypyridin-3-yl)methyl)carbamoyl)pyrrolidine-1-carboxylate (174 mg, yield 143%). The material was used as crude product without further purification. To a solution of (S)-tert-butyl 2-(((6-(4,4-difluoropiperidin-1-yl)-5-fluoro-2-methoxypyridin-3-yl)methyl)carbamoyl)pyrrolidine-1-carboxylate (174 mg, 0.368 mmol) in DCM (10 ml) was added TFA (0.567 ml, 7.37 mmol). The reaction mixture was stirred at room temperature for 2 h. The reaction mixture was diluted with DCM and was washed once with 2M NaOH. The basified aqueous layer was extracted 3× with DCM. The combined organic layers were dried with sodium sulfate, filtered and concentrated. The raw material was taken onto Bulk Isolute Sorbent and purified using flash chromatography (4 g column; n-heptane 100%→n-heptane: (ethyl acetate/EtOH 3:1) 0:100, 18 ml/min) to give (S)—N-((6-(4,4-difluoropiperidin-1-yl)-5-fluoro-2-methoxypyridin-3-yl)methyl)pyrrolidine-2-carboxamide (purity 80%). Preparative HPLC chromatography on a reversed phase column (eluents contained 0.1% TFA) gave the title compound (S)—N-((6-(4,4-difluoropiperidin-1-yl)-5-fluoro-2-methoxypyridin-3-yl)methyl)pyrrolidine-2-carboxamide 2,2,2-trifluoroacetate (52.9 mg, yield 28.1%, purity 95%).

LCMS (ESI⁺) m/z [M+H]⁺: 373.20

¹H NMR (600 MHz, CDCl₃) δ ppm: δ 12.06 (s, 1H), 7.50 (t, J=5.9 Hz, 1H), 7.17 (d, J=12.2 Hz, 1H), 4.68 (t, J=7.0 Hz, 1H), 4.34-4.23 (m, 2H), 3.87 (s, 3H), 3.64 (t, J=5.8 Hz, 4H), 3.43 (dq, J=13.7, 6.5, 5.5 Hz, 2H), 2.44 (dp, J=9.7, 3.9, 3.2 Hz, 1H), 2.13-1.95 (m, 7H).

### Example 135

### (2S)—N-[(5-Fluoro-2-methoxy-6-pyrrolidin-1-yl-3-pyridyl)methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.106)

(S)-tert-Butyl 2-(((6-chloro-5-fluoro-2-methoxypyridin-3-yl)methyl)carbamoyl)pyrrolidine-1-carboxylate (100 mg, 0.258 mmol), pyrrolidine (22.01 mg, 0.309 mmol) and cesium carbonate (210 mg, 0.645 mmol) were suspended in toluene (4 ml) to give a brown suspension. Then 2,2′-bis(diphenylphosphino)-1,1′-binaphthalene (BINAP) (16.06 mg, 0.026 mmol) and bis(dibenzylideneacetone)palladium(0) (14.83 mg, 0.026 mmol) were added and the reaction mixture was flushed with argon. The reaction mixture was stirred in the Microwave at 140° C. for 4 h. The reaction mixture was concentrated and the obtained crude product was taken onto Bulk Isolute Sorbent and purified using flash chromatography (12 g column; n-heptane 100%→n-heptane: (ethyl acetate/EtOH 3:1) 0:100, 30 ml/min) to give (S)-tert-butyl 2-(((5-fluoro-2-methoxy-6-(pyrrolidin-1-yl)pyridin-3-yl)methyl)carbamoyl)pyrrolidine-1-carboxylate (83.8 mg, yield 77%). To a solution of (S)-tert-butyl 2-(((5-fluoro-2-methoxy-6-(pyrrolidin-1-yl)pyridin-3-yl)methyl)carbamoyl)pyrrolidine-1-carboxylate (83.8 mg, 0.198 mmol) in DCM (10 ml) was added TFA (0.306 ml, 3.97 mmol). The reaction mixture was stirred at room temperature for 2h. The reaction mixture was diluted with DCM and was washed once with 2M NaOH. The basified aqueous layer was extracted 3× with DCM. The combined organic layers were dried with sodium sulfate, filtered and concentrated. The raw material was taken onto Bulk Isolute Sorbent and purified using flash chromatography (4 g column; n-heptane 100%→n-heptane: (ethyl acetate/EtOH 3:1) 0:100, 18 ml/min) to give (S)—N-((5-fluoro-2-methoxy-6-(pyrrolidin-1-yl)pyridin-3-yl)methyl)pyrrolidine-2-carboxamide (purity 80%). Preparative HPLC chromatography on a reversed phase column (eluents contained 0.1% TFA) gave the title compound (S)—N-((5-fluoro-2-methoxy-6-(pyrrolidin-1-yl)pyridin-3-yl)methyl)pyrrolidine-2-carboxamide 2,2,2-trifluoroacetate (29.3 mg, yield 33.8%).

LCMS (ESI⁺) m/z [M+H]⁺: 323.30

¹H NMR (600 MHz, CDCl₃) δ ppm: 12.39 (s, 1H), 7.06 (d, J=12.3 Hz, 1H), 6.96 (t, J=5.8 Hz, 1H), 4.66-4.60 (m, 1H), 4.30-4.19 (m, 2H), 3.87 (s, 3H), 3.61 (ddt, J=6.9, 4.5, 2.6 Hz, 4H), 3.51-3.43 (m, 1H), 3.38 (dt, J=11.5, 6.4 Hz, 1H), 2.50-2.36 (m, 1H), 2.19-2.04 (m, 1H), 2.05-1.84 (m, 6H).

### Example 136

### (2S)—N-[[6-[(3,3-Difluorocyclobutyl)methylamino]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is —CH₂-A.10)

(S)-tert-Butyl 2-(((6-chloro-5-fluoro-2-methoxypyridin-3-yl)methyl)carbamoyl)pyrrolidine-1-carboxylate (200 mg, 0.516 mmol), (3,3-difluorocyclobutyl)methane amine hydrochloride (98 mg, 0.619 mmol), 2,2′-bis(diphenylphosphino)-1,1′-binaphthalene (BINAP) (32.1 mg, 0.052 mmol), cesium carbonate (420 mg, 1.289 mmol) and bis(dibenzylideneacetone)palladium(0) (29.7 mg, 0.052 mmol) were suspended in toluene (3 ml) and flushed with argon. The reaction mixture was stirred in the Microwave at 140° C. for 2 h. The reaction mixture was filtered and concentrated to give (S)-tert-butyl 2-(((6-(((3,3-difluorocyclobutyl)methyl)amino)-5-fluoro-2-methoxypyridin-3-yl)methyl)carbamoyl)pyrrolidine-1-carboxylate (780 mg). The material was used as crude product without further purification. To a solution of (S)-tert-butyl 2-(((6-(((3,3-difluorocyclobutyl)methyl)amino)-5-fluoro-2-methoxypyridin-3-yl)methyl)carbamoyl)pyrrolidine-1-carboxylate (780 mg, 1.651 mmol) in DCM (5 ml) was added TFA (800 μl, 10.38 mmol). The reaction mixture was stirred at room temperature over two days and subsequently concentrated under reduced pressure. The obtained crude product (1.4 g) was dissolved in ethyl acetate. Bulk Isolute Sorbent was added and the mixture was purified using flash chromatography (4 g column; DCM 100%→DCM:MeOH 0:100, 18 ml/min) to give (S)—N-((6-(((3,3-difluorocyclobutyl)methyl)amino)-5-fluoro-2-methoxypyridin-3-yl)methyl)pyrrolidine-2-carboxamide (192.9 mg). Preparative HPLC chromatography on a reversed phase column (eluents contained 0.1% TFA) gave the title compound (S)—N-((6-(((3,3-difluorocyclobutyl)methyl)amino)-5-fluoro-2-methoxypyridin-3-yl)methyl)pyrrolidine-2-carboxamide 2,2,2-trifluoroacetate (97.7 mg, yield 12%).

LCMS (ESI⁺) m/z [M+H]⁺: 373.30

¹H NMR (600 MHz, methanol-d₄) δ ppm: 7.17 (d, J=10.8 Hz, 1H), 4.27-4.15 (m, 3H), 3.87 (s, 3H), 3.58-3.53 (m, 2H), 3.43-3.35 (m, 1H), 3.35-3.26 (m, 1H), 2.65-2.52 (m, 2H), 2.54-2.42 (m, 1H), 2.43-2.28 (m, 3H), 2.09-2.00 (m, 2H), 2.00-1.91 (m, 1H).

### Example 137

### (2S)—N-[[5-Fluoro-2-methoxy-6-[methyl(phenethyl)amino]-3-pyridyl]methyl]-pyrrolidine-2-carboxamide Hydrochloride

(Compound of Formula Ia.23, Wherein X is CH, R⁵ is H, R⁶ is F, R⁷ is OCH₃, R^{8b} is CH₃ and R^{8c} is —CH₂CH₂-A.59)

(S)-tert-Butyl 2-(((6-chloro-5-fluoro-2-methoxypyridin-3-yl)methyl)carbamoyl)pyrrolidine-1-carboxylate (100 mg, 0.258 mmol), 2,2′-bis(diphenylphosphino)-1,1′-binaphthalene (BINAP) (16.06 mg, 0.026 mmol), cesium carbonate (215 mg, 0.660 mmol) and tris(dibenzylideneacetone)dipalladium(0) (23.61 mg, 0.026 mmol) were suspended in degassed toluene (3 ml) and flushed with argon. Then N-methyl-2-phenylethane amine (45 μl, 0.309 mmol) was added via syringe to give a brown suspension. The reaction mixture was stirred in the Microwave at 140° C. for 3 h. The reaction mixture was filtered through celite, washed with toluene and subsequently concentrated under reduced pressure. The crude material (285 mg) was purified using flash chromatography (12 g column; DCM 100%→DCM:MeOH 95:5.30 ml/min) to give (S)-tert-butyl 2-(((5-fluoro-2-methoxy-6-(methyl(phenethyl)amino)pyridin-3-yl)methyl)carbamoyl)pyrrolidine-1-carboxylate (71 mg, yield 56.6%). To a solution of (S)-tert-butyl 2-(((5-fluoro-2-methoxy-6-(methyl(phenethyl)amino)pyridin-3-yl)methyl)carbamoyl)pyrrolidine-1-carboxylate (71 mg, 0.146 mmol) in DCM (3 ml) was added TFA (112 μl, 1.459 mmol). The reaction mixture was stirred at room temperature over two days and subsequently concentrated under reduced pressure. The residue was dissolved in ethyl acetate and washed with saturated sodium bicarbonate solution, the organic layer was dried over magnesium sulfate, filtered and concentrated. The obtained crude product (52 mg) was purified using flash chromatography (4 g column; DCM 100%→DCM:MeOH 90:10.18 ml/min) to give (S)—N-((5-fluoro-2-methoxy-6-(methyl(phenethyl)amino)pyridin-3-yl)methyl)pyrrolidine-2-carboxamide (42 mg, yield 74.5%). To a solution of (S)—N-((5-fluoro-2-methoxy-6-(methyl(phenethyl)amino)pyridin-3-yl)methyl)pyrrolidine-2-carboxamide (42 mg, 0.109 mmol) in diethyl ether (10 ml) was added at 0° C. hydrogen chloride 2M in diethyl ether (54 μl, 0.109 mmol) and stirred. The reaction mixture was concentrated to give the title compound (S)-2-(((5-fluoro-2-methoxy-6-(methyl(phenethyl)amino)pyridin-3-yl)methyl)carbamoyl)pyrrolidin-1-ium chloride (47 mg, yield 102%).

LCMS (ESI⁺) m/z [M+H]⁺: 387.20

¹H NMR (500 MHz, DMSO-d₆) δ ppm: 9.64 (s, 1H), 8.81 (t, J=5.5 Hz, 1H), 8.53 (s, 1H), 7.35-7.26 (m, 3H), 7.28-7.16 (m, 3H), 4.17-4.13 (m, 3H), 3.85 (s, 3H), 3.67-3.60 (m, 2H), 3.30-3.11 (m, 2H), 3.08 (d, J=2.9 Hz, 3H), 2.90-2.82 (m, 2H), 2.29 (ddt, J=12.6, 8.4, 6.2 Hz, 1H), 1.90 (d, J=7.2 Hz, 1H), 1.90-1.78 (m, 2H).

### Example 138

### (2S)—N-[(5-Fluoro-6-indolin-1-yl-2-methoxy-3-pyridyl)methyl]pyrrolidine-2-carboxamide Hydrochloride

(Compound of Formula Ia.23, Wherein X is CH, R⁵ is H, R⁶ is F, R⁷ is OCH₃, and NR^{8b}R^{8c} is A.139)

(S)-tert-Butyl 2-(((6-chloro-5-fluoro-2-methoxypyridin-3-yl)methyl)carbamoyl)pyrrolidine-1-carboxylate (100 mg, 0.258 mmol), 2,2′-bis(diphenylphosphino)-1,1′-binaphthalene (BINAP) (16.06 mg, 0.026 mmol), cesium carbonate (225 mg, 0.691 mmol) and tris(dibenzylideneacetone)dipalladium (0) (23.61 mg, 0.026 mmol) were suspended in degassed toluene (3 ml) and flushed with argon. Then indoline (40 μl, 0.357 mmol) was added via syringe. The reaction mixture was stirred in the Microwave at 140° C. for 3.5 h. Charcoal was added to the reaction mixture and stirred 5 minutes. This suspension was filtered through celite and washed with toluene. The solvent was evaporated to obtain 286 mg of brown oil. The crude material was purified using flash chromatography (12 g column; DCM 100%→DCM:MeOH 95:5.30 ml/min) to give (S)-tert-butyl 2-(((5-fluoro-6-(indolin-1-yl)-2-methoxypyridin-3-yl)methyl)carbamoyl)pyrrolidine-1-carboxylate (107 mg, yield 88%). To a solution of (S)-tert-butyl 2-(((5-fluoro-6-(indolin-1-yl)-2-methoxypyridin-3-yl)methyl)carbamoyl)pyrrolidine-1-carboxylate (107 mg, 0.227 mmol) in DCM (3 ml) was added TFA (0.3 ml, 3.89 mmol). The reaction mixture was stirred at room temperature for 4 h and subsequently concentrated under reduced pressure. The residue was dissolved in ethyl acetate and washed with saturated sodium bicarbonate solution, the organic layer was dried over magnesium sulfate, filtered and concentrated. The obtained crude product (85 mg) was purified using flash chromatography (4 g column; DCM 100%→DCM:MeOH 90:10.18 ml/min) to give (S)—N-((5-fluoro-6-(indolin-1-yl)-2-methoxypyridin-3-yl)methyl)pyrrolidine-2-carboxamide (78 mg, yield 93%). (S)—N-((5-fluoro-6-(indolin-1-yl)-2-methoxypyridin-3-yl)methyl)pyrrolidine-2-carboxamide (78 mg, 0.211 mmol) was dissolved in MeOH (ca. 1 mL), HCl was added and the solution was evaporated to dryness. The obtained residue (yellowish oil) was dissolved in DCM (ca. 2 mL) and evaporated, a white foam was formed. This residue was suspended in diethyl ether (ca. 8 mL) and evaporated to dryness. The obtained white solid was dried under high vacuum to give the title compound (S)-2-(((5-fluoro-6-(indolin-1-yl)-2-methoxypyridin-3-yl)methyl)carbamoyl)pyrrolidin-1-ium chloride (85 mg, yield 99%).

LCMS (ESI⁺) m/z [M+H]⁺: 371.30

¹H NMR (600 MHz, DMSO-d₆) δ ppm: 9.59 (s, 1H), 8.95 (t, J=5.7 Hz, 1H), 8.58 (s, 1H), 7.56 (d, J=12.0 Hz, 1H), 7.41-7.36 (m, 1H), 7.21 (dd, J=7.4, 1.2 Hz, 1H), 7.10 (td, J=7.7, 1.3 Hz, 1H), 6.83 (td, J=7.4, 1.0 Hz, 1H), 4.28-4.18 (m, 3H), 4.14 (td, J=8.6, 3.0 Hz, 2H), 3.88 (s, 3H), 3.28-3.17 (m, 2H), 3.14 (t, J=8.5 Hz, 2H), 2.31 (ddt, J=12.4, 8.2, 5.9 Hz, 1H), 1.94-1.80 (m, 3H).

### Example 139

### (2S)—N-[[5-fluoro-2-methoxy-6-[2-(trifluoromethyl)cyclopropyl]-3-pyridyl]methyl]pyrrolidine-2-carboxamide; 2,2,2-trifluoroacetic Acid

(Compound of Formula Ia.24, Wherein X is CH, R⁵ is H, R⁶ is F, R⁷ is OCH₃ and R^{8d} is A.5)

(S)-tert-Butyl 2-(((6-chloro-5-fluoro-2-methoxypyridin-3-yl)methyl)carbamoyl)pyrrolidine-1-carboxylate (50 mg, 0.129 mmol 1), (2-(trifluoromethyl)cyclopropyl)boronate (37.7 mg, 0.142 mmol), cesium carbonate (105 mg, 0.322 mmol) and 1,1-Bis(diphenylphospino)ferrocenedichloropalladium(II) (9.43 mg, 0.013 mmol) were dissolved in dioxane (9 ml) and flushed 3× with argon. The reaction mixture was stirred in the Microwave at 90° C. for 10 h, then 120° C. for 2 h and finally at 130° C. for 6 h. The reaction mixture was filtered, washed and concentrated to give (2S)-tert-butyl 2-(((5-fluoro-2-methoxy-6-(2-(trifluoromethyl)cyclopropyl)pyridin-3-yl)methyl)carbamoyl)pyrrolidine-1-carboxylate (206 mg). The material was used as crude product without further purification. To a solution of (2S)-tert-butyl 2-(((5-fluoro-2-methoxy-6-(2-(trifluoromethyl)cyclopropyl)pyridin-3-yl)methyl)carbamoyl)pyrrolidine-1-carboxylate (206 mg, 0.446 mmol) in DCM (5 ml) was added TFA (0.688 ml, 8.93 mmol). The reaction mixture was stirred at room temperature overnight and subsequently concentrated under reduced pressure. The trifluoroacetate salt was obtained by preparative HPLC chromatography on a reversed phase column (eluents contained 0.1% TFA) gave the title compound (2S)—N-((5-fluoro-2-methoxy-6-(2-(trifluoromethyl)cyclopropyl)pyridin-3-yl)methyl)pyrrolidine-2-carboxamide 2,2,2-trifluoroacetate (8.7 mg, yield 4.1%)

LCMS (ESI⁺) m/z [M+H]⁺: 362.20

¹H NMR (500 MHz, methanol-d₄) δ ppm: 7.41 (d, J=10.6 Hz, 1H), 4.60-4.52 (m, 1H), 4.52-4.43 (m, 1H), 4.25-4.20 (m, 1H), 3.93 (s, 3H), 3.45-3.35 (m, 1H), 3.35-3.25 (m, 1H), 2.68-2.57 (m, 1H), 2.40-2.31 (m, 2H), 2.10-1.91 (m, 3H), 1.46-1.40 (m, 1H), 1.38-1.30 (m, 1H).

### Example 140

### (2S)—N-[[5-Fluoro-2-methoxy-6-[4-(trifluoromethyl)cyclohexen-1-yl]-3-pyridyl]methyl]pyrrolidine-2-carboxamide

(Compound of Formula Ia.24, Wherein X is CH, R⁵ is H, R⁶ is F, R⁷ is OCH₃ and R^{8d} is A.93)

(S)-tert-Butyl 2-(((6-chloro-5-fluoro-2-methoxypyridin-3-yl)methyl)carbamoyl)pyrrolidine-1-carboxylate (250 mg, 0.645 mmol), 4-(trifluoromethyl)cyclohex-1-enylboronic acid (125 mg, 0.645 mmol), cesium carbonate (525 mg, 1.612 mmol) and 1,1-bis(diphenylphospino)ferrocenedichloropalladium(II) (47.2 mg, 0.064 mmol) were flushed with argon and then dissolved in dioxane (5 ml) and water (1.2 ml) and stirred at 100° C. for 13 h. The reaction mixture was filtered, washed with ethyl acetate and concentrated. The crude material (990 mg) was purified using flash chromatography (12 g column; DCM 100%→DCM:ethyl acetate 80:20.30 ml/min) to give (2S)-tert-butyl 2-(((5-fluoro-2-methoxy-6-(4-(trifluoromethyl)cyclohex-1-en-1-yl)pyridin-3-yl)methyl)carbamoyl)pyrrolidine-1-carboxylate (128 mg, yield 39.6%). To a solution of (2S)-tert-butyl 2-(((5-fluoro-2-methoxy-6-(4-(trifluoromethyl)cyclohex-1-en-1-yl)pyridin-3-yl)methyl)carbamoyl)pyrrolidine-1-carboxylate (50 mg, 0.100 mmol) in DCM (10 ml) was added TFA (0.154 ml, 1.998 mmol). The reaction mixture was stirred at room temperature for 2 days and subsequently concentrated under reduced pressure. The reaction mixture was diluted with water and the aqueous layer was basified and extracted 4× with DCM. The combined organic layers were dried with magnesium sulfate, filtered and concentrated. The crude material (42 mg) was purified using flash chromatography (4 g column; DCM 100%→DCM:MeOH 90:10.18 ml/min) to give the title compound (2S)—N-((5-fluoro-2-methoxy-6-(4-(trifluoromethyl)cyclohex-1-en-1-yl)pyridin-3-yl)methyl)pyrrolidine-2-carboxamide (38 mg, yield 85%, purity 90%).

LCMS (ESI⁺) m/z [M+H]⁺: 402.20

¹H NMR (600 MHz, DMSO-d₆) δ ppm: 8.40-8.35 (m, 1H), 7.49 (d, J=11.4 Hz, 1H), 5.75 (d, J=14.1 Hz, 1H), 4.38-4.14 (m, 2H), 3.89 (s, 3H), 3.55-3.50 (m, 1H), 2.85-2.80 (m, 1H), 2.80-2.75 (m, 1H), 2.70-2.65 (m, 1H), 2.60-2.55 (m, 1H), 2.45-2.40 (m, 1H), 2.25-2.20 (m, 1H), 2.10-2.05 (m, 1H), 1.95-1.90 (m, 1H), 1.70-1.55 (m, 3H), 1.25-1.20 (m, 1H), 1.20-1.15 (m, 1H).

### Example 141

### (2S)—N-[[5-Fluoro-2-methoxy-6-[[cis-4-(trifluoromethyl)cyclohexyl]amino]-3-pyridyl]methyl]pyrrolidine-2-carboxamide

(Compound of Formula Ia.23, Wherein X is CH, R⁵ is H, R⁶ is F, R⁷ is OCH₃, R^{8b} is H and R^{8c} is A.36)

The title compound was prepared using the procedure described in example 130 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-(trifluoromethyl)cyclohexanamine (103 mg, 0.619 mmol) to give (2S)-tert-butyl 2-(((5-fluoro-2-methoxy-6-((4-(trifluoromethyl)cyclohexyl)amino)pyridin-3-yl)methyl)carbamoyl)pyrrolidine-1-carboxylate (182.8 mg, yield 41%, purity 60%). To a solution of (2S)-tert-butyl 2-(((5-fluoro-2-methoxy-6-((4-(trifluoromethyl)cyclohexyl)amino)pyridin-3-yl)methyl)carbamoyl)pyrrolidine-1-carboxylate (182.8 mg, 0.282 mmol) in DCM (10 ml) was added TFA (0.453 ml, 5.64 mmol). The reaction mixture was stirred at room temperature for 2 h. The reaction mixture was diluted with DCM and was washed once with 2M NaOH. The basified aqueous layer was extracted 5× with DCM. The combined organic layers were dried with sodium sulfate, filtered and concentrated. The raw material was taken onto Bulk Isolute Sorbent and purified using flash chromatography (12 g column; n-heptane 100%→n-heptane: (ethyl acetate/EtOH 3:1) 0:100, 30 ml/min) to give (2S)—N-((5-fluoro-2-methoxy-6-((4-(trifluoromethyl)cyclohexyl)amino)pyridin-3-yl)methyl)pyrrolidine-2-carboxamide (125 mg, yield 101%, purity 95%). The material was used as crude product without further purification. The separation of the isomers was performed with chiral SFC. Preparative method: SFC (Waters Prep 100q SFC); column: Chiralpak® IC for SFC, 20×250 mm, 5 μm eluent: 80% CO₂; 20% methanol with 0.2% aqueous ammonium hydroxide; flow rate: 100 ml/min: time: 1.686 min. (2S)—N-((5-fluoro-2-methoxy-6-((4-(trifluoromethyl)cyclohexyl)amino)pyridin-3-yl)methyl)pyrrolidine-2-carboxamide (35.8 mg, yield 37%).

LCMS (ESI⁺) m/z [M+H]⁺: 419.30

¹H NMR (500 MHz, CDCl₃) δ ppm: 7.86 (s, 1H), 7.12 (d, J=10.5 Hz, 1H), 4.48-4.42 (m, 1H), 4.22 (t, J=4.5 Hz, 3H), 3.86 (s, 2H), 3.73 (dd, J=9.1, 5.3 Hz, 1H), 2.99 (dt, J=10.1, 6.8 Hz, 1H), 2.87 (dt, J=10.2, 6.3 Hz, 1H), 2.13 (ddt, J=12.8, 9.1, 7.3 Hz, 1H), 2.03 (d, J=3.5 Hz, 1H), 1.97-1.83 (m, 1H), 1.81 (ddt, J=11.7, 5.9, 2.9 Hz, 2H), 1.77-1.55 (m, 10H).

### Example 142

### (2S)—N-[[5-Fluoro-2-methoxy-6-[[cis-4-(trifluoromethyl)cyclohexyl]amino]-3-pyridyl]methyl]pyrrolidine-2-carboxamide Hydrochloride

(Compound of Formula Ia.23, Wherein X is CH, R⁵ is H, R⁶ is F, R⁷ is OCH₃, R^{8b} is H and R^{8c} is A.36)

The title compound was prepared using the procedure described in example 141. (2S)—N-((5-fluoro-2-methoxy-6-((4-(trifluoromethyl)cyclohexyl)amino)pyridin-3-yl)methyl)pyrrolidine-2-carboxamide (30.9 mg, 0.074 mmol) was dissolved in DCM (2 ml) and hydrochloric acid 1.25M in MeOH was added and stirred at room temperature for 30 min and subsequently concentrated under reduced pressure to the title compound give (2S)—N-((5-fluoro-2-methoxy-6-((4-(trifluoromethyl)cyclohexyl)amino)pyridin-3-yl)methyl)pyrrolidine-2-carboxamide hydrochloride (30.3 mg, yield 90%).

LCMS (ESI⁺) m/z [M+H]⁺: 419.30

¹H NMR (600 MHz, CDCl₃) δ ppm: 11.40 (s, 1H), 7.84 (s, 1H), 7.30 (d, J=6.9 Hz, 1H), 7.15 (d, J=10.4 Hz, 1H), 4.67 (s, 1H), 4.29 (dd, J=14.7, 5.9 Hz, 1H), 4.27-4.18 (m, 2H), 3.87 (s, 3H), 3.53 (dq, J=12.1, 6.0 Hz, 1H), 3.45-3.31 (m, 1H), 2.53 (q, J=7.6 Hz, 1H), 2.14-2.08 (m, 1H), 2.07-1.96 (m, 2H), 1.81 (ddd, J=15.7, 6.1, 3.2 Hz, 2H), 1.71-1.57 (m, 3H), 1.62 (s, 4H).

### Example 143

### (2S)—N-[[5-Fluoro-2-methoxy-6-[[trans-4-(trifluoromethyl)cyclohexyl]amino]-3-pyridyl]methyl]pyrrolidine-2-carboxamide

(Compound of Formula Ia.23, Wherein X is CH, R⁵ is H, R⁶ is F, R⁷ is OCH₃, R^{8b} is H and R^{8c} is A.36)

The title compound was prepared using the procedure described in example 141.

The separation of the isomers was performed with chiral SFC. Preparative method: SFC (Waters Prep 100q SFC); column: Chiralpak® IC for SFC, 20×250 mm, 5 μm eluent: 80% CO₂; 20% methanol with 0.2% aqueous ammonium hydroxide; flow rate: 100 ml/min: time: 2.406 min.

(2S)—N-((5-fluoro-2-methoxy-6-((4-(trifluoromethyl)cyclohexyl)amino)pyridin-3-yl)methyl)pyrrolidine-2-carboxamide (7.4 mg, yield 7.65%).

LCMS (ESI⁺) m/z [M+H]⁺: 419.25

¹H NMR (500 MHz, CDCl₃) δ ppm: 7.87 (s, 1H), 7.15-7.07 (m, 1H), 4.28-4.17 (m, 3H), 3.87 (s, 3H), 3.88-3.77 (m, 1H), 3.72 (dd, J=9.1, 5.3 Hz, 1H), 3.09-2.95 (m, 1H), 2.89-2.84 (m, 1H), 2.28 (dd, J=12.9, 3.8 Hz, 2H), 2.20-2.07 (m, 1H), 2.11-1.97 (m, 3H), 1.94-1.88 (m, 1H), 1.74-1.62 (m, 2H), 1.55-1.42 (m, 2H), 1.25 (d, J=25.6 Hz, 2H).

### Example 144

### (2S)—N-[[5-Fluoro-2-methoxy-6-[[trans-4-(trifluoromethyl)cyclohexyl]amino]-3-pyridyl]methyl]pyrrolidine-2-carboxamide Hydrochloride

(Compound of Formula Ia.23, Wherein X is CH, R⁵ is H, R⁶ is F, R⁷ is OCH₃, R^{8b} is H and R^{8c} is A.36)

The free base was prepared using the procedure described in example 143. The intermediate compound was prepared using the procedure described in example 130. (2S)—N-((5-fluoro-2-methoxy-6-((4-(trifluoromethyl)cyclohexyl)amino)pyridin-3-yl)methyl)pyrrolidine-2-carboxamide (30.9 mg, 0.074 mmol) was dissolved in DCM (2 ml) and hydrochloric acid 1.25M in MeOH was added. The mixture was stirred at room temperature for 30 min and subsequently concentrated under reduced pressure to give the title compound (2S)—N-((5-fluoro-2-methoxy-6-((4-(trifluoromethyl)cyclohexyl)amino)pyridin-3-yl)methyl)pyrrolidine-2-carboxamide hydrochloride (2.6 mg, yield 99%, purity 95%).

LCMS (ESI⁺) m/z [M+H]⁺: 419.30

¹H NMR (600 MHz, CDCl₃) δ ppm: 11.45 (s, 1H), 7.64 (s, 1H), 7.15 (s, 1H), 4.67 (s, 1H), 4.27 (s, 2H), 3.86 (s, 3H), 3.83-3.76 (m, 1H), 3.57-3.51 (m, 1H), 3.42-3.33 (m, 1H), 2.56-2.43 (m, 1H), 2.29-2.19 (m, 2H), 2.06-1.94 (m, 4H), 1.49-1.42 (m, 3H), 1.29-1.16 (m, 3H).

### Example 145

### (2S)—N-[(6-Benzyl-5-fluoro-2-methoxy-3-pyridyl)methyl]pyrrolidine-2-carboxamide

(Compound of Formula Ia.24, Wherein X is CH, R⁵ is H, R⁶ is F, R⁷ is OCH₃ and R^{8d} is —CH₂-A.59)

The title compound was prepared using the procedure described in example 140. (S)-tert-butyl 2-(((6-chloro-5-fluoro-2-methoxypyridin-3-yl)methyl)carbamoyl)pyrrolidine-1-carboxylate (200 mg, 0.516 mmol), benzylboronic acid pinacol ester (117 mg, 0.516 mmol), cesium carbonate (420 mg, 1.289 mmol) and 1,1-bis(diphenylphospino)ferrocenedichloropalladium(II) (37.7 mg, 0.052 mmol) were flushed with argon and then dissolved in dioxane (5 ml) and water (1.2 ml) and stirred at 90° C. for 12 h. The reaction mixture was filtered, washed with ethyl acetate and concentrated. The crude material (800 mg) was purified using flash chromatography (12 g column; DCM 100%→DCM:ethyl acetate 80:20.30 ml/min) to give (S)-tert-butyl 2-(((6-benzyl-5-fluoro-2-methoxypyridin-3-yl)methyl)carbamoyl)pyrrolidine-1-carboxylate (215 mg, yield 65.8%, purity 70%). The material was used as crude product without further purification. The Boc-deprotection was prepared using the procedure described in example 140 to give (S)—N-((6-benzyl-5-fluoro-2-methoxypyridin-3-yl)methyl)pyrrolidine-2-carboxamide (68 mg, yield 40.8%).

LCMS (ESI⁺) m/z [M+H]⁺: 344.30

¹H NMR (500 MHz, DMSO-d₆) δ ppm: 8.40-8.35 (m, 1H), 7.43 (d, J=11.3 Hz, 1H), 7.31-7.21 (m, 4H), 7.24-7.14 (m, 1H), 4.30-4.20 (m, 2H), 4.07 (s, 2H), 3.89 (s, 3H), 3.50-3.45 (m, 1H), 2.85-2.80 (m, 1H), 2.77-2.70 (m, 1H), 1.99-1.87 (m, 1H), 1.68-1.50 (m, 3H).

### Example 146

### (2S)—N-[[6-(Cyclobutylamino)-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is A.8)

(S)-tert-Butyl 2-(((6-chloro-5-fluoro-2-methoxypyridin-3-yl)methyl)carbamoyl)pyrrolidine-1-carboxylate (100 mg, 0.258 mmol), cyclobutylamine (18.34 mg, 0.258 mmol), 2,2′-bis(diphenylphosphino)-1,1′-binaphthalene (BINAP) (24.08 mg, 0.039 mmol), sodium 2-methylpropan-2-olate (61.9 mg, 0.645 mmol) and tris(dibenzylideneacetone)dipalladium(0) (35.4 mg, 0.039 mmol) were suspended in toluene (10 ml) and flushed with nitrogen. The reaction mixture stirred at 120° C. for 4 h and at 140° C. for 1 h and subsequently concentrated under reduced pressure. The crude material was washed with ethyl acetate and water and was concentrated to give (S)-tert-butyl 2-(((6-(cyclobutylamino)-5-fluoro-2-methoxypyridin-3-yl)methyl)carbamoyl)pyrrolidine-1-carboxylate (174 mg). The material was used as crude product without further purification. To a solution of (S)-tert-butyl 2-(((6-(cyclobutylamino)-5-fluoro-2-methoxypyridin-3-yl)methyl)carbamoyl)pyrrolidine-1-carboxylate (174 mg, 0.412 mmol) in DCM (10 ml) was added TFA (635 μl, 8.24 mmol). The reaction mixture was stirred at room temperature overnight and subsequently concentrated under reduced pressure. The residue was washed with DCM and water to give 127.3 mg crude material. Preparative HPLC chromatography on a reversed phase column (eluents contained 0.1% TFA) gave the title compound (S)—N-((6-(cyclobutylamino)-5-fluoro-2-methoxypyridin-3-yl)methyl)pyrrolidine-2-carboxamide 2,2,2-trifluoroacetate (26 mg, yield 14.4%).

LCMS (ESI⁺) m/z [M+H]⁺: 323.30

¹H NMR (500 MHz, methanol-d₄) δ ppm: 7.15 (d, J=10.8 Hz, 1H), 4.52-4.42 (m, 1H), 4.28-4.15 (m, 3H), 3.87 (s, 3H), 3.45-3.40 (m, 1H), 2.44-2.30 (m, 3H), 2.08-1.91 (m, 6H), 1.82-1.68 (m, 2H).

### Example 147

### (2S)—N-[[6-[N-Cyclopropyl-N-methyl-amino]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is CH₃ and R^{8c} is A.1)

The title compound was prepared using the procedure described in example 146. (S)-tert-butyl 2-(((6-chloro-5-fluoro-2-methoxypyridin-3-yl)methyl)carbamoyl)pyrrolidine-1-carboxylate (150 mg, 0.387 mmol), N-methylcyclopropaneamine (55 mg, 0.774 mmol), dicyclohexyl(2′,6′-diisopropoxy-[1,1′-biphenyl]-2-yl)phosphine (RuPhos) (27.1 mg, 0.058 mmol), sodium 2-methylpropan-2-olate (93 mg, 0.967 mmol) and bis(dibenzylideneacetone)palladium(0) (33.4 mg, 0.058 mmol) were suspended in toluene (12 ml) and flushed with argon. The reaction mixture was stirred in the Microwave at 120° C. for 2 h. The crude material was washed with ethyl acetate and water and was concentrated to give (S)-tert-butyl 2-(((6-(cyclopropyl(methyl)amino)-5-fluoro-2-methoxypyridin-3-yl)methyl)carbamoyl)pyrrolidine-1-carboxylate (210 mg, yield 96%, purity 75%). The material was used as crude product without further purification. The Boc deprotection was prepared using the procedure described in example 146 to give the title compound (S)—N-((6-(cyclopropyl(methyl)amino)-5-fluoro-2-methoxypyridin-3-yl)methyl)pyrrolidine-2-carboxamide 2,2,2-trifluoroacetate (3.1 mg, yield 1.4%).

LCMS (ESI+) m/z [M+H]⁺: 323.35

¹H NMR (500 MHz, methanol-d₄) δ ppm: 7.19 (d, J=11.6 Hz, 1H), 4.25-4.15 (m, 2H), 3.89 (s, 3H), 3.50-3.40 (m, 1H), 3.05 (s, 3H), 2.45-2.35 (m, 2H), 2.10-1.75 (m, 5H), 0.80-0.75 (m, 2H), 0.55-0.50 (m, 2H).

### Example 148 and Example 149

### Two Diastereomers of (2S)—N-[[5-fluoro-2-methoxy-6-[[4-(trifluoromethyl)cyclohexyl]methylamino]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is —CH₂-A.36)

The title compounds were prepared using the procedure described in example 147 starting from (S)-tert-butyl 2-(((6-chloro-5-fluoro-2-methoxypyridin-3-yl)methyl)carbamoyl)pyrrolidine-1-carboxylate (150 mg, 0.387 mmol) and N-methyl-4-(trifluoromethyl)cyclohexanamine (140 mg, 0.774 mmol) to give (S)-tert-butyl 2-(((5-fluoro-2-methoxy-6-(methyl(4-(trifluoromethyl)cyclohexyl)amino)pyridin-3-yl)methyl)carbamoyl)pyrrolidine-1-carboxylate (553 mg) crude material, followed by Boc deprotection to give (S)—N-((5-fluoro-2-methoxy-6-(methyl(4-(trifluoromethyl)cyclohexyl)amino)pyridin-3-yl)methyl)pyrrolidine-2-carboxamide (225 mg). Preparative HPLC chromatography on a reversed phase column (eluents contained 0.1% TFA) gave one diastereomer of (S)—N-((5-fluoro-2-methoxy-6-((((1s,4R)-4-(trifluoromethyl)cyclohexyl)methyl)amino)pyridin-3-yl)methyl)pyrrolidine-2-carboxamide 2,2,2-trifluoroacetate (17.7 mg, yield 3.1%) and another diastereomer of (S)—N-((5-fluoro-2-methoxy-6-((((1s,4R)-4-(trifluoromethyl)cyclohexyl)methyl)amino)pyridin-3-yl)methyl)pyrrolidine-2-carboxamide 2,2,2-trifluoroacetate (38.4 mg, yield 6.8%).

### Diastereomer I:

LCMS (ESI⁺) m/z [M+H]⁺: 433.30

¹H NMR (500 MHz, methanol-d₄) δ ppm: 7.15 (d, J=10.9 Hz, 1H), 4.25-4.15 (m, 2H), 3.87 (s, 3H), 3.55-3.50 (m, 2H), 3.45-3.27 (m, 1H), 3.35-3.25 (m, 3H), 2.45-2.35 (m, 1H), 2.20-2.10 (m, 1H), 2.05-1.90 (m, 4H), 1.75-1.60 (m, 5H), 1.65-1.50 (m, 2H).

### Diastereomer II:

LCMS (ESI⁺) m/z [M+H]⁺: 433.30

¹H NMR (500 MHz, methanol-d₄) δ ppm: 7.15 (d, J=10.8 Hz, 1H), 4.27-4.15 (m, 2H), 3.87 (s, 3H), 3.45-3.27 (m, 1H), 3.35-3.25 (m, 5H), 2.45-2.35 (m, 1H), 2.10-1.90 (m, 7H), 1.70-1.60 (m, 1H), 1.35-1.25 (m, 2H), 1.10-1.00 (m, 2H).

### Example 150

### (2S)—N-[[6-[N-Allyl-N-benzyl-amino]-5-fluoro-2-methoxy-3-pyridyl]methyl]-pyrrolidine-2-carboxamide Hydrochloride

(Compound of Formula Ia.23, Wherein X is CH, R⁵ is H, R⁶ is F, R⁷ is OCH₃, R^{8b} is allyl and R^{8c} is —CH₂-A.59)

(S)-tert-Butyl 2-(((6-chloro-5-fluoro-2-methoxypyridin-3-yl)methyl)carbamoyl)pyrrolidine-1-carboxylate (100 mg, 0.258 mmol), dicyclohexyl(2′,6′-diisopropoxy-[1,1′-biphenyl]-2-yl)phosphine (RuPhos) (18.05 mg, 0.039 mmol), sodium 2-methylpropan-2-olate (61.9 mg, 0.645 mmol) and bis(dibenzylideneacetone)palladium(0) (22.24 mg, 0.039 mmol) were suspended in toluene (5 ml) and flushed with Argon. N-benzylcyclopropanamine (60 mg, 0.408 mmol was added and the reaction mixture was stirred in the Microwave at 120° C. for 2 h. Charcoal was added to the reaction mixture and stirred 5 minutes. This suspension was filtered through celite and washed with toluene. The solvent was evaporated. The crude material was purified twice using flash chromatography (12 g column; DCM 100%→DCM:MeOH 95:5.30 ml/min) to give (S)-tert-butyl 2-(((6-(benzyl(cyclopropyl)amino)-5-fluoro-2-methoxypyridin-3-yl)methyl)carbamoyl)pyrrolidine-1-carboxylate (22 mg, yield 17.1%). The title compound was prepared using the procedure described in example 138 (5 mg, yield 23.45%, purity 90%).

LCMS (ESI⁺) m/z [M+H]⁺: 399.20

¹H NMR (500 MHz, DMSO-d₆) δ ppm: 9.41 (s, 1H), 8.79 (t, J=5.5 Hz, 1H), 8.55 (s, 1H), 7.38-7.19 (m, 6H), 5.96-5.86 (m, 1H), 5.20-5.11 (m, 2H), 4.67 (s, 2H), 4.21-4.04 (m, 5H), 3.73 (s, 3H), 3.20 (dq, J=17.7, 5.4 Hz, 1H), 2.27 (ddt, J=12.8, 8.4, 6.5 Hz, 1H), 1.88 (p, J=7.1 Hz, 2H), 1.86-1.76 (m, 1H).

### Example 151

### (2S)—N-[[6-[3-(4-Chlorophenoxy)azetidin-1-yl]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.104)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-(4-chlorophenoxy)azetidine hydrochloride (125 mg, 0.567 mmol) to give (S)-tert-butyl 2-(((6-(3-(4-chlorophenoxy)azetidin-1-yl)-5-fluoro-2-methoxypyridin-3-yl)methyl)carbamoyl)pyrrolidine-1-carboxylate (357.7 mg) as crude product followed by Boc deprotection as described in procedure example 136. Preparative HPLC chromatography on a reversed phase column (eluents contained 0.1% TFA) gave the title compound (S)—N-((6-(3-(4-chlorophenoxy)azetidin-1-yl)-5-fluoro-2-methoxypyridin-3-yl)methyl)pyrrolidine-2-carboxamide 2,2,2-trifluoroacetate (15.7 mg, yield 4.3%).

LCMS (ESI⁺) m/z [M+H]⁺: 435.20

¹H NMR (600 MHz, DMSO-d₆) δ ppm: 9.20 (bs, 1H), 8.75-8.70 (m, 1H), 7.35-7.30 (m, 3H), 6.90-6.85 (m, 2H), 5.15-5.10 (m, 1H), 4.55-4.50 (m, 2H), 4.15-4.10 (m, 3H), 4.00-3.95 (m, 2H), 3.82 (s, 3H), 3.25-3.15 (m, 2H), 2.30-2.25 (m, 1H), 1.95-1.85 (m, 2H), 1.85-1.75 (m, 1H).

### Example 152

### (2S)—N-[[5-Fluoro-2-methoxy-6-[[1-(trifluoromethyl)cyclopropyl]methylamino]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is —CH₂-A.4)

The title compound was prepared using the procedure described in example 146 starting from (S)-tert-butyl 2-(((6-chloro-5-fluoro-2-methoxypyridin-3-yl)methyl)carbamoyl)pyrrolidine-1-carboxylate (100 mg, 0.258 mmol) and (1-(trifluoromethyl)cyclopropyl)methanamine hydrochloride (50 mg, 0.29 mmol) to give (S)-tert-butyl 2-(((5-fluoro-2-methoxy-6-(((1-(trifluoromethyl)cyclopropyl)methyl)amino)pyridin-3-yl)methyl)carbamoyl)pyrrolidine-1-carboxylate (122 mg). Boc deprotection of the crude product was carried out according to the procedure described in example 146. Preparative HPLC chromatography on a reversed phase column (eluents contained 0.1% TFA) gave the title compound (S)—N-((5-fluoro-2-methoxy-6-(((1-(trifluoromethyl)cyclopropyl)methyl)amino)pyridin-3-yl)methyl)pyrrolidine-2-carboxamide 2,2,2-trifluoroacetate (12.7 mg, yield 10.2%).

LCMS (ESI⁺) m/z [M+H]⁺: 391.25

¹H NMR (500 MHz, methanol-d₄) δ ppm: 7.20 (d, J=10.8 Hz, 1H), 4.28-4.17 (m, 2H), 3.88 (s, 3H), 3.88-3.76 (m, 2H), 3.45-3.35 (m, 1H), 3.30-3.25 (m, 2H), 2.44-2.33 (m, 1H), 2.09-1.91 (m, 3H), 0.96-0.82 (m, 4H).

### Example 153

### (2S)—N-[[5-Fluoro-2-methoxy-6-[3-(trifluoromethyl)-1-piperidyl]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.125)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-(trifluoromethyl)piperidine (79 mg, 0.516 mmol) to give (2S)-tert-butyl 2-(((5-fluoro-2-methoxy-6-(3-(trifluoromethyl)piperidin-1-yl)pyridin-3-yl)methyl)carbamoyl)pyrrolidine-1-carboxylate (286 mg). Boc deprotection of the crude product was carried out according to the procedure described in example 146. Preparative HPLC chromatography on a reversed phase column (eluents contained 0.1% TFA) gave the title compound (2S)—N((5-fluoro-2-methoxy-6-(3-(trifluoromethyl)piperidin-1-yl)pyridin-3-yl)methyl)pyrrolidine-2-carboxamide 2,2,2-trifluoroacetate (34.4 mg, yield 11.7%).

LCMS (ESI⁺) m/z [M+H]⁺: 405.20

¹H NMR (500 MHz, DMSO-d₆) δ ppm: 8.80-8.75 (m, 1H), 7.39 (d, J=12.9 Hz, 1H), 4.20-4.15 (m, 2H), 3.95-3.90 (m, 1H), 3.84 (s, 3H), 3.29-3.15 (m, 2H), 3.03-2.91 (m, 2H), 2.65-2.55 (m, 1H), 2.33-2.19 (m, 1H), 2.05-1.65 (m, 7H), 1.62-1.46 (m, 2H).

### Example 154

### (2S)—N-[[6-[N-(4,4-Difluorocyclohexyl)-N-methyl-amino]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is methyl and R^{8c} is −A.33)

The title compound was prepared using the procedure described in example 146 starting from (S)-tert-butyl 2-(((6-chloro-5-fluoro-2-methoxypyridin-3-yl)methyl)carbamoyl)pyrrolidine-1-carboxylate (200 mg, 0.516 mmol) and N-4,4-difluoro-N-methylcyclohexane amine hydrochloride (191 mg, 1.031 mmol) to give 263 mg of crude product which was directly taken into Boc deprotection using the procedure described in example 146. Preparative HPLC chromatography on a reversed phase column (eluents contained 0.1% TFA) gave the title compound (S)—N-((6-((4,4-difluorocyclohexyl)(methyl)amino)-5-fluoro-2-methoxypyridin-3-yl)methyl)pyrrolidine-2-carboxamide 2,2,2-trifluoroacetate (5.8 mg, yield 2.1%).

LCMS (ESI⁺) m/z [M+H]⁺: 401.25

¹H NMR (500 MHz, methanol-d₄) δ ppm: 7.25 (d, J=13.0 Hz, 1H), 4.33-4.13 (m, 2H), 3.88 (s, 3H), 3.46-3.26 (m, 1H), 2.95 (d, J=2.9 Hz, 3H), 2.45-2.35 (m, 1H), 2.19-2.11 (m, 2H), 2.09-1.75 (m, 12H).

### Example 155

### (2S)—N-[[6-(Azepan-1-yl)-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.133)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and hexamethyleneimine (59 μl, 0.516 mmol) to give (S)-tert-butyl 2-(((6-(azepan-1-yl)-5-fluoro-2-methoxypyridin-3-yl)methyl)carbamoyl)pyrrolidine-1-carboxylate (300.4 mg) as crude product. Boc deprotection using the procedure described in example 146 followed by the addition of Bulk Isolute Sorbent and purification using flash chromatography (4 g column; DCM 100%→DCM:MeOH 0:100, 18 ml/min) gave 105 mg of crude product. Preparative HPLC chromatography on a reversed phase column (eluents contained 0.1% TFA) gave the title compound (S)—N-((6-(azepan-1-yl)-5-fluoro-2-methoxypyridin-3-yl)methyl)pyrrolidine-2-carboxamide 2,2,2-trifluoroacetate (57.6 mg, yield 18.6%).

LCMS (ESI⁺) m/z [M+H]⁺: 351.30

¹H NMR (500 MHz, CDCl₃) δ ppm: 7.06 (d, J=13.1 Hz, 1H), 6.94 (bs, 1H), 4.65 (bs, 1H), 4.31-4.20 (m, 2H), 3.85 (s, 3H), 3.70-3.65 (m, 4H), 3.50-3.45 (m, 1H), 3.45-3.40 (m, 1H), 2.40-2.30 (m, 2H), 2.15-2.10 (m, 1H), 2.06-1.95 (m, 2H), 1.80-1.75 (m, 4H), 1.60-1.55 (m, 4H).

LJ: 1048/311/3

### Example 156

### (2S)—N-[[6-(3,3-Difluoro-1-piperidyl)-5-fluoro-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.119)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3,3-difluoropiperidine (62.5 mg, 0.516 mmol) to give 317 mg of crude product followed by Boc deprotection using the procedure described in example 155. Preparative HPLC chromatography on a reversed phase column (eluents contained 0.1% TFA) gave the title compound (S)—N((6-(3,3-difluoropiperidin-1-yl)-5-fluoro-2-methoxypyridin-3-yl)methyl)pyrrolidine-2-carboxamide 2,2,2-trifluoroacetate (78.7 mg, yield 19.3%, purity 80%).

LCMS (ESI+) m/z [M+H]⁺: 373.30

¹H NMR (500 MHz, CDCl₃) δ ppm: 7.65-7.60 (m, 1H), 7.16 (d, J=12.2 Hz, 1H), 4.64 (bs, 1H), 4.34-4.20 (m, 2H), 3.87 (s, 3H), 3.75-3.65 (m, 2H), 3.50-3.45 (m, 2H), 3.40-3.35 (m, 2H), 2.45-2.30 (m, 3H), 2.10-1.95 (m, 4H), 1.90-1.80 (m, 2H).

### Example 157

### Diastereomeric Mixture of (2S)—N-[[5-fluoro-2-methoxy-6-[[4-(trifluoromethyl)cyclohexyl]amino]-3-pyridyl]methyl]-N-methyl-pyrrolidine-2-carboxamide; 2,2,2-trifluoroacetic Acid

(Compound of Formula Ia.23, Wherein X is CH, R⁵ is methyl, R⁶ is F, R⁷ is OCH₃, R^{8b} is H and R^{8c} is −A.36)

(S)-tert-Butyl 2-(((6-chloro-5-fluoro-2-methoxypyridin-3-yl)methyl)carbamoyl)pyrrolidine-1-carboxylate (2.0 g, 5.16 mmol) was dissolved in DMF (30 ml) and flushed with argon at 0° C. Sodium hydride (338 mg, 7.74 mmol, 55%) was added at 0° C. and stirred for 1 h. Iodomethane (482 μl, 7.74 mmol) was added and stirred at 0° C. for 3h. The reaction mixture was allowed to warm to room temperature and stirred for 2 h. The reaction mixture was cooled down to 0° C. and water was added. This mixture was basified with 1M NaOH and extracted 3× with MTBE. The organic layer was concentrated. The crude material (1.3 g) was dissolved in ethyl acetate and Bulk Isolute Sorbent was added. The mixture was purified using flash chromatography (12 g column; DCM 100%→DCM:MeOH 0:100, 30 ml/min) to give 821 mg crude product. The product was obtained by preparative HPLC chromatography on a reversed phase column to give (S)-tert-butyl 2-(((6-chloro-5-fluoro-2-methoxypyridin-3-yl)methyl)(methyl)carbamoyl)pyrrolidine-1-carboxylate (167.9 mg). The title compound was prepared using the procedure described in example 128 starting with (S)-tert-butyl 2-(((6-chloro-5-fluoro-2-methoxypyridin-3-yl)methyl)(methyl)carbamoyl)pyrrolidine-1-carboxylate (147 mg, 0.366 mmol) and 4-(trifluoromethyl)cyclohexane amine (73.4 mg, 0.439 mmol). The crude material was washed with ethyl acetate and water and was concentrated to give (S)-tert-butyl 2-(((5-fluoro-2-methoxy-6-((4-(trifluoromethyl)cyclohexyl)amino)pyridin-3-yl)methyl)(methyl)carbamoyl)pyrrolidine-1-carboxylate (260.7 mg) as crude product. Boc deprotection was carried out using the procedure described in example 140 to give 391 mg of crude product. Preparative HPLC chromatography on a reversed phase column (eluents contained 0.1% TFA) gave a diastomeric mixture of (S)—N-((5-fluoro-2-methoxy-6-((4-(trifluoromethyl)cyclohexyl)amino)pyridin-3-yl)methyl)-N-methylpyrrolidine-2-carboxamide 2,2,2-trifluoroacetate (18.4 mg, 6.9%).

LCMS (ESI⁺) m/z [M+H]⁺: 433.30

¹H NMR (500 MHz, methanol d₄) δ ppm: 7.17/7.15 (d, J=10.8 Hz, 1H), 4.50-4.10 (m, 2H), 3.88/3.86 (s, 3H), 3.50-3.40 (m, 1H), 3.40-3.30 (m, 1H), 3.00/2.85 (s, 3H), 2.55-2.45 (m, 1H), 2.25-1.90 (m, 8H), 1.50-1.30 (m, 6H).

### Example 158

### (2S)—N-[(5-Fluoro-2-methoxy-6-tetrahydropyran-3-yl-3-pyridyl)methyl]-pyrrolidine-2-carboxamide Hydrochloride

(Compound of Formula Ia.24, Wherein X is CH, R⁵ is H, R⁶ is F, R⁷ is OCH₃ and R^{8d} is A.95)

(S)-tert-Butyl 2-(((6-chloro-5-fluoro-2-methoxypyridin-3-yl)methyl)carbamoyl)pyrrolidine-1-carboxylate (150 mg, 0.387 mmol), [1,1′-bis(diphenylphospino)ferrocene]dichloropalladium(II) (28.3 mg, 0.039 mmol) and cesium carbonate (315 mg, 0.967 mmol) were suspended in dioxane (2.5 ml) and water (0.6 ml). Then 2-(3,4-dihydro-2H-pyran-5-yl)-4,4,5,5-tetramethyl-1,3,2-dioxaborolane (98 mg, 464 mmol) was added and flushed with argon. The reaction was stirred in the Microwave at 90° C. for 8 h and subsequently concentrated under reduced pressure. The residue was dissolved in ethyl acetate and washed twice with water. The aqueous layer was twice washed with ethyl acetate. The combined organic layers were dried with magnesium sulfate, filtered and concentrated. The crude material was purified using flash chromatography (12 g column; DCM 100%→DCM:MeOH 95:5.30 ml/min) to give (S)-tert-butyl 2-(((6-(3,4-dihydro-2H-pyran-5-yl)-5-fluoro-2-methoxypyridin-3-yl)methyl)carbamoyl)pyrrolidine-1-carboxylate (151 mg, yield 90%). (S)-tert-Butyl 2-(((6-(3,4-dihydro-2H-pyran-5-yl)-5-fluoro-2-methoxypyridin-3-yl)methyl)carbamoyl)pyrrolidine-1-carboxylate (106 mg, 0.243 mmol) was dissolved in EtOH (3.5 ml) and palladium on carbon (10%) (49 mg, 0.046 mmol) was added followed by the addition of ammonium formate (890 mg, 19.33 mmol) in water (2.5 ml) and stirred at 80° C. for 12 h. The reaction mixture was allowed to cool down to room temperature and was diluted with ethyl acetate. The reaction mixture was filtered and the aqueous layer was separated. The organic layer was dried with magnesium sulfate, filtered and concentrated. The crude material was purified using flash chromatography (12 g column; DCM 100%→DCM:MeOH 95:5.30 ml/min) to give (2S)-tert-butyl 2-(((5-fluoro-2-methoxy-6-(tetrahydro-2H-pyran-3-yl)pyridin-3-yl)methyl)carbamoyl)pyrrolidine-1-carboxylate (76 mg, yield 71.4%). The final compound was prepared using the procedure described in example 138 to give the title compound (2S)-2-(((5-fluoro-2-methoxy-6-(tetrahydro-2H-pyran-3-yl)pyridin-3-yl)methyl)carbamoyl)pyrrolidin-1-ium chloride (54 mg, yield 83%).

LCMS (ESI⁺) m/z [M+H]⁺: 338.20

¹H NMR (600 MHz, DMSO-d₆) δ ppm: 9.70 (s, 1H), 9.04 (t, J=5.7 Hz, 1H), 8.56 (s, 1H), 7.49 (d, J=9.5 Hz, 1H), 4.29-4.17 (m, 3H), 3.89 (s, 3H), 3.88 (tt, J=11.2, 3.4 Hz, 2H), 3.50 (td, J=10.8, 1.7 Hz, 1H), 3.39-3.34 (m, 1H), 3.29-3.01 (m, 3H), 2.32 (ddt, J=12.5, 8.3, 5.7 Hz, 1H), 1.96-1.81 (m, 5H), 1.71-1.62 (m, 1H), 1.68 (s, 1H).

### Example 159

### (2S)—N-[(5-Fluoro-2-methoxy-6-tetrahydropyran-4-yl-3-pyridyl)methyl]-pyrrolidine-2-carboxamide Hydrochloride

(Compound of Formula Ia.24, Wherein X is CH, R⁵ is H, R⁶ is F, R⁷ is OCH₃ and R^{8d} is A.96)

The title compound was prepared using the procedure described in example 158 starting from (S)-tert-butyl 2-(((6-chloro-5-fluoro-2-methoxypyridin-3-yl)methyl)carbamoyl)pyrrolidine-1-carboxylate (150 mg, 0.387 mmol) and 2-(3,6-dihydro-2H-pyran-4-yl)-4,4,5,5-tetramethyl-1,3,2-dioxaborolane (98 mg, 0.464 mmol) to give (S)-2-(((5-fluoro-2-methoxy-6-(tetrahydro-2H-pyran-4-yl)pyridin-3-yl)methyl)carbamoyl)pyrrolidin-1-ium chloride (95 mg, yield 75%, purity 95%).

LCMS (ESI⁺) m/z [M+H]⁺: 338.20

¹H NMR (600 MHz, DMSO-d₆) δ ppm: 9.67 (s, 1H), 9.04 (t, J=5.7 Hz, 1H), 8.58 (s, 1H), 7.48 (d, J=9.6 Hz, 1H), 4.31-4.20 (m, 3H), 3.98-3.91 (m, 2H), 3.90 (s, 3H), 3.47 (td, J=11.7, 2.0 Hz, 2H), 3.39-3.33 (m, 1H), 3.26-3.12 (m, 2H), 2.32 (ddt, J=12.4, 8.3, 5.7 Hz, 1H), 1.94-1.81 (m, 5H), 1.61 (ddd, J=12.9, 4.0, 1.9 Hz, 2H).

### Example 160

### (2S)—N-[[5-Fluoro-6-(4-fluoro-1-piperidyl)-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.117)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-fluoropiperidine (53.2 mg, 0.516 mmol) to give (S)-tert-butyl 2-(((5-fluoro-6-(4-fluoropiperidin-1-yl)-2-methoxypyridin-3-yl)methyl)carbamoyl)pyrrolidine-1-carboxylate as crude product (285.6 mg) followed by Boc deprotection using the procedure described in example 136. Preparative HPLC chromatography on a reversed phase column (eluents contained 0.1% TFA) gave the title compound (S)—N-((5-fluoro-6-(4-fluoropiperidin-1-yl)-2-methoxypyridin-3-yl)methyl)pyrrolidine-2-carboxamide 2,2,2-trifluoroacetate (110.2 mg, yield 37.5%).

LCMS (ESI⁺) m/z [M+H]⁺: 355.25

¹H NMR (500 MHz, methanol-d₄) δ ppm: 7.27 (s, 1H), 4.25-4.15 (m, 3H), 3.89 (s, 3H), 3.70-3.60 (m, 2H), 3.50-3.45 (m, 3H), 3.40-3.35 (m, 3H), 2.45-2.35 (m, 1H), 2.10-1.95 (m, 5H), 1.85-1.75 (m, 2H).

### Example 161

### (2S)—N-[[5-Fluoro-6-(3-fluoro-1-piperidyl)-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.116)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-fluoropiperidine (53.2 mg, 0.516 mmol) to give (2S)-tert-butyl 2-(((5-fluoro-6-(3-fluoropiperidin-1-yl)-2-methoxypyridin-3-yl)methyl)carbamoyl)pyrrolidine-1-carboxylate as crude product (309.2 mg) followed by Boc deprotection using the procedure described in example 136. Preparative HPLC chromatography on a reversed phase column (eluents contained 0.1% TFA) gave the title compound (2S)—N-((5-fluoro-6-(3-fluoropiperidin-1-yl)-2-methoxypyridin-3-yl)methyl)pyrrolidine-2-carboxamide 2,2,2-trifluoroacetate (59.8 mg, yield 18.8%).

LCMS (ESI⁺) m/z [M+H]⁺: 355.30

¹H NMR (500 MHz, methanol-d₄) δ ppm: 7.28 (d, J=12.5 Hz, 1H), 4.75-4.65 (m, 1H), 4.25-4.15 (m, 3H), 3.90 (s, 3H), 3.75-3.55 (m, 3H), 3.45-3.35 (m, 2H), 2.45-2.35 (m, 1H), 2.10-1.95 (m, 6H), 1.90-1.80 (m, 1H), 1.65-1.55 (m, 1H).

### Example 162

### (2S)—N-[[5-Fluoro-6-(3-Fluoropyrrolidin-1-yl)-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.108)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-fluoropyrrolidine hydrochloride (78 mg, 0.619 mmol)) to give (2S)-tert-butyl 2-(((5-fluoro-6-(3-fluoropyrrolidin-1-yl)-2-methoxypyridin-3-yl)methyl)carbamoyl)pyrrolidine-1-carboxylate as crude product (329 mg) followed by Boc deprotection using the procedure described in example 136. Preparative HPLC chromatography on a reversed phase column (eluents contained 0.1% TFA) gave the title compound (2S)—N-((5-fluoro-6-(3-fluoropyrrolidin-1-yl)-2-methoxypyridin-3-yl)methyl)pyrrolidine-2-carboxamide 2,2,2-trifluoroacetate (73.6 mg, yield 21.7%).

LCMS (ESI⁺) m/z [M+H]⁺: 341.25

¹H NMR (500 MHz, methanol-d₄) δ ppm: 7.23 (d, J=12.6 Hz, 1H), 5.35-5.35 (m, 1H), 4.25-4.15 (m, 3H), 3.89 (s, 3H), 3.85-3.65 (m, 3H), 3.45-3.35 (m, 2H), 2.45-2.35 (m, 1H), 2.30-1.90 (m, 6H).

### Example 163

### (2S)—N-[[5-Fluoro-2-methoxy-6-[3-(trifluoromethyl)pyrrolidin-1-yl]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.112)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-(trifluoromethyl)pyrrolidine hydrochloride (109 mg, 0.619 mmol) to give (2S)-tert-butyl 2-(((5-fluoro-2-methoxy-6-(3-(trifluoromethyl)pyrrolidin-1-yl)pyridin-3-yl)methyl)carbamoyl)pyrrolidine-1-carboxylate as crude product (525 mg) followed by Boc deprotection using the procedure described in example 136. Preparative HPLC chromatography on a reversed phase column (eluents contained 0.1% TFA) gave the title compound (2S)—N((5-fluoro-2-methoxy-6-(3-(trifluoromethyl)pyrrolidin-1-yl)pyridin-3-yl)methyl)pyrrolidine-2-carboxamide 2,2,2-trifluoroacetate (140.2 mg, yield 26%).

LCMS (ESI⁺) m/z [M+H]⁺: 391.20

¹H NMR (500 MHz, methanol-d₄) δ ppm: 7.25 (d, J=12.6 Hz, 1H), 4.25-4.15 (m, 3H), 3.89 (s, 3H), 3.85-3.60 (m, 5H), 3.45-3.35 (m, 1H), 3.20-3.10 (m, 1H), 2.45-2.35 (m, 1H), 2.30-2.20 (m, 1H), 2.15-2.05 (m, 1H), 2.05-1.90 (m, 3H).

### Example 164

### (2S)—N-[[6-(3,3-Difluoropyrrolidin-1-yl)-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.110)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3,3-difluoropyrrolidine hydrochloride (89 mg, 0.619 mmol) to give (S)-tert-butyl 2-(((6-(3,3-difluoropyrrolidin-1-yl)-5-fluoro-2-methoxypyridin-3-yl)methyl)carbamoyl)pyrrolidine-1-carboxylate as crude product (368 mg) followed by Boc deprotection using the procedure described in example 136. Preparative HPLC chromatography on a reversed phase column (eluens contained 0.1% TFA) gave the title compound (S)—N-((6-(3,3-difluoropyrrolidin-1-yl)-5-fluoro-2-methoxypyridin-3-yl)methyl)pyrrolidine-2-carboxamide 2,2,2-trifluoroacetate (14.7 mg, yield 3.9%).

LCMS (ESI⁺) m/z [M+H]⁺: 359.20

¹H NMR (500 MHz, methanol-d₄) δ ppm: 7.28 (d, J=12.4 Hz, 1H), 4.25-4.15 (m, 3H), 4.05-3.90 (m, 5H), 3.85-3.80 (m, 2H), 3.45-3.35 (m, 1H), 3.35-3.25 (m, 1H), 2.50-2.35 (m, 3H), 2.10-1.90 (m, 3H).

### Example 165

### (2S)—N-[[6-(4,4-Difluoro-1-piperidyl)-5-fluoro-2-methoxy-3-pyridyl]methyl]-N-methyl-pyrrolidine-2-carboxamide; 2,2,2-trifluoroacetic Acid

(Compound of Formula Ia.23, Wherein X is CH, R⁵ is methyl, R⁶ is F, R⁷ is OCH₃, and NR^{8b}R^{8c} is A.120)

The title compound was prepared using the procedure described in example 147 starting from (S)-tert-butyl 2-(((6-chloro-5-fluoro-2-methoxypyridin-3-yl)methyl)(methyl)carbamoyl)pyrrolidine-1-carboxylate (200 mg, 0.498 mmol) and 4,4-difluoropiperidine (60.3 mg, 0.498 mmol) to give (S)-tert-butyl 2-(((6-(4,4-difluoropiperidin-1-yl)-5-fluoro-2-methoxypyridin-3-yl)methyl)(methyl)carbamoyl)pyrrolidine-1-carboxylate as crude product (266 mg) followed by Boc deprotection using the procedure described in example 136. Preparative HPLC chromatography on a reversed phase column (eluents contained 0.1% TFA) gave the title compound (S)—N((6-(4,4-difluoropiperidin-1-yl)-5-fluoro-2-methoxypyridin-3-yl)methyl)-N-methylpyrrolidine-2-carboxamide 2,2,2-trifluoroacetate (71.5 mg, yield 26.1%).

LCMS (ESI⁺) m/z [M+H]⁺: 387.25

¹H NMR (500 MHz, methanol-d₄) δ ppm: 7.42 (d, J=12.7 Hz, 1H), 4.70-4.60 (m, 1H), 4.55-4.25 (m, 2H), 3.85 (s, 3H), 3.60-3.15 (m, 5H), 2.98 (s, 3H), 2.45-2.35 (m, 1H), 2.10-2.00 (m, 1H), 1.95-1.80 (m, 2H), 1.80-1.70 (m, 2H).

### Example 166

### (2S)—N-[[6-[(4,4-Difluorocyclohexyl)amino]-5-fluoro-2-methoxy-3-pyridyl]methyl]-N-methyl-pyrrolidine-2-carboxamide; 2,2,2-trifluoroacetic Acid

(Compound of Formula Ia.23, Wherein X is CH, R⁵ is methyl, R⁶ is F, R⁷ is OCH₃, R^{8b} is H and R^{8c} is −A.33)

The title compound was prepared using the procedure described in example 136 starting from (S)-tert-butyl 2-(((6-chloro-5-fluoro-2-methoxypyridin-3-yl)methyl)(methyl)carbamoyl)pyrrolidine-1-carboxylate and 4,4-difluorocyclohexane amine (67.3 mg, 0.498 mmol) to give (S)-tert-butyl 2-(((6-((4,4-difluorocyclohexyl)amino)-5-fluoro-2-methoxypyridin-3-yl)methyl)(methyl)carbamoyl)pyrrolidine-1-carboxylate as crude product (406 mg) followed by Boc deprotection using the procedure described in example 136. Preparative HPLC chromatography on a reversed phase column (eluens contained 0.1% TFA) gave the title compound (S)—N-((6-((4,4-difluorocyclohexyl)amino)-5-fluoro-2-methoxypyridin-3-yl)methyl)-N-methylpyrrolidine-2-carboxamide 2,2,2-trifluoroacetate (7.8 mg, yield 1.9%).

LCMS (ESI⁺) m/z [M+H]⁺: 401.30

¹H NMR (500 MHz, methanol-d₄) δ ppm: 7.18 (d, J=10.8 Hz, 1H), 4.60-4.50 (m, 1H), 4.50-4.15 (m, 2H), 4.05-4.00 (m, 1H), 3.88 (s, 3H), 3.50-3.40 (m, 1H), 3.40-3.30 (m, 1H), 3.00 (s, 3H), 2.60-2.50 (m, 1H), 2.20-1.85 (m, 7H), 1.70-1.60m (m, 2H), 1.40-1.30 (m, 2H).

### Example 167

### (2S)—N-[[5-Fluoro-2-methoxy-6-(4-methoxycyclohexen-1-yl)-3-pyridyl]methyl]-pyrrolidine-2-carboxamide

(Compound of Formula Ia.24, Wherein X is CH, R⁵ is H, R⁶ is F, R⁷ is OCH₃ and R^{8d} is A.94)

The title compound was prepared using the procedure described in example 136 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-methoxycyclohexene-1-boronic acid pinacol ester (135 mg, 0.567 mmol) to give (2S)-tert-butyl 2-(((5-fluoro-2-methoxy-6-(4-methoxycyclohex-1-en-1-yl)pyridin-3-yl)methyl)carbamoyl)pyrrolidine-1-carboxylate carboxylate as crude product (249 mg). Boc deprotection using the procedure described in example 136 gave the title compound (2S)—N-((5-fluoro-2-methoxy-6-(4-methoxycyclohex-1-en-1-yl)pyridin-3-yl)methyl)pyrrolidine-2-carboxamide (30 mg, yield 63.8%).

LCMS (ESI⁺) m/z [M+H]⁺: 364.20

¹H NMR (500 MHz, DMSO-d₆) δ ppm: 8.45-8.40 (m, 1H), 7.27 (d, J=11.4 Hz, 1H), 6.40 (bs, 1H), 4.20-4.15 (m, 2H), 3.88 (s, 3H), 3.60-3.55 (m, 1H), 3.55-3.50 (m, 1H), 3.30 (s, 3H), 2.90-2.80 (m, 2H), 2.65-2.55 (m, 1H), 2.20-2.10 (m, 2H), 2.00-1.90 (m, 2H), 1.70-1.55 (m, 6H).

### Example 168

### (2S)—N-[[5-Fluoro-2-methoxy-6-(3-methoxypropyl)-3-pyridyl]methyl]pyrrolidine-2-carboxamide

(Compound of Formula Ia.24, Wherein X is CH, R⁵ is H, R⁶ is F, R⁷ is OCH₃ and R^{8d} is —(CH₂)₃—OCH₃)

The title compound was prepared using the procedure described in example 136 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-methoxy-1-propenylboronic acid (65.8 mg, 0.567 mmol) to give (S)-tert-butyl 2-(((5-fluoro-2-methoxy-6-(3-methoxyprop-1-en-1-yl)pyridin-3-yl)methyl)carbamoyl)pyrrolidine-1-carboxylate as product (113 mg, yield 46.6%, purity 90%). (S)-tert-butyl 2-(((5-fluoro-2-methoxy-6-(3-methoxypropyl)pyridin-3-yl)methyl)carbamoyl)pyrrolidine-1-carboxylate (65 mg, 0.153 mmol) was dissolved in MeOH (5 ml). Palladium on carbon (10%) (30 mg, 0.029 mmol) was added followed by the addition of ammonium formate (1.84 ml, 1.84 mmol) and water (1 ml). The mixture was stirred at reflux temperature for 2 h. The reaction mixture was filtered, concentrated, dissolved in water, basified and extracted 4× with DCM, dried over magnesium sulfate, filtered and concentrated to give (S)-tert-butyl 2-(((5-fluoro-2-methoxy-6-(3-methoxypropyl)pyridin-3-yl)methyl)carbamoyl)pyrrolidine-1-carboxylate (55 mg, yield 76%, purity 90%) as crude product. Boc deprotection using the procedure described in example 140 gave the title compound (S)—N-((5-fluoro-2-methoxy-6-(3-methoxypropyl)pyridin-3-yl)methyl)pyrrolidine-2-carboxamide (30 mg, yield 71.3%).

LCMS (ESI⁺) m/z [M+H]⁺: 326.20

¹H NMR (500 MHz, DMSO-d₆) δ ppm: 8.40-8.35 (m, 1H), 7.25 (d, J=12.5 Hz, 1H), 4.15-4.10 (m, 2H), 3.88 (s, 3H), 3.60-3.55 (m, 1H), 3.40-3.30 (m, 2H), 3.23 (s, 3H), 2.90-2.80 (m, 2H), 2.70-2.65 (m, 2H), 2.00-1.85 (m, 3H), 1.70-1.60 (m, 3H).

### Example 169

### (2S)—N-[[6-[(4,4-Difluorocyclohexyl)methylamino]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is —CH₂-A.33)

The title compound was prepared using the procedure described in example 147 starting from (S)-tert-butyl 2-(((6-chloro-5-fluoro-2-methoxypyridin-3-yl)methyl)(methyl)carbamoyl)pyrrolidine-1-carboxylate (200 mg, 0.498 mmol) and (4,4-difluorocyclohexyl)methane amine hydrochloride (105 mg, 0.567 mmol) to give (S)-tert-butyl 2-(((6-(((4,4-difluorocyclohexyl)methyl)amino)-5-fluoro-2-methoxypyridin-3-yl)methyl)carbamoyl)pyrrolidine-1-carboxylate (392 mg) as crude product. Boc deprotection using the procedure described in example 136 gave the title compound (S)—N((6-(((4,4-difluorocyclohexyl)methyl)amino)-5-fluoro-2-methoxypyridin-3-yl)methyl)pyrrolidine-2-carboxamide 2,2,2-trifluoroacetate (181 mg, yield 44.9%).

LCMS (ESI⁺) m/z [M+H]⁺: 401.30

¹H NMR (500 MHz, DMSO-d₆) δ ppm: 8.60-8.55 (m, 1H), 7.22 (d, J=11.0 Hz, 1H), 6.70-6.65 (m, 1H), 4.10-4.05 (m, 3H), 3.80 (s, 3H), 3.75-3.65 (m, 4H), 2.30-2.20 (m, 1H), 2.05-1.95 (m, 2H), 1.90-1.70 (m, 7H), 1.25-1.15 (m, 3H).

### Example 170

### (2S)—N-[[6-(Benzylamino)-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is —CH₂-A.59)

The title compound was prepared using the procedure described in example 150 starting from (S)-tert-butyl 2-(((6-chloro-5-fluoro-2-methoxypyridin-3-yl)methyl)carbamoyl)pyrrolidine-1-carboxylate (170 mg, 0.438 mmol) and benzyl amine (57 μl, 0.526 mmol) to give (S)-tert-butyl 2-(((6-(benzylamino)-5-fluoro-2-methoxypyridin-3-yl)methyl)carbamoyl)pyrrolidine-1-carboxylate (32 mg, yield 15.9%) as product. To a solution of (S)-tert-butyl 2-(((6-(benzylamino)-5-fluoro-2-methoxypyridin-3-yl)methyl)carbamoyl)pyrrolidine-1-carboxylate (32 mg, 0.070 mmol) in DCM (2 ml) was added TFA (0.1 ml, 1.298 mmol) and the mixture was stirred at room temperature overnight. The reaction mixture was concentrated and the residue was dissolved in water and extracted twice with MTBE. The water layer was freeze-dried to give the title compound (S)-2-(((6-(benzylamino)-5-fluoro-2-methoxypyridin-3-yl)methyl)carbamoyl)pyrrolidin-1-ium 2,2,2-trifluoroacetate (23 mg, yield 66.3%, purity 95%).

LCMS (ESI⁺) m/z [M+H]⁺: 359.20

¹H NMR (500 MHz, CDCl₃) δ ppm: 12.05 (s, 1H), 7.84-7.62 (m, 1H), 7.38-7.28 (m, 4H), 7.30-7.22 (m, 1H), 7.12-7.05 (m, 1H), 4.85 (s, 1H), 4.63 (s, 2H), 4.58 (dd, J=8.5, 5.8 Hz, 1H), 4.29-4.17 (m, 2H), 3.82 (d, J=3.2 Hz, 3H), 3.35 (s, 1H), 2.42-2.31 (m, 1H), 1.98 (tdd, J=22.2, 13.0, 6.0 Hz, 3H).

### Example 171

### (2S)—N-[[6-[Benzyl(propyl)amino]-5-fluoro-2-methoxy-3-pyridyl]methyl]-pyrrolidine-2-carboxamide Hydrochloride

(Compound of Formula Ia.23, Wherein X is CH, R⁵ is H, R⁶ is F, R⁷ is OCH₃, R^{8b} is propyl and R^{8c} is —CH₂-A.59)

The title compound was prepared using the procedure described in example 150 starting from (S)-tert-butyl 2-(((6-chloro-5-fluoro-2-methoxypyridin-3-yl)methyl)carbamoyl)pyrrolidine-1-carboxylate (165 mg, 0.425 mmol 1) and N-benzylpropan-1-amine (65 mg, 0.436 mmol) to give (S)-tert-butyl 2-(((6-(benzyl(propyl)amino)-5-fluoro-2-methoxypyridin-3-yl)methyl)carbamoyl)pyrrolidine-1-carboxylate (46 mg, yield 21.6%) followed by Boc deprotection using the procedure in example 170. The HCl salt was obtained by dissolving the residue in MeOH (1 ml), adding HCl (1M, 60 μl) and diluting with water (15-20 ml). Freeze-drying gave the title compound (2S)—N-[[6-[benzyl(propyl)amino]-5-fluoro-2-methoxy-3-pyridyl]methyl]-pyrrolidine-2-carboxamide hydrochloride (20 mg, yield 47.3%, purity 95%).

LCMS (ESI⁺) m/z [M+H]⁺: 401.20

¹H NMR (500 MHz, CDCl₃) δ ppm: 11.20 (s, 1H), 7.99 (t, J=5.8 Hz, 1H), 7.45 (s, 1H), 7.33-7.23 (m, 4H), 7.15 (s, 1H), 4.72 (s, 3H), 4.30 (dd, J=14.8, 5.9 Hz, 1H), 4.22 (dd, J=14.9, 5.7 Hz, 1H), 3.76 (s, 3H), 3.55-3.36 (m, 3H), 2.51 (dd, J=8.6, 5.9 Hz, 1H), 2.12-1.97 (m, 2H), 1.98 (s, 1H), 1.63 (dt, J=14.9, 7.5 Hz, 2H), 0.86 (t, J=7.4 Hz, 4H).

### Example 172

### (2S)—N-[[5-Fluoro-2-methoxy-6-(4-methoxycyclohexyl)-3-pyridyl]methyl]-pyrrolidine-2-carboxamide

(Compound of Formula Ia.24, Wherein X is CH, R⁵ is H, R⁶ is F, R⁷ is OCH₃ and R^{8d} is A.46)

The title compound was prepared using the procedure described in example 168 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-methoxycyclohexene-1-boronic acid pinacol ester (135 mg, 0.567 mmol) to give (2S)-tert-butyl 2-(((5-fluoro-2-methoxy-6-(4-methoxycyclohex-1-en-1-yl)pyridin-3-yl)methyl)carbamoyl)pyrrolidine-1-carboxylate carboxylate as crude product (249 mg). Reduction of the double bond gave (S)-tert-butyl 2-(((5-fluoro-2-methoxy-6-(4-methoxycyclohexyl)pyridin-3-yl)methyl)carbamoyl)pyrrolidine-1-carboxylate (195 mg, yield 91%, purity 70%). The material was used as crude product without further purification. Boc deprotection was carried out using the procedure described in example 140 to give the title compound (2R)—N-((5-fluoro-2-methoxy-6-(4-methoxycyclohexyl)pyridin-3-yl)methyl)pyrrolidine-2-carboxamide (115 mg, yield 75%).

LCMS (ESI⁺) m/z [M+H]⁺: 366.30

¹H NMR (500 MHz, DMSO-d₆) δ ppm: 8.40-8.35 (m, 1H), 7.23 (d, J=9.8 Hz, 1H), 4.15-4.05 (m, 2H), 3.87 (s, 3H), 3.60-3.55 (m, 1H), 3.25 (s, 3H), 2.95-2.80 (m, 4H), 2.10-2.05 (m, 1H), 2.00-1.85 (m, 3H), 1.80-1.45 (m, 6H), 1.30-1.20 (m, 2H).

### Example 173

### (2S)—N-[[5-Fluoro-2-methoxy-6-(2-phenylmorpholin-4-yl)-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.136)

(S)-tert-Butyl 2-(((6-chloro-5-fluoro-2-methoxypyridin-3-yl)methyl)carbamoyl)pyrrolidine-1-carboxylate (100 mg, 0.259 mmol), 2-phenylmorpholine (50.5 mg, 0.309 mmol) and sodium tert-butoxide (61.9 mg, 0.645 mmol) were suspended in toluene (4 ml). Dicyclohexyl(2′,6′-diisopropoxy-[1,1′-biphenyl]-2-yl)phosphine (RuPhos) (18.05 mg, 0.039 mmol) and bis(dibenzylideneacetone)palladium(0) (22.24 mg, 0.039 mmol) were added and flushed with argon. The reaction mixture was stirred in the Microwave at 140° C. for 10 h. The reaction mixture was concentrated and the obtained crude product was taken onto Bulk Isolute Sorbent and purified using flash chromatography (4 g column; DCM 100%→DCM:MeOH 0:100, 18 ml/min) to give (2S)-tert-butyl 2-(((5-fluoro-2-methoxy-6-(2-phenylmorpholino)pyridin-3-yl)methyl)carbamoyl)pyrrolidine-1-carboxylate (89.5 mg, yield 67.5%). Boc deprotection was carried out using the procedure described in example 132. Preparative HPLC chromatography on a reversed phase column (eluents contained 0.1% TFA) gave the title compound (2S)—N-((5-fluoro-2-methoxy-6-(2-phenylmorpholino)pyridin-3-yl)methyl)pyrrolidine-2-carboxamide 2,2,2-trifluoroacetate (1.1 mg, yield 1.1%, purity 90%).

LCMS (ESI⁺) m/z [M+H]⁺: 415.20

¹H NMR (600 MHz, CDCl₃) δ ppm: δ 7.46-7.30 (m, 5H), 7.19 (d, J=12.2 Hz, 1H), 7.15 (s, br, 1H), 4.77-4.63 (m, 2H), 4.34 (d, J=3.6 Hz, 2H), 4.16-4.11 (m, 2H), 4.02-4.90 (m, 3H), 3.99 (s, 3H), 3.50-3.46 (m, 1H), 3.39-3.34 (m, 1H), 3.23-3.16 (m, 1H), 2.98 (ddd, J=13.1, 10.5, 2.5 Hz, 1H), 2.45 (tr, J=8.4 Hz, 1H), 2.17-1.93 (m, 3H).

### Example 174

### (2S)—N-[[6-(2,6-Dimethylmorpholin-4-yl)-5-fluoro-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.135)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (100 mg, 0.258 mmol) and 2,6-dimethylmorpholine (35.6 mg, 0.309 mmol) to give (2S)—N-((6-(2,6-dimethylmorpholino)-5-fluoro-2-methoxypyridin-3-yl)methyl)pyrrolidine-2-carboxamide 2,2,2-trifluoroacetate (1.2 mg, yield 2.2%, purity 90%).

LCMS (ESI⁺) m/z [M+H]⁺: 367.20

### Example 175

### (2S)—N-[[5-Fluoro-2-methoxy-6-(3-phenoxyazetidin-1-yl)-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.103)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-phenoxyazetidine hydrochloride (105 mg, 0.567 mmol) followed by Boc deprotection as described in example 136. Preparative HPLC chromatography on a reversed phase column (eluens contained 0.1% TFA) gave the title compound (S)—N-((5-fluoro-2-methoxy-6-(3-phenoxyazetidin-1-yl)pyridin-3-yl)methyl)pyrrolidine-2-carboxamide 2,2,2-trifluoroacetate (5.3 mg, yield 2.8%).

LCMS (ESI+) m/z [M+H]⁺: 401.00

¹H NMR (600 MHz, DMSO-d₆) δ ppm: 9.20 (bs, 1H), 8.75-8.70 (m, 1H), 8.55 (bs, 1H), 7.35-7.30 (m, 2H), 7.20-7.15 (m, 1H), 7.00-6.95 (m, 1H), 6.90-6.85 (m, 2H), 5.15-5.10 (m, 1H), 4.55-4.50 (m, 2H), 4.15-4.10 (m, 3H), 4.00-3.95 (m, 2H), 3.82 (s, 3H), 3.25-3.15 (m, 2H), 2.30-2.25 (m, 1H), 1.95-1.85 (m, 2H), 1.85-1.75 (m, 1H).

### Example 176

### (2S)—N-[[5-Fluoro-2-methoxy-6-(3-methyl-3-phenoxy-azetidin-1-yl)-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.105)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-methyl-3-phenoxyazetidine hydrochloride (113 mg, 0.567 mmol) to give (S)-tert-butyl 2-(((5-fluoro-2-methoxy-6-(3-methyl-3-phenoxyazetidin-1-yl)pyridin-3-yl)methyl)carbamoyl)pyrrolidine-1-carboxylate (491 mg) as crude product followed by Boc deprotection as described in procedure example 136. Preparative HPLC chromatography on a reversed phase column (eluents contained 0.1% TFA) gave the title compound (S)—N((5-fluoro-2-methoxy-6-(3-methyl-3-phenoxyazetidin-1-yl)pyridin-3-yl)methyl)pyrrolidine-2-carboxamide 2,2,2-trifluoroacetate (95.6 mg, yield 19%).

LCMS (ESI+) m/z [M+H]⁺: 415.30

¹H NMR (500 MHz, DMSO-d₆) δ ppm: 9.20 (bs, 1H), 8.75-8.70 (m, 1H), 8.55 (bs, 1H), 7.40-7.30 (m, 3H), 7.00-6.95 (m, 1H), 6.80-6.75 (m, 2H), 4.25-4.10 (m, 8H), 3.83 (s, 3H), 3.25-3.15 (m, 1H), 2.30-2.25 (m, 1H), 1.95-1.80 (m, 3H), 1.70 (s, 3H).

### Example 177

### (2S)—N-[[5-Fluoro-6-[(4-fluorophenyl)methylamino]-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is —CH₂-A.62)

The title compound was prepared using the procedure described in example 170 starting from (S)-tert-butyl 2-(((6-chloro-5-fluoro-2-methoxypyridin-3-yl)methyl)carbamoyl)pyrrolidine 1-carboxylate (170 mg, 0.438 mmol) and (4-fluorophenyl)methanamine (70 μl, 0.614 mmol) to give (S)-2-(((5-fluoro-6-((4-fluorobenzyl)amino)-2-methoxypyridin-3-yl)methyl)carbamoyl)pyrrolidin-1-ium 2,2,2-trifluoroacetate (17 mg, yield 83%, purity 95%).

LCMS (ESI⁺) m/z [M+H]⁺: 377.35

¹H NMR (600 MHz, CDCl₃) δ ppm: 12.14 (s, 1H), 7.73 (dt, J=35.9, 5.8 Hz, 1H), 7.40-7.28 (m, 2H), 7.09 (dd, J=10.4, 1.6 Hz, 1H), 7.05-6.95 (m, 2H), 4.87 (s, 1H), 4.60 (s, 2H), 4.23 (dd, J=5.8, 2.7 Hz, 2H), 3.81 (d, J=2.3 Hz, 5H), 3.41-3.32 (m, 2H), 2.52-2.24 (m, 1H), 2.08-1.91 (m, 2H), 1.99 (s, 1H).

### Example 178

### (2S)—N-[[5-Fluoro-6-(4-fluoroanilino)-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is H and R^{8c} is −A.62)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-fluoroaniline (57.3 mg, 0.516 mmol) to give (S)-tert-butyl 2-(((5-fluoro-6-((4-fluorophenyl)amino)-2-methoxypyridin-3-yl)methyl)carbamoyl)pyrrolidine-1-carboxylate (352.7 mg) as crude product followed by Boc deprotection as described in procedure example 136. Preparative HPLC chromatography on a reversed phase column (eluents contained 0.1% TFA) gave the title compound (S)—N-((5-fluoro-6-((4-fluorophenyl)amino)-2-methoxypyridin-3-yl)methyl)pyrrolidine-2-carboxamide 2,2,2-trifluoroacetate (31.7 mg, yield 8.7%).

LCMS (ESI⁺) m/z [M+H]⁺: 363.35

¹H NMR (500 MHz, DMSO-d₆) δ ppm: 9.15 (bs, 1H), 8.85 (s, 1H) 8.80-8.75 (m, 1H), 8.55 (bs, 1H), 7.75-7.70 (m, 2H), 7.45 (d, J=10.9 Hz, 1H), 7.15-7.10 (m, 2H), 4.25-4.15 (m, 3H), 3.85 (s, 3H), 3.30-3.20 (m, 2H), 2.35-2.25 (m, 1H), 1.95-1.80 (m, 3H).

### Example 179

### (2S)—N-[[6-[N-Benzyl-N-methyl-amino]-5-fluoro-2-methoxy-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, R^{8b} is methyl and R^{8c} is —CH₂-A.59)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and N-methyl-1-phenylmethanamine (62.5 mg, 0.516 mmol) to give (S)-tert-butyl 2-(((6-(benzyl(methyl)amino)-5-fluoro-2-methoxypyridin-3-yl)methyl)carbamoyl)pyrrolidine-1 carboxylate (451 mg) as crude product followed by Boc deprotection as described in procedure example 136. Preparative HPLC chromatography on a reversed phase column (eluents contained 0.1% TFA) gave the title compound (S)—N-((6-(benzyl(methyl)amino)-5-fluoro-2-methoxypyridin-3-yl)methyl)pyrrolidine-2-carboxamide 2,2,2-trifluoroacetate (3.3 mg, yield 0.7%).

LCMS (ESI⁺) m/z [M+H]⁺: 373.40

¹H NMR (500 MHz, methanol-d₄) δ ppm: 7.30-7.20 (m, 6H), 4.70 (s, 2H), 4.25-4.15 (m, 3H), 3.82 (s, 3H), 3.45-3.35 (m, 1H), 3.35-3.25 (m, 1H), 3.07 (s, 3H), 2.40-2.30 (m, 1H), 2.10-1.95 (m, 3H).

### Example 180

### (2S)—N-[[5-Fluoro-2-methoxy-6-[4-[4-(trifluoromethyl)cyclohexyl]-1-piperidyl]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.131)

The title compound was prepared using the procedure described in example 147 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-(4-(trifluoromethyl)cyclohexyl)piperidine (133 mg, 0.516 mmol) to give (S)-tert-butyl 2-(((5-fluoro-2-methoxy-6-(4-(4-(trifluoromethyl)cyclohexyl)piperidin-1-yl)pyridin-3-yl)methyl)carbamoyl)pyrrolidine-1-carboxylate (497 mg) as crude product followed by Boc deprotection as described in procedure example 136. Preparative HPLC chromatography on a reversed phase column (eluents contained 0.1% TFA) gave the title compound (S)—N((5-fluoro-2-methoxy-6-(4-((1 r,4S)-4-(trifluoromethyl)cyclohexyl)piperidin-1-yl)pyridin-3-yl)methyl)pyrrolidine-2-carboxamide 2,2,2-trifluoroacetate (72 mg, yield 14.1%).

LCMS (ESI⁺) m/z [M+H]⁺: 487.45

¹H NMR (500 MHz, DMSO-d₆) δ ppm: 9.15 (bs, 1H), 8.75-8.70 (m, 1H), 8.55 (bs, 1H), 7.33 (d, J=13.0 Hz, 1H), 4.20-4.10 (m, 3H), 4.05-4.00 (m, 2H), 3.90-3.70 (m, 4H), 3.30-3.20 (m, 2H), 2.85-2.75 (m, 2H), 2.35-2.25 (m, 2H), 1.95-1.75 (m, 4H), 1.65-1.40 (m, 9H), 1.25-1.20 (m, 1H), 1.15-1.05 (m, 2H).

### Example 181

### (2S)—N-[[6-[(3aR,6aS)-5,5-Difluoro-1,3,3a,4,6,6a-hexahydrocyclopenta[c]pyrrol-2-yl]-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.138)

The title compound was prepared using the procedure described in example 170 starting from (S)-tert-butyl 2-(((6-chloro-5-fluoro-2-methoxypyridin-3-yl)methyl)carbamoyl)pyrrolidine-1-carboxylate (202 mg, 0.521 mmol) and (3aR,6aS)-5,5-difluorooctahydrocyclopenta[c]pyrrole hydrochloride (115 mg, 0.625 mmol) to give (S)-2-(((6-((3aR,6aS)-5,5-difluorohexahydrocyclopenta[c]pyrrol-2(1H)-yl)-5-fluoro-2-methoxypyridin-3-yl)methyl)carbamoyl)pyrrolidin-1-ium 2,2,2-trifluoroacetate (47 mg, yield 68.9%, purity 95%).

LCMS (ESI⁺) m/z [M+H]⁺: 399.40

¹H NMR (600 MHz, CDCl₃) δ ppm: 12.33 (s, 1H), 7.65 (t, J=5.9 Hz, 1H), 7.10 (dd, J=12.2, 2.5 Hz, 1H), 4.61 (dd, J=8.6, 5.7 Hz, 1H), 4.31-4.17 (m, 2H), 3.86 (s, 3H), 3.78-3.64 (m, 2H), 3.56 (d, J=11.1 Hz, 2H), 3.39 (q, J=6.7 Hz, 2H), 2.94-2.80 (m, 2H), 2.48-2.33 (m, 3H), 2.13-1.93 (m, 5H).

### Example 182

### (2S)—N-[[5-Fluoro-6-(3,3,4,4,5,5-hexafluoro-1-piperidyl)-2-methoxy-3-pyridyl]-methyl]pyrrolidine-2-carboxamide Hydrochloride

(Compound of Formula Ia.23, Wherein X is CH, R⁵ is H, R⁶ is F, R⁷ is OCH₃, and NR^{8b}R^{8c} is A.121)

(S)-tert-Butyl 2-(((6-chloro-5-fluoro-2-methoxypyridin-3-yl)methyl)carbamoyl)pyrrolidine-1-carboxylate (150 mg, 0.387 mmol), 2,2′-bis(diphenylphosphino)-1,1′-binaphthalene (BINAP) (37 mg, 0.059 mmol), bis(dibenzylideneacetone)palladium(0) (33.4 mg, 0.058 mmol) and cesium carbonate (315 mg, 0.967 mmol) were suspended in toluene (5 ml) to give a brown suspension. The reaction mixture was flushed with argon and 3,3,4,4,5,5-hexafluoropiperidine was added. The reaction mixture was stirred in the Microwave at 140° C. for 4 h, then was concentrated and the residue was dissolved in ethyl acetate and washed twice with water and 1× with brine, dried with magnesium sulfate, filtered and concentrated. The crude material was purified using flash chromatography (12 g column; DCM 100%→DCM:MeOH 95:5.20 ml/min) to give (S)-tert-butyl 2-(((5-fluoro-6-(3,3,4,4,5,5-hexafluoropiperidin-1-yl)-2-methoxypyridin-3-yl)methyl)carbamoyl)pyrrolidine-1-carboxylate (185 mg, yield 88%). To a solution of (S)-tert-butyl 2-(((5-fluoro-6-(3,3,4,4,5,5-hexafluoropiperidin-1-yl)-2-methoxypyridin-3-yl)methyl)carbamoyl)pyrrolidine-1-carboxylate (185 mg, 0.340 mmol 1) in DCM (5 ml) was added TFA (0.4 ml, 5.19 mmol) and stirred at room temperature for 3 h and subsequently concentrated under reduced pressure. The residue was extracted between MTBE and water and the MTBE layer was washed sodium bicarbonate solution and the aqueous layer was washed twice with MTBE. The combined organic layers were dried over magnesium sulfate, filtered and concentrated. The crude material was purified using flash chromatography (12 g column; DCM 100%→DCM:MeOH 90:10) to give 115 mg of (2S)—N-[[5-fluoro-6-(3,3,4,4,5,5-hexafluoro-1-piperidyl)-2-methoxy-3-pyridyl]methyl]pyrrolidine-2-carboxamide. The residue was dissolved in MeOH (1 ml) and HCl (1M, 259 μl) were added and this solution was evaporated to dryness to obtain a yellow oil, which contains remaining methanol. To minimize the remaining methanol, the obtained oil was dissolved in DCM, evaporated again, and dried under high vacuum to give the title compound (S)-2-(((5-fluoro-6-(3,3,4,4,5,5-hexafluoropiperidin-1-yl)-2-methoxypyridin-3-yl)methyl)carbamoyl)pyrrolidin-1-ium chloride as yellow foam (124 mg, yield 72.1%, purity 95%)

LCMS (ESI⁺) m/z [M+H]⁺: 445.30

¹H NMR (500 MHz, CDCl₃) δ ppm: 10.85 (s, 1H), 8.65 (s, 1H), 7.54 (s, 1H), 7.37 (d, J=12.2 Hz, 1H), 4.79 (s, 1H), 4.39-4.23 (m, 2H), 4.07 (d, J=10.2 Hz, 4H), 3.89 (s, 3H), 3.49-3.41 (m, 2H), 2.56 (s, 1H), 2.02 (s, 3H).

### Example 183

### (2S)—N-[[6-(6,6-Difluoro-3-azabicyclo[3.1.0]hexan-3-yl)-5-fluoro-2-methoxy-3-pyridyl]methyl]pyrrolidine-2-carboxamide

(Compound of Formula Ia.23, Wherein X is CH, R⁵ is H, R⁶ is F, R⁷ is OCH₃, and NR^{8b}R^{8c} is A.137)

(S)-tert-Butyl 2-(((6-chloro-5-fluoro-2-methoxypyridin-3-yl)methyl)carbamoyl)pyrrolidine-1-carboxylate (200 mg, 0.516 mmol), 6,6-difluoro-3-azabicyclo[3.1.0]-hexane hydrochloride (80 mg, 0.516 mmol), dicyclohexyl(2′,6′-diisopropoxy-[1,1′-biphenyl]-2-yl)phosphine (RuPhos) (36.1 mg, 0.077 mmol), bis(dibenzylideneacetone)palladium(0) (44.4 mg, 0.077 mmol) and sodium 2-methylpropan-2-olate (124 mg, 1.289 mmol) were suspended in toluene (10 ml) and flushed with nitrogen and stirred in Q-tube at 120° C. for 3 h. The reaction mixture was extracted between ethyl acetate and water, dried with sodium sulfate, filtered and concentrated. The material was used as crude product without further purification. To a solution of (2S)-tert-butyl 2-(((6-(6,6-difluoro-3-azabicyclo[3.1.0]hexan-3-yl)-5-fluoro-2-methoxypyridin-3-yl)methyl)carbamoyl)pyrrolidine-1-carboxylate (311 mg, 0.661 mmol) in DCM (20 ml) was added TFA (1 ml, 12.98 mmol) and stirred at room temperature for 3 h and subsequently concentrated under reduced pressure. The crude material was purified twice using flash chromatography (15 g column; DCM 100%→DCM:MeOH 75:25) to give (2S)—N-((6-(6,6-difluoro-3-azabicyclo[3.1.0]hexan-3-yl)-5-fluoro-2-methoxypyridin-3-yl)methyl)pyrrolidine-2-carboxamide (52 mg, yield 21.2%, purity 95%).

LCMS (ESI⁺) m/z [M+H]⁺: 371.30

¹H NMR (500 MHz, DMSO-d₆) δ ppm: 8.40-8.35 (m, 1H), 7.75 (d, J=12.8 Hz, 1H), 4.10-4.05 (m, 2H), 3.95-3.90 (m, 2H), 3.85-3.80 (m, 5H), 3.70-3.65 (m, 1H), 2.95-2.85 (m, 2H), 2.65-2.60 (m, 2H), 2.10-2.00 (m, 1H), 1.70-1.60 (m, 3H).

### Example 184

### (2S)—N-[[6-(7,7-Difluoro-4-azabicyclo[4.1.0]heptan-4-yl)-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.140)

The title compound was prepared using the procedure described in example 183 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 7,7-difluoro-3-azabicyclo[4.1.0]heptane hydrochloride (87 mg, 0.516 mmol). Preparative HPLC chromatography on a reversed phase column (eluents contained 0.1% TFA) gave the title compound (2S)—N-((6-(7,7-difluoro-3-azabicyclo[4.1.0]heptan-3-yl)-5-fluoro-2-methoxypyridin-3-yl)methyl)pyrrolidine-2-carboxamide 2,2,2-trifluoroacetate (22 mg, 7.1%).

LCMS (ESI⁺) m/z [M+H]⁺: 385.35

¹H NMR (500 MHz, DMSO-d₆) δ ppm: 9.15 (bs, 1H), 8.80-8.75 (m, 1H), 8.55 (bs, 1H), 4.20-4.10 (m, 2H), 4.00-3.95 (m, 1H), 3.85 (s, 3H), 3.60-3.50 (m, 2H), 3.35-3.10 (m, 6H), 2.30-2.25 (m, 1H), 2.00-1.70 (m, 5H).

### Example 185

### (2S)—N-[[5-Fluoro-2-methoxy-6-(3-phenylpropylamino)-3-pyridyl]methyl]-pyrrolidine-2-carboxamide; 2,2,2-trifluoroacetic acid

(Compound of Formula Ia.23, Wherein X is CH, R⁵ is H, R⁶ is F, R⁷ is OCH₃, R^{8b} is H and R^{8c} is —(CH₂)₃-A.59)

(S)-tert-Butyl 2-(((6-chloro-5-fluoro-2-methoxypyridin-3-yl)methyl)carbamoyl)pyrrolidine-1-carboxylate (388 mg, 1.0 mmol), 3-phenylpropylamine (171 μl, 1.2 mmol), sodium 2-methylpropan-2-olate (144 mg, 1.50 mmol), di-tert-butyl(2′,4′,6′-triisopropyl-[1,1′-biphenyl]-2-yl)phosphine (t-BuXPhos) (18.69 mg, 0.044 mmol), and bis[cinnamyl palladium(II) chloride] were suspended in a 2% TPGS-750-M in water solution (3 ml) and stirred (1200 r.p.m.) at room temperature over the weekend and subsequently concentrated under reduced pressure to give 608.3 mg as crude product. The crude material was purified using flash chromatography (4 g column; cyclohexane 100%→cyclohexane:ethyl acetate 0:100.18 ml/min) to give (S)-tert-butyl 2-(((5-fluoro-2-methoxy-6-((3-phenylpropyl)amino)pyridin-3-yl)methyl)carbamoyl)pyrrolidine-1-carboxylate (450.2 mg, yield 92%). To a solution of (S)-tert-butyl 2-(((5-fluoro-2-methoxy-6-((3-phenylpropyl)amino)pyridin-3-yl)methyl)carbamoyl)pyrrolidine-1-carboxylate (447 mg, 0.919 mmol) in DCM (10 ml) was added TFA (1,416 ml, 18.37 mmol) and stirred at room temperature overnight and subsequently concentrated under reduced pressure to give 772 mg as crude product. The residue was dissolved in ethyl acetate and Bulk Isolute Sorbent was added. The mixture was purified using flash chromatography (4 g column; cyclohexane 100%→cyclohexane:ethyl acetate 0:100→MeOH 100.18 ml/min) to give (S)—N-((5-fluoro-2-methoxy-6-((3-phenylpropyl)amino)pyridin-3-yl)methyl)pyrrolidine-2-carboxamide 2,2,2-trifluoroacetate (306.9 mg, yield 66.7%).

LCMS (ESI⁺) m/z [M+H]⁺: 387.30

¹H NMR (600 MHz, DMSO-d₆) δ ppm: 9.25 (bs, 1H), 8.70-8.65 (m, 1H), 8.55 (bs, 1H), 7.30-7.25 (m, 2H), 7.25-7.20 (m, 3H), 7.20-7.15 (m, 1H), 6.70-6.65 (m, 1H), 4.15-4.05 (m, 3H), 3.72 (s, 3H), 3.40-3.30 (m, 2H), 3.25-3.15 (m, 2H), 2.65-2.60 (m, 2H), 2.30-2.20 (m, 1H), 1.90-1.75 (m, 5H).

### Example 186

### (2S)—N-[[5-Fluoro-2-methoxy-6-[[2-(trifluoromethyl)cyclobutyl]amino]-3-pyridyl]-methyl]pyrrolidine-2-carboxamide

(Compound of Formula Ia.23, Wherein X is CH, R⁵ is H, R⁶ is F, R⁷ is OCH₃, R^{8b} is H and R^{8c} is −A.12)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (100 mg, 0.258 mmol 1) and 2-(trifluoromethyl)cyclobutan-1-amine hydrochloride (45.3 mg, 0.258 mmol) to give (2S)-tert-butyl 2-(((5-fluoro-2-methoxy-6-(((1S)-2-(trifluoromethyl)cyclobutyl)amino)pyridin-3-yl)methyl)carbamoyl)pyrrolidine-1-carboxylate (3.5 mg, yield 2.8%). To a solution of (2S)-tert-butyl 2-(((5-fluoro-2-methoxy-6-(((1S)-2-(trifluoromethyl)cyclobutyl)amino)pyridin-3-yl)methyl)carbamoyl)pyrrolidine-1-carboxylate (3.5 mg, 7.14 μmol) in DCM (2 ml) was added TFA (5.50 μl, 0.071 mmol) and stirred at 25° C. for 2 h. To the reaction mixture was added 3 ml 1M NaOH and stirred 3 min. The layers were separated and the organic layer was dried and concentrated to give the title compound (2S)—N-((5-fluoro-2-methoxy-6-(((1S)-2-(trifluoromethyl)cyclobutyl)amino)pyridin-3-yl)methyl)pyrrolidine-2-carboxamide (2.2 mg, yield 63.2%, purity 80%).

LCMS (ESI⁺) m/z [M+H]⁺: 391.20

¹H NMR (500 MHz, CDCl₃) δ ppm: 7.81 (d, J=7.0 Hz, 1H), 7.13 (d, J=10.4 Hz, 1H), 4.77-4.70 (m, 1H), 4.64-4.58 (m, 1H), 4.22 (d, J=6.1 Hz, 2H), 3.89 (s, 3H), 3.78 (ddd, J=9.0, 5.5, 3.7 Hz, 1H), 3.07-2.85 (m, 3H), 2.44-2.27 (m, 1H), 2.18-2.10 (m, 1H), 2.08-1.94 (m, 2H), 1.91 (ddt, J=20.4, 14.7, 7.1 Hz, 3H), 1.74-1.67 (m, 2H).

### Example 187

### (2S)—N-[[5-Fluoro-2-methoxy-6-[3-(2,2,2-trifluoroethyl)pyrrolidin-1-yl]-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.113)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-(2,2,2-trifluoroethyl)pyrrolidine hydrochloride (117 mg, 0.62 mmol) to give (2S)-tert-butyl 2-(((5-fluoro-2-methoxy-6-(3-(2,2,2-trifluoroethyl)pyrrolidin-1-yl)pyridin-3-yl)methyl)carbamoyl)pyrrolidine-1-carboxylate (212.6 mg, yield 40.9%, purity 50%). The material was used as crude product without further purification followed by Boc deprotection using the procedure described in example 186. Preparative HPLC chromatography on a reversed phase column (eluents contained 0.1% TFA) gave the title compound (2S)—N((5-fluoro-2-methoxy-6-(3-(2,2,2-trifluoroethyl)pyrrolidin-1-yl)pyridin-3-yl)methyl)pyrrolidine-2-carboxamide 2,2,2-trifluoroacetate (28 mg, yield 25.6%).

LCMS (ESI⁺) m/z [M+H]⁺: 405.20

¹H NMR (600 MHz, CDCl₃) δ ppm: 12.53 (s, 1H), 7.12-7.05 (m, 2H), 4.63 (t, J=6.9 Hz, 1H), 4.29-4.19 (m, 2H), 3.93-3.89 (m, 1H), 3.86 (s, 3H), 3.76 (dddt, J=11.1, 8.2, 5.7, 2.8 Hz, 1H), 3.64-3.55 (m, 1H), 3.48-3.42 (m, 1H), 3.40-3.36 (m, 1H), 3.30 (m, 1H), 2.51 (p, J=8.2 Hz, 1H), 2.49-2.37 (m, 1H), 2.32-2.17 (m, 3H), 2.14-2.05 (m, 1H), 2.06-1.94 (m, 2H), 1.68 (dq, J=12.5, 9.5 Hz, 1H).

### Example 188

### (2S)—N-[[6-(2,2-Difluoro-7-azaspiro[2.5]octan-7-yl)-5-fluoro-2-methoxy-3-pyridyl]methyl]pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.141)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 1,1-difluoro-5-azaspiro[2.5]octane hydrochloride (114 mg, 0.619 mmol) to give (S)-tert-butyl 2-(((6-((S)-1,1-difluoro-5-azaspiro[2.5]octan-5-yl)-5-fluoro-2-methoxypyridin-3-yl)methyl)carbamoyl)pyrrolidine-1-carboxylate (242.8 mg, yield 9.4%, purity 10%). The material was used as crude product without further purification followed by Boc deprotection using the procedure described in example 186. Preparative HPLC chromatography on a reversed phase column (eluents contained 0.1% TFA) gave the title compound (S)—N((6-((S)-1,1-difluoro-5-azaspiro[2.5]octan-5-yl)-5-fluoro-2-methoxypyridin-3-yl)methyl)pyrrolidine-2-carboxamide 2,2,2-trifluoroacetate (7.7 mg, yield 30.9%).

LCMS (ESI⁺) m/z [M+H]⁺: 399.20

¹H NMR (600 MHz, CDCl₃) δ ppm: 11.96 (s, 1H), 7.44 (t, J=5.7 Hz, 1H), 7.20 (s, 2H), 7.13 (dd, J=12.4, 1.5 Hz, 1H), 4.65 (s, 1H), 4.32-4.20 (m, 2H), 3.86 (d, J=1.1 Hz, 3H), 3.61-3.45 (m, 3H), 3.41 (d, J=7.7 Hz, 2H), 2.47-2.35 (m, 1H), 2.13-1.94 (m, 3H), 1.79 (qd, J=8.5, 6.8, 3.9 Hz, 3H), 1.66 (d, J=10.6 Hz, 1H), 1.25 (dddd, J=13.3, 7.6, 5.9, 3.4 Hz, 1H), 1.08 (ddd, J=11.7, 7.7, 3.4 Hz, 1H).

### Example 189

### (2S)—N-[[5-Fluoro-2-methoxy-6-(tetrahydropyran-4-ylmethyl)-3-pyridyl]methyl]-pyrrolidine-2-carboxamide; 2,2,2-trifluoroacetic Acid

(Compound of Formula Ia.24, Wherein X is CH, R⁵ is H, R⁶ is F, R⁷ is OCH₃ and R^{8d} is —CH₂-A.96)

(S)-tert-Butyl 2-(((6-chloro-5-fluoro-2-methoxypyridin-3-yl)methyl)carbamoyl)pyrrolidine-1-carboxylate (400 mg, 1.031 mmol), 2,2′-bis(diphenylphosphino)-1,1′-binaphthalene (BINAP) (64.2 mg, 0.103 mmol), bis(dibenzylideneacetone)palladium(0) (59.3 mg, 0.103 mmol) and cesium carbonate (840 mg, 2.58 mmol) were suspended in toluene (2 ml). The reaction mixture was flushed with argon. 4-Methylenetetrahydro-2H-pyran (152 mg, 1.547 mmol) was added and stirred in the Microwave at 140° C. for 4 h and then at 160° C. for 2 h and subsequently concentrated under reduced pressure. The crude material was taken onto Bulk Isolute Sorbent and purified using flash chromatography (12 g column; DCM 100%→DCM:MeOH 70:30, 30 ml/min). The product was obtained by preparative HPLC chromatography on a reversed phase column to give to give (S)-tert-butyl 2-(((6-((dihydro-2H-pyran-4(3H)-ylidene)methyl)-5-fluoro-2-methoxypyridin-3-yl)methyl)carbamoyl)pyrrolidine-1-carboxylate (50.9 mg, yield 28.5%, purity 85%). The material was used as crude product without further purification. (S)-tert-Butyl 2-(((6-((dihydro-2H-pyran-4(3H)-ylidene)methyl)-5-fluoro-2-methoxypyridin-3-yl)methyl)carbamoyl)pyrrolidine-1-carboxylate (35.4 mg, 0.067 mmol), ammonium formate (127 mg, 2.008 mmol) and palladium on carbon 10% (7.12 mg, 6.69 μmol) were suspended in ethanol (2 ml) and water (2 ml). The reaction mixture was stirred in the Microwave at 130° C. for 16h. The reaction mixture was filtered over celite and washed with MeOH. The filtrate was concentrated. The trifluoroacetate salt was obtained by preparative HPLC chromatography on a reversed phase column to give (S)—N-((5-fluoro-2-methoxy-6-((tetrahydro-2H-pyran-4-yl)methyl)pyridin-3-yl)methyl)pyrrolidine-2-carboxamide 2,2,2-trifluoroacetate (3.3 mg, yield 10.6%).

LCMS (ESI⁺) m/z [M+H]⁺: 352.10

¹H NMR (600 MHz, DMSO-d₆) δ ppm: 9.18 (s, 1H), 8.90 (t, J=5.8 Hz, 1H), 8.58 (s, 1H), 7.45 (d, J=9.2 Hz, 1H), 4.34-4.19 (m, 3H), 3.88 (s, 3H), 3.81 (ddd, J=11.4, 4.5, 1.8 Hz, 2H), 3.25 (td, J=11.7, 2.1 Hz, 3H), 2.64-2.59 (m, 2H), 2.35-2.26 (m, 1H), 1.96 (dqt, J=12.4, 8.5, 4.4 Hz, 1H), 1.94-1.80 (m, 3H), 1.55-1.46 (m, 2H), 1.26 (qd, J=12.1, 4.4 Hz, 2H).

### Example 190

### (2S)—N-[[6-[3-(Difluoromethyl)-4,4-difluoro-1-piperidyl]-5-fluoro-2-methoxy-3-pyridyl]methyl]pyrrolidine-2-carboxamide Hydrochloride

(Compound of Formula Ia.23, Wherein X is CH, R⁵ is H, R⁶ is F, R⁷ is OCH₃, and NR^{8b}R^{8c} is A.122)

The title compound was prepared using the procedure described in example 136 starting from (S)-tert-butyl 2-(((6-chloro-5-fluoro-2-methoxypyridin-3-yl)methyl)carbamoyl)pyrrolidine-1-carboxylate (110 mg, 0.284 mmol) and 3-(difluoromethyl)-4,4-difluoropiperidin-1-ium chloride (70 mg, 0.337 mmol) to give (2S)-tert-butyl 2-(((6-(3-(difluoromethyl)-4,4-difluoropiperidin-1-yl)-5-fluoro-2-methoxypyridin-3-yl)methyl)carbamoyl)pyrrolidine-1-carboxylate (120 mg, yield 81%). To a solution of (2S)-tert-butyl 2-(((6-(3-(difluoromethyl)-4,4-difluoropiperidin-1-yl)-5-fluoro-2-methoxypyridin-3-yl)methyl)carbamoyl)pyrrolidine-1-carboxylate (120 mg, 0.230 mmol) in DCM (3 ml) was added TFA (0.3 ml, 3.89 mmol) and stirred at room temperature for 3 h and then warmed up to 40° C. for 1.5 h. The reaction mixture was diluted with DCM and was washed with sodium bicarbonate solution (pH>=8). The aqueous layer was washed twice with DCM, dried with magnesium sulfate, filtered and concentrated. The crude material was purified using flash chromatography (4 g column; DCM 100%→DCM:MeOH 90:10, 18 ml/min) to give (2S)—N-((6-(3-(difluoromethyl)-4,4-difluoropiperidin-1-yl)-5-fluoro-2-methoxypyridin-3-yl)methyl)pyrrolidine-2-carboxamide (79 mg, yield 81%). The residue was dissolved in MeOH (1 ml) and HCl (1M) was added and diluted with DCM and subsequently concentrated under vacuum. The obtained residue was dissolved in DCM (2 ml) and added drop wise to diethyl ether (15 ml). A turbid mixture was formed, which was evaporated cold and dried under high vacuum to give the title compound (2S)-2-(((6-(3-(difluoromethyl)-4,4-difluoropiperidin-1-yl)-5-fluoro-2-methoxypyridin-3-yl)methyl)carbamoyl)pyrrolidin-1 ium chloride (83 mg, yield 97%).

LCMS (ESI⁺) m/z [M+H]⁺: 423.30

¹H NMR (500 MHz, CDCl₃) δ ppm: 10.88 (s, 1H), 8.55 (d, J=16.1 Hz, 1H), 7.59 (s, 1H), 7.29 (dd, J=12.2, 1.7 Hz, 1H), 6.33-5.93 (m, 1H), 4.78 (s, 1H), 4.45-4.09 (m, 3H), 4.17-3.89 (m, 1H), 3.88 (s, 3H), 3.66-3.35 (m, 2H), 3.38-3.08 (m, 2H), 2.85-2.35 (m, 2H), 2.03 (ddq, J=45.2, 31.2, 13.5, 12.9 Hz, 5H).

### Example 191

### (2S)—N-[[6-(1,1-Difluoro-2-phenyl-ethyl)-5-fluoro-2-methoxy-3-pyridyl]methyl]-N-methyl-pyrrolidine-2-carboxamide Hydrochloride

(Compound of Formula Ia.24, Wherein X is CH, R⁵ is methyl, R⁶ is F, R⁷ is OCH₃ and R^{8d} is —CF₂CH₂-A.59)

To a suspension of 1-(6-chloro-5-fluoro-2-methoxypyridin-3-yl)-N-methylmethanamine hydrochloride (1.00 g, 4.15 mmol) (see example 114, step 114.2) in DCM (10.4 mL) was added N-ethyl-N-isopropylpropan-2-amine (2.90 mL, 16.6 mmol) followed by ditert-butyl dicarbonate (1.09 g, 4.98 mmol). The resulting mixture was stirred at room temperature for 16 h before being diluted with DCM. The DCM layer was washed with aqueous sodium bicarbonate solution followed by aqueous ammonium chloride solution and dried (Na₂SO₄). The solvent was removed in vacuo and the crude material was purified using flash chromatography on silica (heptane 100%→heptane:EtOAc 80:20) to give tert-butyl ((6-chloro-5-fluoro-2-methoxypyridin-3-yl)methyl)(methyl)carbamate (1.27 g, yield 100%). A mixture of cesium carbonate (947 mg, 2.91 mmol), 2,2-difluoroacetophenone (192 μl, 1.45 mmol) and tert-butyl ((6-chloro-5-fluoro-2-methoxypyridin-3-yl)methyl)(methyl)carbamate (443 mg, 1.45 mmol) in toluene (7.3 mL) was degassed for 10 min by bubbling through Ar. Bis(dibenzylideneacetone)palladium (41.8 mg, 0.073 mmol) and butyldi-1-adamantylphosphine (31.3 mg, 0.087 mmol) were added and the mixture was heated to 100° C. for 5 h. Water was added and the product extracted into EtOAc (3×). The combined organic phases were dried (brine, Na₂SO₄), concentrated and purified by column chromatography on silica (heptane 100%→heptane:EtOAc 80:20) to give tert-butyl ((6-(1,1-difluoro-2-oxo-2-phenylethyl)-5-fluoro-2-methoxypyridin-3-yl)methyl)(methyl)carbamate (260 mg, yield 42%).

To a solution of tert-butyl ((6-(1,1-difluoro-2-oxo-2-phenylethyl)-5-fluoro-2-methoxypyridin-3-yl)methyl)(methyl)carbamate (160 mg, 0.38 mmol) in methanol (1.9 mL) was added NaBH₄ (31.4 mg, 0.83 mmol). This was stirred at room temperature for 45 min. Water was added and the product extracted into EtOAc (3×). The combined organic phases were dried (brine, Na₂SO₄) and the solvent was removed in vacuo. This was used directly in the next step.

To a solution of tert-butyl ((6-(1,1-difluoro-2-hydroxy-2-phenylethyl)-5-fluoro-2-methoxypyridin-3-yl)methyl)(methyl)carbamate (crude, from last step, 0.38 mmol) in thionyl chloride (2.0 ml, 27 mmol) was added pyridine (2 ml, 24 mmol). This mixture was heated to 70° C. for 30 min before being diluted with EtOAc. The organic layer was washed with 5% aqueous citric acid and aqueous sodium bicarbonate solution and dried (Na₂SO₄). The solvent was removed in vacuo and the resulting brown oil was used directly in the next step.

A solution of tert-butyl ((6-(2-chloro-1,1-difluoro-2-phenylethyl)-5-fluoro-2-methoxypyridin-3-yl)methyl)(methyl)carbamate (crude from last step, 0.38 mmol) in EtOAc (10 ml) was run through the H-cube with a 10% Pd/C cartridge under 80 bar H₂ at 60° C. at 1 mL/min and then at 0.5 mL/min. The solvent was removed and the residue purified by column chromatography on silica (heptane 100%→heptane:EtOAc 80:20) to give tert-butyl ((6-(1,1-difluoro-2-phenylethyl)-5-fluoro-2-methoxypyridin-3-yl)methyl)(methyl)carbamate (31.4 mg, yield 20% over three steps).

To a solution of tert-butyl ((6-(1,1-difluoro-2-phenylethyl)-5-fluoro-2-methoxypyridin-3-yl)methyl)(methyl)carbamate (31.4 mg, 0.077 mmol) in EtOAc (4 ml) was added HCl (5-6M in iPrOH) (1 ml). This mixture was stirred overnight, the solvent removed and taken directly to the next step.

1-(6-(1,1-Difluoro-2-phenylethyl)-5-fluoro-2-methoxypyridin-3-yl)-N-methylmethanamine hydrochloride (from last step, 0.077 mmol) was dissolved in DMF (1.8 mL). 2-(1H-Benzo[d][1,2,3]triazol-1-yl)-1,1,3,3-tetramethylisouronium hexafluorophosphate(V)/HBTU (35.9 mg, 0.091 mmol), DIPEA (40.1 μl, 0.23 mmol) and (S)-1-(tert-butoxycarbonyl)pyrrolidine-2-carboxylic acid (19.8 mg, 0.091 mmol) were added and the reaction mixture was stirred at room temperature for 90 min. Water was added, the reaction mixture was basified with NaHCO₃ and the product extracted into EtOAc (3×). The combined organic layers were dried (brine, Na₂SO₄), concentrated and purified by column chromatography on silica (heptane 100%→heptane:EtOAc 70:30) to give (S)-tert-butyl 2-(((6-(1,1-difluoro-2-phenylethyl)-5-fluoro-2-methoxypyridin-3-yl)methyl)(methyl)carbamoyl)pyrrolidine-1-carboxylate which was not clean but taken directly to the next step.

To a solution of (S)-tert-butyl 2-(((6-(1,1-difluoro-2-phenylethyl)-5-fluoro-2-methoxypyridin-3-yl)methyl)(methyl)carbamoyl)pyrrolidine-1-carboxylate (0.077 mmol, from last step) in EtOAc (4 ml) was added HCl (5-6M in iPrOH) (1 ml). This mixture was stirred overnight. Water was added, the mixture basified with NaHCO₃ and the product extracted into EtOAc (3×). The combined organic layers were dried (brine, Na₂SO₄), concentrated and purified by column chromatography on silica (DCM 100%→DCM:MeOH 80:20) followed by preparative HPLC chromatography on a reversed phase column. The product was dissolved in DCM and HCl (1.0 M in Et₂O) was added to give (S)—N-((6-(1,1-difluoro-2-phenylethyl)-5-fluoro-2-methoxypyridin-3-yl)methyl)-N-methylpyrrolidine-2-carboxamide hydrochloride as a white solid (13.3 mg, yield 39% over three steps).

Mixture of two rotamers A:B, 1:2.7

¹H NMR (500 MHz, methanol-d₄) δ 7.51 (d, J=9.9 Hz, 1H, A), 7.43 (d, J=10.1 Hz, 1H, B), 7.26-7.18 (m, 5H, A and B), 4.77-4.69 (m, 1H, A and B), 4.68-4.38 (m, 2H, A and B), 3.97 (s, 3H, A), 3.95 (s, 3H, B), 3.65 (t, J=16.7, 2H, A), 3.64 (t, J=16.7, 2H, B), 3.43 (dt, J=11.5, 7.0 Hz, 1H, A and B), 3.38-3.32 (m, 1H, A and B), 2.64-2.49 (m, 1H, B), 2.49-2.37 (m, 1H, A), 2.16-2.01 (m, 2H, A and B), 2.01 1.90 (m, 1H, A and B).

### Example 192

### N-[[6-(2-Chloro-1,1-difluoro-2-phenyl-ethyl)-5-fluoro-2-methoxy-3-pyridyl]-methyl]-N-methyl-pyrrolidine-2-carboxamide Dihydrochloride

(Compound of Formula Ia.24, Wherein X is CH, R⁵ is methyl, R⁶ is F, R⁷ is OCH₃ and R^{8d} is —CF₂CHCl-A.59)

To a solution of tert-butyl ((6-(2-chloro-1,1-difluoro-2-phenylethyl)-5-fluoro-2-methoxypyridin-3-yl)methyl)(methyl)carbamate (10 mg, 0.022 mmol; example 191) in EtOAc (2 ml) was added HCl (5-6M in iPrOH) (0.5 ml). This mixture was stirred overnight, the solvent removed and taken directly to the next step. 1-(6-(2-Chloro-1,1-difluoro-2-phenylethyl)-5-fluoro-2-methoxypyridin-3-yl)-N-methylmethanamine hydrochloride (from last step, 0.022 mmol) was dissolved in DMF (0.5 mL). 2-(1H-Benzo[d][1,2,3]triazol-1-yl)-1,1,3,3-tetramethylisouronium hexafluorophosphate(V)/HBTU (10.3 mg, 0.026 mmol), DIPEA (11.5 μl, 0.066 mmol) and (S)-1-(tert-butoxycarbonyl)pyrrolidine-2-carboxylic acid (5.7 mg, 0.026 mmol) were added and the reaction mixture was stirred at room temperature for 90 min. Water was added, the reaction mixture was basified with NaHCO₃ and the product extracted into EtOAc (3×). The combined organic layers were dried (brine, Na₂SO₄), concentrated and purified by column chromatography on silica (heptane 100%→heptane:EtOAc 70:30) to give tert-butyl ((6-(2-chloro-1,1-difluoro-2-phenylethyl)-5-fluoro-2-methoxypyridin-3-yl)methyl)(methyl)carbamate which was not clean but taken directly to the next step. To a solution of (S)-tert-butyl 2-(((6-(2-chloro-1,1-difluoro-2-phenylethyl)-5-fluoro-2-methoxypyridin-3-yl)methyl)(methyl)carbamoyl)pyrrolidine-1-carboxylate (0.022 mmol, from last step) in EtOAc (2 ml) was added HCl (5-6M in iPrOH) (0.5 ml). This mixture was stirred overnight. Water was added, the mixture basified with NaHCO₃ and the product extracted into EtOAc (3×). The combined organic layers were dried (brine, Na₂SO₄), concentrated and purified by column chromatography on silica (DCM 100%→DCM:MeOH 80:20) followed by preparative HPLC chromatography on a reversed phase column. The product was dissolved in DCM and HCl (1.0 M in Et₂O) was added to give (S)—N-((6-(1,1-difluoro-2-phenylethyl)-5-fluoro-2-methoxypyridin-3-yl)methyl)-N-methylpyrrolidine-2-carboxamide hydrochloride as a white solid (6.3 mg, yield 59% over three steps).

Mixture of two rotamers A:B, 1:3 and two diastereomers

¹H NMR (500 MHz, methanol-d₄) δ 7.51 (dd, J=10.1, 5.5 Hz, 1H, A), 7.48-7.45 (m, 2H, A and B), 7.43 (d, J=10.1 Hz, 1H, B), 7.36-7.26 (m, 3H, A and B), 5.79 (t, J=12.6, 1H, A), 5.78 (t, J=12.6, 1H, B), 4.71 (t, J=7.8 Hz, 1H, A and B), 4.65-4.39 (m, 2H, A and B), 3.96 (d, J=3.5 Hz, 3H, A), 3.95 (d, J=3.0 Hz, 3H, B), 3.49-3.38 (m, 1H, A and B), 3.36-3.32 (m, 1H, A and B), 3.10 (d, J=3.4 Hz, 3H, B), 2.88 (d, J=4.0 Hz, 3H, A), 2.62-2.50 (m, 1H, B), 2.47-2.36 (m, 1H, A), 2.15-2.02 (m, 2H, A and B), 2.02-1.89 (m, 1H, A and B).

### Example 193

### (2S)—N-[[5-Fluoro-2-methoxy-6-(4-methoxybutyl)-3-pyridyl]methyl]pyrrolidine-2-carboxamide

(Compound of Formula Ia.24, Wherein X is CH, R⁵ is H, R⁶ is F, R⁷ is OCH₃ and R^{8d} is —(CH₂)₄—OCH₃)

The title compound was prepared using the procedure described in example 139 starting from (S)-tert-butyl 2-(((6-chloro-5-fluoro-2-methoxypyridin-3-yl)methyl)carbamoyl)pyrrolidine-1-carboxylate (200 mg, 0.516 mmol) and (4-methoxybutyl) boronic acid (74.9 mg, 0.567 mmol) to give (S)-tert-butyl 2-(((5-fluoro-2-methoxy-6-(4-methoxybutyl)pyridin-3-yl)methyl)carbamoyl)pyrrolidine-1-carboxylate (35 mg, yield 15.4%) followed by Boc deprotection using the procedure described in example 140 to give the title compound (S)—N-((5-fluoro-2-methoxy-6-(4-methoxybutyl)pyridin-3-yl)methyl)pyrrolidine-2-carboxamide (18 mg, yield 66.6%).

LCMS (ESI⁺) m/z [M+H]⁺: 340.10

¹H NMR (600 MHz, DMSO-d₆) δ ppm: 8.45-8.40 (m, 1H), 7.25 (d, J=9.4 Hz, 1H), 4.15-4.10 (m, 2H), 3.88 (s, 3H), 3.60-3.55 (m, 1H), 3.40-3.35 (m, 2H), 3.21 (s, 3H), 2.90-2.85 (m, 2H), 2.65-2.60 (m, 2H), 2.00-1.95 (m, 1H), 1.70-1.60 (m, 5H), 1.55-1.50 (m, 2H).

### Example 194

### (2S)—N-[[6-(3-Benzylazetidin-1-yl)-5-fluoro-2-methoxy-3-pyridyl]methyl]-pyrrolidine-2-carboxamide

(Compound of Formula Ia.23, Wherein X is CH, R⁵ is H, R⁶ is F, R⁷ is OCH₃, and NR^{8b}R^{8c} is A.102)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 3-benzylazetidine (110 mg, 0.747 mmol) to give (S)-tert-butyl 2-(((6-(3-benzylazetidin-1-yl)-5-fluoro-2-methoxypyridin-3-yl)methyl)carbamoyl)pyrrolidine-1-carboxylate (226.8 mg, yield 70.6%, purity 80%) as crude product followed by Boc deprotection using the procedure described in example 134 to give the title compound (S)—N-((6-(3-benzylazetidin-1-yl)-5-fluoro-2-methoxypyridin-3-yl)methyl)pyrrolidine-2-carboxamide (87.5 mg, yield 48.3%).

LCMS (ESI⁺) m/z [M+H]⁺: 399.30

¹H NMR (600 MHz, DMSO-d₆) δ ppm: 8.27 (t, J=6.0 Hz, 1H), 7.33-7.25 (m, 2H), 7.27-7.15 (m, 4H), 4.11-3.98 (m, 4H), 3.80 (s, 3H), 3.82-3.71 (m, 2H), 3.60 (dd, J=8.8, 5.6 Hz, 1H), 3.05-2.95 (m, 1H), 2.93 (d, J=7.7 Hz, 2H), 2.91-2.78 (m, 2H), 2.02-1.93 (m, 1H), 1.70-1.56 (m, 3H).

### Example 195

### (2S)—N-[[5-Fluoro-2-methoxy-6-(4-phenoxy-1-piperidyl)-3-pyridyl]methyl]-pyrrolidine-2-carboxamide; 2,2,2-trifluoroacetic Acid

(Compound of Formula Ia.23, Wherein X is CH, R⁵ is H, R⁶ is F, R⁷ is OCH₃, and NR^{8b}R^{8c} is A.132)

The title compound was prepared using the procedure described in example 173 starting from (S)-tert-butyl 2-(((6-chloro-5-fluoro-2-methoxypyridin-3-yl)methyl)carbamoyl)pyrrolidine-1-carboxylate (200 mg, 0.516 mmol) and 4-phenoxypiperidine (110 mg, 0.619 mmol) to give (S)-tert-butyl 2-(((5-fluoro-2-methoxy-6-(4-phenoxy-piperidin-1-yl)pyridin-3-yl)methyl)carbamoyl)pyrrolidine-1-carboxylate (187.2 mg, yield 20.6%, purity 30%) as crude product followed by Boc deprotection as described in procedure example 136. Preparative HPLC chromatography on a reversed phase column (eluents contained 0.1% TFA) gave the title compound (S)—N-((5-fluoro-2-methoxy-6-(4-phenoxypiperidine-1-yl)pyridin-3-yl)methyl)pyrrolidine-2-carboxamide 2,2,2-trifluoroacetate (64.9 mg, yield 75%).

LCMS (ESI⁺) m/z [M+H]⁺: 429.35

¹H NMR (500 MHz, DMSO-d₆) δ ppm: 9.18 (s, 1H), 8.77 (t, J=5.6 Hz, 1H), 8.56 (s, 1H), 7.37 (d, J=13.1 Hz, 1H), 7.33-7.23 (m, 2H), 7.01-6.94 (m, 2H), 6.93 (t, J=7.3 Hz, 1H), 4.62 (tt, J=8.2, 3.9 Hz, 1H), 4.23-4.11 (m, 3H), 3.84 (s, 3H), 3.83-3.78 (m, 2H), 3.33-3.13 (m, 4H), 2.29 (ddt, J=12.7, 8.6, 6.3 Hz, 1H), 2.04 (ddt, J=13.0, 6.4, 3.5 Hz, 2H), 1.89 (p, J=7.0, 6.6 Hz, 2H), 1.88-1.77 (m, 1H), 1.68 (dtd, J=12.7, 8.8, 3.6 Hz, 2H).

### II. Biological Tests

### Functional Activity

### 1. Human 5-HT_{2C} Functional Assay

The functional activity of compounds of formula I was assayed by incubation with U2OS_HTR_{2C}_{_}β-Arrestin cells (DiscoverX, 93-0289C3) to induce beta-arrestin2 recruitment to the 5-HT_{2C} receptor. The agonist-induced recruitment and proximity of the receptor and beta-arrestin2 leads to complementation and formation of active β-galactosidase. The enzyme complementation results in enzyme activity, which is measured following the termination of the agonist incubation using DiscoveRx's detection reagent, which contains a chemiluminescent substrate which produces a high intensity signal. Cells were plated and a medium-change to a 1% serum containing medium was performed 24h later. The next day, test compounds were added and incubated for 1.5 h before addition of detection reagent.

The response produced was measured and compared with the response produced by 10 [mu]M 5-HT or the maximal effect induced by 5-HT (defined as 100%) to which it was expressed as a percentage response (relative efficacy). Dose response curves were constructed using Graphpad Prism (Graph Software Inc.) or using in house adapted software using a 4 parameter dose response model with variable slope (fit=(Bottom+(Top-Bottom)/(1+10^{∧}((Log EC50−x)*HillSlope))res=(y-fit)). Results are compiled in the table below.

### 2. Human 5-HT_{2A} Functional Assay

Functional activity on the 5-HT_{2A} receptor was determined by testing the effect of the compounds I on calcium mobilisation in CHO-K1 cells, stably transfected with human 5-HT_{2A} receptor. Cells were seeded into sterile black 384-well plates with clear bottom at 25,000 cells/well in a volume of 25 μl and grown for 5-6 hours at 37° C., in 5% CO₂ in tissue culture medium (“Ultra CHO” by LONZA), containing 1% dialysed FCS and 50 μg/ml gentamicin (Invitrogen). After this incubation, medium was replaced by a serum free version of the same tissue culture medium followed by incubation overnight at 37° C. and in 5% CO₂. Cells were then loaded with a fluorescent calcium-sensitive dye in the presence of 0.07% probenecid for an hour at 37° C., according to the manufacturer's protocol (Ca5-Assay Kit, Molecular Devices), followed by an additional 60 min incubation at room temperature. Serial compound dilutions (final concentrations of 10⁻¹⁰ to 10⁻⁵M, prepared in HBSS+50 mM HEPES) were first added to the cells alone (“first addition” to assess agonism on the 5-HT_{2A} receptor), then after 8 min, serotonin was added to the same wells at a final concentration of 3×10⁻⁸ M (“second addition” to see potential antagonistic effect) and the maximal calcium response was determined using a FLIPR® Tetra instrument (Molecular Devices) in each of the two steps. The relative efficacy of the compounds was calculated as a percentage of the maximal effect induced by serotonin alone (defined as 100%). To determine EC₅₀/IC₅₀ values, concentration-response curves were fitted using a four-parameter logistic equation (IDBS Biobook™). K_{b} values were calculated from IC₅₀ values, according to Cheng & Prusoff.

### 3. Human 5-HT_{2B} Functional Assay

Functional activity on the 5-HT_{2B} receptor was determined by testing the effect of the compounds I on calcium mobilisation in CHO-FlpIn cells, stably transfected with human 5-HT_{2B} receptor. Cells were seeded into sterile black 384-well plates with clear bottom at 30,000 cells/well in a volume of 25 μl and grown overnight at 37° C., in 5% CO₂ in tissue culture medium (“CHO-S-SFM II” by Invitrogen), containing 1% dialysed FCS and 50 μg/ml gentamicin (Invitrogen). On the next morning, medium was replaced by a serum free version of the same tissue culture medium for a further incubation for 4 hours at 37° C. and in 5% CO₂. Cells were then loaded with a fluorescent calcium-sensitive dye in the presence of 0.07% probenecid for an hour at 37° C., according to the manufacturer's protocol (Ca5-Assay Kit, Molecular Devices), followed by an additional 60 min incubation at room temperature. Serial compound dilutions (final concentrations of 10⁻¹⁰ to 10⁻⁵M, prepared in HBSS+50 mM HEPES) were first added to the cells alone (“first addition” to assess agonism on the 5-HT_{2B} receptor), then after 8 min, serotonin was added to the same wells at a final concentration of 10⁻⁸ M (“second addition” to see potential antagonistic effect) and the maximal calcium response was determined using a FLIPR® Tetra instrument (Molecular Devices) in each of the two steps. The relative efficacy of the compounds was calculated as a percentage of the maximal effect induced by serotonin alone (defined as 100%). To determine EC₅₀/IC₅₀ values, concentration-response curves were fitted using a four-parameter logistic equation (IDBS Biobook™). K_{b} values were calculated from IC₅₀ values, according to Cheng & Prusoff.

### 4. Metabolic Stability

Samples of the tested compounds (0.5 μM) were preincubated together with human liver microsomes (0.25 mg of microsomal protein/mL) in 0.05 M potassium phosphate buffer of pH 7.4 in microtiter plates at 37° C. for 5 minutes. The reaction was started by adding NADPH (1.0 mM). After 0, 5, 10, 15, 20 and 30 minutes, an aliquot was removed, the reaction was cooled and stopped by adding twice the amount of quench solution consisting of acetonitrile/methanol 1:1, and containing 0.2 μM carbutamide as internal standard. The samples were frozen until analyzed. The remaining concentration of undegraded test substance was determined by liquid chromatography-tandem mass spectrometry (LC-MS/MS). The half-life (t_{1/2}) was determined from the gradient of the ratio of the signal of (test substance/internal standard)/unit time plot, allowing the calculation of the half-life of the test substance, assuming first order kinetics, from the decrease in the concentration of the compound with time. The microsomal clearance (mClint) was calculated as follows: mClint=((ln(2)/t ½)/Microsomal Protein Concentration (mg/ml))*1000, leading to the unit of uL/min/mg. The scaled clearance (mClin_scaled) was calculated as mCLint_scaled=m CLint*(Microsomal Yield (mg/kg BW))/1000000*60, leading to the units L/h/kg. The Microsomal Yield is defined by the specifics of the used microsomes. Calculations were modified from references: Di, The Society for Biomolecular Screening, 2003, 453-462; Obach, D M D, 1999 vol 27. N 11, 1350-1359.

### Unbound Fraction in Microsomes (Fu Mic)

A suspension of 0.25 mg/ml microsomal protein spiked with 0.5 μM of test compound was pipetted on one side of a HTDialysis device (HTDialysis LLC, 37 Ledgewood Drive, Gales Fery CT 06335) separated by a membrane with a MWcut off 12-14 K. 50 mM K-Phosphate buffer (pH 7.4) was added on the other side of the well. After incubation at 37° C. for 4 h while shaking at 150 rpm, aliquots of both sides were taken, quenched with MeOH/ACN 1:1 and 0.2 μM of internal standard and frozen until analysis by LCMSMS

### Calculation of Unbound Intrinsic Clearance

Cl int unbound=Cl int/fu mic

| | | | Cl int, mic unb.² |
|---|---|---|---|
| # | EC50 5-HT_{2C}¹ | % efficacy | (h) [l/h/kg] |
| 1 | +++ | +++ | +++ |
| 2 | +++ | +++ | +++ |
| 3 | +++ | +++ | ++ |
| 4 | +++ | +++ | ++ |
| 5 | +++ | +++ | |
| 6 | +++ | +++ | +++ |
| 7 | +++ | +++ | +++ |
| 8 | +++ | +++ | ++ |
| 9 | +++ | +++ | ++ |
| 10 | +++ | +++ | ++ |
| 11 | +++ | +++ | ++ |
| 12 | ++ | +++ | +++ |
| 13 | ++ | +++ | +++ |
| 14 | +++ | +++ | + |
| 15 | +++ | +++ | + |
| 16 | +++ | +++ | |
| 17 | ++ | ++ | ++ |
| 18 | +++ | +++ | +++ |
| 19 | +++ | +++ | ++ |
| 20 | +++ | +++ | ++ |
| 21 | ++ | +++ | |
| 22 | +++ | +++ | |
| 23 | +++ | +++ | +++ |
| 24 | +++ | +++ | +++ |
| 25 | ++ | ++ | +++ |
| 26 | +++ | +++ | +++ |
| 27 | +++ | +++ | +++ |
| 28 | +++ | +++ | +++ |
| 29 | +++ | +++ | +++ |
| 30 | +++ | +++ | +++ |
| 31 | +++ | +++ | +++ |
| 32 | +++ | ++ | +++ |
| 33 | +++ | +++ | +++ |
| 34 | +++ | +++ | +++ |
| 35 | +++ | +++ | +++ |
| 36 | +++ | ++ | ++ |
| 37 | +++ | ++ | +++ |
| 38 | ++ | ++ | ++ |
| 39 | ++ | ++ | |
| 40 | +++ | +++ | +++ |
| 41 | +++ | +++ | |
| 42 | +++ | +++ | +++ |
| 43 | ++ | +++ | +++ |
| 44 | +++ | +++ | +++ |
| 45 | +++ | +++ | |
| 46 | +++ | +++ | +++ |
| 47 | +++ | +++ | +++ |
| 48 | +++ | +++ | +++ |
| 49 | +++ | +++ | ++ |
| 50 | +++ | +++ | |
| 52 | ++ | +++ | +++ |
| 53 | +++ | +++ | +++ |
| 54 | +++ | +++ | +++ |
| 55 | +++ | +++ | +++ |
| 56 | +++ | +++ | ++ |
| 57 | +++ | +++ | |
| 58 | +++ | +++ | +++ |
| 59 | +++ | ++ | +++ |
| 60 | ++ | ++ | + |
| 61 | ++ | +++ | ++ |
| 62 | ++ | +++ | + |
| 63 | +++ | +++ | +++ |
| 64 | +++ | +++ | |
| 65 | ++ | ++ | +++ |
| 66 | +++ | +++ | ++ |
| 67 | +++ | +++ | |
| 68 | +++ | +++ | +++ |
| 69 | +++ | +++ | |
| 70 | +++ | +++ | |
| 71 | +++ | +++ | +++ |
| 72 | +++ | +++ | ++ |
| 73 | + | +++ | ++ |
| 74 | +++ | +++ | ++ |
| 75 | +++ | +++ | ++ |
| 76 | +++ | +++ | +++ |
| 77 | +++ | +++ | +++ |
| 78 | +++ | +++ | +++ |
| 79 | ++ | + | + |
| 80 | ++ | +++ | |
| 81 | + | +++ | +++ |
| 82 | ++ | +++ | ++ |
| 85 | +++ | + | |
| 86 | +++ | +++ | +++ |
| 87 | +++ | +++ | +++ |
| 88 | +++ | +++ | ++ |
| 89 | +++ | +++ | ++ |
| 90 | +++ | +++ | +++ |
| 91 | +++ | +++ | +++ |
| 92 | +++ | +++ | +++ |
| 93 | +++ | +++ | +++ |
| 94 | +++ | +++ | +++ |
| 95 | +++ | +++ | ++ |
| 96 | +++ | +++ | +++ |
| 97 | +++ | +++ | +++ |
| 98 | ++ | ++ | +++ |
| 99 | ++ | ++ | +++ |
| 100 | +++ | +++ | +++ |
| 101 | +++ | +++ | +++ |
| 102 | + | ++ | +++ |
| 103 | ++ | ++ | +++ |
| 104 | +++ | +++ | +++ |
| 105 | +++ | +++ | ++ |
| 106 | ++ | + | +++ |
| 107 | + | +++ | +++ |
| 108 | ++ | +++ | +++ |
| 109 | +++ | +++ | + |
| 110 | +++ | +++ | ++ |
| 111 | +++ | +++ | ++ |
| 112 | +++ | +++ | +++ |
| 113 | + | + | +++ |
| 114 | +++ | +++ | ++ |
| 115 | +++ | ++ | +++ |
| 116 | ++ | ++ | ++ |
| 117 | +++ | ++ | ++ |
| 118 | +++ | +++ | ++ |
| 119 | ++ | +++ | ++ |
| 120 | +++ | ++ | |
| 121 | +++ | ++ | |
| 122 | +++ | +++ | |
| 123 | +++ | ++ | |
| 124 | ++ | ++ | |
| 125 | + | +++ | ++ |
| 126 | +++ | +++ | +++ |
| 127 | + | +++ | ++ |
| 128 | ++ | +++ | +++ |
| 129 | +++ | +++ | |
| 130 | +++ | +++ | ++ |
| 131 | +++ | +++ | ++ |
| 132 | +++ | +++ | + |
| 133 | +++ | +++ | ++ |
| 134 | +++ | +++ | +++ |
| 135 | ++ | +++ | |
| 136 | +++ | +++ | +++ |
| 137 | + | +++ | + |
| 138 | + | +++ | ++ |
| 139 | + | + | ++ |
| 140 | +++ | +++ | ++ |
| 141 | +++ | +++ | + |
| 142 | +++ | +++ | ++ |
| 143 | +++ | +++ | + |
| 144 | ++ | +++ | ++ |
| 145 | +++ | +++ | +++ |
| 146 | ++ | +++ | +++ |
| 147 | + | ++ | ++ |
| 148 | ++ | +++ | + |
| 149 | ++ | +++ | |
| 150 | ++ | +++ | ++ |
| 151 | + | +++ | + |
| 152 | +++ | +++ | +++ |
| 153 | ++ | +++ | ++ |
| 154 | ++ | +++ | ++ |
| 155 | ++ | +++ | ++ |
| 156 | ++ | ++ | +++ |
| 157 | + | ++ | ++ |
| 158 | ++ | +++ | +++ |
| 159 | + | +++ | +++ |
| 160 | ++ | ++ | +++ |
| 161 | ++ | ++ | +++ |
| 162 | ++ | ++ | ++ |
| 163 | +++ | +++ | ++ |
| 164 | ++ | +++ | +++ |
| 165 | ++ | ++ | +++ |
| 166 | ++ | ++ | +++ |
| 167 | + | +++ | +++ |
| 168 | + | +++ | +++ |
| 169 | +++ | +++ | +++ |
| 170 | ++ | +++ | +++ |
| 171 | + | +++ | ++ |
| 172 | ++ | +++ | +++ |
| 173 | + | ++ | ++ |
| 174 | + | + | +++ |
| 175 | ++ | ++ | ++ |
| 176 | +++ | +++ | ++ |
| 177 | ++ | +++ | +++ |
| 178 | + | | +++ |
| 179 | + | ++ | ++ |
| 180 | + | +++ | + |
| 181 | +++ | | |
| 182 | +++ | +++ | ++ |
| 183 | ++ | +++ | ++ |
| 184 | ++ | +++ | ++ |
| 185 | ++ | +++ | ++ |
| 186 | ++ | +++ | +++ |
| 187 | ++ | +++ | ++ |
| 188 | ++ | +++ | ++ |
| 189 | + | ++ | +++ |
| 190 | +++ | +++ | +++ |
| 191 | + | + | ++ |
| 192 | + | ++ | |
| 193 | + | +++ | +++ |
| 194 | ++ | +++ | + |
| 195 | ++ | +++ | + |

¹Potency (EC50 5-HT_{2C}) in functional assay
²unbound intrinsic clearance (human)
Potency (EC50):
+ from 200 nM to <1 μM
++ from 20 nM to <200 nM
+++ <20 nM
% Efficacy:
+ from 30 to 50%
++ from >50 to 70%
+++ >70%
Unbound intrinsic clearance:
+ from 50 to 500 l/h/kg
++ from 5 to <50 l/h/kg
+++ <5 l/h/kg

We claim:

1. A compound of formula (I) 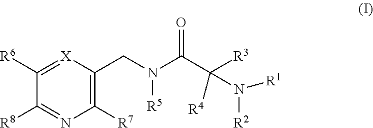
wherein
X is CH or N;
R¹ is selected from the group consisting of hydrogen and C₁-C₄-alkyl;
R² is selected from the group consisting of hydrogen and C₁-C₄-alkyl; or
R¹ and R², together with the nitrogen atom they are bound to, form an N-bound 4-, 5- or 6-membered saturated heterocyclic ring which may carry 1 or 2 substituents selected from the group consisting of halogen, C₁-C₄-alkyl and fluorinated C₁-C₄-alkyl;
R³ is selected from the group consisting of hydrogen and C₁-C₄-alkyl; or
R² and R³, together with the atoms they are bound to, form a 4-, 5- or 6-membered saturated or partially unsaturated heteromonocyclic ring which may carry 1 or 2 substituents selected from the group consisting of halogen, C₁-C₄-alkyl and fluorinated C₁-C₄-alkyl, or form a 4-, 5-, 6-, 7-, 8-, 9- or 10-membered saturated or partially unsaturated heterobicyclic ring which may carry 1 or 2 substituents selected from the group consisting of halogen, C₁-C₄-alkyl and fluorinated C₁-C₄-alkyl;
R⁴ is selected from the group consisting of hydrogen, C₁-C₄-alkyl and C₃-C₆-cycloalkyl;
R⁵ is selected from the group consisting of hydrogen and C₁-C₄-alkyl;
R⁶ is hydrogen or halogen;
R⁷ is selected from the group consisting of C₁-C₄-alkoxy, fluorinated C₁-C₄-alkoxy, hydroxyl, C₁-C₄-alkyl and fluorinated C₁-C₄-alkyl;
R⁸ is selected from the group consisting of —OR^{8a}, —NR^{8b}R^{8c} and —R^{8d};
R^{8a} is selected from the group consisting of C₁-C₈-alkyl; C₁-C₄-alkyl which carries a radical R^{9a}; fluorinated C₁-C₈-alkyl; C₃-C₆-cycloalkyl which may carry one or more substituents R¹⁰; C₅-C₁₀-bicycloalkyl which may carry one or more substituents R¹⁰; and phenyl which may carry one or more substituents R¹²;
R^{8b} is selected from the group consisting of hydrogen, C₁-C₄-alkyl and C₂-C₄-alkenyl;
R^{8c} is selected from the group consisting of C₁-C₄-alkyl which carries a radical R^{9b}; C₃-C₆-cycloalkyl which may carry one or more substituents R¹¹; and phenyl which may carry one or more substituents R¹²; or
R^{8b} and R^{8c}, together with the nitrogen atom they are bound to, form a 4-, 5-, 6- or 7-membered saturated heteromonocyclic ring which may additionally contain a further heteroatom selected from the group consisting of N, O and S as ring member, where the heteromonocyclic ring may carry one or more substituents R¹⁴; or form a 6-, 7-, 8-, 9- or 10-membered saturated or unsaturated heterobicyclic ring which may carry one or more substituents R¹⁴;
R^{8d} is a C-bound radical selected from the group consisting of C₁-C₄-alkyl which carries a radical R^{9c}; C₁-C₄-haloalkyl which carries a radical R^{9c}; C₃-C₆-cycloalkyl which may carry one or more substituents R¹¹; C₃-C₆-cycloalkenyl which may carry one or more substituents R¹¹; phenyl which may carry one or more substituents R¹²; and a C-bound 5- or 6-membered saturated heteromonocyclic ring containing a heteroatom selected from the group consisting of N, O and S as ring member, where the heteromonocyclic ring may carry one or more substituents R¹²;
R^{9a} is selected from the group consisting of C₃-C₆-cycloalkyl which may carry one or more substituents R¹¹; C₅-C₁₀-bicycloalkyl which may carry one or more substituents R¹¹; adamantyl which may carry one or more substituents R¹¹; and phenyl which may carry one or more substituents R¹²;
R^{9b} is selected from the group consisting of C₃-C₆-cycloalkyl which may carry one or more substituents R¹¹; and phenyl which may carry one or more substituents R¹²;
R^{9c} is selected from the group consisting of C₁-C₄-alkoxy; C₁-C₄-haloalkoxy C₃-C₆-cycloalkyl which may carry one or more substituents R¹¹; phenyl which may carry one or more substituents R¹²; and a 5- or 6-membered saturated heteromonocyclic ring containing a heteroatom selected from the group consisting of N, O) and S as ring member, where the heteromonocyclic ring may carry one or more substituents R¹²;
each R¹⁰ is independently selected from the group consisting of halogen, hydroxyl, C₁-C₄-alkyl, C₁-C₄-haloalkyl, C₁-C₄-alkoxy, C₁-C₄-haloalkoxy and phenyl which may carry 1, 2, 3, 4 or 5 substituents R¹³; or
two substituents R¹⁰ bound on the same carbon atom of the cycloalkyl or bicycloalkyl ring, together with the carbon atom they are bound to, form a 3-, 4-, 5- or 6-membered saturated carbocyclic ring, where the carbocyclic ring may carry one or more substituents selected from the group consisting of halogen, C₁-C₄-alkyl and C₁-C₄-haloalkyl;
where two substituents R¹⁰ bound on the same carbon atom of the cycloalkyl or bicycloalkyl ring R^{8a} are not both C₁-C₄-alkyl;
each R¹¹ is independently selected from the group consisting of halogen, C₁-C₄-alkyl, C₁-C₄-haloalkyl, C₁-C₄-alkoxy and C₁-C₄-haloalkoxy;
R¹² and R¹³, independently of each other and independently of each occurrence, are selected from the group consisting of halogen, CN, C₁-C₄-alkyl, C₁-C₄-haloalkyl, C₁-C₄-alkoxy and C₁-C₄-haloalkoxy;
each R¹⁴ is independently selected from the group consisting of halogen; C₁-C₄-alkyl; C₁-C₄-haloalkyl; C₃-C₆-cycloalkyl which may carry one or more substituents selected from the group consisting of halogen and C₁-C₄-haloalkyl; phenyl which may carry one or more substituents R¹²; phenoxy which may carry one or more substituents R¹²; and benzyl which may carry one or more substituents R¹²;
or an N-oxide, tautomer, or stereoisomer thereof; or a pharmaceutically acceptable salt thereof; or the compound of the general formula (I), wherein at least one of the atoms has been replaced by its stable, non-radioactive isotope.

2. The compound as claimed in claim 1, where
R² and R³, together with the atoms they are bound to, form a 4- or 5-membered saturated or partially unsaturated heteromonocyclic ring which may carry 1 substituent selected from the group consisting of halogen, C₁-C₄-alkyl and fluorinated C₁-C₄-alkyl;
R¹ is hydrogen or methyl; and
R⁴ is hydrogen or methyl.

3. The compound as claimed in claim 2, where
R² and R³, together with the atoms they are bound to, form a 5-membered saturated heteromonocyclic ring which may carry 1 substituent selected from the group consisting of halogen, C₁-C₄-alkyl and fluorinated C₁-C₄-alkyl;
R¹ is hydrogen or methyl; and
R⁴ is hydrogen or methyl.

4. The compound as claimed in claim 1, of formula (I.1) 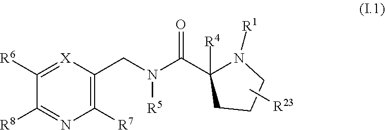
wherein
R²³ is selected from the group consisting of hydrogen, halogen, C₁-C₄-alkyl and fluorinated C₁-C₄-alkyl.

5. The compound as claimed in claim 1, where
R² and R³, together with the atoms they are bound to, form a 6-, 7-, 8-, 9- or 10-membered saturated heterobicyclic ring which may carry 1 substituent selected from the group consisting of halogen, C₁-C₄-alkyl and fluorinated C₁-C₄-alkyl;
R¹ is hydrogen or methyl; and
R⁴ is hydrogen or methyl.

6. The compound as claimed in claim 1, where R³ and R⁴ are independently hydrogen or methyl, and R¹ and R², together with the nitrogen atom they are bound to, form an N-bound 4- or 5-membered saturated heterocyclic ring which may carry 1 substituent selected from the group consisting of halogen, C₁-C₂-alkyl and fluorinated C₁-C₂-alkyl.

7. The compound as claimed in claim 1, where R⁵ is hydrogen or methyl.

8. The compound as claimed in claim 1, where R⁶ is F if X is CH and simultaneously R⁷ is C₁-C₄-alkoxy, fluorinated C₁-C₄-alkoxy or hydroxyl; and is hydrogen if X is N.

9. The compound as claimed in claim 1, where R⁷ is C₁-C₄-alkoxy; in particular methoxy.

10. The compound as claimed in claim 1, where R⁷ is C₁-C₄-alkyl.

11. The compound as claimed in claim 1, where X is CH.

12. The compound as claimed in claim 1, where R⁸ is —OR^{8a}.

13. The compound as claimed in claim 12, where
R^{8a} is selected from the group consisting of C₁-C₂-alkyl which carries a radical R^{9a}; fluorinated C₁-C₈-alkyl; C₃-C₆-cycloalkyl which carries one or more substituents R¹⁰; and C₆-C₁₀-bicycloalkyl which carries one or more substituents R¹⁰; where
R^{9a} is selected from the group consisting of C₃-C₆-cycloalkyl which may carry 1, 2, 3, 4, 5 or 6 substituents R¹¹; C₅-C₁₀-bicycloalkyl which may carry 1, 2, 3, 4, 5 or 6 substituents R¹¹; adamantyl; and phenyl which may carry 1, 2 or 3 substituents R¹²;
each R¹⁰ is independently selected from the group consisting of fluorine, hydroxyl, fluorinated C₁-C₄-alkyl, C₁-C₄-alkoxy and fluorinated C₁-C₄-alkoxy; or two substituents R¹⁰ bound on the same carbon atom of the cycloalkyl or bicycloalkyl ring, together with the carbon atom they are bound to, form a 3- or 4-membered saturated carbocyclic ring, where the carbocyclic ring may carry one or more substituents selected from the group consisting of halogen, C₁-C₄-alkyl and C₁-C₄-haloalkyl;
each R¹¹ is independently selected from the group consisting of fluorine and fluorinated C₁-C₄-alkyl; and
each R¹² is independently selected from the group consisting of halogen, CN, C₁-C₄-alkyl, C₁-C₄-haloalkyl, C₁-C₄-alkoxy and C₁-C₄-haloalkoxy.

14. The compound as claimed in claim 13, where
R^{8a} is selected from the group consisting of —CH₂—R^{9a}; fluorinated C₁-C₆-alkyl; C₃-C₆-cycloalkyl which carries 1, 2, 3 or 4 substituents R¹⁰; and C₆-C₁₀-bicycloalkyl which carries 1, 2, 3 or 4 substituents R¹⁰; where
R^{9a} is selected from the group consisting of C₃-C₆-cycloalkyl which may carry 1, 2, 3, 4, 5 or 6 substituents R¹¹; C₅-C₁₀-bicycloalkyl which may carry 1, 2, 3 or 4 substituents R¹¹; adamantyl; and phenyl which may carry 1, 2 or 3 substituents R¹²;
each R¹⁰ is independently selected from the group consisting of fluorine, fluorinated C₁-C₄-alkyl, C₁-C₄-alkoxy and fluorinated C₁-C₄-alkoxy; or
two substituents R¹⁰ bound on the same carbon atom of the cycloalkyl or bicycloalkyl ring, together with the carbon atom they are bound to, form a 3-membered saturated carbocyclic ring, where the carbocyclic ring may carry one or more substituents selected from halogen;
each R¹¹ is independently selected from the group consisting of fluorine and fluorinated C₁-C₄-alkyl; and
each R¹² is independently halogen.

15. The compound as claimed in claim 1, where R⁸ is —NR^{8b}R^{8c}.

16. The compound as claimed in claim 15, where
R^{8b} is selected from the group consisting of hydrogen, C₁-C₄-alkyl and C₂-C₃-alkenyl;
R^{8c} is selected from the group consisting of C₁-C₃-alkyl which carries a radical R^{9b}; C₃-C₆-cycloalkyl which may carry 1 to 6 substituents R¹¹; and phenyl which may carry one or more halogen atoms; or
R^{8b} and R^{8c}, together with the nitrogen atom they are bound to, form a 4-, 5-, 6- or 7-membered saturated heteromonocyclic ring which may additionally contain an oxygen atom as ring member, where the heteromonocyclic ring may carry one or more substituents R¹⁴; or form a 6-, 7-, 8-, 9- or 10-membered saturated or unsaturated heterobicyclic ring which may carry one or more substituents R¹⁴; where
R^{9b} is selected from the group consisting of C₃-C₆-cycloalkyl which may carry one or more substituents selected from the group consisting of halogen and C₁-C₂-haloalkyl; and phenyl which may carry one or more halogen atoms;
each R¹¹ is independently selected from the group consisting of halogen and C₁-C₂-haloalkyl; and
each R¹⁴ is independently selected from the group consisting of halogen; C₁-C₂-alkyl; C₁-C₂-haloalkyl; C₃-C₆-cycloalkyl which may carry one or more substituents selected from the group consisting of halogen and C₁-C₂-haloalkyl; phenyl; phenoxy; and benzyl, where the phenyl ring in the three last-mentioned radicals may carry one or more halogen atoms.

17. The compound as claimed in claim 1, where R⁸ is —R^{8d}.

18. The compound as claimed in claim 17, where
R^{8d} is a C-bound radical selected from the group consisting of C₁-C₄-alkyl which carries a radical R^{9c}; C₁-C₂-haloalkyl which carries a radical R^{9c}; C₃-C₆-cycloalkyl which may carry one or more substituents R¹¹; C₆-cycloalkenyl which may carry one or more substituents R¹¹; phenyl; and a C-bound 6-membered saturated heteromonocyclic ring containing an oxygen atom as ring member; where
R^{9c} is selected from the group consisting of C₁-C₄-alkoxy; C₃-C₆-cycloalkyl which may carry one or more substituents selected from the group consisting of halogen and C₁-C₂-haloalkyl; phenyl; and a 6-membered saturated heteromonocyclic ring containing an oxygen atom as ring member; and
each R¹¹ is independently selected from the group consisting of C₁-C₂-haloalkyl and C₁-C₂-alkoxy.

19. A compound selected from the group consisting of
(2S)—N-[[6-(4,4-Difluorocyclohexoxy)-5-fluoro-2-methoxy-3-pyridyl]methyl]pyrrolidine-2-carboxamide;
(2S)—N-[(6-Benzyloxy-5-fluoro-2-methoxy-3-pyridyl)methyl]pyrrolidine-2-carboxamide; 2
(2S)—N-[[6-(Cyclohexylmethoxy)-5-fluoro-2-methoxy-3-pyridyl]methyl]pyrrolidine-2-carboxamide;
(2S)—N-[[6-[(4,4-Difluorocyclohexyl)methoxy]-5-fluoro-2-methoxy-3-pyridyl]-methyl]pyrrolidine-2-carboxamide;
(S)—N-((6-((3,5-Difluorobenzyl)oxy)-5-fluoro-2-methoxypyridin-3-yl)methyl)pyrrolidine-2-carboxamide;
(2S)—N-[[6-[(3,3-Difluorocyclopentyl)methoxy]-5-fluoro-2-methoxy-3-pyridyl]-methyl]pyrrolidine-2-carboxamide;
(2S)—N-[[6-[(3,3-Difluorocyclopentyl)methoxy]-5-fluoro-2-methoxy-3-pyridyl]methyl]-1-methyl-pyrrolidine-2-carboxamide;
(2S)—N-[[5-Fluoro-2-methoxy-6-[4-(trifluoromethyl)cyclohexoxy]-3-pyridyl]methyl]-1-methyl-pyrrolidine-2-carboxamide;
(2S)—N-[[6-[(4,4-Difluorocyclohexyl)methoxy]-5-fluoro-2-methoxy-3-pyridyl]methyl]-1-methyl-pyrrolidine-2-carboxamide;
(2S)—N-[[6-(4,4-Difluorocyclohexoxy)-5-fluoro-2-methoxy-3-pyridyl]methyl]-1-methyl-pyrrolidine-2-carboxamide;
(2S)—N-[[5-Fluoro-2-methoxy-6-[trans-4-(trifluoromethyl)cyclohexoxy]-3-pyridyl]methyl]pyrrolidine-2-carboxamide;
(2S)—N-[[5-Fluoro-2-methoxy-6-(4-methoxycyclohexoxy)-3-pyridyl]methyl]pyrrolidine-2-carboxamide;
(2S)—N-[[5-Fluoro-2-methoxy-6-(4-methoxycyclohexoxy)-3-pyridyl]methyl]-1-methyl-pyrrolidine-2-carboxamide;
(2S)—N-[[5-Fluoro-2-methoxy-6-[[4-(trifluoromethyl)cyclohexyl]methoxy]-3-pyridyl]methyl]pyrrolidine-2-carboxamide;
(2S)—N-[[5-Fluoro-2-methoxy-6-[[4-(trifluoromethyl)cyclohexyl]methoxy]-3-pyridyl]methyl]-1-methyl-pyrrolidine-2-carboxamide;
(2S)—N-[[6-[[(1R)-3,3-Difluorocyclopentyl]methoxy]-5-fluoro-2-methoxy-3-pyridyl]methyl]-1-methyl-pyrrolidine-2-carboxamide;
(2S)—N-[[6-[[(1S)-3,3-Difluorocyclopentyl]methoxy]-5-fluoro-2-methoxy-3-pyridyl]methyl]-1-methyl-pyrrolidine-2-carboxamide;
(2S)—N-[[5-Fluoro-2-methoxy-6-(2,2,3,3,3-pentafluoropropoxy)-3-pyridyl]methyl]-pyrrolidine-2-carboxamide;
(2S)—N-[[5-fluoro-2-methoxy-6-[4-(trifluoromethyl)cyclohexoxy]-3-pyridyl]methyl]-1-methyl-pyrrolidine-2-carboxamide;
(2S)—N-[[5-fluoro-2-methoxy-6-[4-(trifluoromethyl)cyclohexoxy]-3-pyridyl]methyl]-pyrrolidine-2-carboxamide;
(2S)—N-[[6-(4,4-Difluorocyclohexoxy)-5-fluoro-2-methoxy-3-pyridyl]methyl]-1-methyl-2,5-dihydropyrrole-2-carboxamide;
(S)—N-((6-(Bicyclo[1.1.1]pentan-1-ylmethoxy)-5-fluoro-2-methoxypyridin-3-yl)methyl)pyrrolidine-2-carboxamide;
(2S)—N-[[6-(3,3-difluorocyclopentoxy)-5-fluoro-2-methoxy-3-pyridyl]methyl]-1-methyl-pyrrolidine-2-carboxamide;
(2S)—N-[[6-(3,3-Difluorocyclopentoxy)-5-fluoro-2-methoxy-3-pyridyl]methyl]pyrrolidine-2-carboxamide;
(2R)—N-[[6-[[(1R)-3,3-Difluorocyclopentyl]methoxy]-5-fluoro-2-methoxy-3-pyridyl]methyl]-1-methyl-pyrrolidine-2-carboxamide;
(2S)—N-[[6-[(1S)-3,3-Difluorocyclopentoxy]-5-fluoro-2-methoxy-3-pyridyl]methyl]-1-methyl-pyrrolidine-2-carboxamide;
(2S)—N-[[6-[(1R)-3,3-Difluorocyclopentoxy]-5-fluoro-2-methoxy-3-pyridyl]methyl]-1-methyl-pyrrolidine-2-carboxamide;
(2S)—N-[[5-fluoro-6-(4-Fluorocyclohexoxy)-2-methoxy-3-pyridyl]methyl]-1-methyl-pyrrolidine-2-carboxamide;
(2S)—N-[[5-Fluoro-6-(4-fluorocyclohexoxy)-2-methoxy-3-pyridyl]methyl]-1-methyl-pyrrolidine-2-carboxamide;
(2S)—N-[[5-Fluoro-6-(4-fluorocyclohexoxy)-2-methoxy-3-pyridyl]methyl]pyrrolidine-2-carboxamide;
(2S)—N-[[5-Fluoro-6-(4-fluorocyclohexoxy)-2-methoxy-3-pyridyl]methyl]pyrrolidine-2-carboxamide;
(2S)—N-[[6-[[(1S)-3,3-Difluorocyclopentyl]methoxy]-5-fluoro-2-methoxy-3-pyridyl]methyl]pyrrolidine-2-carboxamide;
(2S)—N-[[6-[[(1R)-3,3-Difluorocyclopentyl]methoxy]-5-fluoro-2-methoxy-3-pyridyl]methyl]pyrrolidine-2-carboxamide;
(2 S,4R)—N-[[6-(4,4-Difluorocyclohexoxy)-5-fluoro-2-methoxy-3-pyridyl]methyl]-4-fluoro-pyrrolidine-2-carboxamide;
(2S,4R)-4-Fluoro-N-[[5-fluoro-2-methoxy-6-[4-(trifluoromethyl)cyclohexoxy]-3-pyridyl]methyl]pyrrolidine-2-carboxamide;
(2S)—N-[[5-Fluoro-2-methoxy-6-[4-(trifluoromethyl)cyclohexoxy]-3-pyridyl]methyl]-azetidine-2-carboxamide;
(2S)—N-[[6-(4,4-difluorocyclohexoxy)-5-fluoro-2-methoxy-3-pyridyl]methyl]azetidine-2-carboxamide;
(2S)—N-[[5-Fluoro-2-methoxy-6-[4-(trifluoromethyl)cyclohexoxy]-3-pyridyl]methyl]-1-methyl-azetidine-2-carboxamide;
(2S)—N-[[6-(4,4-Difluorocyclohexoxy)-5-fluoro-2-methoxy-3-pyridyl]methyl]-1-methyl-azetidine-2-carboxamide;
(2S)—N-[[5-fluoro-2-methoxy-6-(4,4,4-trifluorobutoxy)-3-pyridyl]methyl]pyrrolidine-2-carboxamide;
(2S)—N-[[6-[(2,2-Difluorocyclopropyl)methoxy]-5-fluoro-2-methoxy-3-pyridyl]methyl]pyrrolidine-2-carboxamide;
(2S)—N-[[6-[(3,3-Difluorocyclobutyl)methoxy]-5-fluoro-2-methoxy-3-pyridyl]methyl]-pyrrolidine-2-carboxamide;
2-(Azetidin-1-yl)-N-[[6-(4,4-difluorocyclohexoxy)-5-fluoro-2-methoxy-3-pyridyl]-methyl]propanamide;
(2S)—N-[[6-(3,3-Difluorocyclobutoxy)-5-fluoro-2-methoxy-3-pyridyl]methyl]pyrrolidine-2-carboxamide;
(2S)—N-[[5-Fluoro-2-methoxy-6-(3,3,4,4,4-pentafluorobutoxy)-3-pyridyl]methyl]-pyrrolidine-2-carboxamide;
(2S)—N-[[5-Fluoro-2-methoxy-6-(2,2,3,3-tetrafluoropropoxy)-3-pyridyl]methyl]-pyrrolidine-2-carboxamide;
(2S)—N-[[5-Fluoro-2-methoxy-6-[(2,2,3,3-tetrafluorocyclobutyl)methoxy]-3-pyridyl]-methyl]pyrrolidine-2-carboxamide;
(2S)—N-[[5-Fluoro-2-methoxy-6-[[1-(trifluoromethyl)cyclopropyl]methoxy]-3-pyridyl]methyl]pyrrolidine-2-carboxamide;
(2S)—N-[[5-Fluoro-2-methoxy-6-(4,4,4-trifluoro-2-methyl-butoxy)-3-pyridyl]methyl]-pyrrolidine-2-carboxamide;
(2S)—N-[[5-Fluoro-2-methoxy-6-(2,2,3,3,3-pentafluoro-1-methyl-propoxy)-3-pyridyl]methyl]pyrrolidine-2-carboxamide;
(2R)—N-[[6-(4,4-Difluorocyclohexoxy)-5-fluoro-2-methoxy-3-pyridyl]methyl]pyrrolidine-2-carboxamide;
(2R)—N-[[6-(4,4-Difluorocyclohexoxy)-5-fluoro-2-methoxy-3-pyridyl]methyl]-1-methyl-pyrrolidine-2-carboxamide;
(2S)—N-[[5-Fluoro-2-methoxy-6-(2,2,3,3-tetrafluoro-1-methyl-propoxy)-3-pyridyl]-methyl]pyrrolidine-2-carboxamide;
(2S)—N-[[6-[(6,6-Difluoro-3-bicyclo[3.1.0]hexanyl)oxy]-5-fluoro-2-methoxy-3-pyridyl]methyl]pyrrolidine-2-carboxamide;
(2S)—N-[[5-Fluoro-6-[(1R,2R)-2-fluorocyclohexoxy]-2-methoxy-3-pyridyl]methyl]pyrrolidine-2-carboxamide;
(2S)—N-[[6-[(6,6-Difluoro-3-bicyclo[3.1.0]hexanyl)methoxy]-5-fluoro-2-methoxy-3-pyridyl]methyl]pyrrolidine-2-carboxamide;
(2S)—N-[[5-Fluoro-2-methoxy-6-[2-(trifluoromethyl)cyclohexoxy]-3-pyridyl]methyl]pyrrolidine-2-carboxamide;
(2S)—N-[[6-(3,3-Difluorocyclohexoxy)-5-fluoro-2-methoxy-3-pyridyl]methyl]pyrrolidine-2-carboxamide;
(2 S,4R)—N-[[6-(4,4-Difluorocyclohexoxy)-5-fluoro-2-methoxy-3-pyridyl]methyl]-4-methyl-pyrrolidine-2-carboxamide;
(2S,4R)—N-[[5-Fluoro-2-methoxy-6-[4-(trifluoromethyl)cyclohexoxy]-3-pyridyl]methyl]-4-methyl-pyrrolidine-2-carboxamide;
(2S,4R)—N-[[6-(4,4-Difluorocyclohexoxy)-5-fluoro-2-methoxy-3-pyridyl]methyl]-1,4-dimethyl-pyrrolidine-2-carboxamide;
(2S,4R)—N-[[5-Fluoro-2-methoxy-6-[4-(trifluoromethyl)cyclohexoxy]-3-pyridyl]methyl]-1,4-dimethyl-pyrrolidine-2-carboxamide;
(2S)—N-[[5-Fluoro-2-methoxy-6-[[1-(trifluoromethyl)cyclopropyl]methoxy]-3-pyridyl]methyl]-1-methyl-pyrrolidine-2-carboxamide;
N-[[6-[(6,6-Difluoro-3-bicyclo[3.1.0]hexanyl)oxy]-5-fluoro-2-methoxy-3-pyridyl]methyl]-1-methyl-pyrrolidine-2-carboxamide;
(S)—N-((2-Methyl-6-(((1R,4S)-4-(trifluoromethyl)cyclohexyl)oxy)pyridin-3-yl)methyl)pyrrolidine-2-carboxamide;
(2S)—N-[[6-[4-(Difluoromethyl)cyclohexoxy]-5-fluoro-2-methoxy-3-pyridyl]methyl]pyrrolidine-2-carboxamide;
(2S)—N-[[6-[4-(Difluoromethoxy)cyclohexoxy]-5-fluoro-2-methoxy-3-pyridyl]methyl]pyrrolidine-2-carboxamide;
(2S)—N-[[6-[(2,2-Difluorocyclopropyl)methoxy]-5-fluoro-2-methoxy-3-pyridyl]methyl]-1-methyl-pyrrolidine-2-carboxamide;
(S)—N-((5-Fluoro-2-methoxy-6-(2,3,3,3-tetrafluoro-2-(trifluoromethyl)propoxy)pyridin-3-yl)methyl)pyrrolidine-2-carboxamide;
(2S)—N-[[6-(1-Adamantylmethoxy)-5-fluoro-2-methoxy-3-pyridyl]methyl]pyrrolidine-2-carboxamide;
(2S)—N-[[6-(3,3-Difluorocyclobutoxy)-5-fluoro-2-methoxy-3-pyridyl]methyl]-1-methyl-pyrrolidine-2-carboxamide;
(2S)—N-((6-((4-(1,1-Difluoroethyl)cyclohexyl)oxy)-5-fluoro-2-methoxypyridin-3-yl)methyl)pyrrolidine-2-carboxamide;
(2S)—N-[[6-[4-(1,1-Difluoroethyl)cyclohexoxy]-5-fluoro-2-methoxy-3-pyridyl]methyl]-pyrrolidine-2-carboxamide;
(2S)—N-[[5-fluoro-6-[4-(fluoromethyl)cyclohexoxy]-2-methoxy-3-pyridyl]methyl]-pyrrolidine-2-carboxamide;
(2S)—N-[[5-Fluoro-2-methoxy-6-[[1-(trifluoromethyl)cyclopropyl]methoxy]-3-pyridyl]methyl]-N-methyl-pyrrolidine-2-carboxamide;
(2S)—N-[[6-[(2,2-Difluorocyclopropyl)methoxy]-5-fluoro-2-methoxy-3-pyridyl]methyl]-N-methyl-pyrrolidine-2-carboxamide;
(2S)—N-[[6-(3,3-Difluorocyclobutoxy)-5-fluoro-2-methoxy-3-pyridyl]methyl]-N-methyl-pyrrolidine-2-carboxamide;
(2S,3R)—N-[[5-Fluoro-2-methoxy-6-[4-(trifluoromethyl)cyclohexoxy]-3-pyridyl]methyl]-3-methyl-azetidine-2-carboxamide;
(2S,3 S)—N-[[5-Fluoro-2-methoxy-6-[4-(trifluoromethyl)cyclohexoxy]-3-pyridyl]methyl]-3-methyl-azetidine-2-carboxamide;
(2S)—N-[[5-Fluoro-6-(4-hydroxycyclohexoxy)-2-methoxy-3-pyridyl]methyl]pyrrolidine-2-carboxamide;
(1R,3 S,5R)—N-[[5-Fluoro-2-methoxy-6-[4-(trifluoromethyl)cyclohexoxy]-3-pyridyl]methyl]-4-azabicyclo[3.1.0]hexane-3-carboxamide;
N-[[5-Fluoro-2-methoxy-6-[4-(trifluoromethyl)cyclohexoxy]-3-pyridyl]methyl]-4-azabicyclo[3.1.0]hexane-5-carboxamide;
(1S,4S,5R)—N-[[5-Fluoro-2-methoxy-6-[4-(trifluoromethyl)cyclohexoxy]-3-pyridyl]methyl]-3-azabicyclo[3.1.0]hexane-4-carboxamide;
(1R,4S,5 S)—N-[[5-Fluoro-2-methoxy-6-[4-(trifluoromethyl)cyclohexoxy]-3-pyridyl]methyl]-3-azabicyclo[3.1.0]hexane-4-carboxamide;
(2S)—N-[[6-(2,2-Difluoroethoxy)-5-fluoro-2-methoxy-3-pyridyl]methyl]pyrrolidine-2-carboxamide;
(2S)—N-[[5-Fluoro-2-methoxy-6-(2,2,2-trifluoroethoxy)-3-pyridyl]methyl]pyrrolidine-2-carboxamide;
(2S)—N-[[5-Fluoro-2-methoxy-6-[(1S,3R)-3-(trifluoromethyl)cyclohexoxy]-3-pyridyl]methyl]pyrrolidine-2-carboxamide;
(2S)—N-[[5-Fluoro-2-methoxy-6-[(1S,3S)-3-(trifluoromethyl)cyclohexoxy]-3-pyridyl]methyl]pyrrolidine-2-carboxamide;
(2S)—N-[[5-Fluoro-2-methoxy-6-[(2,2,3,3-tetrafluorocyclobutyl)methoxy]-3-pyridyl]methyl]-1-methyl-pyrrolidine-2-carboxamide;
(2S)—N-[[5-Fluoro-2-methoxy-6-(2,2,3,3-tetrafluoropropoxy)-3-pyridyl]methyl]-1-methyl-pyrrolidine-2-carboxamide;
(2S)—N-[[6-[(3,3-Difluorocyclobutyl)methoxy]-5-fluoro-2-methoxy-3-pyridyl]methyl]-1-methyl-pyrrolidine-2-carboxamide;
(2S)—N-[[5-Fluoro-6-(2-fluorocyclopentoxy)-2-methoxy-3-pyridyl]methyl]pyrrolidine-2-carboxamide;
(2S,4R)—N-[[6-(3,3-Difluorocyclobutoxy)-5-fluoro-2-methoxy-3-pyridyl]methyl]-4-fluoro-pyrrolidine-2-carboxamide;
(2S)—N-[[6-(7,7-Difluoronorcaran-2-yl)oxy-5-fluoro-2-methoxy-3-pyridyl]methyl]-pyrrolidine-2-carboxamide;
(2S)—N-[[6-[[1-(Difluoromethyl)cyclopropyl]methoxy]-5-fluoro-2-methoxy-3-pyridyl]methyl]-1-methyl-pyrrolidine-2-carboxamide;
(2S)—N-[[6-[[1-(Difluoromethyl)cyclopropyl]methoxy]-5-fluoro-2-methoxy-3-pyridyl]methyl]pyrrolidine-2-carboxamide;
(2S)—N-[[5-Fluoro-2-methoxy-6-[[1-(trifluoromethyl)cyclopropyl]methoxy]-3-pyridyl]methyl]-N,1-dimethyl-pyrrolidine-2-carboxamide;
(2S)—N-[[6-(3,3-Difluorocyclobutoxy)-5-fluoro-2-methoxy-3-pyridyl]methyl]-N,1-dimethyl-pyrrolidine-2-carboxamide;
(2S)—N-[[5-Fluoro-2-methoxy-6-(2,2,2-trifluoroethoxy)-3-pyridyl]methyl]-1-methyl-pyrrolidine-2-carboxamide;
(2S)-2-(Azetidin-1-yl)-N-[[6-(3,3-difluorocyclobutoxy)-5-fluoro-2-methoxy-3-pyridyl]methyl]propanamide;
(2S)—N-[[5-Fluoro-2-methoxy-6-(2,2,3,3-tetrafluoropropoxy)-3-pyridyl]methyl]-N,1-dimethyl-pyrrolidine-2-carboxamide;
(2S)—N-[[5-Fluoro-2-methoxy-6-[(2,2,3,3-tetrafluorocyclobutyl)methoxy]-3-pyridyl]methyl]-N,1-dimethyl-pyrrolidine-2-carboxamide;
(2S)—N-[[5-Fluoro-2-methoxy-6-(3-methoxycyclobutoxy)-3-pyridyl]methyl]pyrrolidine-2-carboxamide;
(2S)—N-[[5-Fluoro-2-methoxy-6-[3-(trifluoromethyl)cyclobutoxy]-3-pyridyl]methyl]-pyrrolidine-2-carboxamide;
(2S)—N-[[6-[(3,3-Difluorocyclobutyl)methoxy]-5-fluoro-2-methoxy-3-pyridyl]methyl]-N,1-dimethyl-pyrrolidine-2-carboxamide;
(2S)—N-[[5-(3,3-Difluorocyclobutoxy)-3-methoxy-pyrazin-2-yl]methyl]pyrrolidine-2-carboxamide;
(2S)—N-[[5-(4,4-Difluorocyclohexoxy)-3-methoxy-pyrazin-2-yl]methyl]pyrrolidine-2-carboxamide;
(2S)—N-[[5-Fluoro-2-methoxy-6-[3,3,3-trifluoro-2-methyl-2-(trifluoromethyl)propoxy]-3-pyridyl]methyl]pyrrolidine-2-carboxamide;
(2S)—N-[[5-Fluoro-6-(2,2,3,3,4,4,4-heptafluorobutoxy)-2-methoxy-3-pyridyl]-methyl]pyrrolidine-2-carboxamide;
(2S)—N-[[5-Fluoro-2-methoxy-6-[2,2,2-trifluoro-1-(trifluoromethyl)ethoxy]-3-pyridyl]methyl]pyrrolidine-2-carboxamide;
(2S)—N-[[5-Fluoro-2-methoxy-6-(2,2,3,3-tetrafluorocyclobutoxy)-3-pyridyl]methyl]pyrrolidine-2-carboxamide;
(2S)—N-[[5-Fluoro-2-methoxy-6-(2,2,2-trifluoroethoxy)-3-pyridyl]methyl]-N,1-dimethyl-pyrrolidine-2-carboxamide;
(2S)—N-[[6-(4,4-Difluorocyclohexoxy)-5-fluoro-2-methoxy-3-pyridyl]methyl]-N-methyl-pyrrolidine-2-carboxamide;
(2S)—N-[[6-[(3,3-Difluorocyclobutyl)methoxy]-5-fluoro-2-methoxy-3-pyridyl]methyl]-N-methyl-pyrrolidine-2-carboxamide;
(2S)—N-[[5-Fluoro-2-methoxy-6-[trans-4-(trifluoromethyl)cyclohexoxy]-3-pyridyl]methyl]-N-methyl-pyrrolidine-2-carboxamide;
(2S)—N-[[5-Fluoro-6-(2,2,3,3,4,4,4-heptafluorobutoxy)-2-methoxy-3-pyridyl]methyl]-N-methyl-pyrrolidine-2-carboxamide;
(2S)—N-[[6-(2,2-Difluorospiro[2.5]octan-6-yl)oxy-5-fluoro-2-methoxy-3-pyridyl]methyl]pyrrolidine-2-carboxamide;
(2S)—N-[[5-Fluoro-2-methoxy-6-[cis-4-(trifluoromethyl)cyclohexoxy]-3-pyridyl]methyl]-N-methyl-pyrrolidine-2-carboxamide;
(2S)—N-[[5-Fluoro-2-methoxy-6-[trans-3-(trifluoromethyl)cyclobutoxy]-3-pyridyl]methyl]-N-methyl-pyrrolidine-2-carboxamide;
(2S)—N-[[5-Fluoro-2-methoxy-6-[cis-3-(trifluoromethyl)cyclobutoxy]-3-pyridyl]methyl]-N-methyl-pyrrolidine-2-carboxamide;
(2S)—N-[[6-[(3,5-Difluorophenyl)methoxy]-5-fluoro-2-methoxy-3-pyridyl]methyl]-N-methyl-pyrrolidine-2-carboxamide;
(2S)—N-[[6-(3,3-Difluorocyclopentoxy)-5-fluoro-2-methoxy-3-pyridyl]methyl]-N-methyl-pyrrolidine-2-carboxamide;
(2S)—N-[[5-Fluoro-6-[(4-fluorophenyl)methoxy]-2-methoxy-3-pyridyl]methyl]-N-methyl-pyrrolidine-2-carboxamide;
(2S)—N-[(5-Fluoro-2-methoxy-6-phenyl-3-pyridyl)methyl]pyrrolidine-2-carboxamide;
(2S)—N-[[6-[(4,4-Difluorocyclohexyl)amino]-5-fluoro-2-methoxy-3-pyridyl]methyl]pyrrolidine-2-carboxamide;
(2S)—N-[(6-Cyclopropyl-5-fluoro-2-methoxy-3-pyridyl)methyl]pyrrolidine-2-carboxamide;
(2S)—N-[[5-Fluoro-2-methoxy-6-(1-piperidyl)-3-pyridyl]methyl]pyrrolidine-2-carboxamide;
(2S)—N-[[6-(Cyclohexylmethyl)-5-fluoro-2-methoxy-3-pyridyl]methyl]pyrrolidine-2-carboxamide;
(2S)—N-[[5-Fluoro-2-methoxy-6-[[4-(trifluoromethyl)cyclohexyl]amino]-3-pyridyl]methyl]pyrrolidine-2-carboxamide;
(2S)—N-[[6-(Cyclohexylamino)-5-fluoro-2-methoxy-3-pyridyl]methyl]pyrrolidine-2-carboxamide;
(2S)—N-[(6-Cyclohexyl-5-fluoro-2-methoxy-3-pyridyl)methyl]pyrrolidine-2-carboxamide;
(2S)—N-[[5-Fluoro-2-methoxy-6-[4-(trifluoromethyl)-1-piperidyl]-3-pyridyl]methyl]pyrrolidine-2-carboxamide;
(2S)—N-[[6-(4,4-Difluoro-1-piperidyl)-5-fluoro-2-methoxy-3-pyridyl]methyl]pyrrolidine-2-carboxamide;
(2S)—N-[(5-Fluoro-2-methoxy-6-pyrrolidin-1-yl-3-pyridyl)methyl]pyrrolidine-2-carboxamide;
(2S)—N-[[6-[(3,3-Difluorocyclobutyl)methylamino]-5-fluoro-2-methoxy-3-pyridyl]methyl]pyrrolidine-2-carboxamide;
(2S)—N-[[5-Fluoro-2-methoxy-6-[methyl(phenethyl)amino]-3-pyridyl]methyl]pyrrolidine-2-carboxamide;
(2S)—N-[(5-Fluoro-6-indolin-1-yl-2-methoxy-3-pyridyl)methyl]pyrrolidine-2-carboxamide;
(2S)—N-[[5-fluoro-2-methoxy-6-[2-(trifluoromethyl)cyclopropyl]-3-pyridyl]methyl]pyrrolidine-2-carboxamide;
(2S)—N-[[5-Fluoro-2-methoxy-6-[4-(trifluoromethyl)cyclohexen-1-yl]-3-pyridyl]methyl]pyrrolidine-2-carboxamide;
(2S)—N-[[5-Fluoro-2-methoxy-6-[[cis-4-(trifluoromethyl)cyclohexyl]amino]-3-pyridyl]methyl]pyrrolidine-2-carboxamide;
(2S)—N-[[5-Fluoro-2-methoxy-6-[[trans-4-(trifluoromethyl)cyclohexyl]amino]-3-pyridyl]methyl]pyrrolidine-2-carboxamide;
(2S)—N-[(6-benzyl-5-fluoro-2-methoxy-3-pyridyl)methyl]pyrrolidine-2-carboxamide;
(2S)—N-[[6-(Cyclobutylamino)-5-fluoro-2-methoxy-3-pyridyl]methyl]pyrrolidine-2-carboxamide;
(2S)—N-[[6-[Cyclopropyl(methyl)amino]-5-fluoro-2-methoxy-3-pyridyl]-methyl]pyrrolidine-2-carboxamide;
(2S)—N-[[5-fluoro-2-methoxy-6-[[4-(trifluoromethyl)cyclohexyl]methylamino]-3-pyridyl]methyl]pyrrolidine-2-carboxamide;
(2S)—N-[[6-[Allyl(benzyl)amino]-5-fluoro-2-methoxy-3-pyridyl]methyl]pyrrolidine-2-carboxamide;
(2S)—N-[[6-[3-(4-Chlorophenoxy)azetidin-1-yl]-5-fluoro-2-methoxy-3-pyridyl]methyl]pyrrolidine-2-carboxamide;
(2S)—N-[[5-Fluoro-2-methoxy-6-[[1-(trifluoromethyl)cyclopropyl]methylamino]-3-pyridyl]methyl]pyrrolidine-2-carboxamide;
(2S)—N-[[5-Fluoro-2-methoxy-6-[3-(trifluoromethyl)-1-piperidyl]-3-pyridyl]methyl]pyrrolidine-2-carboxamide;
(2S)—N-[[6-[(4,4-Difluorocyclohexyl)-methyl-amino]-5-fluoro-2-methoxy-3-pyridyl]methyl]pyrrolidine-2-carboxamide;
(2S)—N-[[6-(Azepan-1-yl)-5-fluoro-2-methoxy-3-pyridyl]methyl]pyrrolidine-2-carboxamide;
(2S)—N-[[6-(3,3-Difluoro-1-piperidyl)-5-fluoro-2-methoxy-3-pyridyl]methyl]pyrrolidine-2-carboxamide;
(2S)—N-[[5-fluoro-2-methoxy-6-[[4-(trifluoromethyl)cyclohexyl]amino]-3-pyridyl]methyl]-N-methyl-pyrrolidine-2-carboxamide;
(2S)—N-[(5-Fluoro-2-methoxy-6-tetrahydropyran-3-yl-3-pyridyl)methyl]pyrrolidine-2-carboxamide;
(2S)—N-[(5-Fluoro-2-methoxy-6-tetrahydropyran-4-yl-3-pyridyl)methyl]pyrrolidine-2-carboxamide;
(2S)—N-[[5-Fluoro-6-(4-fluoro-1-piperidyl)-2-methoxy-3-pyridyl]methyl]pyrrolidine-2-carboxamide;
(2S)—N-[[5-Fluoro-6-(3-fluoro-1-piperidyl)-2-methoxy-3-pyridyl]methyl]pyrrolidine-2-carboxamide;
(2S)—N-[[5-Fluoro-6-(3-Fluoropyrrolidin-1-yl)-2-methoxy-3-pyridyl]methyl]pyrrolidine-2-carboxamide;
(2S)—N-[[5-Fluoro-2-methoxy-6-[3-(trifluoromethyl)pyrrolidin-1-yl]-3-pyridyl]methyl]pyrrolidine-2-carboxamide;
(2S)—N-[[6-(3,3-Difluoropyrrolidin-1-yl)-5-fluoro-2-methoxy-3-pyridyl]methyl]-pyrrolidine-2-carboxamide;
(2S)—N-[[6-(4,4-Difluoro-1-piperidyl)-5-fluoro-2-methoxy-3-pyridyl]methyl]-N-methyl-pyrrolidine-2-carboxamide;
(2S)—N-[[6-[(4,4-Difluorocyclohexyl)amino]-5-fluoro-2-methoxy-3-pyridyl]methyl]-N-methyl-pyrrolidine-2-carboxamide;
(2S)—N-[[5-Fluoro-2-methoxy-6-(4-methoxycyclohexen-1-yl)-3-pyridyl]-methyl]pyrrolidine-2-carboxamide;
(2S)—N-[[5-Fluoro-2-methoxy-6-(3-methoxypropyl)-3-pyridyl]methyl]pyrrolidine-2-carboxamide;
(2S)—N-[[6-[(4,4-Difluorocyclohexyl)methylamino]-5-fluoro-2-methoxy-3-pyridyl]methyl]pyrrolidine-2-carboxamide;
(2S)—N-[[6-(Benzylamino)-5-fluoro-2-methoxy-3-pyridyl]methyl]pyrrolidine-2-carboxamide;
(2S)—N-[[6-[Benzyl(propyl)amino]-5-fluoro-2-methoxy-3-pyridyl]methyl]pyrrolidine-2-carboxamide;
(2S)—N-[[5-Fluoro-2-methoxy-6-(4-methoxycyclohexyl)-3-pyridyl]methyl]pyrrolidine-2-carboxamide;
(2S)—N-[[5-Fluoro-2-methoxy-6-(2-phenylmorpholin-4-yl)-3-pyridyl]methyl]pyrrolidine-2-carboxamide;
(2S)—N-[[6-(2,6-Dimethylmorpholin-4-yl)-5-fluoro-2-methoxy-3-pyridyl]methyl]-pyrrolidine-2-carboxamide;
(2S)—N-[[5-Fluoro-2-methoxy-6-(3-phenoxyazetidin-1-yl)-3-pyridyl]methyl]pyrrolidine-2-carboxamide;
(2S)—N-[[5-Fluoro-2-methoxy-6-(3-methyl-3-phenoxy-azetidin-1-yl)-3-pyridyl]methyl]pyrrolidine-2-carboxamide;
(2S)—N-[[5-Fluoro-6-[(4-fluorophenyl)methylamino]-2-methoxy-3-pyridyl]methyl]pyrrolidine-2-carboxamide;
(2S)—N-[[5-Fluoro-6-(4-fluoroanilino)-2-methoxy-3-pyridyl]methyl]pyrrolidine-2-carboxamide;
(2S)—N-[[6-[Benzyl(methyl)amino]-5-fluoro-2-methoxy-3-pyridyl]methyl]pyrrolidine-2-carboxamide;
(2S)—N-[[5-Fluoro-2-methoxy-6-[4-[4-(trifluoromethyl)cyclohexyl]-1-piperidyl]-3-pyridyl]methyl]pyrrolidine-2-carboxamide;
(2S)—N-[[6-[(3aR,6aS)-5,5-Difluoro-1,3,3a,4,6,6a-hexahydrocyclopenta[c]pyrrol-2-yl]-5-fluoro-2-methoxy-3-pyridyl]methyl]pyrrolidine-2-carboxamide;
(2S)—N-[[5-Fluoro-6-(3,3,4,4,5,5-hexafluoro-1-piperidyl)-2-methoxy-3-pyridyl]methyl]pyrrolidine-2-carboxamide;
(2S)—N-[[6-(6,6-Difluoro-3-azabicyclo[3.1.0]hexan-3-yl)-5-fluoro-2-methoxy-3-pyridyl]methyl]pyrrolidine-2-carboxamide;
(2S)—N-[[6-(7,7-Difluoro-4-azabicyclo[4.1.0]heptan-4-yl)-5-fluoro-2-methoxy-3-pyridyl]methyl]pyrrolidine-2-carboxamide;
(2S)—N-[[5-Fluoro-2-methoxy-6-(3-phenylpropylamino)-3-pyridyl]methyl]pyrrolidine-2-carboxamide;
(2S)—N-[[5-Fluoro-2-methoxy-6-[[2-(trifluoromethyl)cyclobutyl]amino]-3-pyridyl]methyl]pyrrolidine-2-carboxamide;
(2S)—N-[[5-Fluoro-2-methoxy-6-[3-(2,2,2-trifluoroethyl)pyrrolidin-1-yl]-3-pyridyl]methyl]pyrrolidine-2-carboxamide;
(2S)—N-[[6-(2,2-Difluoro-7-azaspiro[2.5]octan-7-yl)-5-fluoro-2-methoxy-3-pyridyl]methyl]pyrrolidine-2-carboxamide;
(2S)—N-[[5-Fluoro-2-methoxy-6-(tetrahydropyran-4-ylmethyl)-3-pyridyl]-methyl]pyrrolidine-2-carboxamide;
(2S)—N-[[6-[3-(Difluoromethyl)-4,4-difluoro-1-piperidyl]-5-fluoro-2-methoxy-3-pyridyl]methyl]pyrrolidine-2-carboxamide;
(2S)—N-[[6-(1,1-Difluoro-2-phenyl-ethyl)-5-fluoro-2-methoxy-3-pyridyl]methyl]-N-methyl-pyrrolidine-2-carboxamide;
N-[[6-(2-Chloro-1,1-difluoro-2-phenyl-ethyl)-5-fluoro-2-methoxy-3-pyridyl]methyl]-N-methyl-pyrrolidine-2-carboxamide;
(2S)—N-[[5-Fluoro-2-methoxy-6-(4-methoxybutyl)-3-pyridyl]methyl]pyrrolidine-2-carboxamide;
(2S)—N-[[6-(3-Benzylazetidin-1-yl)-5-fluoro-2-methoxy-3-pyridyl]methyl]pyrrolidine-2-carboxamide; and
(2S)—N-[[5-Fluoro-2-methoxy-6-(4-phenoxy-1-piperidyl)-3-pyridyl]methyl]pyrrolidine-2-carboxamide;
or an N-oxide, tautomer, or stereoisomer thereof; or a pharmaceutically acceptable salt thereof.

20. A pharmaceutical composition comprising a therapeutically effective amount of at least one compound as claimed in claim 1 or an N-oxide, a tautomer, a stereoisomer or a pharmaceutically acceptable salt thereof; in combination with at least one pharmaceutically acceptable carrier and/or auxiliary substance.

21.-23. (canceled)

24. A method for treating disorders which respond to the modulation of the 5-HT_{2c} receptor, which method comprises administering to a subject in need thereof at least one compound as claimed in claim 1 or an N-oxide, a tautomeric form, a stereoisomer or a pharmaceutically acceptable salt thereof.

25. The method as claimed in claim 24, where the disorders are selected from the group consisting of damage of the central nervous system, disorders of the central nervous system, eating disorders, ocular hypertension, cardiovascular disorders, gastrointestinal disorders and diabetes.

26. The method as claimed in claim 25, where the disorders are selected from the group consisting of bipolar disorder, depression, atypical depression, mood episodes, adjustment disorders, anxiety, panic disorders, post-traumatic syndrome, psychoses, schizophrenia, cognitive deficits of schizophrenia, memory loss, dementia of aging, Alzheimer's disease, neuropsychiatric symptoms in Alzheimer's disease, behavioral disorders associated with dementia, social phobia, mental disorders in childhood, attention deficit hyperactivity disorder, organic mental disorders, autism, mutism, disruptive behavior disorder, impulse control disorder, borderline personality disorder, obsessive compulsive disorder, migraine and other conditions associated with cephalic pain or other pain, raised intracranial pressure, seizure disorders, epilepsy, substance use disorders, alcohol abuse, cocaine abuse, tobacco abuse, smoking cessation, sexual dysfunction/erectile dysfunction in males, sexual dysfunction in females, premenstrual syndrome, late luteal phase syndrome, chronic fatigue syndrome, sleep disorders, sleep apnoea, chronic fatigue syndrome, psoriasis, Parkinson's disease, psychosis in Parkinson's disease, neuropsychiatric symptoms in Parkinson's disease, Lewy Body dementia, neuropsychiatric symptoms in Lewy Body dementia, spinal cord injury, trauma, stroke, pain, bladder dysfunction/urinary incontinence, encephalitis, meningitis, eating disorders, obesity, bulimia, weight loss, anorexia nervosa, ocular hypertension, cardiovascular disorders, gastrointestinal disorders, diabetes insipidus, diabetes mellitus, type I diabetes, type II diabetes, type III diabetes, diabetes secondary to pancreatic diseases, diabetes related to steroid use, diabetes complications, hyperglycemia and insulin resistance.

27. The method as claimed in claim 26, where the disorders are selected from the group consisting of schizophrenia, depression, bipolar disorders, obesity, substance use disorders, neuropsychiatric symptoms in Alzheimer's disease and neuropsychiatric symptoms in Parkinson's disease.
